(19) United States
(12) Patent Application Publication
(10) Pub. No.: US 2014/0105898 A1
(43) Pub. Date: Apr. 17, 2014

(54) APOPTOSIS-INDUCING MOLECULES AND USES THEREFOR

(71) Applicants: Merlin C. Thomas, Melbourne, Victoria (AU); Giorgio Zauli, Trieste (IT); Paola Secchiero, Ferrara (IT); Bruno Fabris, Trieste (IT); Stella Bernardi, Trieste (IT)

(72) Inventors: Merlin C. Thomas, Melbourne, Victoria (AU); Giorgio Zauli, Trieste (IT); Paola Secchiero, Ferrara (IT); Bruno Fabris, Trieste (IT); Stella Bernardi, Trieste (IT)

(73) Assignee: Istituto Di Ricovero E Cura A Carattere Scientific Materno-Infantile Burlo Garo, Trieste (IT)

(21) Appl. No.: 14/002,100

(22) PCT Filed: Feb. 27, 2012
(86) PCT No.: PCT/IB12/50896

§ 371 Date: Dec. 4, 2013

Related U.S. Application Data
(60) Provisional application No. 61/447,653, filed on Feb. 28, 2011.

(51) Int. Cl. C07D 495/04; C07D 405/12; A61K 39/395; C07D 473/06; A61K 31/7088; A61K 38/17; C07C 251/24; C07D 263/28

(52) U.S. Cl. CPC C07D 495/04; C07C 251/24; C07D 405/12; C07D 263/28; C07D 473/06; A61K 31/7088; A61K 38/17; A61K 39/39558
USPC 424/135.1; 562/440; 514/567; 548/251; 514/382; 544/235; 514/248; 544/137; 514/236.8; 544/405; 514/255.05; 544/271; 514/263.36; 536/23.5; 530/300; 530/387.3; 530/388.22; 530/389.7; 514/44 R; 514/18.9; 424/143.1; 424/174.1; 514/4.8; 514/6.9

(57) ABSTRACT

This invention relates generally to methods and agents for modulating adiposity-related conditions. More particularly, the present invention relates to the use of TRAIL death receptor agonists, including nucleic acids such as TRAIL polynucleotides, peptides and polypeptides including TRAIL polypeptides, TRAIL DR agonist antigen-binding molecules, TRAIL DR peptide agonists as well as small molecule TRAIL DR agonists in compositions and methods for treating or preventing adiposity-related conditions such as obesity, diabetes mellitus and metabolic syndrome.

### FIELD OF THE INVENTION

This invention relates generally to methods and agents for modulating adiposity-related conditions. More particularly, the present invention relates to the use of TRAIL death receptor agonists, including nucleic acids such as TRAIL polynucleotides, peptides and polypeptides including TRAIL polypeptides, TRAIL DR agonist antigen-binding molecules, TRAIL DR peptide agonists as well as small molecule TRAIL DR agonists in compositions and methods for treating or preventing adiposity-related conditions such as obesity, diabetes mellitus and metabolic syndrome.

### BACKGROUND OF THE INVENTION

Tumor necrosis factor (TNF)-related apoptosis-inducing ligand (TRAIL), also known as Apo2 ligand, belongs to the TNF superfamily and has been identified as an activator of programmed cell death in tumor cells. TRAIL is predominantly but not exclusively expressed in cells of the immune system including natural killer (NK) cells, T cells, macrophages, and dendritic cells and is located in the cell membrane. TRAIL can be processed by cysteine proteases, which generate a soluble form of the protein corresponding to its extracellular domain (e.g., amino acids 114 to 281). Both the membrane-bound and soluble forms of TRAIL function as trimers that are able to trigger apoptosis via interaction with TRAIL receptors located on target cells. TRAIL is physiologically present in the plasma/serum as well as in other body fluids, such as saliva and tears.

Known members of the TRAIL family of receptors include TRAIL-R1 (also known as TNFRSF10A, DR4, APO2 or the like) and TRAIL-R2 (also known as TNFRSF10B, DR5 or the like) death receptors, which have a death domain (DD) and transduce an apoptotic signal. The family also includes decoy receptors DcR1 (also referred to as TNFRSF10C, TRAIL-R3, LIT, TRID or the like) and DcR2 (also known as TNFRSF10D, TRUNDD, or TRAIL-R4), which do not transduce apoptotic signals, and a soluble receptor osteoprotegerin (also known as OPG, TNFRSF11B, or OCIF), which has no membrane-bound domain. These latter three receptors lack functional DDs and are thought to be mainly involved in negatively regulating apoptosis by sequestering TRAIL or stimulating pro-survival signals.

Unlike other members of the TNF superfamily, such as TNF and CD95L, TRAIL and other TRAIL death receptor agonists such as TRAIL-R1 and TRAIL-R2 agonistic antibodies do not induce cell death at normal tissues. In light of this activity, several TRAIL-based therapeutic approaches are being pursued including use of TRAIL death receptor agonists as anti-cancer agents for treating a variety of solid tumors including colon carcinoma, glioma, lung carcinoma, prostate carcinoma, brain tumors and multiple myeloma. TRAIL-mediated apoptosis has also been observed in virally infected cells and over-activated immune cells and based on these observations, TRAIL death receptor agonists have been proposed for use in treating viral infections as well as T cell-mediated autoimmune disorders such as experimental autoimmune encephalomyelitis and rheumatoid arthritis.

### SUMMARY OF THE INVENTION

The present inventors have observed that rTRAIL significantly (1) reduces fasting hyperinsulinemia, (2) reduces glucose levels after a hyperglycemic stimulus, (3) lowers hyperinsulinemia after a hyperglycemic stimulus, (4) improves peripheral response to insulin, (5) reduces increased adiposity in response to high fat diet, (6) improves mitochondrial fatty acid oxidative capacity of muscle tissue, (7) reduces circulating levels of pro-inflammatory cytokines [both after prolonged (IL-6) and after short-term (IL-6, IL-1alpha, G-CSF, MCP-1) treatment], (8) reduces lipopolysaccaride (LPS)- and muramildipeptide (MDP)-induced proinflammatory activity and body temperature elevation. Based on these observations, the present inventors propose that rTRAIL as well as other TRAIL death receptor (DR) agonists are useful in methods and compositions for treating or preventing adiposity-related conditions including obesity, diabetes mellitus and metabolic syndrome, as described hereafter.

Accordingly, in one aspect, the present invention provides TRAIL DR agonists for controlling adiposity in a subject, including use, also intended as use in the preparation of a medicament, in the treatment or prevention of adiposity-related conditions (e.g., obesity and conditions of localized, abnormal increases in adiposity such as, but not limited to, lipoma and lipomatosis, as well as diabetes mellitus and metabolic syndrome). Non limiting examples of suitable TRAIL DR agonists include nucleic acids such as TRAIL polynucleotides, peptides and polypeptides including TRAIL polypeptides, chimeric polypeptides comprising a trimerizing domain and at least one C-type lectin like domain that binds to at least one TRAIL DR and TRAIL DR agonist antigen-binding molecules, TRAIL DR peptide agonists as well as small molecule TRAIL DR agonists.

In another aspect, the present invention provides compositions for controlling adiposity, including use in the treatment or prevention of adiposity-related conditions. These compositions generally comprise a TRAIL DR agonist and a pharmaceutically acceptable carrier or diluent. The compositions may be administered by injection, by topical or mucosal application, by inhalation or via the oral route including modified-release modes of administration in liquid formulas or other liquids, over a period of time and in amounts which are effective to ameliorate, inhibit or otherwise reduce adiposity and/or to treat or prevent the adiposity related condition. In specific embodiments, the composition is administered systemically.

Thus, in a related aspect, the present invention provides methods for controlling adiposity, including in the treatment or prevention of adiposity-related conditions, in a subject. These methods generally comprise administering to the subject an effective amount of a TRAIL DR agonist, and optionally a pharmaceutically acceptable carrier or diluent.

In another aspect of the present invention provides the use of a TRAIL DR agonist in the preparation of a medicament for controlling adiposity including treating or preventing an adiposity-related condition.

Still in another aspect, the present invention provides the use of a TRAIL DR agonist for inhibiting the inflammation response, including the recruitment of leukocytes and release of acute phase proteins, mediated by key pro-inflammatory cytokines, such as IL-6, IL-1alpha, G-CSF, MCP-1, which are known to contribute to the pathogenesis of obesity related disease, such as diabetes type II and cardiovascular disease in a cross-talk between adipocytes, or precursor thereof such as a preadipocytes and cells of the monocytic/macrophagic lineage.

### BRIEF DESCRIPTION OF THE DRAWINGS

FIG. 1 is a diagrammatic representation showing the results of a CLUSTAL W (1.83) multiple sequence alignment of the following TRAIL polypeptides: polypeptide corresponding to amino acids 114-281 of a human TRAIL isoform 1, as set forth in NCBI Accession: NP_{—}003801 [SEQ ID NO:2]; a putative full-length synthetic TRAIL, as set forth in NCBI Accession: AAV38370 [SEQ ID NO: 4]; a putative full-length human TRAIL isoform 1, as set forth in NCBI Accession: NP_{—}003801 [SEQ ID NO: 6]; a putative full-length synthetic TRAIL, as set forth in NCBI Accession: AAX29952 [SEQ ID NO: 8]; a putative full-length human TRAIL isoform CRA_b, as set forth in NCBI Accession: EAW78466 [SEQ ID NO: 10]; a putative full-length Pan troglodytes TRAIL, as set forth in NCBI Accession: XP 516879 [SEQ ID NO: 12]; polypeptide corresponding to a human TRAIL fragment, as set forth in NCBI Accession: 1DG6 [SEQ ID NO: 16]; a putative full-length Macaca mulatta TRAIL, as set forth in NCBI Accession: XP_{—}001084768 [SEQ ID NO: 18]; a putative full-length Crassostrea ariakensis TRAIL, as set forth in NCBI Accession: ABU39827 [SEQ ID NO: 20]; a putative full-length Pongo abelii TRAIL, as set forth in NCBI Accession: XP_{—}002814335 [SEQ ID NO: 24]; a putative full-length Callithrix jacchus TRAIL, as set forth in NCBI Accession: XP_{—}002759427 [SEQ ID NO: 26]; a putative full-length Felis catus TRAIL, as set forth in NCBI Accession: NP_{—}001124316 [SEQ ID NO: 28]; a putative full-length Ailuropoda melanoleuca TRAIL, as set forth in NCBI Accession: XP_{—}002921635 [SEQ ID NO: 30]; a putative full-length Equus caballus TRAIL, as set forth in NCBI Accession: XP_{—}001494138 [SEQ ID NO: 32]; a putative full-length Ailuropoda melanoleuca TRAIL, as set forth in NCBI Accession: EFB16787 [SEQ ID NO: 34]; a putative full-length Bos taurus TRAIL, as set forth in NCBI Accession: XP 583785 [SEQ ID NO: 36]; a putative full-length Sus scrofa TRAIL, as set forth in NCBI Accession: NP_{—}001019867 [SEQ ID NO: 38]; a putative full-length Canis lupis familiaris TRAIL, as set forth in NCBI Accession: NP_{—}001124308 [SEQ ID NO: 40]; a putative full-length Oryctolagus cuniculus TRAIL, as set forth in NCBI Accession: XP_{—}002716472 [SEQ ID NO: 42]; a putative full-length Rattus novegicus TRAIL, as set forth in NCBI Accession: EDM01114 [SEQ ID NO: 44]; a putative full-length Rattus novegicus TRAIL, as set forth in NCBI Accession: NP 663714 [SEQ ID NO: 46]; a putative full-length Rattus novegicus TRAIL, as set forth in NCBI Accession: ABK32522 [SEQ ID NO: 48]; a putative full-length Mus musculus TRAIL, as set forth in NCBI Accession: BAE34141 [SEQ ID NO: 50]; and a putative full-length Mus musculus TRAIL, as set forth in NCBI Accession: NP_{—}033451 [SEQ ID NO: 52].

FIG. 2 is a graphical representation showing (A) a schematic diagram of the injection protocol adopted for repeated TRAIL administration in C57black mice. B and C are graphical representations showing metabolic parameters at the end of the study (12 weeks) performed in C57black mice. In B, data of fasting insulin are expressed as means±SEM, *p<0.05 vs C57 HF+TRAIL and vs C57 chow. In C, data of circulating lipids are expressed as means±SEM. *p<0.05 vs C57 chow.

FIG. 3 is a graphical representation showing glucose levels during an IPGTT at 12 weeks of the study in C57black mice. Data are expressed as means±SEM, *p<0.05 vs C57 HF.

FIG. 4 is a graphical representation showing insulin levels during an IPGTT at 12 weeks of the study in C57black mice. Data are expressed as means±SEM, *p<0.05 vs C57 HF.

FIG. 5 is a graphical representation showing glucose levels during an IPITT at 12 weeks of the study in C57 black mice. Data are expressed as means±SEM; *p<0.05 vs C57 HF.

FIG. 6 is a graphical representation showing food (A) and energy (B) intake. Data are expressed as means±SEM. In A, *p<0.05 vs C57 HF+TRAIL and C57 chow. Data are expressed as means±SEM. In B, *p<0.05 vs C57 HF (and C57 chow only at 1 and 4 weeks).

FIG. 7 is a graphical representation of the levels of serum IL-6 analyzed at the end of the study in C57 black mice. Data are expressed as means±SEM; *p<0.05 vs C57 HF+TRAIL and vs C57 chow.

FIG. 8 is a graphical representation showing (A) a schematic diagram of the injected protocol adopted for MDP/LPS±TRAIL short-term trail administration. In B are shown the levels of body temperature, serum amyloid A and number of peritoneal cells, analyzed two hours after MDP or LPS treatment in BALB/c mice left untreated or treated with TRAIL. Horizontal bars are median, upper and lower edges of box are 75th and 25th percentiles; lines extending from box are 10th and 90th percentiles.

FIG. 9 is a graphical representation showing the serum levels of IL-1alpha, IL-6, G-CSF, MCP-1, analyzed two hours after MDP or LPS treatment in BALB/c mice left untreated or treated with TRAIL. Horizontal bars are median, upper and lower edges of box are 75th and 25th percentiles; lines extending from box are 10th and 90th percentiles.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

By “antigen” is meant all, or part of, a molecule (e.g., a protein, peptide, or other molecule or macromolecule) capable of being bound by an antibody or a T cell receptor (TCR) if presented by MHC molecules. An antigen may be additionally capable of being recognized by the immune system and/or being capable of stimulating or inducing a humoral immune response and/or cellular immune response leading to the activation of B- and/or T-lymphocytes. An antigen may have one or more epitopes (B- and T-epitopes). Antigens as used herein may also be mixtures of several individual antigens.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

The term “apoptosis” is used herein in its broadest sense and refers to the orderly or controlled form of cell death in mammals that is typically accompanied by one or more characteristic cell changes, including condensation of cytoplasm, loss of plasma membrane micro-villi, segmentation of the nucleus, degradation of chromosomal DNA or loss of mitochondrial function. This activity can be determined and measured using well known art methods, for instance, by cell viability assays, FACS analysis or DNA electrophoresis, binding of annexin V, fragmentation of DNA, cell shrinkage, dilation of endoplasmic reticulum, cell fragmentation, and/or formation of membrane vesicles (called apoptotic bodies).

The term “biologically active fragment,” as applied to fragments of a reference or full-length polynucleotide or polypeptide sequence, refers to a fragment that has at least about 0.1, 0.5, 1, 2, 5, 10, 12, 14, 16, 18, 20, 22, 24, 26, 28, 30, 35, 40, 45, 50, 55, 60, 65, 70, 75, 80, 85, 90, 95, 96, 97, 98, 99% of the activity of a reference sequence. Included within the scope of the present invention are biologically active fragments of at least about 18, 19, 20, 21, 22, 23, 24, 25, 26, 27, 28, 29, 30, 40, 50, 60, 70, 80, 90, 100, 120, 140, 160, 180, 200, 250, 300, 400, 500, 600, 700, 800, 900 nucleotides or residues in length, which comprise or encode an activity of a reference polynucleotide or polypeptide. Representative biologically active fragments generally participate in an interaction, e.g., an intramolecular or an inter-molecular interaction. For example, biologically active portions of TRAIL polypeptides include peptides or polypeptides that interact with a TRAIL DR and comprise an amino acid sequence with sufficient similarity or identity to or derived from the amino acid sequence of a TRAIL polypeptide, illustrative examples of which include those set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102, and comprise at least one activity selected from: stimulating or otherwise inducing apoptosis of an adipose cell or tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, and reducing circulating levels of the proinflammatory cytokines.

By “coding sequence” is meant any nucleic acid sequence that contributes to the code for the polypeptide product of a gene. By contrast, the term “non-coding sequence” refers to any nucleic acid sequence that does not contribute to the code for the polypeptide product of a gene.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The phrase “conditions of localized, abnormal increases in adiposity” as used herein includes pathologies characterized by and/or associated with anatomically localized, disregulated adiposity that lead to circumscribed depositions of fat tissue. Such conditions include but are not limited to lipoma and lipomatosis.

By “corresponds to” or “corresponding to” is meant an amino acid sequence that displays substantial sequence similarity or identity to a reference amino acid sequence. In general, the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to the reference amino acid sequence.

By “effective amount”, in the context of modulating an activity or of treating or preventing a condition is meant the administration of that amount of agent to an individual in need of such modulation, treatment or prophylaxis, either in a single dose or as part of a series, that is effective for modulation of that effect or for treatment or prophylaxis or improvement of that condition. Non-limiting examples of such improvements in an individual suffering conditions of localized, abnormal increases in adiposity include reduced fat deposits, increased leanness, weight loss and an improvement in the symptoms relating to cardiovascular disease and diabetes. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “gene” is meant a unit of inheritance that occupies a specific locus on a chromosome and consists of transcriptional and/or translational regulatory sequences and/or a coding region and/or non-translated sequences (i.e., introns, 5′ and 3′ untranslated sequences).

The term “group” as applied to chemical species refers to a set of atoms that forms a portion of a molecule. In some instances, a group can include two or more atoms that are bonded to one another to form a portion of a molecule. A group can be monovalent or polyvalent (e.g., bivalent) to allow bonding to one or more additional groups of a molecule. For example, a monovalent group can be envisioned as a molecule with one of its hydrogen atoms removed to allow bonding to another group of a molecule. A group can be positively or negatively charged. For example, a positively charged group can be envisioned as a neutral group with one or more protons (i.e., H⁺) added, and a negatively charged group can be envisioned as a neutral group with one or more protons removed. Non-limiting examples of groups include, but are not limited to, alkyl groups, alkylene groups, alkenyl groups, alkenylene groups, alkynyl groups, alkynylene groups, aryl groups, arylene groups, iminyl groups, iminylene groups, hydride groups, halo groups, hydroxy groups, alkoxy groups, carboxy groups, thio groups, alkylthio groups, disulfide groups, cyano groups, nitro groups, amino groups, alkylamino groups, dialkylamino groups, silyl groups, and siloxy groups.

The term “host cell” includes an individual cell or cell culture, which can be or has been a recipient of any recombinant vector(s) or isolated polynucleotide of the invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with a recombinant vector or a polynucleotide of the invention. A host cell, which comprises a recombinant vector of the invention, is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently.

The term “hyperinsulinemia” refers to a state in an individual in which the level of insulin in the blood is higher than normal.

Reference herein to “immuno-interactive” includes reference to any interaction, reaction, or other form of association between molecules and in particular where one of the molecules is, or mimics, a component of the immune system.

The term “insulin resistance” refers to a state in which a normal amount of insulin produces a subnormal biologic response relative to the biological response in a subject that does not have insulin resistance.

“Insulin Resistance Syndrome,” as used herein, refers to various abnormalities associated with insulin resistance/compensatory hyperinsulinemia, which include the following: some degree of glucose intolerance (impaired fasting glucose and impaired glucose tolerance); dyslipidemia (increased triglycerides, decreased high-density lipoprotein cholesterol (HDL-C), decreased low-density lipoprotein (LDL)-particle diameter (small, dense LDL particles), and increased postprandial accumulation of triglyceride-rich lipoproteins); endothelial dysfunction (increased mononuclear cell adhesion, increased plasma concentration of cellular adhesion molecules, increased plasma concentration of asymmetric dimethylarginine, and decreased endothelial-dependent vasodilatation); procoagulant factors (increased plaminogen activator inhibitor-1 and increased fibrinogen); hemodynamic changes (sympathetic nervous system activity and renal sodium retention); markers of inflammation (increased C-reactive protein, white blood cell count, etc.); abnormal uric acid metabolism (increased plasma uric acid concentration and renal uric acid clearance); increased testosterone secretion (ovary); and sleep-disordered breathing. Further, some of the clinical syndromes associated with insulin resistance include the following: diabetes mellitus, cardiovascular disease, essential hypertension, polycystic ovary syndrome, nonalcoholic fatty liver disease, certain forms of cancer, and sleep apnea.

By “isolated” is meant material that is substantially or essentially free from components that normally accompany it in its native state. For example, an “isolated polynucleotide,” as used herein, refers to a polynucleotide, which has been purified from the sequences, which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell, i.e., it is not associated with in vivo substances. Similarly, an “isolated” or “purified” proteinaceous molecule (e.g., peptide, polypeptide, protein etc.) is substantially free of cellular material or other contaminating molecules from the cell or tissue source from which the proteinaceous molecule is derived, or substantially free from chemical precursors or other chemicals when chemically synthesized. “Substantially free” means that a preparation of a TRAIL polypeptide is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, a preparation of TRAIL polypeptide has less than about 30, 25, 20, 15, 10, 9, 8, 7, 6, 5, 4, 3, 2 or 1% (by dry weight), of non-TRAILS (also referred to herein as a “contaminating molecules”), or of chemical precursors or non-TRAIL chemicals. When the TRAIL polypeptide is recombinantly produced, it is also desirably substantially free of culture medium, i.e., culture medium represents less than about 20, 15, 10, 5, 4, 3, 2, 1% of the volume of the TRAIL polypeptide preparation. The invention includes isolated or purified preparations of at least 0.01, 0.1, 1.0, and 10 milligrams in dry weight.

By “linker,” is meant a molecule or group of molecules (such as a monomer or polymer) that connects two molecules and often serves to place the two molecules in a desirable configuration.

“Metabolic Syndrome,” as used herein, refers to a combination of medical disorders that increases the risk to a person for cardiovascular disease and diabetes. Other known names referring to such syndrome is syndrome X, insulin resistance syndrome, Reaven's syndrome. Several features of the syndromes include: fasting hyperglycemia, high blood pressure, central obesity (also known as visceral obesity), decreased High Density Lipoprotein (HDL), elevated triglycerides, elevated uric acid levels. Fasting hyperglycemia, listed above, includes diabetes mellitus type II or impaired fasting glucose and impaired glucose tolerance or insulin resistance. In addition to metabolic syndrome, the TRAIL DR agonists may have indications for pre-diabetic states.

By “modulating” is meant increasing or decreasing, either directly or indirectly, the death of an adipose cell of an individual or the adiposity in a subject. In certain embodiments, “modulation” or “modulating” means that a desired/selected activity (e.g., adipose cell death or apoptosis) is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than in the absence of a TRAIL DR agonist.

The term “obesity” as used herein includes conditions where there is an increase in body fat beyond the physical requirement as a result of excess accumulation of adipose tissue in the body. The term obesity includes, but is not limited to, the following conditions: adult-onset obesity; alimentary obesity; endogenous or metabolic obesity; endocrine obesity; familial obesity; hyperinsulinar obesity; hyperplastic-hypertrophic obesity; hypogonadal obesity; hypothyroid obesity; lifelong obesity; morbid obesity and exogenous obesity.

By “obtained from” is meant that a sample such as, for example, a polynucleotide extract or polypeptide extract is isolated from, or derived from, a particular source.

The term “operably connected” or “operably linked” as used herein means placing a structural gene under the regulatory control of a regulatory element including but not limited to a promoter, which then controls the transcription and optionally translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e. the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e. the genes from which it is derived.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of treatment or prophylaxis for an adiposity-related condition. However, it will be understood that the aforementioned terms do not imply that symptoms are present.

By “pharmaceutically acceptable carrier” is meant a solid or liquid filler, diluent or encapsulating substance that can be safely used in topical or systemic administration to an animal, preferably a mammal, including humans.

The term “polynucleotide” or “nucleic acid” as used herein designates mRNA, RNA, cRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide,” “peptide,” “protein” and “proteinaceous molecule” are used interchangeably herein to refer to molecules comprising or consisting of a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers.

The terms “peptide variant” and “polypeptide variant” and the like refer to peptides and polypeptides that are distinguished from a reference peptide or polypeptide by the addition, deletion or substitution of at least one amino acid residue. In certain embodiments, a peptide or polypeptide variant is distinguished from a reference peptide or polypeptide by one or more substitutions, which may be conservative or non-conservative. In certain embodiments, the peptide or polypeptide variant comprises conservative substitutions and, in this regard, it is well understood in the art that some amino acids may be changed to others with broadly similar properties without changing the nature of the activity of the peptide or polypeptide. Peptide and polypeptide variants also encompass peptides and polypeptides in which one or more amino acids have been added or deleted, or replaced with different amino acid residues.

By “primer” is meant an oligonucleotide which, when paired with a strand of DNA, is capable of initiating the synthesis of a primer extension product in the presence of a suitable polymerizing agent. The primer is preferably single-stranded for maximum efficiency in amplification but can alternatively be double-stranded. A primer must be sufficiently long to prime the synthesis of extension products in the presence of the polymerization agent. The length of the primer depends on many factors, including application, temperature to be employed, template reaction conditions, other reagents, and source of primers. For example, depending on the complexity of the target sequence, the oligonucleotide primer typically contains 15 to 35 or more nucleotide residues, although it can contain fewer nucleotide residues. Primers can be large polynucleotides, such as from about 200 nucleotide residues to several kilobases or more. Primers can be selected to be “substantially complementary” to the sequence on the template to which it is designed to hybridize and serve as a site for the initiation of synthesis. By “substantially complementary”, it is meant that the primer is sufficiently complementary to hybridize with a target polynucleotide. Preferably, the primer contains no mismatches with the template to which it is designed to hybridize but this is not essential. For example, non-complementary nucleotide residues can be attached to the 5′ end of the primer, with the remainder of the primer sequence being complementary to the template. Alternatively, non-complementary nucleotide residues or a stretch of non-complementary nucleotide residues can be interspersed into a primer, provided that the primer sequence has sufficient complementarity with the sequence of the template to hybridize therewith and thereby form a template for synthesis of the extension product of the primer.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

By “regulatory element” or “regulatory sequence” is meant nucleic acid sequences (e.g., DNA) necessary for expression of an operably linked coding sequence in a particular host cell. The regulatory sequences that are suitable for prokaryotic cells for example, include a promoter, and optionally a cis-acting sequence such as an operator sequence and a ribosome binding site. Control sequences that are suitable for eukaryotic cells include promoters, polyadenylation signals, transcriptional enhancers, translational enhancers, leader or trailing sequences that modulate mRNA stability, as well as targeting sequences that target a product encoded by a transcribed polynucleotide to an intracellular compartment within a cell or to the extracellular environment.

The term “sequence identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

“Similarity” refers to the percentage number of amino acids that are identical or constitute conservative substitutions as defined in Tables 1 and 2 infra. Similarity may be determined using sequence comparison programs such as GAP (Deveraux et al. 1984, Nucleic Acids Research 12:387-395). In this way, sequences of a similar or substantially different length to those cited herein might be compared by insertion of gaps into the alignment, such gaps being determined, for example, by the comparison algorithm used by GAP.

Terms used to describe sequence relationships between two or more polynucleotides or polypeptides include “reference sequence,” “comparison window”, “sequence identity,” “percentage of sequence identity” and “substantial identity”. A “reference sequence” is at least 12 but frequently 15 to 18 and often at least 25 monomer units, inclusive of nucleotides and amino acid residues, in length. Because two polynucleotides may each comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., “Current Protocols in Molecular Biology”, John Wiley & Sons Inc, 1994-1998, Chapter 15.

“Stringency” as used herein, refers to the temperature and ionic strength conditions, and presence or absence of certain organic solvents, during hybridization and washing procedures. The higher the stringency, the higher will be the degree of complementarity between immobilized target nucleotide sequences and the labeled probe polynucleotide sequences that remain hybridized to the target after washing. The term “high stringency” refers to temperature and ionic conditions under which only nucleotide sequences having a high frequency of complementary bases will hybridize. The stringency required is nucleotide sequence dependent and depends upon the various components present during hybridization. Generally, stringent conditions are selected to be about 10 to 20° C. lower than the thermal melting point (Tₘ) for the specific sequence at a defined ionic strength and pH. The Tₘ is the temperature (under defined ionic strength and pH) at which 50% of a target sequence hybridizes to a complementary probe.

The term “TRAIL polypeptides,” as used herein encompasses, without limitation, polypeptides having an amino acid sequence that shares at least 70% (and at least 71% to at least 99% and all integer percentages in between) sequence identity or similarity with the sequence set forth in any one of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102. It further encompasses natural allelic variation of TRAIL polypeptides that may exist and occur from one organism to another. Also, degree and location of glycosylation or other post-translation modifications may vary depending on the chosen host and the nature of the hosts cellular environment. The term “TRAIL polypeptides” is also intended to encompass TRAIL polypeptides in their precursor form, as well as those that have been processed to yield their respective bioactive forms. It further encompasses TRAIL polypeptides that have either been chemically modified relative to a reference or naturally-occurring TRAIL polypeptide and/or contain one or more amino acid sequence alterations relative to a reference or naturally-occurring TRAIL polypeptide and/or contain truncated amino acid sequences relative to a reference or naturally-occurring full-length or precursor TRAIL polypeptide. Alternatively, or in addition, TRAIL polypeptides may exhibit different properties relative to a reference or naturally-occurring TRAIL polypeptide, including stability and an altered specific activity selected from stimulating or otherwise inducing apoptosis of an adipose cell or tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, reducing circulating levels of the proinflammatory cytokines IL-6, IL-1alpha and MCP-1, and counteracting the lipopolysaccaride- and muramildipeptide-induced fever. The term “TRAIL polypeptide” also encompasses proteinaceous molecules with a slightly modified amino acid sequence, for instance, polypeptides having a modified N-terminal end including N-terminal amino acid deletions or additions, and/or polypeptides that have been chemically modified relative to a reference or naturally-occurring TRAIL polypeptide. TRAIL polypeptides also encompass proteinaceous molecules exhibiting substantially the same or better bioactivity than a reference or naturally-occurring TRAIL polypeptide, or, alternatively, exhibiting substantially modified or reduced bioactivity relative to a reference or naturally-occurring TRAIL polypeptide. They also include, without limitation, polypeptides having an amino acid sequence that differs from the sequence of a reference or naturally-occurring TRAIL polypeptide by insertion, deletion, or substitution of one or more amino acids and in illustrative examples, encompass proteinaceous molecules that exhibit at least about 10%, 20%, 30%, 40%, 50%, 60%, 70%, 80%, 90%, 100%, 110%, 120%, and 130% of the specific activity of a reference or naturally-occurring TRAIL polypeptide that has been produced in the same cell. TRAIL polypeptides having substantially the same or improved biological activity relative to a reference or naturally-occurring TRAIL polypeptide, encompass molecules that exhibit at least about 10%, 20%, 30%, 40%, 50%, 60%, 70%, 80%, 90%, 100%, 110%, 120%, and 130% of the specific biological activity of the reference or naturally-occurring TRAIL polypeptide that has been produced in the same cell type.

The term “TRAIL death receptor” as used herein refers to a protein that binds TRAIL and, upon binding TRAIL, activates programmed cell death (apoptosis) in tumor cells. Certain non-limiting examples of a TRAIL death receptor include either of the receptor proteins commonly referred to as TRAIL-R1 (DR4) or TRAIL-R2 (DR5).

The term “TRAIL decoy receptor” as used herein refers to a protein that binds TRAIL and, upon binding TRAIL, does not activate programmed cell death (apoptosis) in tumor cells. Accordingly, TRAIL decoy receptors are believed to function as inhibitors, rather than transducers of programmed cell death signaling. Certain non-limiting examples of a TRAIL decoy receptor include any of the receptor proteins commonly referred to as TRAIL-R3 (also DcR1, TRID, LIT or TNFRSF10c) [(Pan et al., Science 276:111-113, 1997; Sheridan et al., Science 277:818-821, 1997; McFarlane et al., J Biol Chem 272:25417-25420, 1997; Schneider et al., FEBS Letters 416:329-334, 1997; Degli-Esposti et al. J Exp Med 186:1165-1170, 1997; and Mongkolsapaya et al., J Immunol 160:3-6, 1998], TRAIL-R4 (also DcR2, TRUNDD and TNFRSF10d), [Marsters et al., Curr Biol 7:1003-1006, 1997; Pan et al., FEBS Letters 424:41-45, 1998; Degli-Esposti et al., Immunity 7:813-820, 1997] and circulating osteoprotegerin (also OPG, TNFRSF11b), each of which is incorporated herein by reference in its entirety.

The terms “TRAIL-R1,” “DR4” and “DR4 receptor” are used interchangeably herein to refer to the full length TRAIL receptor sequence and soluble, extracellular domain forms of the receptor described in Pan et al., Science 276:111-113, 1997; WO98/32856 published Jul. 30, 1998; U.S. Pat. No. 6,342,363 issued Jan. 29, 2002; and WO99/37684 published Jul. 29, 1999, each of which is incorporated herein by reference in its entirety.

The term “TRAIL-R2,” “DR5” and “DR5 receptor” are used interchangeably herein to refer to the full length TRAIL receptor sequence and soluble, extracellular domain forms of the receptor described in Sheridan et al., Science 277:818-821, 1997; Pan et al., Science 277:815-818, 1997, U.S. Pat. No. 6,072,047 issued Jun. 6, 2000; U.S. Pat. No. 6,342,369, WO98/51793 published Nov. 19, 1998; WO98/41629 published Sep. 24, 1998; Screaton et al., Curr Biol 7:693-696, 1997; Walczak et al., EMBO J. 16:5386-5387, 1997; Wu et al., Nature Genetics, 17:141-143, 1997; WO98/35986 published Aug. 20, 1998; EP870,827 published Oct. 14, 1998; WO98/46643 published Oct. 22, 1998; WO99/02653 published Jan. 21, 1999; WO99/09165 published Feb. 25, 1999; WO99/11791 published Mar. 11, 1999, each of which is incorporated herein by reference in its entirety.

The terms “TRAIL receptor agonist,” “TRAIL death receptor agonist” and “agonist” are used interchangeably herein in a broad sense, and include any molecule or compound that partially or fully enhances, stimulates or activates one or more biological activities of TRAIL-R1 or TRAIL-R2, and biologically active variants thereof, whether in vitro, in situ, in vivo or ex vivo. Examples of such biological activities include apoptosis as well as those further reported in the literature. An agonist may function in a direct or indirect manner. For instance, a “TRAIL death receptor agonist” may function to partially or fully enhance, stimulate or activate one or more biological activities of TRAIL-R1 or TRAIL-R2, in vitro, in situ, in vivo or ex vivo as a result of its direct binding to one or both of those receptors, which causes receptor activation or signal transduction. TRAIL receptor agonists include TRAIL polypeptides as defined herein as well as peptides and polypeptides that bind to TRAIL receptors that would not be considered a TRAIL polypeptide (e.g., peptides or polypeptides that specifically bind a TRAIL DR but not a TRAIL decoy receptor) as well as small molecules that agonize a TRAIL DR.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

“Diabetes mellitus” refers to a group of diseases characterized by high blood glucose levels that result from defects in the body's ability to produce and/or use insulin. This term comprises different types of diabetes.

“Type I diabetes” is usually diagnosed in children and young adults, and was previously known as juvenile diabetes. In type I diabetes, the body does not produce insulin. Insulin is a hormone that is needed to convert sugar (glucose), starches and other food into energy needed for daily life.

“Type II diabetes” or “non-insulin dependent diabetes mellitus” refers to an insulin-related disorder in which there is a relative disparity between endogenous insulin production and insulin requirements, leading to elevated hepatic glucose production, elevated blood glucose levels, inappropriate insulin secretion, and peripheral insulin resistance. Type II diabetes has been regarded as a relatively distinct disease entity, but type II diabetes is often a manifestation of a much broader underlying disorder (Zimmet et al., Nature 414:782-787, 2001), which may include metabolic syndrome (syndrome X), diabetes (e.g., type II diabetes, type II diabetes, gestational diabetes, autoimmune diabetes), hyperinsulinemia, hyperglycemia, impaired glucose tolerance (IGT), hypoglycemia, B-cell failure, insulin resistance, dyslipidemias, atheroma, insulinoma, hypertension, hypercoagulability, microalbuminuria, and obesity and other adiposity-related conditions such as visceral obesity, central fat, obesity-related type II diabetes, obesity-related atherosclerosis, heart disease, obesity-related insulin resistance, obesity-related hypertension, microangiopathic lesions resulting from obesity-related type II diabetes, ocular lesions caused by microangiopathy in obese individuals with obesity-related type U diabetes, and renal lesions caused by microangiopathy in obese individuals with obesity-related type II diabetes.

In the context of the present invention, “diabetes mellitus” comprises type I diabetes, type II diabetes and mixed forms thereof. Type III diabetes is also included in the definition. In a particular embodiment, the present invention refers to type II diabetes, but other forms of diabetes mellitus are included.

By “vector” is meant a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is preferably a viral or viral-derived vector, which is operably functional in animal and preferably mammalian cells. Such vector may be derived from a poxvirus, an adenovirus or yeast. The vector can also include a selection marker such as an antibiotic resistance gene that can be used for selection of suitable transformants Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type” and “naturally occurring” are used interchangeably to refer to a gene or gene product that has the characteristics of that gene or gene product when isolated from a naturally occurring source. A wild type gene or gene product (e.g., a polypeptide) is that which is most frequently observed in a population and is thus arbitrarily designed the “normal” or “wild-type” form of the gene.

As used herein, underscoring or italicising the name of a gene shall indicate the gene, in contrast to its protein product, which is indicated by the name of the gene in the absence of any underscoring or italicising. For example, “TRAIL” shall mean the TRAIL gene or TRAIL polynucleotides, whereas “TRAIL” shall indicate the protein product or products generated from transcription and translation and alternative splicing of the “TRAIL” gene.

### 2. Abbreviations

The following abbreviations are used throughout the application:

| | |
|---|---|
| HFD = | high fat diet |
| nts = | nucleotides |
| d = | day |
| h = | hour |
| s = | seconds |

### 3. TRAIL DR Agonists for Use in Treating or Preventing Adiposity-Related Conditions

The present invention is based in part on the determination that when TRAIL polypeptides are administered to animals, they are effective in eliciting at least one of the following: (a) stimulating apoptosis of adipose cells or tissues; (b) reducing fasting hyperinsulinemia, (c) reducing glucose levels after a hyperglycemic stimulus; (d) reducing hyperinsulinemia after a hyperglycemic stimulus, (e) enhancing peripheral response to insulin; (f) reducing increased adiposity in response to high fat diet, (g) improving mitochondrial fatty acid oxidative capacity of muscle tissue, (h) reducing circulating levels of the proinflammatory cytokines IL-6, IL-1alpha and MCP-1, (i) counteracting lipopolysaccaride- and muramildipeptide-induced proinflammatory activity and body temperature elevation. The present inventors thus consider that these TRAIL polypeptides, as well as other TRAIL DR agonists, will be useful in controlling adiposity including the treatment or prevention of adiposity-related conditions (e.g., obesity and conditions of localized, abnormal increases in adiposity such as, but not limited to, lipoma and lipomatosis, as well as type II diabetes and metabolic syndrome).

Accordingly, the present invention provides TRAIL DR agonists in methods and compositions for controlling adiposity in a subject including adiposity related conditions such as obesity and conditions of localized, abnormal increases in adiposity. When included in compositions, the TRAIL DR agonists are suitably combined with a pharmaceutically acceptable carrier or diluent. Conditions contemplated in such treatment regimes include conditions or pathologies which are associated with or secondary to obesity, such but not limited to type II diabetes, overeating, binge eating, and bulimia, hypertension, elevated plasma insulin concentrations and insulin resistance, dyslipidemia, hyperlipidemia, obstructive sleep apnea, heart disease, abnormal heart rhythms and arrhythmias, myocardial infarction, congestive heart failure, coronary heart disease, sudden death, stroke and other pathological conditions showing reduced metabolic activity or a decrease in resting energy expenditure as a percentage of total fat-free mass, e.g., children with acute lymphoblastic leukemia. Further examples of adiposity-related conditions are metabolic syndrome, insulin resistance syndrome, reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-hypoventilation syndrome (Pickwickian syndrome), breathlessness, cardiovascular disorders, inflammation, such as systemic inflammation of the vasculature, arteriosclerosis, hypercholesterolemia, lower back pain, gallbladder disease, hyperuricemia, gout, and kidney cancer, and increased anesthetic risk. Conditions of localized, abnormal increases in adiposity may include adipose tumors (lipomas and liposarcomas) and lipomatosis. In specific embodiments, the adiposity related condition is selected from obesity, diabetes mellitus and metabolic syndrome.

The TRAIL DR agonists of the present invention can be administered by any suitable route include for example by injection, by topical or mucosal application, by inhalation or via the oral route including modified-release modes of administration to control excess adiposity and/or to treat or prevent an adiposity-related condition in a subject.

Suitable TRAIL DR agonists include TRAIL polypeptides, TRAIL polynucleotides, chimeric polypeptides comprising a trimerizing domain and at least one C-type lectin like domain that binds to at least one TRAIL DR and TRAIL DR agonist antigen-binding molecules, TRAIL DR agonist peptides as well as small molecule TRAIL DR agonists.

3.1 TRAIL Polypeptides

In some embodiments, the TRAIL DR agonist is selected from TRAIL polypeptides, which are suitably in isolated, synthetic, recombinant or purified form. The present invention contemplates full-length TRAIL polypeptides as well as their biologically active fragments. Typically, biologically active fragments of a full-length TRAIL polypeptide may participate in an interaction, for example, an intra-molecular or an inter-molecular interaction (e.g., an interaction with a TRAIL DR, illustrative examples of which include TRAIL R-1 and TRAIL R-2) and/or may display any one or more of activities (a) to (g) noted above. Such biologically active fragments include peptides or polypeptides comprising amino acid sequences sufficiently similar to or derived from the amino acid sequences of a (putative) full-length TRAIL polypeptide, which include less amino acids than the putatively full-length TRAIL polypeptide, and exhibit at least one activity of that polypeptide (e.g., any one or more of activities (a) to (g) defined above. Non-limiting examples of putatively full-length TRAIL polypeptides include:

MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVG
[SEQ ID NO: 4]; (corresponding to a putative full-length synthetic TRAIL,
as set forth in NCBI Accession: AAV38370);
MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVGL
[SEQ ID NO: 6] (corresponding to a putative full-length human TRAIL
isoform 1, as set forth in NCBI Accession: NP_003801);
MAMMEVQGGPSLGQTCVLIVIFTVLLQSLCVAVTYVYFTNELKQMQDKYSK
SGIACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFLGAFLVGL
[SEQ ID NO: 8] (corresponding to a putative full-length synthetic
TRAIL, as set forth in NCBI Accession: AAX29952);
KEKQQNISPLVRERGPQRVAAHITGTRGRSNTLSSPNSKNEKALGRKINSWESS
RSGHSFLSNLHLRNGELVIHEKGFYYIYSQTYFRFQEEIKENTKNDKQMVQYIYKYTS
YPDPILLMKSARNSCWSKDAEYGLYSIYQGGIFELKENDRIFVSVTNEHLIDMDHEAS
FFGAFLVG [SEQ ID NO: 10] (corresponding to a putative full-length human TRAIL
isoform CRA_b, as set forth in NCBI Accession: EAW78466);
MAMMEVQGGPSLGQTCVLIVVFTVLLQSLCVAVTYVYFTNELKQMQDKYSK
SGIACFLKEDDSYWDPNDEDSMNSPCWQVKWQLRQLVRKMILRTSEETISTVQEKQ
QNISPLVRERGPQRVAAHITGTRGRSNTLSSPNSKNEKALGHKINSWESSRSGHSFLSN
LHLRNGELVIHEKGFYYIYSQTYFRFQEEIKENTKNDKQMVQYIYKYTSYPDPILLMK
SARNSCWSKDAEYGLYSIYQGGIFELKENDRIFVSVTNEHLIDMDHEASFFGAFLVG
[SEQ ID NO: 12] (corresponding to a putative full-length Pan troglodytes
TRAIL, as set forth in NCBI Accession: XP_516879);
MILRTSEETISTVQEKQQNISPLVRERGPQRVAAHITGTRGRSNTLSSPNSKNEK
ALGRKINSWESSRSGHSFLSNLHLRNGELVIHEKGFYYIYSQTYFRFQEEIKENTKNDK
QMVQYIYKYTSYPAPILLMKSARNSCWSKDAEYGLYSIYQGGIFELKENDRIFVSVTN
EHLIDMDHEASFFGAFLVG [SEQ ID NO: 16] (corresponding to a human TRAIL
fragment, as set forth in NCBI Accession: 1DG6);
MAMMEAQGGPSPGQTCVLILIFTVLLQSLCAAVTYVYFTNELKQMQDKYSKS
GIACFLKEDDSSWDPNDEESMKSPCWQVKWQLRQLVRKMILRTSEETISTVQEKQQN
TSPLVRERGPQRVAAHITGTRGRSNTLSSPNSKNEKALGRKINSWESSRSGHSFLSNL
HLRNGELVIQEKGFYYIYSQTYFRFQEEIKENTKNDKQMVQYIYKYTSYPDPILLMKS
ARNSCWSKDAEYGLYSIYQGGLFELKKDDRIFVSVTNEHLIDMDHEASFFGAFLVG
[SEQ ID NO: 18] (corresponding to a putative full-length Macaca mulatta
TRAIL, as set forth in NCBI Accession: XP_001084768);
MVRERGPQRVAAHITGTRGRSNTLSSPNSKNEKALGRKINSWESSRSGHSFQS
NLHLRNGELVIHEKGFYYIYSQTYFRFQEEIKENAKNDKQMVQYIYKYTSYPDPILLM
KSARNSCWSKDAEYGLYSIYQGGIFELKENDRIFVSVTNEHLIDMDHEASFFGAF
[SEQ ID NO: 20] (corresponding a putative full-length Crassostrea ariakensis
TRAIL, as set forth in NCBI Accession: ABU39827);
MAMMEVQGGPSLGQTCVLIVIFTVLLQSLCVAVTYVYFTNELKQMQDKYSK
SGIACFLKEDDSSWDPNDEDSMNSPCWQVKWQLRQLVRKMILRTSEETISTVQEKQQ
NVSPLVRERGPQRVAAHITGTRGRSNTLSSPSKRNNKXXXRKINSWESSRSGHSFLSN
LHLRNGELVIHEKGFYYIYSQTYFRFQEEIKENTKNDKQMVQYIYKYTSYPDPILLMK
SARNSCWSKDAEYGLYSIYQGGIFELKENDRIFVSVTNEHLIDMDHEASFFGAFLVG
[SEQ ID NO: 24] (corresponding to a putative full-length Pongo abelii
TRAIL, as set forth in NCBI Accession: XP_002814335);
MAMMEGQGGPSPGQTCVLILIFTVLLQSLCVAVTYLYFTNELKQMQDKYSKS
GIACFLKEDGSSWDPSDEESMNSPCWEVKWQLRQLVRKMILRTSEETISTVQEKQRGI
SPQVRERGPQRVAAHITGTRGSSNTLPIPNSKNEKALGRKINSWESSRSGHSFLSNLHL
RNGELVIHEKGLYYIYCQVYFRFQEEIQENRKNDKQMVQYIYKYTSYPDPILLMKSA
RNNCWSKDAEYGLYSIYQGGIFELKENDRIFVSVTNGQLIDMDHEASFFGAFLVG
[SEQ ID NO: 26] (corresponding to a putative full-length Callithrix jacchus
TRAIL, as set forth in NCBI Accession: XP_002759427);
MQAPAGPSPGQTCVLILIFTVLLQSLCVAVTYMYFTSELRQMQDKYSQSGIAC
FLKEDDIPWDPNDEESMNTPCWQVKWQLRQFVRKILRTYEETIPTVPEKQLNIPYLVR
ERGPQRVAAHITGTSRRRSTFPVPSSKNEKALGQKINSWESSRKGHSFLNNLHLRNGE
LVIHQRGFYYIYSQTYFRFQEPEEIPTGQNRKRNKQMVQYIYKHTSYPDPILLMKSAR
NSCWSKDSEYGLYSIYQGGIFELKENDRIFVSVSNEQLIDMDQEASFFGAFLIG [SEQ
ID NO: 28] (corresponding to a putative full-length Felis catus TRAIL,
as set forth in NCBI Accession: NP_001124316);
MQAPGGPSPGQTCVLTLIFTVLLQSLCVAVTYMYFTRELKQMQDKYSQSGIA
CFLKEDDIPWDPNDEESMNNPCWQVKWQLRQFVRKMILKTYEETIPSIPEKQLNIPYV
VNERGLQRVAAHITGTSRRRSTFPVPSSKNEKALGQKINSWESSRKGHSFLSNLHLRN
GELVIHQSGFYYIYSQTYFRFQEPEETSGPISKEQNRKKNKQMVQYIYKYTSYPDPILL
MKSARNSCWSKDSEYGLYSIYQGGIFELKENDRIFVSVNNEQLIDMDQEASFFGAFLI
G [SEQ ID NO: 30] (corresponding to a putative full-length Ailuropoda melanoleuca
TRAIL, as set forth in NCBI Accession: XP_002921635);
MAMMQASGGPSPGQTCVLILIFTVLLQALCVAVTYLYFTNELKQMQIKYSKS
GIACFLKEDDSDWDPNDEESMNSPCWQVKWQLRQFVRKMILRTYEESIPTTSEKRQN
IPPLVRERGLQRVAAHITGTSRRRSTVSIPRSKNEKALGQKINAWETSRKGHSFLNNL
HLRNGELVIHQTGFYYIYSQTYFRFQEPEEILGTVATEENRRKNKQMVQYIYKSTDYP
DPILLMKSARNSCWSKDSEYGLYSIYQGGIFELKENDRIFVSVTNEQLIDMDQEASFF
GAFLIG [SEQ ID NO: 32] (corresponding to a putative full-length Equus caballus
TRAIL, as set forth in NCBI Accession: XP_001494138);
MQAPGGPSPGQTCVLTLIFTVLLQSLCVAVTYMYFTRELKQMQDKYSQSGIA
CFLKEDDIPWDPNDEESMNNPCWQVKWQLRQFVRKMILKTYEETIPSIPEKQLNIPYV
VNERGLQRVAAHITGTSRRRSTFPVPSSKNEKALGQKINSWESSRKGHSFLSNLHLRN
GELVIHQSGFYYIYSQTYFRFQEPEETSGPISKEQNRKKNKQMVQYIYKYTSYPDPILL
MKSARNSCWSKDSEYGLYSIYQGGIFELKENDRIFVSVNNEQLIDMDQEASFFGAF
[SEQ ID NO: 34] (corresponding to a putative full-length Ailuropoda melanoleuca
TRAIL, as set forth in NCBI Accession: EFB16787);
MALKQAPGSRLGQICMPILIFTVLLQAFGMAVFYMYFNKELKQMQNKYFKSG
LACFLEEDDRSWDSRDDESIINPCWELKSQLYLFVKKMTLRTFEEMIPTNPEKQYNPY
LEREKGPKRVAAHITGSNRKKSTLPVPGSKNEKAVGHKINSWESSRKGHSFLNNLYL
RNGELVILQTGFYYIYSQTYFRFQEPEEVLGTVSTEENRKKIKQMVQYIYKYTNYPDPI
LLMKSARNSCWSKDSEYGLYSIYQGGIFELKENDRIFVSVTNERLVDLDQEASFFGAF
LIG [SEQ ID NO: 36] (corresponding to a putative full-length Bos taurus
TRAIL, as set forth in NCBI Accession: XP_583785);
MAVMQTPGGPSPGQTCVLILIFTVLLQALCVALTYVYFTNELKQMQDKYSKS
GIACFLKEDDSFWDPTDDERMLSPCWQVKWQLRQFVRKMILRTYEETISTVSEKQQG
IPHLEREKGPQRVAAHITGTSRKRSTFPSLSSKYEKALGQKINSWESSRKGHSFLNNFH
LRNGELVIHQTGFYYIYSQTYFRFQEPEEILGTVSTEGNRKKNRQMIQYIYKWTSYPD
PILLMKSARNSCWSKDSEYGLYSIYQGGIFELKEDDRIFVSVTNEQLIDMDQEASFFG
AFLIG [SEQ ID NO: 38] (corresponding to a putative full-length Sus scrofa
TRAIL, as set forth in NCBI Accession: NP_001019867);
MQAPGGPSLGLTCVLILIFTVLLQSLCVAVTYMYFTRELKQMQDKYSQSGIAC
FLKEDDIPWDPSDEESMNNPCWQVKWQLRQFVRKMILKTYEETIPTAPEKQLNIPYV
VSDRGSQRVAAHITGTSRRSMFPIPSSKNDKALGHKINSWDSTRKGHSFLNNLHLRN
GELVIHQRGFYYIYSQTYFRFQEPEEIPTGQNRKRNKQMVQYIYKHTSYPDPILLMKS
ARNSCWSKDSEYGLYSIYQGGIFELKENDRIFVSVSNEQLIDMDQEASFFGAFLIG
[SEQ ID NO: 40] (corresponding to a putative full-length Canis lupis familiaris
TRAIL, as set forth in NCBI Accession: NP_001124308);
MSSVQALGGPSAGQTCVLILIFTVLLQSLCVAVTYLYFTNELKQMQDKYSKSG
IACLLKEDDSSWDSIDEENMNSPCWQAKWQLRQFIRKMLLRTYEETIPTVEEKPQTIP
SLVREKERERGPQRVAAHLTGNSWRSFISVPAPGSQSGKNLGQKISSWESSRKGHSFL
NNLHLRNGELVIHQTGLYYIYSQTYFRFQELEEISGTISREEIKKRNKQMVQYIYKWTS
YPDPILLMKSARNSCWSKDSEYGLYSIYQGGIFELKENDRIFVSVTNEQLIDMNQESSF
FGAFLIG [SEQ ID NO: 42] (corresponding to a putative full-length Oryctolagus
cuniculus TRAIL, as set forth in NCBI Accession: XP_002716472);
MPSTGNLKGPSFSQHFTMTVICIVLLQVLLQALTVAVTYMYFNNEVKQLQDN
YSKIGLACFSKEDGDFWDSTDEGILNRPCLQVKRQLYQLIEEVTLRTFEKTISTVPEKQ
LSTPPLPRGRRPQRVAAHITGITRRSNLALIPISKDGKTLGQKIETWESSRRGHSFLNHV
HLRNGELVIQEEGLYYIYSQTYYRFKEAKEASKTVSKDGGRIKQMVQYIYKYTSYPD
PILLMKSARNSCWSREAEYGLYSIYQGGLFELKENDRIFVSVTNEHLMDLDQEASFFG
AFLIN [SEQ ID NO: 44] (corresponding to a putative full-length Rattus novegicus
TRAIL, as set forth in NCBI Accession: EDM01114);
MASTGNLKGPSFSQHFTMTVICIVLLQVLLQALTVAVTYMYFNNEVKQLQDN
YSKIGLACFSKEDGDFWDSTDEGILNRPCLQVKRQLYQLIEEVTLRTFEKTISTVPEKQ
LSTPPLPRGRRPQRVAAHITGITRRSNLALIPISKDGKTLGQKIETWESSRRGHSFLNHV
HLRNGELVIQEEGLYYIYSQTYYRFKEAKEASKTVSKDGGRIKQMVQYIYKYTSYPD
PILLMKSARNSCWSREAEYGLYSIYQGGLFELKENDRIFVSVTNEHLMDLDHEASFFG
A [SEQ ID NO: 46] (corresponding to a putative full-length Rattus novegicus
TRAIL, as set forth in NCBI Accession: NP_663714);
MASTGNLKGPSFSQHFTMTVICIVLLQVLLQALTVAVTYMYFNNEVKQLQDN
YSKIGLACFSKEDGDFWDSTDEGILNRPCLQVKRQLYQLIEEVTLRTFEKTISTVPEKQ
LSTPPLPRGRRPQRVAAHITGITRRSNLALIPISKDGKTLGQKIETWESSRRGHSFLNHV
HLRNGELVIQEEGLYYIYSQTYYRFKEAKEASKTVSKDGGRIKQMVQYIYKYTSYPD
PILLMKSARNSCWSREAEYGLYSIYQGGLFELKENDRIFVSVTNEHLMDLDQEASFFG
A [SEQ ID NO: 48] (corresponding to a putative full-length Rattus novegicus
TRAIL, as set forth in NCBI Accession: ABK32522);
MPSSGALKDLSFSQHFRMMVICIVLLQVLLQAVSVAVTYMYFTSEMKQLQDN
YSKIGLACFSKTDEDFWDSTDGEILNRPCLQVKRQLYQLIEEVTLRTFQDTISTVPEKQ
LSTPPLPRGGRPQKVAAHITGITRRSNSALIPISKDGKTLGQKIESWESSRKGHSFLNHV
LFRNGELVIEQEGLYYIYSQTYFRFQEAKDASKMVSKDKVRTKQLVQYIYKYTSYPD
PIVLMKSARNSCWSRDAEYGLYSIYQGGLFELKKNDRIFVSVTNEHLMDLDQEASFF
GAFLIN [SEQ ID NO: 50] (corresponding to a putative full-length Mus musculus
TRAIL, as set forth in NCBI Accession: BAE34141);
MPSSGALKDLSFSQHFRMMVICIVLLQVLLQAVSVAVTYMYFTNEMKQLQD
NYSKIGLACFSKTDEDFWDSTDGEILNRPCLQVKRQLYQLIEEVTLRTFQDTISTVPEK
QLSTPPLPRGGRPQKVAAHITGITRRSNSALIPISKDGKTLGQKIESWESSRKGHSFLNH
VLFRNGELVIEQEGLYYIYSQTYFRFQEAEDASKMVSKDKVRTKQLVQYIYKYTSYP
DPIVLMKSARNSCWSRDAEYGLYSIYQGGLFELKKNDRIFVSVTNEHLMDLDQEASF
FGAFLIN [SEQ ID NO: 52] (corresponding to a putative full-length Mus musculus
TRAIL, as set forth in NCBI Accession: NP_033451).

A biologically active fragment of a full-length TRAIL polypeptide can be a polypeptide which is, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more amino acid residues in length. Suitably, the biologically-active fragment has no less than about 1%, 10%, 25% 50% of an activity of the full-length TRAIL polypeptide from which it is derived.

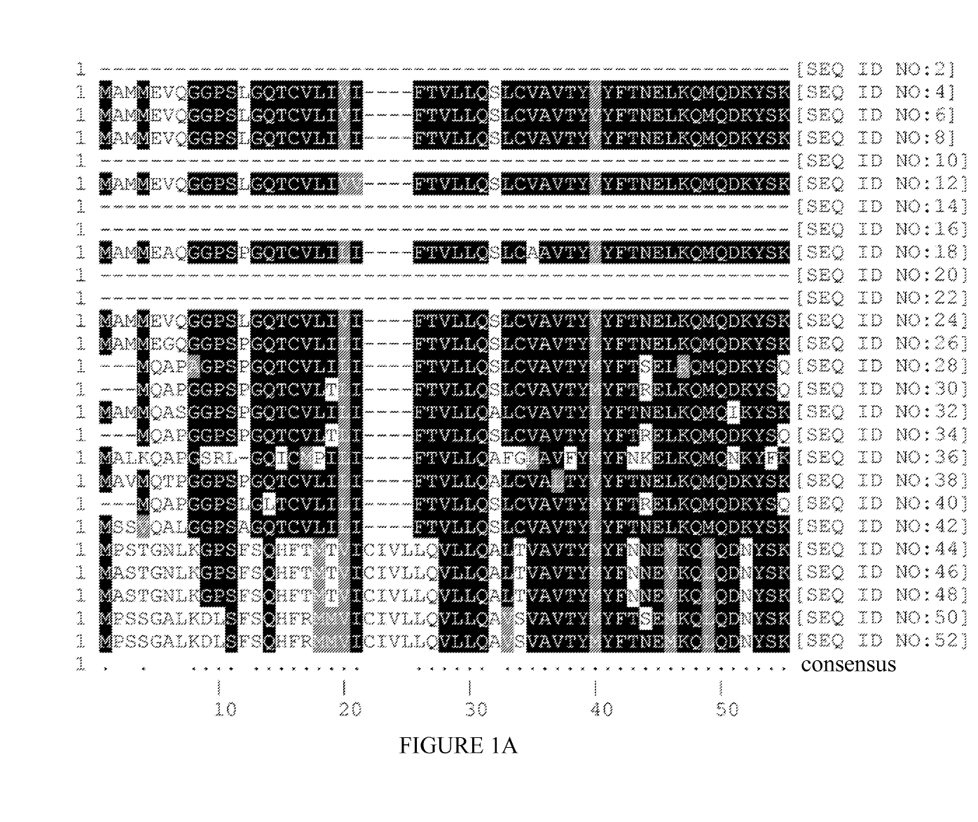

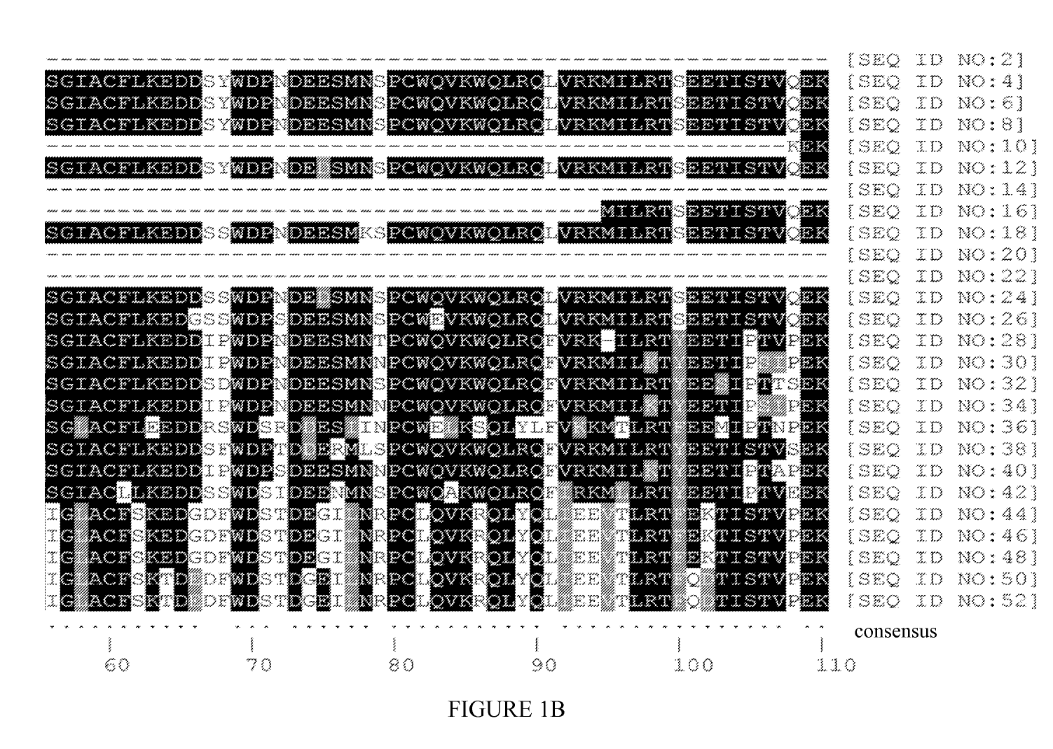

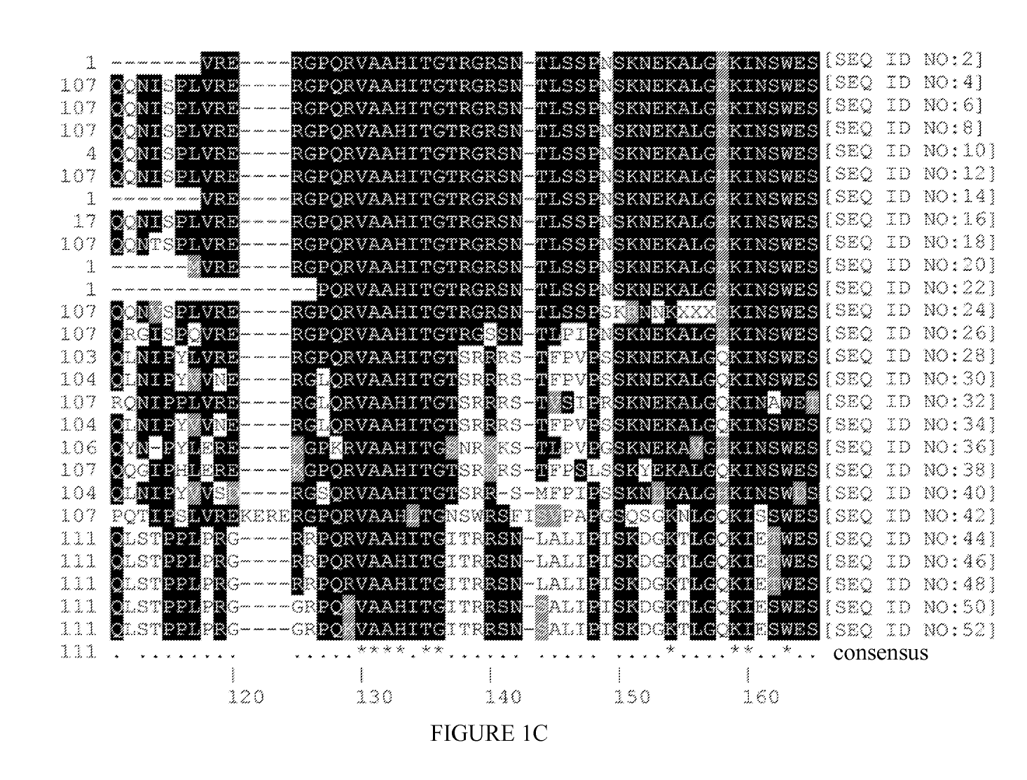

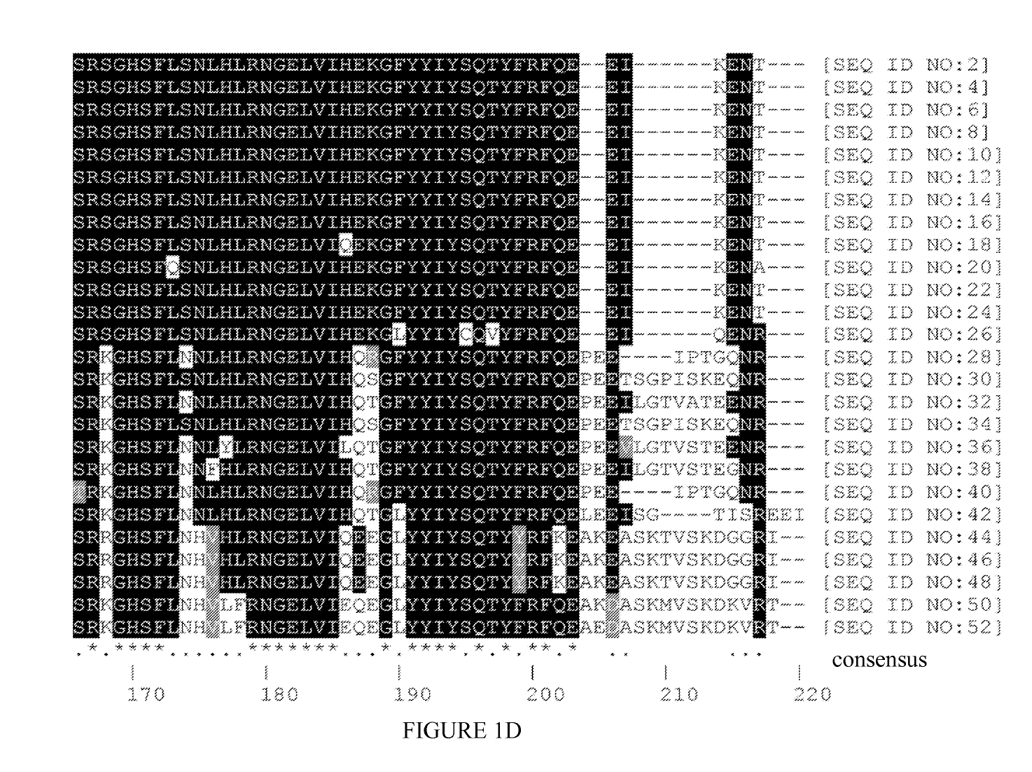

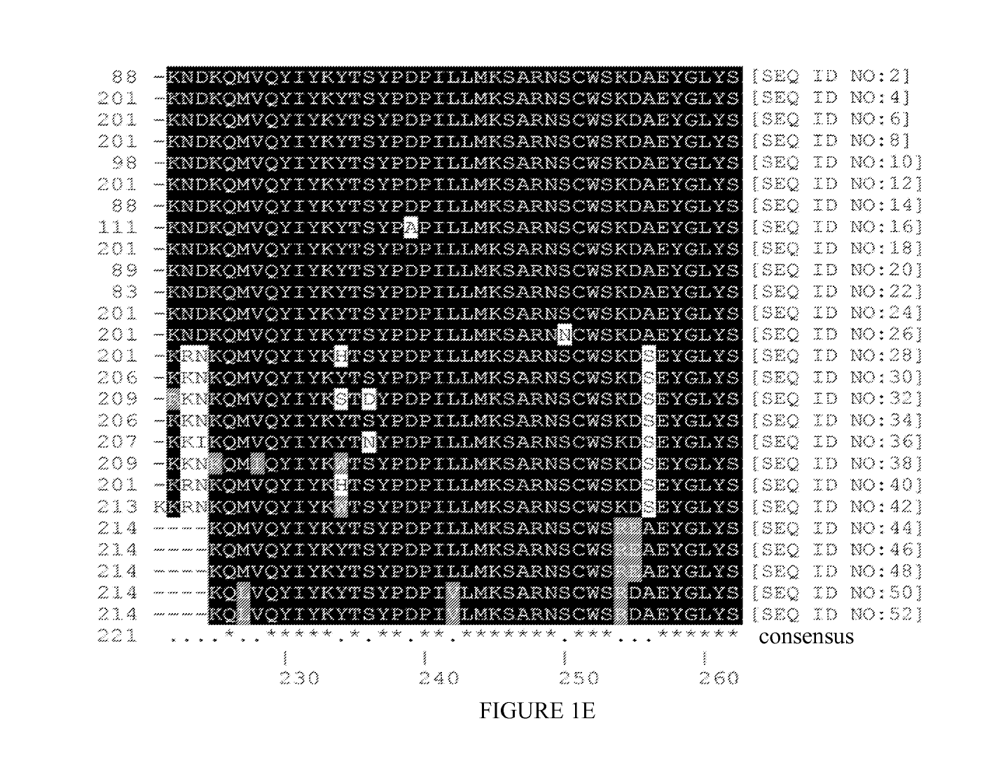

Typically, biologically active fragments will comprise a domain or motif with at least one activity of a putatively full-length TRAIL polypeptide and may include all or part of a TRAIL extracellular domain (e.g., from about amino acid 43 to about amino acid 301, relative to the consensus numbering shown in FIG. 1). Suitably, the TRAIL extracellular domain comprises, consists, or consists essentially of an amino acid sequence spanning from about amino acid 43 to about amino acid 301 (relative to the consensus numbering shown in FIG. 1). In illustrative examples, the soluble fragment comprises, consists or consists essentially of amino acid X to amino acid Y, wherein X represents any of the amino acids at about position 43 to about position 132 relative to the same consensus numbering shown in FIG. 1 and Y represents any of the amino acids at about position 297 to position 303 relative to the same consensus numbering.

In some embodiments, the TRAIL polypeptides will comprise relative to the consensus numbering shown in FIG. 1:

an A, or modified form thereof, at position 132; a H, or modified form thereof, at position 133; an hydrophobic amino acid residue (e.g., selected from aliphatic amino acid residues such as I or L, or modified form thereof) at position 134; a T, or modified form thereof, at position 135; G, or modified form thereof, at position 136; any amino acid residue (e.g., selected from small amino acid residues such as T, or modified form thereof; neutral/polar amino acid residues such as N, or modified form thereof; or hydrophobic amino acid residues including aliphatic amino acid residues such as I, or modified form thereof) at position 137; a basic amino acid residue (e.g., R, or modified form thereof) or a small amino acid residue (e.g., S or T, or modified form thereof) at position 138; any amino acid residue (e.g., selected from small amino acid residues such as G, or modified form thereof; basic amino acid residues such as R, or modified form thereof; or hydrophobic amino acid residues including aromatic amino acid residues such as W, or modified form thereof) at position 139; a basic amino acid residue (e.g., R or K, or modified form thereof); or a small amino acid residue (e.g., S, or modified form thereof) at position 140;

a small amino acid residue (e.g., S, or modified form thereof) or a basic amino acid residue (e.g., R or K, or modified form thereof) at position 141, which is optionally present; any amino acid residue (e.g., selected from neutral/polar amino acid residues such as N, or modified form thereof; or small amino acid residues such as S, or modified form thereof; or hydrophobic amino acid residues including aromatic amino acid residues such as F, or modified form thereof) at position 142; an hydrophobic amino acid residue (e.g., selected from aliphatic amino acid residues such as I, or modified form thereof) at position 143, which is optionally present; a small amino acid residue (e.g., T or S, or modified form thereof) or an hydrophobic amino acid residues (e.g., selected from aliphatic amino acid residues such as M or L, or modified form thereof) at position 144; an hydrophobic amino acid residue (e.g., selected from aliphatic amino acid residues such as L or V, or modified form thereof; or aromatic amino acid residues such as F, or modified form thereof) or a small amino acid residue (e.g., A, or modified form thereof) at position 145; a small amino acid residue (e.g., S or P, or modified form thereof) or an hydrophobic amino acid residue (e.g., selected from aliphatic amino acid residues such as L, or modified form thereof) at position 146; a small amino acid residue (e.g., S or A, or modified form thereof) or an hydrophobic amino acid residue (e.g., selected from aliphatic amino acid residues such as I or V, or modified form thereof) at position 147; a small amino acid residue (e.g., P, or modified form thereof) or an hydrophobic amino acid residue (e.g., aliphatic amino acid residues such as L, or modified form thereof) at position 148; any amino acid residue (e.g., selected from neutral/polar amino acid residues such as N, or modified form thereof; small amino acid residues such as S or G, or modified form thereof; or hydrophobic amino acid residues including aliphatic amino acid residues such as I, or modified form thereof; or basic amino acid residues such as R, or modified form thereof) at position 149; a small amino acid residue (e.g., S, or modified form thereof) or a basic amino acid residue (e.g., K, or modified form thereof) at position 150;

a basic amino acid residue (e.g., K or R, or modified form thereof) or a neutral/polar amino acid residue (e.g., N, or modified form thereof) at position 151; any amino acid residue (e.g., selected from neutral/polar amino acid residues such as N, or modified form thereof; acidic amino acid residues such as D, or modified form thereof; or small amino acid residues such as S, or modified form thereof; or hydrophobic amino acid residues including aromatic amino acid residues such as Y, or modified form thereof) at position 152; any amino acid residue (e.g., selected from acidic amino acid residues such as E or D, or modified form thereof; small amino acid residues such as G, or modified form thereof; or neutral/polar amino acid residues such as N, or modified form thereof) at position 153; a K, or modified form thereof, at position 154; a small amino acid residue (e.g., A or T, or modified form thereof) or a neutral polar amino acid residue (e.g., N, or modified form thereof) at position 155; an hydrophobic residue (e.g., selected from aliphatic amino acid residues such as L or V, or modified forms thereof) at position 156; G, or modified form thereof, at position 157; a basic amino acid residue (e.g., R or H, or modified form thereof) or a neutral/polar amino acid residue (e.g., Q, or modified form thereof) at position 158; a K, or modified form thereof, at position 159, an I, or modified form thereof, at position 160;

any amino acid residue (e.g., neutral/polar amino acid residues such as N, or modified form thereof; acidic amino acid residues such as E, or modified form thereof; or small amino acid residues such as S, or modified form thereof) at position 161; a small amino acid residue (e.g., S or A, or modified form thereof) at position 162; a W, or modified form thereof, at position 163; an acidic amino acid residue (e.g., E or D, or modified form thereof) at position 164; a small amino acid residue (e.g., S or T, or modified form thereof) at position 165; a small amino acid residue (e.g., S or T, or modified form thereof) at position 166; a R, or modified form thereof, at position 167; a small amino acid residue (e.g., S, or modified form thereof) or a basic amino acid residue (e.g., K or R, or modified form thereof) at position 168; a G, or modified form thereof, at position 169; a H, or modified form thereof, at position 170;

a S, or modified form thereof, at position 171; a F, or modified form thereof, at position 172; an hydrophobic amino acid residue (e.g., selected from aliphatic amino acid residues such as L, or modified form thereof) or a neutral/polar amino acid residue (e.g., Q, or modified form thereof) at position 173; a small amino acid residue (e.g., S, or modified form thereof) or neutral/polar amino acid residue (e.g., N, or modified form thereof) at position 174; a neutral/polar amino acid residue (e.g., N, or modified form thereof) or a basic amino acid residue (e.g., H, or modified form thereof) at position 175; an hydrophobic residue (e.g., selected from aliphatic amino acid residues such as L or V, or modified form thereof or aromatic amino acid residues such as F, or modified form thereof) at position 176; a basic amino acid residue (e.g., H, or modified form thereof) or an hydrophobic amino acid residue (e.g., selected from aliphatic amino acid residues such as L, or modified form thereof, or aromatic amino acid residues such as Y, or modified form thereof) at position 177; an hydrophobic amino acid residue (e.g., selected from aliphatic amino acid residues such as L, or modified form thereof, or aromatic amino acid residues such as F, or modified form thereof) at position 178; a R, or modified form thereof, at position 179, a N, or modified form thereof, at position 180;

a G, or modified form thereof, at position 181; an E, or modified form thereof, at position 182; a L, or modified form thereof, at position 183; a V, or modified form thereof, at position 184; an I, or modified form thereof, at position 185; any amino acid residue (e.g., selected from basic amino acid residues such as H, or modified form thereof; neutral/polar amino acid residues such as Q, or modified form thereof; acidic amino acid residues such as E, or modified form thereof; or hydrophobic amino acid residues including aliphatic amino acid residues such as L, or modified form thereof) at position 186; an acidic amino acid residue (e.g., E, or modified form thereof) or a neutral/polar amino acid residue (e.g., Q, or modified form thereof) at position 187; any amino acid residue (e.g., selected from basic amino acid residues such as K or R, or modified form thereof; acidic amino acid residues such as E, or modified form thereof; or small amino acid residues such as T or S, or modified form thereof) at position 188; a G, or modified form thereof, at position 189; an hydrophobic amino acid residue (e.g., selected from aromatic amino acid residues such as F, or modified form thereof, or aliphatic amino acid residues such as L, or modified form thereof) at position 190;

a Y, or modified form thereof, at position 191; a Y, or modified form thereof, at position 192; an I, or modified form thereof, at position 193; a Y, or modified form thereof, at position 194; a small amino acid residue (e.g., S, or modified form thereof) or a neutral/polar amino acid residue (e.g., C, or modified form thereof) at position 195; a Q, or modified form thereof, at position 196; a small amino acid residue (e.g., T, or modified form thereof) or an hydrophobic amino acid residue (e.g., selected from aliphatic amino acid residues such as V, or modified form thereof) at position 197; a Y, or modified form thereof, at position 198; an hydrophobic amino acid residue (e.g., selected from aromatic amino acid residues such as F or Y, or modified form thereof) at position 199; a R, or modified form thereof, at position 200;

a F, or modified form thereof, at position 201; a neutral/polar amino acid residue (e.g., Q, or modified form thereof) or a basic amino acid residue (e.g., K, or modified form thereof) at position 202; an E, or modified form thereof, at position 203; a small amino acid residue (e.g., P or A, or modified form thereof) or an hydrophobic amino acid residue (e.g., selected from aliphatic amino acid residues such as L, or modified form thereof) at position 204, which is optionally present; a charged amino acid residue (e.g., selected from acidic amino acid residues such as E, or modified form thereof; or basic amino acid residues such as K, or modified form thereof) at position 205, which is optionally present; an acidic amino acid residue (e.g., E or D, or modified form thereof) at position 206; an hydrophobic amino acid residue (e.g., selected from aliphatic amino acid residues such as I or V, or modified form thereof), or a small amino acid residue (e.g., T or A, or modified form thereof) at position 207, which is optionally present; a small amino acid residue (e.g., S, or modified form thereof) or an hydrophobic amino acid residue (e.g., selected from aliphatic amino acid residues such as L, or modified form thereof) at position 208, which is optionally present; a small amino acid residue (e.g., G, or modified form thereof) or a basic amino acid residue (e.g., K, or modified form thereof) at position 209, which is optionally present; a small amino acid residue (e.g., P or T, or modified form thereof) or an hydrophobic amino acid residue (e.g., selected from aliphatic amino acid residues such as M, or modified form thereof) at position 210, which is optionally present;

an hydrophobic amino acid residue (e.g., selected from aliphatic amino acid residues such as V or I, or modified form thereof) at position 211, which is optionally present; a small amino acid residue (e.g., P, S or A, or modified form thereof) at position 212, which is optionally present; a basic amino acid residue (e.g., K, or modified form thereof) or a small amino acid residue (e.g., T, or modified form thereof) at position 213, which is optionally present; any amino acid residue (e.g., selected from basic amino acid residues such as K, or modified form thereof acidic amino acid residues such as D or E, or modified form thereof small amino acid residues such as G or T, or modified form thereof or neutral/polar amino acid residues such as Q, or modified form thereof) at position 214; any amino acid residue (e.g., selected from acidic amino acid residues such as E, or modified form thereof; small amino acid residues such as G or T, or modified form thereof; basic amino acid residues such as K, or modified form thereof; neutral/polar amino acid residues such as Q, or modified form thereof, or hydrophobic amino acid residues including aliphatic amino acid residues such as I, or modified form thereof) at position 215; any amino acid residue (e.g., selected from neutral/polar amino acid residues such as N, or modified form thereof; small amino acid residues such as G or S, or modified form thereof; or hydrophobic amino acid residues including aliphatic amino acid residues such as V, or modified form thereof) at position 216; a small amino acid residue (e.g., T, or modified form thereof) or a basic amino acid residue (e.g., R, or modified form thereof) at position 217; any amino acid residue (e.g., selected from hydrophobic amino acid residues including aliphatic amino acid residues such as I, or modified form thereof; small amino acid residues such as T, or modified form thereof; or acidic amino acid residues such as E, or modified form thereof) at position 218, which is optionally present; an acidic amino acid residue (e.g., E, or modified form thereof) at position 219, which is optionally present; an hydrophobic amino acid residue (e.g., selected from aliphatic amino acid residues such as I, or modified form thereof) at position 220;

a basic amino acid residue (e.g., K, or modified form thereof) at position 221, which is optionally present; a basic amino acid residue (e.g., K, or modified form thereof) at position 222, which is optionally present; a neutral/polar amino acid residue (e.g., N, or modified form thereof) or a basic amino acid residue (e.g., K or R, or modified form thereof) at position 223, which is optionally present; any amino acid residue (e.g., selected from acidic amino acid residues such as D, or modified form thereof), or neutral/polar amino acid residues (e.g., N or modified form thereof) or hydrophobic amino acid residues (e.g., aliphatic amino acid residues such as I, or modified form thereof) at position 224; a basic amino acid residue (e.g., K or R, or modified forms thereof) at position 225; a Q, or modified form thereof, at position 226; an hydrophobic amino acid residue (e.g., aliphatic amino acid residues such as M or L, or modified form thereof) at position 227; an hydrophobic amino acid residue (e.g., selected from aliphatic amino acid residues such as V or I, or modified form thereof) at 228; Q, or modified form thereof, at position 229; a Y, or modified form thereof, at position 230;

an I, or modified form thereof, at position 231; a Y, or modified form thereof, at position 232; a K, or modified form thereof, at position 233; any amino acid residue (e.g., selected from hydrophobic amino acid residues including aromatic amino acid residues such as Y or W, or modified form thereof; basic amino acid residues such as H, or modified form thereof; or small amino acid residues such as S, or modified form thereof) at position 234; T, or modified form thereof, at position 235; any amino acid residue (e.g., selected from small amino acid residues such as S, or modified form thereof; acidic amino acid residues such as D, or modified form thereof; or neutral/polar amino acid residues such as N, or modified form thereof) at position 236; a Y, or modified form thereof, at position 237; a P, or modified form thereof, at position 238; an acidic amino acid residue (e.g., D, or modified form thereof) or a small amino acid residue (e.g., A, or modified form thereof) at position 239; a P, or modified form thereof, at position 240;

an I, or modified form thereof, at position 241; an hydrophobic amino acid residue (e.g., selected from aliphatic amino acid residues such as L or V, or modified form thereof) at position 242; a L, or modified form thereof, at position 243; a M, or modified form thereof, at position 244; a K, or modified form thereof, at position 245; a S, or modified form thereof, at position 246; an A, or modified form thereof, at position 247; a R, or modified form thereof, at position 248; a N, or modified form thereof, at position 249; a small amino acid residue (e.g., S, or modified form thereof) or a neutral/polar amino acid residue (e.g., N, or modified form thereof) at position 250;

a C, or modified form thereof, at position 251; a W, or modified form thereof, at position 252; a S, or modified form thereof, at position 253; a basic amino acid residue (e.g., K or R, or modified forms thereof) at position 254; an acidic amino acid residue (e.g., D or E, or modified form thereof) at position 255; a small amino acid residue (e.g., A or S, or modified form thereof) at position 256; an E, or modified form thereof, at position 257; a Y, or modified form thereof, at position 258; a G, or modified form thereof, at position 259; a L, or modified form thereof, at position 260;

a Y, or modified form thereof, at position 261; a S, or modified form thereof, at position 262; an I, or modified form thereof, at position 263; a Y, or modified form thereof, at position 264; a Q, or modified form thereof, at position 265; a G, or modified form thereof, at position 266; a G, or modified form thereof, at position 267; an hydrophobic amino acid residue (e.g., selected from aliphatic amino acid residues such as I or L, or modified form thereof) at position 268; a F, or modified form thereof, at position 269; an E, or modified form thereof, at position 270;

a L, or modified form thereof, at position 271; a K, or modified form thereof, at position 272; a charged amino acid residue (e.g., selected from acidic amino acid residues such as E, or modified form thereof; or basic amino acid residues such as K, or modified form thereof) at position 273; a neutral/polar amino acid residue (e.g., N, or modified form thereof) or an acidic amino acid residue (e.g., D, or modified form thereof) at position 274; a D, or modified form thereof, at position 275; a R, or modified form thereof, at position 276; an I, or modified form thereof, at position 277; a F, or modified form thereof, at position 278; a V, or modified form thereof, at position 279; a S, or modified form thereof, at position 280;

a V, or modified form thereof, at position 281; a small amino acid residue (e.g., T, or modified form thereof) or a neutral/polar amino acid residue (e.g., N, or modified form thereof) at position 282; a N, or modified form thereof, at position 283; an acidic amino acid residues (e.g., E, or modified form thereof) or small amino acid residues (e.g., G, or modified form thereof) at position 284; a basic amino acid residue (e.g., H, or modified form thereof) or a neutral/polar amino acid residue (e.g., Q, or modified form thereof) at position 285; a L, or modified form thereof, at position 286; an hydrophobic amino acid residue (e.g., selected from aliphatic amino acid residues such as I, M or V, or modified form thereof) at position 287; a D, or modified form thereof, at position 288; an hydrophobic amino acid residue (e.g., selected from aliphatic amino acid residues such as M or L, or modified form thereof) at position 289; an acidic amino acid residue (e.g., D, or modified form thereof) or a neutral/polar amino acid residue (e.g., N, or modified form thereof) at position 290;

a basic amino acid residue (e.g., H, or modified form thereof) or a neutral/polar amino acid residues (e.g., Q, or modified form thereof) at position 291; a E, or modified form thereof, at position 292; a small amino acid residue (e.g., A or S, or modified form thereof) at position 293; a S, or modified form thereof, at position 294; a F, or modified form thereof, at position 295; an hydrophobic amino acid residue (e.g., selected from aromatic amino acid residues such as F, or modified form thereof; or aliphatic amino acid residues such as L, or modified form thereof) at position 296; and G, or modified form thereof at position 297.

In illustrative examples of this type, the biologically active fragment comprises, consists or consists essentially of an amino acid sequence selected from:

AHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFFG [SEQ ID NO: 54]
(corresponding to amino acids 124-276 of a human TRAIL isoform 1, as set forth in NCBI
Accession: NP_003801);
AHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFFG [SEQ ID NO: 56]
(corresponding to amino acids 124-276 of a synthetic TRAIL, as set forth in NCBI
Accession: AAV38370);
AHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFLG [SEQ ID NO: 58]
(corresponding to amino acids 124-276 of a synthetic TRAIL, as set forth in NCBI
Accession: AAX29952);
AHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFFG [SEQ ID NO: 60]
(corresponding to amino acids 21-173 of a human TRAIL isoform CRA_b, as set forth in
NCBI Accession: EAW78466);
AHITGTRGRSNTLSSPNSKNEKALGHKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFFG [SEQ ID NO: 62]
(corresponding to amino acids 124-276 of a Pan troglodytes TRAIL, as set forth in NCBI
Accession: XP_516879);
AHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKD
AEYGLYSIYQGGIFELKENDRIFVSVTNEHLIDMDHEASFFG [SEQ ID NO: 64]
(corresponding to amino acids 11-163 of a human TRAIL fragment, as set forth in NCBI
Accession: 1D0G_A);
AHITGTRGRSNTLSSPNSKNEKALGRKINSWESSRSGHSFLSNLHLRNGELVIH
EKGFYYIYSQTYFRFQEEIKENTKNDKQMVQYIYKYTSYPAPILLMKSARNSCWSKD
AEYGLYSIYQGGIFELKENDRIFVSVTNEHLIDMDHEASFFG [SEQ ID NO: 66]
(corresponding to amino acids 34-186 of a human TRAIL fragment, as set forth in NCBI
Accession: 1DG6);
AHITGTRGRSNTLSSPNSKNEKALGRKINSWESSRSGHSFLSNLHLRNGELVIQ
EKGFYYIYSQTYFRFQEEIKENTKNDKQMVQYIYKYTSYPDPILLMKSARNSCWSKD
AEYGLYSIYQGGLFELKKDDRIFVSVTNEHLIDMDHEASFFG [SEQ ID NO: 68]
(corresponding to amino acids 124-276 of a Macaca mulatta TRAIL, as set forth in NCBI
Accession: XP_001084768);
AHITGTRGRSNTLSSPNSKNEKALGRKINSWESSRSGHSFQSNLHLRNGELVIH
EKGFYYIYSQTYFRFQEEIKENAKNDKQMVQYIYKYTSYPDPILLMKSARNSCWSKD
AEYGLYSIYQGGIFELKENDRIFVSVTNEHLIDMDHEASFFG [SEQ ID NO: 70]
(corresponding to amino acids 7-164 of a Crassostrea ariakensis TRAIL, as set forth
in NCBI Accession: ABU39827);
PQRVAAHITGTRGRSNTLSSPNSKNEKALGRKINSWESSRSGHSFLSNLHLRN
GELVIHEKGFYYIYSQTYFRFQEEIKENTKNDKQMVQYIYKYTSYPDPILLMKSARNS
CWSKDAEYGLYSIYQGGIFELKENDRIFVSVTNEHLIDMDHEASFFG [SEQ ID NO: 72]
(corresponding to amino acids 1-158 of a human TRAIL fragment, as set forth in NCBI
Accession: 1D4V_B);
PQRVAAHITGTRGRSNTLSSPSKRNNKXXXRKINSWESSRSGHSFLSNLHLRN
GELVIHEKGFYYIYSQTYFRFQEEIKENTKNDKQMVQYIYKYTSYPDPILLMKSARNS
CWSKDAEYGLYSIYQGGIFELKENDRIFVSVTNEHLIDMDHEASFFG [SEQ ID NO: 74]
(corresponding to amino acids 119-276 of a Pongo abelii TRAIL, as set forth in NCBI
Accession: XP_002814335);
PQRVAAHITGTRGSSNTLPIPNSKNEKALGRKINSWESSRSGHSFLSNLHLRNG
ELVIHEKGLYYIYCQVYFRFQEEIQENRKNDKQMVQYIYKYTSYPDPILLMKSARNN
CWSKDAEYGLYSIYQGGIFELKENDRIFVSVTNGQLIDMDHEASFFG [SEQ ID NO: 76]
(corresponding to amino acids 119-276 of a Callithrix jacchus TRAIL, as set forth
in NCBI Accession: XP_002759427);
PQRVAAHITGTSRRRSTFPVPSSKNEKALGQKINSWESSRKGHSFLNNLHLRN
GELVIHQRGFYYIYSQTYFRFQEPEEIPTGQNRKRNKQMVQYIYKHTSYPDPILLMKS
ARNSCWSKDSEYGLYSIYQGGIFELKENDRIFVSVSNEQLIDMDQEASFFG [SEQ ID
NO: 78] (corresponding to amino acids 115-276 of a Felis catus TRAIL, as set forth
in NCBI Accession: NP_001124316);
LQRVAAHITGTSRRRSTFPVPSSKNEKALGQKINSWESSRKGHSFLSNLHLRN
GELVIHQSGFYYIYSQTYFRFQEPEETSGPISKEQNRKKNKQMVQYIYKYTSYPDPILL
MKSARNSCWSKDSEYGLYSIYQGGIFELKENDRIFVSVNNEQLIDMDQEASFFG [SEQ
ID NO: 80] (corresponding to amino acids 116-281 of an Ailuropoda melanoleuca TRAIL,
as set forth in NCBI Accession: XP_002921635);
LQRVAAHITGTSRRRSTVSIPRSKNEKALGQKINAWETSRKGHSFLNNLHLRN
GELVIHQTGFYYIYSQTYFRFQEPEEILGTVATEENRRKNKQMVQYIYKSTDYPDPILL
MKSARNSCWSKDSEYGLYSIYQGGIFELKENDRIFVSVTNEQLIDMDQEASFFG [SEQ
ID NO: 82] (corresponding to amino acids 124-284 of an Equus caballus TRAIL,
as set forth in NCBI Accession: XP_001494138);
AHITGTSRRRSTFPVPSSKNEKALGQKINSWESSRKGHSFLSNLHLRNGELVIH
QSGFYYIYSQTYFRFQEPEETSGPISKEQNRKKNKQMVQYIYKYTSYPDPILLMKSAR
NSCWSKDSEYGLYSIYQGGIFELKENDRIFVSVNNEQLIDMDQEASFFG [SEQ ID NO:
84] (corresponding to amino acids 121-276 of an Ailuropoda melanoleuca TRAIL,
as set forth in NCBI Accession: EFB16787);
AHITGSNRKKSTLPVPGSKNEKAVGHKINSWESSRKGHSFLNNLYLRNGELVI
LQTGFYYIYSQTYFRFQEPEEVLGTVSTEENRKKIKQMVQYIYKYTNYPDPILLMKSA
RNSCWSKDSEYGLYSIYQGGIFELKENDRIFVSVTNERLVDLDQEASFFG [SEQ ID
NO: 86] (corresponding to amino acids 122-282 of a Bos taurus TRAIL, as set forth
in NCBI Accession: XP_583785);
AHITGTSRKRSTFPSLSSKYEKALGQKINSWESSRKGHSFLNNFHLRNGELVIH
QTGFYYIYSQTYFRFQEPEEILGTVSTEGNRKKNRQMIQYIYKWTSYPDPILLMKSAR
NSCWSKDSEYGLYSIYQGGIFELKEDDRIFVSVTNEQLIDMDQEASFFG [SEQ ID NO:
88] (corresponding to amino acids 124-284 of a Sus scrofa TRAIL, as set forth
in NCBI Accession: NP_001019867);
AHITGTSRRSMFPIPSSKNDKALGHKINSWDSTRKGHSFLNNLHLRNGELVIHQ
RGFYYIYSQTYFRFQEPEEIPTGQNRKRNKQMVQYIYKHTSYPDPILLMKSARNSCWS
KDSEYGLYSIYQGGIFELKENDRIFVSVSNEQLIDMDQEASFFG [SEQ ID NO: 90]
(corresponding to amino acids 124-276 of a Canis lupis familiaris TRAIL, as set forth in
NCBI Accession: NP_001124308);
AHLTGNSWRSFISVPAPGSQSGKNLGQKISSWESSRKGHSFLNNLHLRNGELVI
HQTGLYYIYSQTYFRFQELEEISGTISREEIKKRNKQMVQYIYKWTSYPDPILLMKSAR
NSCWSKDSEYGLYSIYQGGIFELKENDRIFVSVTNEQLIDMNQESSFFG [SEQ ID NO:
92] (corresponding to amino acids 128-289 of an Oryctolagus cuniculus TRAIL,
as set forth in NCBI Accession: XP_002716472);
AHITGITRRSNLALIPISKDGKTLGQKIETWESSRRGHSFLNHVHLRNGELVIQE
EGLYYIYSQTYYRFKEAKEASKTVSKDGGRIKQMVQYIYKYTSYPDPILLMKSARNS
CWSREAEYGLYSIYQGGLFELKENDRIFVSVTNEHLMDLDQEASFFG [SEQ ID NO:
94] (corresponding to amino acids 128-286 of a Rattus novegicus TRAIL, as set forth in
NCBI Accession: EDM01114);
AHITGITRRSNLALIPISKDGKTLGQKIETWESSRRGHSFLNHVHLRNGELVIQE
EGLYYIYSQTYYRFKEAKEASKTVSKDGGRIKQMVQYIYKYTSYPDPILLMKSARNS
CWSREAEYGLYSIYQGGLFELKENDRIFVSVTNEHLMDLDHEASFFG [SEQ ID NO:
96] (corresponding to amino acids 128-286 of a Rattus novegicus TRAIL, as set forth in
NCBI Accession: NP_663714);
AHITGITRRSNLALIPISKDGKTLGQKIETWESSRRGHSFLNHVHLRNGELVIQE
EGLYYIYSQTYYRFKEAKEASKTVSKDGGRIKQMVQYIYKYTSYPDPILLMKSARNS
CWSREAEYGLYSIYQGGLFELKENDRIFVSVTNEHLMDLDQEASFFG [SEQ ID NO:
98] (corresponding to amino acids 128-286 of a Rattus novegicus TRAIL, as set forth in
NCBI Accession: ABK32522);
AHITGITRRSNSALIPISKDGKTLGQKIESWESSRKGHSFLNHVLFRNGELVIEQ
EGLYYIYSQTYFRFQEAKDASKMVSKDKVRTKQLVQYIYKYTSYPDPIVLMKSARNS
CWSRDAEYGLYSIYQGGLFELKKNDRIFVSVTNEHLMDLDQEASFFG [SEQ ID NO:
100] (corresponding to amino acids 128-286 of a Mus musculus TRAIL, as set forth
in NCBI Accession: BAE34141); and
AHITGITRRSNSALIPISKDGKTLGQKIESWESSRKGHSFLNHVLFRNGELVIEQ
EGLYYIYSQTYFRFQEAEDASKMVSKDKVRTKQLVQYIYKYTSYPDPIVLMKSARNS
CWSRDAEYGLYSIYQGGLFELKKNDRIFVSVTNEHLMDLDQEASFFG [SEQ ID NO:
102] (corresponding to amino acids 128-286 of a Mus musculus TRAIL, as set forth
in NCBI Accession: NP_033451).

In some embodiments, the biologically active fragment further comprises upstream (e.g., immediately upstream) of position 132, about 1, 2, 3, 4, 5, 6, 7, 8, 9, 10, 11, 12, 13, 14, 15 or more additional amino acids. For example, the biologically active fragment may further comprise, relative to the consensus numbering shown in FIG. 1, any one or more of: an A, or modified form thereof, at position 131; a V, or modified form thereof, at position 130; a basic amino acid residue (e.g., K or R, or modified forms thereof) at position 129; a neutral/polar amino acid residue (e.g., Q, or modified form thereof) or a basic amino acid residue (e.g., K, or modified form thereof) at position 128; a small amino acid residue (e.g., P or S, or modified form thereof) or an hydrophobic amino acid residue (e.g., selected from aliphatic amino acid residues such as L, or modified form thereof) at position 127; a small amino acid residue (e.g., G, or modified form thereof) or a basic amino acid residue (e.g., R, or modified form thereof) at position 126; a basic amino acid residue (e.g., R or K, or modified form thereof) or a small amino acid residue (e.g., G, or modified form thereof) at position 125; an acidic amino acid residue (e.g., E, or modified form thereof) at position 124, which is optionally present; a basic amino acid residue (e.g., R, or modified form thereof) at position 123, which is optionally present; an acidic amino acid residue (e.g., E, or modified form thereof) at position 122, which is optionally present; a basic amino acid residue (e.g., K, or modified form thereof) at position 121, which is optionally present; an acidic amino acid residue (e.g., E or D, or modified form thereof) or a small amino acid residue (e.g., G, or modified form thereof) at position 120; any amino acid residue (e.g., selected from basic amino acid residues such as R, or modified form thereof; neutral/polar amino acid residues such as N, or modified form thereof, or small amino acid residues such as S, or modified form thereof) at position 119; and any amino acid residue (e.g., selected from hydrophobic amino acid residues including aliphatic amino acid residues such as V, or modified form thereof; acidic amino acid residues such as E, or modified form thereof, or small amino acid residues such as P, or modified form thereof) at position 118.

Illustrative upstream sequences of this type may be selected from VRERGPQRVA [SEQ ID NO:104], PQRVA [SEQ ID NO: 106], VNERGLQRVA [SEQ ID NO: 108], VRERGLQRVA [SEQ ID NO: 110], EREKGPKRVA [SEQ D NO: 112], EREKGPQRVA [SEQ ID NO: 114], VSDRGSQRVA [SEQ ID NO: 116], VREKERERGPQRVA [SEQ ID NO: 118], PRGRRPQRVA [SEQ ID NO: 120] or PRGGRPQRVA [SEQ ID NO: 122].

In some embodiments, the biologically active fragment further comprises downstream (e.g., immediately downstream) of position 297, about 1, 2, 3, 4, 5, 6 or more additional amino acids. For example, the biologically active fragment may further comprise, relative to the consensus numbering shown in FIG. 1, any one or more of: an A, or modified form thereof, at position 298; a F, or modified form thereof at position 299; an hydrophobic amino acid residue (e.g., selected from aliphatic amino acid residues such as L, or modified form thereof) at position 300; an hydrophobic amino acid residue (e.g., selected from aliphatic amino acid residues such as V or I, or modified form thereof) at position 301, a small amino acid residue (e.g., G, or modified form thereof) or neutral/polar amino acid residue (e.g., N, or modified form thereof), at position 302; and an hydrophobic amino acid residue (e.g., selected from aliphatic amino acid residues such as L, or modified form thereof) at position 303.

Non-limiting examples of such downstream sequences may be selected from: AFLVG [SEQ ID NO: 124], AFLVGL [SEQ ID NO: 126], AF [SEQ ID NO: 128], AFLIG [SEQ ID NO: 130] or AFLIN [SEQ ID NO: 132].

Illustrative examples of biologically active fragments comprising additional upstream and/or downstream amino acids include:

VRERGPQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVG
[SEQ ID NO: 2] (corresponding to amino acids 114-281 of a human
TRAIL isoform 1, as set forth in NCBI Accession: NP_003801);
PQRVAAHITGTRGRSNTLSSPNSKNEKALGRKINSWESSRSGHSFLSNLHLRN
GELVIHEKGFYYIYSQTYFRFQEEIKENTKNDKQMVQYIYKYTSYPDPILLMKSARNS
CWSKDAEYGLYSIYQGGIFELKENDRIFVSVTNEHLIDMDHEASFFGAFLVG[SEQ ID
NO: 22] (corresponding to a human TRAIL fragment, as set forth in
NCBI Accession: 1D4V_B); and
VRERGPQRVAAHITGTRGRSNTLSSPNSKNEKALGRKINSWESSRSGHSFLSNL
HLRNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVG
[SEQ ID NO: 14] (corresponding to a human TRAIL fragment, as set
forth in NCBI Accession: 1D0G_A).

In some embodiments, the biologically active fragments comprise one or more amino acids that stimulate or are otherwise involved in trimerization (e.g., 1, 2, 3, 4, 5 or each of the amino acids at positions 133, 192, 261, 269, 295 and 299, relative to the consensus numbering shown in FIG. 1). In some embodiments, the biologically active fragments comprise one or more amino acids that stimulate or are otherwise involved in interaction with a TRAIL DR (e.g., 1, 2, 3, 4, 5 or each of the amino acids at positions 164, 165, 171, 228, 223595 and 239, relative to the consensus numbering shown in FIG. 1).

Specific examples of TRAIL polypeptides including soluble TRAIL fragments and TRAIL oligomers are disclosed in US 2010/0323399, which is incorporated by reference herein in its entirety.

The present invention also contemplates TRAIL, polypeptides that are variants of wild-type or naturally-occurring TRAIL polypeptides or their fragments. Such “variant” peptides or polypeptides include proteins derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Non-limiting examples of such variant TRAIL polypeptides include TRAIL polypeptides lacking a transmembrane region (e.g., from about residue 18 to about residue 42, relative to the consensus numbering shown in FIG. 1).

Variant proteins encompassed by the present invention are biologically active, that is, they continue to possess the desired biological activity of the native protein. Such variants may result from, for example, genetic polymorphism or from human manipulation.

A TRAIL polypeptide may be altered in various ways including amino acid substitutions, deletions, truncations, and insertions. Methods for such manipulations are generally known in the art. For example, amino acid sequence variants of a TRAIL polypeptide can be prepared by mutations in the DNA. Methods for mutagenesis and nucleotide sequence alterations are well known in the art. See, for example, Kunkel et al. Proc Natl Acad Sci 82:488-492, 1985, Kunkel et al., Methods in Enymol 154:367-382, 1987, U.S. Pat. No. 4,873,192, Watson et al., “Molecular Biology of the Gene”, Fourth Edition, Benjamin/Cummings, Menlo Park, Calif., 1987, and the references cited therein. Guidance as to appropriate amino acid substitutions that do not affect biological activity of the protein of interest may be found in the model of Dayhoff et al., Atlas of Protein Sequence and Structure, Natl Biomed Res Found, Washington, D.C., 1978). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of TRAIL polypeptides. Recursive ensemble mutagenesis (REM), a technique which enhances the frequency of functional mutants in the libraries, can be used in combination with the screening assays to identify TRAIL variants (Arkin and Yourvan. Proc Natl Acad Sci USA 89:7811-7815, 1992; Delgrave et al., Protein Engineering 6:327-331, 1993). Conservative substitutions, such as exchanging one amino acid with another having similar properties, may be desirable as discussed in more detail below.

Variant TRAIL polypeptides may contain conservative amino acid substitutions at various locations along their sequence, as compared to a parent (e.g., naturally-occurring or reference) TRAIL amino acid sequence. A “conservative amino acid substitution” is one in which the amino acid residue is replaced with an amino acid residue having a similar side chain. Families of amino acid residues having similar side chains have been defined in the art, which can be generally sub-classified as follows:

Acidic: The residue has a negative charge due to loss of H ion at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with H ion at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residues are charged at physiological pH and, therefore, include amino acids having acidic or basic side chains (i.e., glutamic acid, aspartic acid, arginine, lysine and histidine).

Hydrophobic: The residues are not charged at physiological pH and the residue is repelled by aqueous solution so as to seek the inner positions in the conformation of a peptide in which it is contained when the peptide is in aqueous medium. Amino acids having a hydrophobic side chain include tyrosine, valine, isoleucine, leucine, methionine, phenylalanine and tryptophan.

Neutral/polar: The residues are not charged at physiological pH, but the residue is not sufficiently repelled by aqueous solutions so that it would seek inner positions in the conformation of a peptide in which it is contained when the peptide is in aqueous medium Amino acids having a neutral/polar side chain include asparagine, glutamine, cysteine, histidine, serine and threonine.

This description also characterizes certain amino acids as “small” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the alpha-amino group, as well as alpha-carbon. Several amino acid similarity matrices (e.g., PAM120 matrix and PAM250 matrix as disclosed for example by Dayhoff et al., A model of evolutionary change in proteins. Matrices for determining distance relationships In M. O. Dayhoff, (ed.), Atlas of protein sequence and structure, Vol. 5, pp. 345-358, National Biomedical Research Foundation, Washington D.C., 1978; and by Gonnet et al., Science 256:14430-1445, 1992, however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small residues are, of course, always non-aromatic. Dependent on their structural properties, amino acid residues may fall in two or more classes. For the naturally-occurring protein amino acids, sub-classification according to this scheme is presented in Table 1.

TABLE 1
Amino acid sub-classification

| Sub-classes | Amino acids |
|---|---|
| Acidic | Aspartic acid, Glutamic acid |
| Basic | Noncyclic: Arginine, Lysine; Cyclic: Histidine |
| Charged | Aspartic acid, Glutamic acid, Arginine, Lysine, |
| | Histidine |
| Small | Glycine, Serine, Alanine, Threonine, Proline |
| Polar/neutral | Asparagine, Histidine, Glutamine, Cysteine, |
| | Serine, Threonine |
| Polar/large | Asparagine, Glutamine |
| Hydrophobic | Tyrosine, Valine, Isoleucine, Leucine, |
| | Methionine, Phenyl-alanine, Tryptophan |
| Aromatic | Tryptophan, Tyrosine, Phenylalanine |
| Residues that influence | Glycine and Proline |
| chain orientation | |

Conservative amino acid substitution also includes groupings based on side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartate with a glutamate, a threonine with a serine, or a similar replacement of an amino acid with a structurally related amino acid will not have a major effect on the properties of the resulting variant polypeptide. Whether an amino acid change results in a functional TRAIL polypeptide can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
Exemplary and Preferred Amino Acid Substitutions

| Original | | |
|---|---|---|
| Residue | Exemplary Substitutions | Preferred Substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleu | Leu |

Alternatively, similar amino acids for making conservative substitutions can be grouped into three categories based on the identity of the side chains. The first group includes glutamic acid, aspartic acid, arginine, lysine, histidine, which all have charged side chains; the second group includes glycine, serine, threonine, cysteine, tyrosine, glutamine, asparagine; and the third group includes leucine, isoleucine, valine, alanine, proline, phenylalanine, tryptophan, methionine, as described in Zubay, G., Biochemistry, third edition, Wm. C. Brown Publishers (1993).

Thus, a predicted non-essential amino acid in a TRAIL polypeptide is typically replaced with another amino acid from the same side chain family. Alternatively, mutations can be introduced randomly along all or part of a TRAIL gene coding sequence, such as by saturation mutagenesis, and the resultant mutants can be screened for an activity of the parent polypeptide, as described for example herein, to identify mutants which retain that activity. Following mutagenesis of the coding sequences, the encoded polypeptide can be expressed recombinantly and its activity determined A “non-essential” amino acid is one that can be altered from the wild-type sequence of an embodiment polypeptide without abolishing or substantially altering one or more of its activities. Suitably, the alteration does not substantially alter one of these activities, for example, the activity is at least 20%, 40%, 60%, 70% or 80% of wild-type. Illustrative non-essential amino acids include any one or more of the amino acids that differ at the same position (e.g., amino acids at positions 118, 119, 120, 121, 122, 123, 124, 125, 126, 127, 128, 129, 134, 137, 138, 139, 140, 141, 142, 143, 144, 145, 146, 147, 148, 149, 150, 151, 152, 153, 155, 156, 158, 161, 162, 164, 165, 166, 168, 173, 174, 175, 176, 177, 178, 186, 187, 188, 190, 195, 197, 199, 202, 204, 205, 206, 207, 208, 209, 210, 211, 212, 213, 214, 215, 216, 217, 218, 219, 220, 221, 222, 223, 224, 225, 227, 228, 234, 236, 239, 242, 250, 254, 255, 256, 268, 273, 274, 282, 284, 285, 287, 289, 290, 291, 293, 296, 300, 301, 302 and 303, relative to the consensus numbering shown in FIG. 1) between the reference TRAIL polypeptides (which include naturally-occurring and synthetic forms of TRAIL polypeptides) shown in FIG. 1. By contrast, an “essential” amino acid is one that, when altered from the wild-type sequence of a reference TRAIL polypeptide, results in abolition of an activity of the parent molecule such that less than 20% of the wild-type activity is present. For example, such essential amino acids include those that are conserved in TRAIL polypeptides across different species, e.g., V (or modified form thereof) at position 130, A (or modified form thereof) at position 131, A (or modified form thereof) at position 132, H (or modified form thereof) at position 133, T (or modified form thereof) at position 135, G (or modified form thereof) at position 136, K (or modified form thereof) at position 154, G (or modified form thereof) at position 157, K (or modified form thereof) at position 159, I (or modified form thereof) at position 160, W (or modified form thereof) at position 163, R (or modified form thereof) at position 167, G (or modified form thereof) at position 169, H (or modified form thereof) at position 170, S (or modified form thereof) at position 171, F (or modified form thereof) at position 172, R (or modified form thereof) at position 179, N (or modified form thereof) at position 180, G (or modified form thereof) at position 181, E (or modified form thereof) at position 182, L (or modified form thereof) at position 183, V (or modified form thereof) at position 184, I (or modified form thereof) at position 185, G (or modified form thereof) at position 189, Y (or modified form thereof) at position 191, Y (or modified form thereof) at position 192, I (or modified form thereof) at position 193, Y (or modified form thereof) at position 194, Q (or modified form thereof) at position 196, Y (or modified form thereof) at position 198, R (or modified form thereof) at position 200, F (or modified form thereof) at position 201, E (or modified form thereof) at position 203, Q (or modified form thereof) at position 226, Q (or modified form thereof) at position 229, Y (or modified form thereof) at position 230, I (or modified form thereof) at position 231, Y (or modified form thereof) at position 232, K (or modified form thereof) at position 233, T (or modified form thereof) at position 235, Y (or modified form thereof) at position 237, P (or modified form thereof) at position 238, P (or modified form thereof) at position 240, I (or modified form thereof) at position 241, L (or modified form thereof) at position 243, M (or modified form thereof) at position 244, K (or modified form thereof) at position 245, S (or modified form thereof) at position 246A (or modified form thereof) at position 247, R (or modified form thereof) at position 248, N (or modified form thereof) at position 249, C (or modified form thereof) at position 251, W (or modified form thereof) at position 252, S (or modified form thereof) at position 253, E (or modified form thereof) at position 257, Y (or modified form thereof) at position 258, G (or modified form thereof) at position 259, L (or modified form thereof) at position 260, Y (or modified form thereof) at position 261, S (or modified form thereof) at position 2621 (or modified form thereof) at position 262, Y (or modified form thereof) at position 264, Q (or modified form thereof) at position 265, G (or modified form thereof) at position 266, G (or modified form thereof) at position 267, F (or modified form thereof) at position 269, E (or modified form thereof) at position 270, L (or modified form thereof) at position 271, K (or modified form thereof) at position 272, D (or modified form thereof) at position 275, R (or modified form thereof) at position 276, I (or modified form thereof) at position 277, F (or modified form thereof) at position 278, V (or modified form thereof) at position 279, S (or modified form thereof) at position 280, V (or modified form thereof) at position 281, N (or modified form thereof) at position 283, L (or modified form thereof) at position 286, D (or modified form thereof) at position 288, E (or modified form thereof) at position 292, S (or modified form thereof) at position 294, F (or modified form thereof) at position 295, G (or modified form thereof) at position 297, A (or modified form thereof) at position 298, F (or modified form thereof) at position 299, relative to the consensus numbering shown in FIG. 1. These amino acids are conserved across several animal species including Homo sapiens, Pan troglodytes, Macaca mulatta, Crassostrea ariakensis, Pongo abelii, Callithrix jacchus, Felis catus, Ailuropoda melanoleuca, Equus caballus, Sus scrofa, Canis lupis familiaris, Oryctolagus cuniculus, Rattus novegicus and Mus musculus.

Accordingly, the present invention also contemplates as TRAIL polypeptides, variants of the naturally-occurring TRAIL polypeptide sequences or their biologically-active fragments, wherein the variants are distinguished from the naturally-occurring sequence by the addition, deletion, or substitution of one or more amino acid residues. In general, variants will display at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% similarity to a parent or reference TRAIL polypeptide sequence as, for example, set forth in SEQ ID NO: 2, 4, 6, 8, 10, 12, 14, 16, 17, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102, as determined by sequence alignment programs described elsewhere herein using default parameters. Desirably, variants will have at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to a reference TRAIL polypeptide sequence as, for example, set forth in SEQ ID NO: 2, 4, 6, 8, 10, 12, 14, 16, 17, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102, as determined by sequence alignment programs described elsewhere herein using default parameters. Variants of a wild-type or reference TRAIL polypeptide, which fall within the scope of a variant polypeptide, may differ from the wild-type or reference molecule generally by as much 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, or 11 amino acid residues or suitably by as few as 10, 9, 8, 7, 6, 5 4, 3, 2, or 1 amino acid residue(s). In some embodiments, a variant polypeptide differs from the corresponding sequences in SEQ ID NO: 2, 4, 6, 8, 10, 12, 14, 16, 17, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102 by at least 1 but by less than or equal to 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3 or 2 amino acid residues. In other embodiments, it differs from the corresponding sequence in any one of SEQ ID NO: 2, 4, 6, 8, 10, 12, 14, 16, 17, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102 by at least one 1% but less than or equal to 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3% or 2% of the residues. If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution, as discussed above.

TRAIL polypeptides in accordance with the present invention also encompass TRAIL polypeptides comprising amino acids with modified side chains, incorporation of unnatural amino acid residues and/or their derivatives during peptide, polypeptide or protein synthesis and the use of cross-linkers and other methods which impose conformational constraints on the peptides, portions and variants of the invention. Examples of side chain modifications include modifications of amino groups such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulfonic acid (TNBS).

The carboxyl group may be modified by carbodiimide activation via O-acylisourea formation followed by subsequent derivatization, by way of example, to a corresponding amide.

The guanidine group of arginine residues may be modified by formation of heterocyclic condensation products with reagents such as 2,3-butanedione, phenylglyoxal and glyoxal.

Sulfhydryl groups may be modified by methods such as performic acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulfides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulfonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in Table 3.

TABLE 3
Non-Conventional Amino acids

| | |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

TRAIL variant polypeptides also encompass: (1) polypeptides whose amino group at the N-terminal amino acid residue (e.g., methionine residue) is protected with a protecting group (e.g., a C₁₋₆ acyl group such as a C₁₋₆ alkanoyl group, e.g., formyl group, acetyl group, etc.); (2) polypeptides whose N-terminal region is cleaved in vivo and the glutamyl group thus formed is pyroglutaminated; (3) polypeptides whose substituents (e.g., —OH, —SH, amino group, imidazole group, indole group, guanidino group, etc.) on the side chains of amino acids in the molecule are protected with suitable protecting groups (e.g., a C₁₋₆ acyl group such as a C₁₋₆ alkanoyl group, e.g., formyl group, acetyl group, etc.), (4) polypeptides whose carboxyl group at the C-terminal amino acid residue is protected by a protecting group (e.g., an ester or ketone-forming alkyl groups, such as lower (C₁ to C₆) alkyl groups, for example methyl, ethyl and propyl, and amide-forming amino groups, such as primary amines (—NH₂), and mono- and di-alkylamino groups, such as methylamino, ethylamino, dimethylamino, diethylamino, methylethylamino, and the like); (5) polypeptides whose C-terminus comprises a descarboxylated amino acid analogue; and (6) polypeptides whose side chains have been modified to include a carbohydrate, polyethylene glycol (PEG) or other polymer; etc.

The TRAIL polypeptides of the present invention also include polypeptides that are encoded by polynucleotides that hybridize under stringency conditions as defined herein, especially medium or high stringency conditions, to TRAIL-encoding polynucleotide sequences, or the non-coding strand thereof, as described below.

In some embodiments, calculations of sequence similarity or sequence identity between sequences are performed as follows:

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 30%, usually at least 40%, more usually at least 50%, 60%, and even more usually at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percent identity between the two sequences is a function of the number of identical amino acid residues shared by the sequences at individual positions, taking into account the number of gaps, and the length of each gap, which need to be introduced for optimal alignment of the two sequences. By contrast, the percent similarity between the two sequences is a function of the number of identical and similar amino acid residues shared by the sequences at individual positions, taking into account the number of gaps, and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and Wünsch, (1970, J. Mol. Biol. 48: 444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In specific embodiments, the percent identity between nucleotide sequences is determined using the GAP program in the GCG software package (available at http://www.gcg.com), using a NWS-gapdna.CMP matrix and a gap weight of 40, 50, 60, 70, or 80 and a length weight of 1, 2, 3, 4, 5, or 6. An non-limiting set of parameters (and the one that should be used unless otherwise specified) includes a Blossum 62 scoring matrix with a gap penalty of 12, a gap extend penalty of 4, and a frameshift gap penalty of 5.

In some embodiments, the percent identity or similarity between amino acid or nucleotide sequences can be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., J Mol Biol 215:403-410, 1990). BLAST nucleotide searches can be performed with the NBLAST program, score=100, wordlength=12 to obtain nucleotide sequences homologous to 53010 nucleic acid molecules of the invention. BLAST protein searches can be performed with the XBLAST program, score=50, wordlength=3 to obtain amino acid sequences homologous to 53010 protein molecules of the invention. To obtain gapped alignments for comparison purposes, Gapped BLAST can be utilized as described in Altschul et al., Nucleic Acids Res 25:3389-3402, 1997). When utilizing BLAST and Gapped BLAST programs, the default parameters of the respective programs (e.g., XBLAST and NBLAST) can be used.

Variants of a reference TRAIL polypeptide can be identified by screening combinatorial libraries of mutants, e.g., truncation mutants, of a TRAIL polypeptide. Libraries or fragments e.g., N terminal, C terminal, or internal fragments, of a TRAIL coding sequence can be used to generate a variegated population of fragments for screening and subsequent selection of variants of a reference TRAIL.

Methods for screening gene products of combinatorial libraries made by point mutation or truncation, and for screening cDNA libraries for gene products having a selected property are known in the art. Such methods are adaptable for rapid screening of the gene libraries generated by combinatorial mutagenesis of TRAIL polypeptides.

TRAIL polypeptides in accordance with the present invention may be prepared by any suitable procedure known to those of skill in the art. For example, the TRAIL polypeptides may be produced by any convenient method such as by purifying the peptides or polypeptides from naturally-occurring reservoirs including helminths. Methods of purification include size exclusion, affinity or ion exchange chromatography/separation. The identity and purity of derived TRAIL is determined for example by SDS-polyacrylamide electrophoresis or chromatographically such as by high performance liquid chromatography (HPLC). Alternatively, the TRAIL polypeptides may be synthesized by chemical synthesis, e.g., using solution synthesis or solid phase synthesis as described, for example, in Chapter 9 of Atherton and Shephard (supra) and in Roberge et al., Science 269:202, 1995.

In some embodiments, the TRAIL polypeptides are prepared by recombinant techniques. For example, the TRAIL polypeptides of the invention may be prepared by a procedure including the steps of: (a) preparing a construct comprising a polynucleotide sequence that encodes a TRAIL polypeptide and that is operably linked to a regulatory element; (b) introducing the construct into a host cell; (c) culturing the host cell to express the polynucleotide sequence to thereby produce the encoded TRAIL polypeptide; and (d) isolating the TRAIL polypeptide from the host cell. In illustrative examples, the nucleotide sequence encodes at least a biologically active portion of the sequences set forth in SEQ ID NO: 2, 4, 6, 8, 10, 12, 14, 16, 17, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100 or 102, or a variant thereof. Recombinant TRAIL polypeptides can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6.

Exemplary nucleotide sequences that encode TRAIL polypeptides of the invention encompass full-length TRAIL genes as well as portions of the full-length or substantially full-length nucleotide sequences of the TRAIL genes or their transcripts or DNA copies of these transcripts. Portions of a TRAIL nucleotide sequence may encode polypeptide portions or segments that retain the biological activity of the native polypeptide. A portion of a TRAIL nucleotide sequence that encodes a biologically active fragment of a TRAIL polypeptide may encode at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250 or more contiguous amino acid residues, or almost up to the total number of amino acids present in a full-length TRAIL polypeptide.

Illustrative TRAIL polynucleotide sequences include:

atggctatgatggaggtccaggggggacccagcctgggacagacctgcgtgctgatcgtgatcttcacagtgctcctgcag
tctctctgtgtggctgtaacttacgtgtactttaccaacgagctgaagcagatgcaggacaagtactccaaaagtggcattgcttgtttctta
aaagaagatgacagttattgggaccccaatgacgaagagagtatgaacagcccctgctggcaagtcaagtggcaactccgtcagctc
gttagaaagatgattttgagaacctctgaggaaaccatttctacagttcaagaaaagcaacaaaatatttctcccctagtgagagaaagag
gtcctcagagagtagcagctcacataactgggaccagaggaagaagcaacacattgtcttctccaaactccaagaatgaaaaggctct
gggccgcaaaataaactcctgggaatcatcaaggagtgggcattcattcctgagcaacttgcacttgaggaatggtgaactggtcatcc
atgaaaaagggttttactacatctattcccaaacatactttcgatttcaggaggaaataaaagaaaacacaaagaacgacaaacaaatgg
tccaatatatttacaaatacacaagttatcctgaccctatattgttgatgaaaagtgctagaaatagttgttggtctaaagatgcagaatatgg
actctattccatctatcaagggggaatatttgagcttaaggaaaatgacagaatttttgtttctgtaacaaatgagcacttgatagacatgga
ccatgaagccagttttttcggggcctttttagttggcttg [SEQ ID NO: 3]; (corresponding to a nucleotide
sequence from NCBI Accession: BT019563, encoding a putative full-length synthetic TRAIL,
as set forth in NCBI Accession: AAV38370);
atggctatgatggaggtccaggggggacccagcctgggacagacctgcgtgctgatcgtgatcttcacagtgctcctgcag
tctctctgtgtggctgtaacttacgtgtactttaccaacgagctgaagcagatgcaggacaagtactccaaaagtggcattgcttgtttctta
aaagaagatgacagttattgggaccccaatgacgaagagagtatgaacagcccctgctggcaagtcaagtggcaactccgtcagctc
gttagaaagatgattttgagaacctctgaggaaaccatttctacagttcaagaaaagcaacaaaatatttctcccctagtgagagaaagag
gtcctcagagagtagcagctcacataactgggaccagaggaagaagcaacacattgtcttctccaaactccaagaatgaaaaggctct
gggccgcaaaataaactcctgggaatcatcaaggagtgggcattcattcctgagcaacttgcacttgaggaatggtgaactggtcatcc
atgaaaaagggttttactacatctattcccaaacatactttcgatttcaggaggaaataaaagaaaacacaaagaacgacaaacaaatgg
tccaatatatttacaaatacacaagttatcctgaccctatattgttgatgaaaagtgctagaaatagttgttggtctaaagatgcagaatatgg
actctattccatctatcaagggggaatatttgagcttaaggaaaatgacagaatttttgtttctgtaacaaatgagcacttgatagacatgga
ccatgaagccagtttttttggggcctttttagttggctaa [SEQ ID NO: 5] (corresponding to a nucleotide
sequence from NCBI Accession: NM_003810, encoding a putative full-length human TRAIL
isoform 1, as set forth in NCBI Accession: NP_003801);
atggctatgatggaggtccaggggggacccagcctgggacagacctgcgtgctgatcgtgatcttcacagtgctcctgcag
tctctctgtgtggctgtaacttacgtgtactttaccaacgagctgaagcagatgcaggacaagtactccaaaagtggcattgcttgtttctta
aaagaagatgacagttattgggaccccaatgacgaagagagtatgaacagcccctgctggcaagtcaagtggcaactccgtcagctc
gttagaaagatgattttgagaacctctgaggaaaccatttctacagttcaagaaaagcaacaaaatatttctcccctagtgagagaaagag
gtcctcagagagtagcagctcacataactgggaccagaggaagaagcaacacattgtcttctccaaactccaagaatgaaaaggctct
gggccgcaaaataaactcctgggaatcatcaaggagtgggcattcattcctgagcaacttgcacttgaggaatggtgaactggtcatcc
atgaaaaagggttttactacatctattcccaaacatactttcgatttcaggaggaaataaaagaaaacacaaagaacgacaaacaaatgg
tccaatatatttacaaatacacaagttatcctgaccctatattgttgatgaaaagtgctagaaatagttgttggtctaaagatgcagaatatgg
actctattccatctatcaagggggaatatttgagcttaaggaaaatgacagaatttttgtttctgtaacaaatgagcacttgatagacatgga
ccatgaagccagttttttaggggcctttttagttggcttg [SEQ ID NO: 7] (corresponding to a nucleotide
sequence from NCBI Accession: AY893035, encoding a putative full-length synthetic
TRAIL, as set forth in NCBI Accession: AAX29952);
caggatcatggctatgatggaggtccaggggggacccagcctgggacagacctgcgtgctgatcgtgatcttcacagtgct
cctgcagtctctctgtgtggctgtaacttacgtgtactttaccaacgagctgaagcagaaaagcaacaaaatatttctcccctagtgagag
aaagaggtcctcagagagtagcagctcacataactgggaccagaggaagaagcaacacattgtcttctccaaactccaagaatgaaa
aggctctgggccgcaaaataaactcctgggaatcatcaaggagtgggcattcattcctgagcaacttgcacttgaggaatggtgaactg
gtcatccatgaaaaagggttttactacatctattcccaaacatactttcgatttcaggaggaaataaaagaaaacacaaagaacgacaaa
caaatggtccaatatatttacaaatacacaagttatcctgaccctatattgttgatgaaaagtgctagaaatagttgttggtctaaagatgca
gaatatggactctattccatctatcaagggggaatatttgagcttaaggaaaatgacagaatttttgtttctgtaacaaatgagcacttgata
gacatggaccatgaagccagtttttttggggcctttttagttggctaa [SEQ ID NO: 9] (corresponding to a
nucleotide sequence from NCBI Accession: CH471052, encoding a putative full-length
human TRAIL isoform CRA_b, as set forth in NCBI Accession: EAW78466);
atggctatgatggaggtccaggggggacccagcctgggacagacctgcgtgctgatcgtggtcttcacagtgctcctgcag
tctctctgtgtggctgtaacttacgtgtactttaccaacgagctgaagcagatgcaggacaagtactccaaaagtggcattgcttgtttctta
aaagaagatgacagttattgggaccccaatgacgaagacagtatgaacagcccctgctggcaagtcaagtggcaactccgtcagctc
gttagaaagatgattttgagaacctctgaggaaaccatttctacagttcaagaaaagcaacaaaatatttctcccctagtgagagaaagag
gtcctcagagagtagcagctcacataactggaaccagaggaagaagcaacacattgtcttctccaaactccaagaatgaaaaggctct
gggccacaaaataaactcctgggaatcatcaaggagtgggcattcattcctgagcaacttgcacttgaggaatggcgaactggtcatcc
atgaaaaagggttttactacatctattcccaaacatactttcgatttcaggaggaaataaaagaaaacacaaagaacgacaaacaaatgg
tccaatatatttacaaatacacaagttatcctgaccctatattgttgatgaaaagcgctagaaatagttgttggtctaaagatgcagaatatg
gactctattccatctatcaagggggaatatttgagcttaaggaaaatgacagaatttttgtttctgtaacaaatgagcacttgatagacatgg
accatgaagccagttttttcggggcctttttagttggctaa [SEQ ID NO: 11] (corresponding to a nucleotide
sequence from NCBI Accession: XM_516879, encoding a putative full-length Pan
troglodytes TRAIL, as set forth in NCBI Accession: XP_516879);
atggctatgatggaggcccaggggggacccagcccggggcagacctgcgtgctgatcctgatcttcacggtgctcctgca
gtccctctgtgcagctgtaacttacgtgtacttcaccaacgagctgaagcagatgcaggacaagtactccaaaagtggcattgcttgtttc
ttgaaagaagatgacagttcttgggatcccaatgacgaagagagtatgaagagcccctgctggcaagtcaagtggcaactccgtcaac
tcgttagaaagatgattttgagaacctctgaggaaaccatttctacagttcaagaaaagcaacaaaatacttctcccctagtgagagaaag
aggtcctcagagagtagcagctcacataactgggaccagaggaagaagcaacacattgtcttctccaaactccaagaatgaaaaggct
ctgggccgcaaaataaactcctgggaatcatcaaggagtgggcattcattcctgagcaacttgcacttgaggaatggcgaactggtcat
ccaagaaaaggggttttactacatctattcccaaacatactttcgatttcaggaggaaataaaagaaaacacaaagaacgacaaacaaat
ggtccaatatatttacaaatacacaagttatcctgaccctatactgctgatgaaaagcgctagaaatagttgttggtctaaagatgcagaat
acggactctattccatctatcaagggggattatttgagcttaagaaagatgacagaatttttgtttctgtaacaaatgagcacttgatagaca
tggaccatgaagccagctttttcggggcctttttggttggctaa [SEQ ID NO: 17] (corresponding to a nucleotide
sequence from NCBI Accession: XM_001084768, encoding a putative full-length Macaca
mulatta TRAIL, as set forth in NCBI Accession: XP_001084768);
atggtgagagaaagaggtcctcagagagtagcagctcacataactgggaccagaggaagaagcaacacattgtcttctcc
aaactccaagaatgaaaaggctctgggccgcaaaataaactcctgggaatcatcaaggagtgggcattcattccagagcaacttgcac
ttgaggaatggtgaactggtcatccatgaaaaagggttttactacatctattcccaaacatactttcgatttcaggaggaaataaaagaaaa
cgcaaagaacgacaaacaaatggtccaatatatttacaaatacacaagttatcctgaccctatattgttgatgaaaagtgctagaaatagtt
gttggtctaaagatgcagaatatggactctattccatctatcaagggggaatatttgagcttaaggaaaatgacagaatttttgtttctgtaac
aaatgagcacttgatagacatggaccatgaagccagttttttcggggccttttaa [SEQ ID NO: 19] (corresponding a
nucleotide sequence from NCBI Accession: EF541151, encoding a putative full-length
Crassostrea ariakensis TRAIL, as set forth in NCBI Accession: ABU39827);
ccncarmgngtngcngcncayathacnggnacnmgnggnmgnwsnaayacnytnwsnwsnccnaaywsnaa
raaygaraargcnytnggnmgnaarathaaywsntgggarwsnwsnmgnwsnggncaywsnttyytnwsnaayytncay
ytnmgnaayggngarytngtnathcaygaraarggnttytaytayathtaywsncaracntayttymgnttycargargarathaa
rgaraayacnaaraaygayaarcaratggtncartayathtayaartayacnwsntayccngayccnathytnytnatgaarwsng
cnmgnaaywsntgytggwsnaargaygcngartayggnytntaywsnathtaycarggnggnathttygarytnaargaraay
gaymgnathttygtnwsngtnacnaaygarcayytnathgayatggaycaygargcnwsnttyttyggngcnttyytngtnggn
trr [SEQ ID NO: 21] (corresponding to a degenerate nucleotide sequence encoding a human
TRAIL fragment, as set forth in NCBI Accession: 1D4V_B);
atggctatgatggaggtccaggggggacccagcctggggcagacctgcgtgctgatcgtgatcttcacagtgctcctgcag
tctctctgtgtggctgtaacttacgtgtactttaccaacgagctgaagcagatgcaggacaagtactccaaaagtggcattgcttgtttctta
aaagaagatgacagctcttgggaccctaatgacgaagacagtatgaacagcccctgctggcaagtcaagtggcaactccgtcagctc
gttagaaagatgattttgagaacctctgaggaaaccatttctacagttcaagaaaagcaacaaaatgtttctcccctagtgagagaaaga
ggtcctcagagagtagcagctcacataactgggaccagaggaagaagcaacacattgtcttctccaagtaagagaaacaacaaannn
nnnnnncgcaaaataaactcctgggaatcatcaaggagtgggcattcattcctgagcaacttgcacttgaggaatggcgaactggtca
tccatgaaaaagggttttactacatctattcccaaacatactttcgatttcaggaggaaataaaagaaaacacaaagaacgacaaacaaat
ggtccaatatatttacaaatacacaagttatcctgatcctatattgctgatgaaaagcgctagaaatagttgttggtctaaagatgcagaata
tggactctattccatctatcaagggggaatatttgagcttaaggaaaatgacagaatttttgtttctgtaacaaatgagcacttgatagacat
ggaccatgaagccagttttttcggggcctttttagttggctaa [SEQ ID NO: 23] (corresponding to a nucleotide
sequence from NCBI Accession: XM_002814289, encoding a putative full-length Pongo
abelii TRAIL, as set forth in NCBI Accession: XP_002814335);
atggctatgatggagggtcaggggggacccagcccggggcagacctgcgtgctgatcctgatcttcacagtgctcctgca
gtccctctgtgtggccgtaacttacctgtacttcaccaatgagctgaagcagatgcaggacaagtactccaaaagcggcattgcttgtttc
ttaaaagaagatggcagctcctgggaccccagtgacgaagagagtatgaatagcccctgctgggaagtcaagtggcaactccgtcag
ctcgttagaaagatgattttgagaacctctgaagaaaccatttctacagttcaagaaaagcaacgaggtatttctccccaagtgagagaaa
gaggtcctcagagagtagcagctcacataactgggaccagaggaagtagcaacacgttgcctattccaaactccaagaatgaaaagg
ctctgggccgcaaaataaactcctgggaatcatcaaggagtggacattattcctgagcaacttgcacttgaggaatggcgagctggtc
atccatgaaaaagggctgtattacatctattgccaagtatactttcgatttcaggaggaaatccaagaaaacagaaagaacgacaaacaa
atggtccagtatatttacaaatacacaagttatcctgaccccatactgctgatgaagagtgctagaaataattgttggtctaaagatgcaga
atatggactctattccatctatcaagggggaatatttgagcttaaggaaaacgacagaatttttgtttctgtaacaaatgggcagttgataga
catggaccatgaagccagttttttcggggcctttttagttggctaa [SEQ ID NO: 25] (corresponding to a
nucleotide sequence from NCBI Accession: XM_002814289, encoding a putative full-length
Callithrix jacchus TRAIL, as set forth in NCBI Accession: XP_002759427);
atgcaggccccggcgggccccagtcccgggcagacctgcgtgctgatcctgatcttcactgtgctcctgcagtccctctgc
gtggccgtgacttacatgtacttcaccagtgaactgaggcagatgcaggacaaatactcccaaagtggcattgcttgtttcttaaaggaa
gacgatatcccttgggaccccaatgatgaagagagtatgaacaccccgtgctggcaagtgaaatggcagctccgtcagtttgttagaaa
gattttgagaacctatgaggaaaccattcctacagttccagaaaagcagctaaatattccttacctagtaagagaaagaggtcctcagag
agtagcagctcacataactggaaccagtcggagaagaagcacattcccagttccaagctccaagaatgaaaaagctttgggtcagaaa
ataaactcctgggagtcatcaagaaaaggacattcattcttgaataatttgcacttgaggaatggtgagctggttattcatcagagggggtt
ttattacatctattcccaaacatactttcgatttcaggaacctgaggaaattccaacaggacagaacagaaagagaaacaaacaaatggt
ccaatatatttacaaacacacgagttatccggaccctatactgctgatgaaaagtgctagaaatagttgttggtctaaagattctgaatatg
gactctattccatctatcaaggtgggatatttgagcttaaggaaaacgatagaatttttgtctctgtatctaacgagcaattgattgacatgga
ccaagaagccagttttttcggggcctttttaatcggctaa [SEQ ID NO: 27] (corresponding to a nucleotide
sequence from NCBI Accession: NM_001130844, encoding a putative full-length Felis catus
TRAIL, as set forth in NCBI Accession: NP_001124316);
atgcaggccccggggggccccagccctgggcagacgtgcgtgttgaccctcatcttcacagtgctcctgcagtccctctgt
gtggcggtgacctacatgtacttcaccagggagctgaagcagatgcaggacaagtactcccaaagcggcatcgcttgtttcttaaagga
agatgatattccttgggacccaaatgatgaagagagtatgaacaatccttgctggcaagtgaagtggcaactccgtcagtttgttagaaa
gatgattttgaaaacctatgaggaaaccattccttcaattccagaaaagcagctaaatattccttacgtagtaaatgaaagaggtcttcaga
gagtagcagctcacataactggaaccagtcggagaagaagcacgtttccagttccaagctccaagaatgaaaaagctttgggccaga
aaataaactcctgggagtcatcaagaaaaggacattcattcttgagtaatttgcacttgaggaatggagagctggttatccatcaaagtgg
gttttattacatctattcccaaacatactttcgatttcaggaacctgaggaaacttcgggaccaatttcaaaggaacaaaacagaaagaaa
aacaaacaaatggtacaatatatttacaaatacacaagttatcctgaccctatactgctgatgaaaagtgctagaaatagttgctggtctaa
agattctgagtatggactctattccatctatcaaggtgggatatttgagcttaaggaaaatgatagaatttttgtctctgtaaataatgagcaa
ttgattgacatggaccaagaagccagttttttcggggcctttttaattggctaa [SEQ ID NO: 29] (corresponding to a
nucleotide sequence from NCBI Accession: XM_002921589, encoding a putative full-length
Ailuropoda melanoleuca TRAIL, as set forth in NCBI Accession: XP_002921635);
atggccatgatgcaggcatcagggggtcccagccccgggcagacctgcgtgctgatcctgatcttcacagtgctcctgcag
gccctctgtgtggctgtgacttatttgtacttcaccaacgagctgaagcagatgcagatcaaatactccaaaagtggcattgcctgtttctta
aaggaagatgacagcgattgggacccaaatgacgaagagagtatgaacagcccctgctggcaagtcaagtggcagctgcgtcagttt
gttagaaagatgattttgagaacctatgaggaatccattcctacaacttcagaaaagcgacaaaatattcctcccttagtaagagaaagag
gtcttcagagagtagcagctcacataactgggaccagtcggagaagaagcacagtctcaattccacgctccaagaatgaaaaagcact
gggccagaaaataaacgcctgggagacatcaagaaaaggacattcgttcttgaataatttacacttgaggaatggagagctggttatcc
atcaaacagggttttattacatctattcccaaacatactttcgatttcaggaacctgaggaaattttgggaacagttgcaacagaagagaac
agaaggaaaaataaacaaatggtacaatatatttacaaaagcacagactatcctgaccctatactgctgatgaaaagtgctagaaatagtt
gttggtctaaagattcagaatacggactctattccatctatcaaggtggaatatttgagcttaaggaaaatgacagaatttttgtctctgtaac
taatgagcaattgattgacatggaccaagaagccagtttcttcggggcctttttaatcggctaa [SEQ ID NO: 31]
(corresponding to a nucleotide sequence from NCBI Accession: XM_001494088, encoding a
putative full-length Equus caballus TRAIL, as set forth in NCBI Accession: XP_001494138);
atgcaggccccggggggccccagccctgggcagacgtgcgtgttgaccctcatcttcacagtgctcctgcagtccctctgt
gtggcggtgacctacatgtacttcaccagggagctgaagcagatgcaggacaagtactcccaaagcggcatcgcttgtttcttaaagga
agatgatattccttgggacccaaatgatgaagagagtatgaacaatccttgctggcaagtgaagtggcaactccgtcagtttgttagaaa
gatgattttgaaaacctatgaggaaaccattccttcaattccagaaaagcagctaaatattccttacgtagtaaatgaaagaggtcttcaga
gagtagcagctcacataactggaaccagtcggagaagaagcacgtttccagttccaagctccaagaatgaaaaagctttgggccaga
aaataaactcctgggagtcatcaagaaaaggacattcattcttgagtaatttgcacttgaggaatggagagctggttatccatcaaagtgg
gttttattacatctattcccaaacatactttcgatttcaggaacctgaggaaacttcgggaccaatttcaaaggaacaaaacagaaagaaa
aacaaacaaatggtacaatatatttacaaatacacaagttatcctgaccctatactgctgatgaaaagtgctagaaatagttgctggtctaa
agattctgagtatggactctattccatctatcaaggtgggatatttgagcttaaggaaaatgatagaatttttgtctctgtaaataatgagcaa
ttgattgacatggaccaagaagccagttttttcggggccttt [SEQ ID NO: 33] (corresponding to a nucleotide
sequence from NCBI Accession: GL192841, encoding a putative full-length Ailuropoda
melanoleuca TRAIL, as set forth in NCBI Accession: EFB16787);
atggccctgaagcaggctccgggctccagacttgggcagatctgcatgccgatcctcatcttcacagtgctgctgcaggcttt
tggtatggccgtgttttacatgtatttcaacaaagagctgaagcagatgcagaacaaatacttcaaaagtggcttggcttgcttcttggagg
aagatgaccgttcctgggactccagagatgatgagagtataatcaatccctgctgggaactaaagtcccaactctatctgtttgttaaaaa
gatgactttgagaacctttgaggaaatgattcctacaaatccagaaaagcaatataatccttacctagagagagaaaagggtcctaagag
ggtagctgctcatataactggaagcaatcggaaaaaaagtacgttgccagttccaggctccaagaatgaaaaagctgtgggccataaa
ataaattcctgggagtcatcaagaaaaggacattcgttcttgaataatttgtacttaaggaatggagagctggttatccttcaaacaggattt
tattacatctattcccaaacatactttcgatttcaggaacctgaggaagttttgggaactgtttcaacagaagagaacagaaaaaaaatcaa
acaaatggtacaatatatttacaaatacacaaactatcctgaccctatactgctgatgaaaagtgctagaaatagttgttggtctaaagattc
agaatatggactctattccatctatcaaggaggaatatttgagcttaaggaaaatgatcgaatttttgtctctgtaactaatgaacgattggtt
gacctggaccaagaagccagttttttcggagcctttttaattggctaa [SEQ ID NO: 35] (corresponding to a
nucleotide sequence from NCBI Accession: GL192841, encoding a putative full-length
Bos taurus TRAIL, as set forth in NCBI Accession: XP_583785);
atggcggtgatgcagactccaggaggccccagccccgggcagacctgtgtgttgatcctgatcttcacagtgctcctgcaa
gccctctgtgtggccttgacttacgtgtacttcaccaatgaactgaaacagatgcaggacaagtactccaaaagcggtatagcttgcttct
taaaggaagatgacagtttctgggatcccaccgatgacgagagaatgctcagcccctgctggcaggtgaagtggcagctacgtcagtt
tgtgagaaagatgattttgagaacctatgaggaaaccatttctacagtttcagaaaagcaacaaggcattcctcacctagaaagagaaaa
aggtccacagagagtggctgctcacataactggaaccagtaggaaaagaagcacatttccatctctaagctccaaatatgaaaaagctt
tgggccagaaaataaactcctgggaatcatcaagaaaaggacattcattcttgaataattttcacttgaggaatggagagctggttatcca
tcaaacagggttttactacatctattcccaaacatactttcgatttcaggaacctgaggaaattttgggaacggtttctacagaagggaaca
gaaagaaaaacaggcaaatgatacagtatatttacaaatggacaagctatcctgaccctatactgctgatgaaaagtgctagaaatagtt
gttggtctaaagattcagaatatggactctattccatctatcaaggtggaatatttgagcttaaggaagatgaccgaatttttgtctctgttact
aatgagcaactgattgacatggaccaagaagccagttttttcggggcctttttaattggctaa [SEQ ID NO: 37]
(corresponding to a nucleotide sequence from NCBI Accession: GL192841, encoding a
putative full-length Sus scrofa TRAIL, as set forth in NCBI Accession: NP_001019867);
atgcaggccccggggggccccagcctcgggctgacgtgcgtgctgatcctcatcttcactgtgctgctccagtccctctgcg
tggccgtcacctacatgtacttcaccagggagctgaagcagatgcaggacaagtactcccaaagtggcatcgcttgtttcttaaaggaa
gatgatatcccctgggaccccagtgatgaagagagtatgaacaacccctgctggcaagtgaagtggcaactccgccagtttgttagaa
agatgattttgaaaacctatgaggaaaccattcctacagctccagaaaagcagctaaatattccttacgtagtaagcgaccgaggttctca
gagagtagctgctcacataactggaaccagtcggagaagcatgtttccaattccaagctccaagaatgataaagctttgggccacaaaa
taaactcctgggattccacaagaaaaggacattcattcttgaataatttgcacttgaggaacggagagctggttatccatcaaagggggtt
ttattacatctattcccaaacatactttcgatttcaggaacctgaggaaattccaacaggacagaacagaaagagaaacaaacaaatggt
ccaatatatttacaaacacacgagttatccggaccctatactgctgatgaaaagtgctagaaatagttgttggtctaaagattctgaatatg
gactctattccatctatcaaggtgggatatttgagcttaaggaaaacgatagaatttttgtctctgtatctaacgagcaattgattgacatgga
ccaagaagccagttttttcggggcctttttaatcggctaa [SEQ ID NO: 39] (corresponding to a nucleotide
sequence from NCBI Accession: NM_001130836, encoding a putative full-length Canis lupis
familiaris TRAIL, as set forth in NCBI Accession: NP_001124308);
atgtcctctgtgcaggccctggggggccccagtgccgggcagacctgcgtgctgatcctgatcttcacagtgctcctgcagt
ccctctgtgtggccgtgacttacctgtacttcaccaacgaactgaagcagatgcaggacaagtactccaaaagtggcatcgcttgtctctt
aaaggaggatgacagttcctgggactccatcgacgaagagaacatgaacagcccctgctggcaggccaagtggcagctgcggcagt
tcattcgaaagatgcttttgagaacctatgaggaaaccattcctacggttgaagaaaagccacaaactattccttccctagtaagagaaaa
agaaagagaaagagggcctcagagagtagcagctcacctaactgggaacagctggagaagctttatctcagtccctgctccaggctc
ccagagtggaaagaatttgggccagaaaataagctcctgggaatcatcaaggaaaggacattcattcctgaacaatttgcacctgagga
atggagagctggttatccatcaaacaggactttattacatctactcccaaacatactttcgatttcaggaacttgaagaaatttcaggaaca
atttcaagagaagagatcaaaaagaggaacaaacaaatggtacaatatatttacaaatggacaagctaccctgaccctatacttctgatg
aaaagtgctagaaatagttgttggtctaaggattcggaatatggactctattccatctatcaaggaggaatatttgagcttaaggaaaatga
ccgaattttcgtctctgtaacgaatgagcagttgattgacatgaaccaagaatccagtttttttggggcctttttgattggctaa [SEQ ID
NO: 41] (corresponding to a nucleotide sequence from NCBI Accession: XM_002716426,
encoding a putative full-length Oryctolagus cuniculus TRAIL, as set forth in NCBI
Accession: XP_002716472);
atgccttccaccgggaacctgaagggccccagcttcagtcagcacttcacgatgacggtgatctgcatagtgctcctgcagg
tgctcctgcaggccttgactgtggctgtgacttacatgtacttcaacaacgaggtgaaacagctacaggacaattactccaaaatcggac
tagcttgcttctcaaaagaagatggggatttttgggactccactgacgaggggattttgaacagaccttgcttgcaggtcaagaggcaac
tgtatcagctcattgaagaggtgactttgagaacctttgagaaaaccatctctacagttccagaaaagcagctaagcactcctcccttgcc
cagaggtagaagaccccagagagtggcagctcacattaccgggatcactcggagaagcaacttagccttaattccaatctccaaggat
ggaaagaccttgggccagaagatagaaacctgggagtcctctcggagagggcattcatttctcaaccatgtgcacttgagaaacggag
agctggtgatccaggaggagggcctgtattacatctactcccaaacgtactaccggttcaaggaggctaaagaagcttccaagacagt
ctcgaaggacggagggaggatcaaacagatggtgcagtacatctacaaatacaccagctaccccgatcccatactgctgatgaagag
tgccagaaatagctgctggtccagagaagctgagtacggactgtactccatctatcagggggggctgttcgagctcaaagaaaatgac
aggatttttgtttccgtgacgaatgagcatttgatggacctggatcaagaagccagtttattggagcctttttaattaactag [SEQ ID
NO: 43] (corresponding to a nucleotide sequence from NCBI Accession: CH473961,
encoding a putative full-length Rattus novegicus TRAIL, as set forth in NCBI Accession:
EDM01114);
atggcttccaccgggaacctgaagggccccagcttcagtcagcacttcacgatgacggtgatctgcatagtgctcctgcag
gtgctcctgcaggccttgactgtggctgtgacttacatgtacttcaacaacgaggtgaaacagctacaggacaattactccaaaatcgga
ctagcttgcttctcaaaagaagatggggatttttgggactccactgacgaggggattttgaacagaccttgcttgcaggtcaagaggcaa
ctgtatcagctcattgaagaggtgactttgagaacctttgagaaaaccatctctacagttccagaaaagcagctaagcactcctcccttgc
ccagaggtagaagaccccagagagtggcagctcacattaccgggatcactcggagaagcaacttagccttaattccaatctccaagga
tggaaagaccttgggccagaagatagaaacctgggagtcctctcggagagggcattcatttctcaaccatgtgcacttgagaaacgga
gagctggtgatccaggaggagggcctgtattacatctactcccaaacgtactaccggttcaaggaggctaaagaagcttccaagacag
tctcgaaggacggagggaggatcaaacagatggtgcagtacatctacaaatacaccagctaccccgatcccatactgctgatgaaga
gtgccagaaatagctgctggtccagagaagctgagtacggactgtactccatctatcagggggggctgttcgagctcaaagaaaatga
caggatttttgtttccgtgacgaatgagcatttgatggacctggaccatgaagccagcttattggagcctaa [SEQ ID NO: 45]
(corresponding to a nucleotide sequence from NCBI Accession: NM_145681, encoding a
putative full-length Rattus novegicus TRAIL, as set forth in NCBI Accession: NP_663714);
atggcttccaccgggaacctgaagggccccagcttcagtcagcacttcacgatgacggtgatctgcatagtgctcctgcag
gtgctcctgcaggccttgactgtggctgtgacttacatgtacttcaacaacgaggtgaaacagctacaggacaattactccaaaatcgga
ctagcttgcttctcaaaagaagatggggatttttgggactccactgacgaggggattttgaacagaccttgcttgcaggtcaagaggcaa
ctgtatcagctcattgaagaggtgactttgagaacctttgagaaaaccatctctacagttccagaaaagcagctaagcactcctcccttgc
ccagaggtagaagaccccagagagtggcagctcacattaccgggatcactcggagaagcaacttagccttaattccaatctccaagga
tggaaagaccttgggccagaagatagaaacctgggagtcctctcggagagggcattcatttctcaaccatgtgcacttgagaaacgga
gagctggtgatccaggaggagggcctgtattacatctactcccaaacgtactaccggttcaaggaggctaaagaagcttccaagacag
tctcgaaggacggagggaggatcaaacagatggtgcagtacatctacaaatacaccagctaccccgatcccatactgctgatgaaga
gtgccagaaatagctgctggtccagagaagctgagtacggactgtactccatctatcagggggggctgttcgagctcaaagaaaatga
caggatttttgtttccgtgacgaatgagcatttgatggacctggatcaagaagccagcttctttggagcctaa [SEQ ID NO: 47]
(corresponding to a nucleotide sequence from NCBI Accession: EF030546, encoding a
putative full-length Rattus novegicus TRAIL, as set forth in NCBI Accession: ABK32522);
atgccttcctcaggggccctgaaggacctcagcttcagtcagcacttcaggatgatggtgatttgcatagtgctcctgcaggt
gctcctgcaggctgtgtctgtggctgtgacttacatgtacttcaccagcgagatgaagcagctgcaggacaattactccaaaattggact
agcttgcttctcaaagacggatgaggatttctgggactccactgatggagagatcttgaacagaccctgcttgcaggttaagaggcaact
gtatcagctcattgaagaggtgactttgagaacctttcaggacaccatttctacagttccagaaaagcagctaagtactcctcccttgccc
agaggtggaagacctcagaaagtggcagctcacattactgggatcactcggagaagcaactcagctttaattccaatctccaaggatg
gaaagaccttaggccagaagattgagtcctgggagtcctctcggaaagggcattcatttctcaaccacgtgctctttaggaatggagag
ctggtcattgagcaggagggcctgtattacatctattcccaaacatacttccgatttcaggaagctaaagacgcttccaagatggtctcaa
aggacaaggtgagaaccaaacagctggtgcagtacatctacaagtacaccagctatccggatcccatagtgctcatgaagagcgcca
gaaacagctgttggtccagagatgccgagtacggactgtactccatctatcagggaggactgttcgagctaaaaaaaaatgacaggatt
tttgtttctgtgacaaatgaacatttgatggacctggatcaagaagccagcttctttggagcctttttaattaactaa [SEQ ID NO:
49] (corresponding to a nucleotide sequence from NCBI Accession: AKI57633, encoding a
putative full-length Mus musculus TRAIL, as set forth in NCBI Accession: BAE34141); and
atgccttcctcaggggccctgaaggacctcagcttcagtcagcacttcaggatgatggtgatttgcatagtgctcctgcaggt
gctcctgcaggctgtgtctgtggctgtgacttacatgtacttcaccaacgagatgaagcagctgcaggacaattactccaaaattggact
agcttgcttctcaaagacggatgaggatttctgggactccactgatggagagatcttgaacagaccctgcttgcaggttaagaggcaact
gtatcagctcattgaagaggtgactttgagaacctttcaggacaccatttctacagttccagaaaagcagctaagtactcctcccttgccc
agaggtggaagacctcagaaagtggcagctcacattactgggatcactcggagaagcaactcagctttaattccaatctccaaggatg
gaaagaccttaggccagaagattgaatcctgggagtcctctcggaaagggcattcatttctcaaccacgtgctctttaggaatggagag
ctggtcatcgagcaggagggcctgtattacatctattcccaaacatacttccgatttcaggaagctgaagacgcttccaagatggtctcaa
aggacaaggtgagaaccaaacagctggtgcagtacatctacaagtacaccagctatccggatcccatagtgctcatgaagagcgcca
gaaacagctgttggtccagagatgccgagtacggactgtactccatctatcagggaggattgttcgagctaaaaaaaaatgacaggatt
tttgtttctgtgacaaatgaacatttgatggacctggatcaagaagccagcttctttggagcctttttaattaactaa [SEQ ID NO:
51] (corresponding to a nucleotide sequence from NCBI Accession: NM_009425, encoding a
putative full-length Mus musculus TRAIL, as set forth in NCBI Accession: NP_033451);

or

a complement of any one of SEQ ID NO: 3, 5, 7, 9, 11, 17, 19, 21, 23, 25, 27, 29, 31, 33, 35, 37, 39, 41, 43, 45, 47, 49 or 51;

or a portion any one of SEQ ID NO: 3, 5, 7, 9, 11, 17, 19, 21, 23, 25, 27, 29, 31, 33, 35, 37, 39, 41, 43, 45, 47, 49 or 51, encoding a biologically active fragment of a TRAIL polypeptide.

Non-limiting portions of this type include:

gctcacataactgggaccagaggaagaagcaacacattgtcttctccaaactccaagaatgaaaaggctctgggccgcaaa
ataaactcctgggaatcatcaaggagtgggcattcattcctgagcaacttgcacttgaggaatggtgaactggtcatccatgaaaaagg
gttttactacatctattcccaaacatactttcgatttcaggaggaaataaaagaaaacacaaagaacgacaaacaaatggtccaatatattt
acaaatacacaagttatcctgaccctatattgttgatgaaaagtgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agtttttttggg [SEQ ID NO: 53] (corresponding to a nucleotide sequence from NCBI Accession:
NM_003810, encoding amino acids 124-276 of a human TRAIL isoform 1, as set forth in
NCBI Accession: NP_003801);
gctcacataactgggaccagaggaagaagcaacacattgtcttctccaaactccaagaatgaaaaggctctgggccgcaaa
ataaactcctgggaatcatcaaggagtgggcattcattcctgagcaacttgcacttgaggaatggtgaactggtcatccatgaaaaagg
gttttactacatctattcccaaacatactttcgatttcaggaggaaataaaagaaaacacaaagaacgacaaacaaatggtccaatatattt
acaaatacacaagttatcctgaccctatattgttgatgaaaagtgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agttttttcggg [SEQ ID NO: 55] (corresponding to a nucleotide sequence from NCBI Accession:
BT019563, encoding amino acids 124-276 of a synthetic TRAIL, as set forth in NCBI
Accession: AAV38370);
gctcacataactgggaccagaggaagaagcaacacattgtcttctccaaactccaagaatgaaaaggctctgggccgcaaa
ataaactcctgggaatcatcaaggagtgggcattcattcctgagcaacttgcacttgaggaatggtgaactggtcatccatgaaaaagg
gttttactacatctattcccaaacatactttcgatttcaggaggaaataaaagaaaacacaaagaacgacaaacaaatggtccaatatattt
acaaatacacaagttatcctgaccctatattgttgatgaaaagtgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agttttttaggg [SEQ ID NO: 57] (corresponding to a nucleotide sequence from NCBI Accession:
AY893035, encoding amino acids 124-276 of a synthetic TRAIL, as set forth in NCBI
Accession: AAX29952);
gctcacataactgggaccagaggaagaagcaacacattgtcttctccaaactccaagaatgaaaaggctctgggccgcaaa
ataaactcctgggaatcatcaaggagtgggcattcattcctgagcaacttgcacttgaggaatggtgaactggtcatccatgaaaaagg
gttttactacatctattcccaaacatactttcgatttcaggaggaaataaaagaaaacacaaagaacgacaaacaaatggtccaatatattt
acaaatacacaagttatcctgaccctatattgttgatgaaaagtgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agtttttttggg [SEQ ID NO: 59] (corresponding to a nucleotide sequence from NCBI Accession:
CH471052, encoding amino acids 21-173 of a human TRAIL isoform CRA b, as set forth in
NCBI Accession: EAW78466);
gctcacataactggaaccagaggaagaagcaacacattgtcttctccaaactccaagaatgaaaaggctctgggccacaaa
ataaactcctgggaatcatcaaggagtgggcattcattcctgagcaacttgcacttgaggaatggcgaactggtcatccatgaaaaagg
gttttactacatctattcccaaacatactttcgatttcaggaggaaataaaagaaaacacaaagaacgacaaacaaatggtccaatatattt
acaaatacacaagttatcctgaccctatattgttgatgaaaagcgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agttttttcggg [SEQ ID NO: 61] (corresponding to a nucleotide sequence from NCBI Accession:
XM_516879, encoding amino acids 124-276 of a Pan troglodytes TRAIL, as set forth in
NCBI Accession: XP_516879);
gcncayathacnggnacnmgnggnmgnwsnaayacnytnwsnwsnccnaaywsnaaraaygaraargcnytn
ggnmgnaarathaaywsntgggarwsnwsnmgnwsnggncaywsnttyytnwsnaayytncayytnmgnaayggngar
ytngtnathcaygaraarggnttytaytayathtaywsncaracntayttymgnttycargargarathaargaraayacnaaraay
gayaarcaratggtncartayathtayaartayacnwsntayccngayccnathytnytnatgaarwsngcnmgnaaywsntgyt
ggwsnaargaygcngartayggnytntaywsnathtaycarggnggnathttygarytnaargaraaygaymgnathttygtnw
sngtnacnaaygarcayytnathgayatggaycaygargcnwsnttyttyggn [SEQ ID NO: 63] (degenerate
nucleotide sequence encoding amino acids 11-163 of a human TRAIL fragment, as set forth
in NCBI Accession: 1D0G_A);
gcncayathacnggnacnmgnggnmgnwsnaayacnytnwsnwsnccnaaywsnaaraaygaraargcnytn
ggnmgnaarathaaywsntgggarwsnwsnmgnwsnggncaywsnttyytnwsnaayytncayytnmgnaayggngar
ytngtnathcaygaraarggnttytaytayathtaywsncaracntayttymgnttycargargarathaargaraayacnaaraay
gayaarcaratggtncartayathtayaartayacnwsntayccngcnccnathytnytnatgaarwsngcnmgnaaywsntgyt
ggwsnaargaygcngartayggnytntaywsnathtaycarggnggnathttygarytnaargaraaygaymgnathttygtnw
sngtnacnaaygarcayytnathgayatggaycaygargcnwsnttyttyggn [SEQ ID NO: 65] (degenerate
nucleotide sequence encoding amino acids 34-186 of a human TRAIL fragment, as set forth
in NCBI Accession: 1DG6);
gctcacataactgggaccagaggaagaagcaacacattgtcttctccaaactccaagaatgaaaaggctctgggccgcaaa
ataaactcctgggaatcatcaaggagtgggcattcattcctgagcaacttgcacttgaggaatggcgaactggtcatccaagaaaaggg
gttttactacatctattcccaaacatactttcgatttcaggaggaaataaaagaaaacacaaagaacgacaaacaaatggtccaatatattt
acaaatacacaagttatcctgaccctatactgctgatgaaaagcgctagaaatagttgttggtctaaagatgcagaatacggactctattc
catctatcaagggggattatttgagcttaagaaagatgacagaatttttgtttctgtaacaaatgagcacttgatagacatggaccatgaag
ccagctttttcggg [SEQ ID NO: 67] (corresponding to a nucleotide sequence from NCBI
Accession: XM_001084768, encoding amino acids 124-276 of a Macaca mulatta TRAIL, as
set forth in NCBI Accession: XP_001084768);
gctcacataactgggaccagaggaagaagcaacacattgtcttctccaaactccaagaatgaaaaggctctgggccgcaaa
ataaactcctgggaatcatcaaggagtgggcattcattccagagcaacttgcacttgaggaatggtgaactggtcatccatgaaaaagg
gttttactacatctattcccaaacatactttcgatttcaggaggaaataaaagaaaacgcaaagaacgacaaacaaatggtccaatatattt
acaaatacacaagttatcctgaccctatattgttgatgaaaagtgctagaaatagttgttggtctaaagatgcagaatatggactctattcca
tctatcaagggggaatatttgagcttaaggaaaatgacagaatttttgtttctgtaacaaatgagcacttgatagacatggaccatgaagcc
agttttttcggg [SEQ ID NO: 69] (corresponding to a nucleotide sequence from NCBI Accession:
EF541151, encoding amino acids 11-164 of a Crassostrea ariakensis TRAIL, as set forth in
NCBI Accession: ABU39827);
gcncayathacnggnacnmgnggnmgnwsnaayacnytnwsnwsnccnaaywsnaaraaygaraargcnytn
ggnmgnaarathaaywsntgggarwsnwsnmgnwsnggncaywsnttyytnwsnaayytncayytnmgnaayggngar
ytngtnathcaygaraarggnttytaytayathtaywsncaracntayttymgnttycargargarathaargaraayacnaaraay
gayaarcaratggtncartayathtayaartayacnwsntayccngayccnathytnytnatgaarwsngcnmgnaaywsntgyt
ggwsnaargaygcngartayggnytntaywsnathtaycarggnggnathttygarytnaargaraaygaymgnathttygtnw
sngtnacnaaygarcayytnathgayatggaycaygargcnwsnttyttyggn [SEQ ID NO: 71] (degenerate
nucleotide sequence encoding amino acids 6-158 of a human TRAIL fragment, as set forth in
NCBI Accession: 1D4V_B);
gctcacataactgggaccagaggaagaagcaacacattgtcttctccaagtaagagaaacaacaaannnnnnnnncgca
aaataaactcctgggaatcatcaaggagtgggcattcattcctgagcaacttgcacttgaggaatggcgaactggtcatccatgaaaaa
gggttttactacatctattcccaaacatactttcgatttcaggaggaaataaaagaaaacacaaagaacgacaaacaaatggtccaatata
tttacaaatacacaagttatcctgatcctatattgctgatgaaaagcgctagaaatagttgttggtctaaagatgcagaatatggactctattc
catctatcaagggggaatatttgagcttaaggaaaatgacagaatttttgtttctgtaacaaatgagcacttgatagacatggaccatgaag
ccagttttttcggg [SEQ ID NO: 73] (corresponding to a nucleotide sequence from NCBI
Accession: XM_002814289, encoding amino acids 124-276 of a Pongo abelii TRAIL, as set
forth in NCBI Accession: XP_002814335);
gctcacataactgggaccagaggaagtagcaacacgttgcctattccaaactccaagaatgaaaaggctctgggccgcaa
aataaactcctgggaatcatcaaggagtggacattctttcctgagcaacttgcacttgaggaatggcgagctggtcatccatgaaaaagg
gctgtattacatctattgccaagtatactttcgatttcaggaggaaatccaagaaaacagaaagaacgacaaacaaatggtccagtatattt
acaaatacacaagttatcctgaccccatactgctgatgaagagtgctagaaataattgttggtctaaagatgcagaatatggactctattcc
atctatcaagggggaatatttgagcttaaggaaaacgacagaatttttgtttctgtaacaaatgggcagttgatagacatggaccatgaag
ccagttttttcggg [SEQ ID NO: 75] (corresponding to a nucleotide sequence from NCBI
Accession: XM_002759381, encoding amino acids 124-276 of a Callithrix jacchus TRAIL, as
set forth in NCBI Accession: XP_002759427);
gctcacataactggaaccagtcggagaagaagcacattcccagttccaagctccaagaatgaaaaagctttgggtcagaaa
ataaactcctgggagtcatcaagaaaaggacattcattcttgaataatttgcacttgaggaatggtgagctggttattcatcagagggggtt
ttattacatctattcccaaacatactttcgatttcaggaacctgaggaaattccaacaggacagaacagaaagagaaacaaacaaatggt
ccaatatatttacaaacacacgagttatccggaccctatactgctgatgaaaagtgctagaaatagttgttggtctaaagattctgaatatg
gactctattccatctatcaaggtgggatatttgagcttaaggaaaacgatagaatttttgtctctgtatctaacgagcaattgattgacatgga
ccaagaagccagttttttcggg [SEQ ID NO: 77] (corresponding to a nucleotide sequence from NCBI
Accession: NM_001130844, encoding amino acids 120-276 of a Felis catus TRAIL, as set
forth in NCBI Accession: NP_001124316);
gctcacataactggaaccagtcggagaagaagcacgtttccagttccaagctccaagaatgaaaaagctttgggccagaaa
ataaactcctgggagtcatcaagaaaaggacattcattcttgagtaatttgcacttgaggaatggagagctggttatccatcaaagtgggt
tttattacatctattcccaaacatactttcgatttcaggaacctgaggaaacttcgggaccaatttcaaaggaacaaaacagaaagaaaaa
caaacaaatggtacaatatatttacaaatacacaagttatcctgaccctatactgctgatgaaaagtgctagaaatagttgctggtctaaag
attctgagtatggactctattccatctatcaaggtgggatatttgagcttaaggaaaatgatagaatttttgtctctgtaaataatgagcaattg
attgacatggaccaagaagccagttttttcggg [SEQ ID NO: 79 (corresponding to a nucleotide sequence
from NCBI Accession: XM_002921589, encoding amino acids 121-281 of an Ailuropoda
melanoleuca TRAIL, as set forth in NCBI Accession: XP_002921635);
gctcacataactgggaccagtcggagaagaagcacagtctcaattccacgctccaagaatgaaaaagcactgggccagaa
aataaacgcctgggagacatcaagaaaaggacattcgttcttgaataatttacacttgaggaatggagagctggttatccatcaaacagg
gttttattacatctattcccaaacatactttcgatttcaggaacctgaggaaattttgggaacagttgcaacagaagagaacagaaggaaa
aataaacaaatggtacaatatatttacaaaagcacagactatcctgaccctatactgctgatgaaaagtgctagaaatagttgttggtctaa
agattcagaatacggactctattccatctatcaaggtggaatatttgagcttaaggaaaatgacagaatttttgtctctgtaactaatgagca
attgattgacatggaccaagaagccagtttcttcggg [SEQ ID NO: 81] (corresponding to a nucleotide
sequence from NCBI Accession: XM_001494088, encoding amino acids 124-284 of an
Equus caballus TRAIL, as set forth in NCBI Accession: XP_001494138);
gctcacataactggaaccagtcggagaagaagcacgtttccagttccaagctccaagaatgaaaaagctttgggccagaaa
ataaactcctgggagtcatcaagaaaaggacattcattcttgagtaatttgcacttgaggaatggagagctggttatccatcaaagtgggt
tttattacatctattcccaaacatactttcgatttcaggaacctgaggaaacttcgggaccaatttcaaaggaacaaaacagaaagaaaaa
caaacaaatggtacaatatatttacaaatacacaagttatcctgaccctatactgctgatgaaaagtgctagaaatagttgctggtctaaag
attctgagtatggactctattccatctatcaaggtgggatatttgagcttaaggaaaatgatagaatttttgtctctgtaaataatgagcaattg
attgacatggaccaagaagccagttttttcggg [SEQ ID NO: 83] (corresponding to a nucleotide sequence
from NCBI Accession: GL192841, encoding amino acids 121-276 of an Ailuropoda
melanoleuca TRAIL, as set forth in NCBI Accession: EFB16787);
gctgctcatataactggaagcaatcggaaaaaaagtacgttgccagttccaggctccaagaatgaaaaagctgtgggccat
aaaataaattcctgggagtcatcaagaaaaggacattcgttcttgaataatttgtacttaaggaatggagagctggttatccttcaaacagg
attttattacatctattcccaaacatactttcgatttcaggaacctgaggaagttttgggaactgtttcaacagaagagaacagaaaaaaaat
caaacaaatggtacaatatatttacaaatacacaaactatcctgaccctatactgctgatgaaaagtgctagaaatagttgttggtctaaag
attcagaatatggactctattccatctatcaaggaggaatatttgagcttaaggaaaatgatcgaatttttgtctctgtaactaatgaacgatt
ggttgacctggaccaagaagccagttttttcgga [SEQ ID NO: 85] (corresponding to a nucleotide
sequence from NCBI Accession: XM_583785, encoding amino acids 122-282 of a Bos taurus
TRAIL, as set forth in NCBI Accession: XP_583785);
gctcacataactggaaccagtaggaaaagaagcacatttccatctctaagctccaaatatgaaaaagctttgggccagaaaa
taaactcctgggaatcatcaagaaaaggacattcattcttgaataattttcacttgaggaatggagagctggttatccatcaaacagggtttt
actacatctattcccaaacatactttcgatttcaggaacctgaggaaattttgggaacggtttctacagaagggaacagaaagaaaaaca
ggcaaatgatacagtatatttacaaatggacaagctatcctgaccctatactgctgatgaaaagtgctagaaatagttgttggtctaaagat
tcagaatatggactctattccatctatcaaggtggaatatttgagcttaaggaagatgaccgaatttttgtctctgttactaatgagcaactga
ttgacatggaccaagaagccagttttttcggg [SEQ ID NO: 87] (corresponding to a nucleotide sequence
from NCBI Accession: NM_001024696, encoding amino acids 124-284 of a Sus scrofa
TRAIL, as set forth in NCBI Accession: NP_001019867);
gctcacataactggaaccagtcggagaagcatgtttccaattccaagctccaagaatgataaagctttgggccacaaaataa
actcctgggattccacaagaaaaggacattcattcttgaataatttgcacttgaggaacggagagctggttatccatcaaagggggttttat
tacatctattcccaaacatactttcgatttcaggaacctgaggaaattccaacaggacagaacagaaagagaaacaaacaaatggtcca
atatatttacaaacacacgagttatccggaccctatactgctgatgaaaagtgctagaaatagttgttggtctaaagattctgaatatggact
ctattccatctatcaaggtgggatatttgagcttaaggaaaacgatagaatttttgtctctgtatctaacgagcaattgattgacatggacca
agaagccagttttttcggg [SEQ ID NO: 89] (corresponding to a nucleotide sequence from NCBI
Accession: NM_001130836, encoding amino acids 121-276 of a Canis lupis familiaris
TRAIL, as set forth in NCBI Accession: NP_001124308);
gctcacctaactgggaacagctggagaagctttatctcagtccctgctccaggctcccagagtggaaagaatttgggccaga
aaataagctcctgggaatcatcaaggaaaggacattcattcctgaacaatttgcacctgaggaatggagagctggttatccatcaaacag
gactttattacatctactcccaaacatactttcgatttcaggaacttgaagaaatttcaggaacaatttcaagagaagagatcaaaaagagg
aacaaacaaatggtacaatatatttacaaatggacaagctaccctgaccctatacttctgatgaaaagtgctagaaatagttgttggtctaa
ggattcggaatatggactctattccatctatcaaggaggaatatttgagcttaaggaaaatgaccgaattttcgtctctgtaacgaatgagc
agttgattgacatgaaccaagaatccagtttttttggg [SEQ ID NO: 91] (corresponding to a nucleotide
sequence from NCBI Accession: XM_002716426, encoding amino acids 128-289 of an
Oryctolagus cuniculus TRAIL, as set forth in NCBI Accession: XP_002716472);
gctcacattaccgggatcactcggagaagcaacttagccttaattccaatctccaaggatggaaagaccttgggccagaaga
tagaaacctgggagtcctctcggagagggcattcatttctcaaccatgtgcacttgagaaacggagagctggtgatccaggaggaggg
cctgtattacatctactcccaaacgtactaccggttcaaggaggctaaagaagcttccaagacagtctcgaaggacggagggaggatc
aaacagatggtgcagtacatctacaaatacaccagctaccccgatcccatactgctgatgaagagtgccagaaatagctgctggtccag
agaagctgagtacggactgtactccatctatcagggggggctgttcgagctcaaagaaaatgacaggatttttgtttccgtgacgaatga
gcatttgatggacctggatcaagaagccagtttctttgga [SEQ ID NO: 93] (corresponding to a nucleotide
sequence from NCBI Accession: CH473961, encoding amino acids 128-286 of a Rattus
novegicus TRAIL, as set forth in NCBI Accession: EDM01114);
gctcacattaccgggatcactcggagaagcaacttagccttaattccaatctccaaggatggaaagaccttgggccagaaga
tagaaacctgggagtcctctcggagagggcattcatttctcaaccatgtgcacttgagaaacggagagctggtgatccaggaggaggg
cctgtattacatctactcccaaacgtactaccggttcaaggaggctaaagaagcttccaagacagtctcgaaggacggagggaggatc
aaacagatggtgcagtacatctacaaatacaccagctaccccgatcccatactgctgatgaagagtgccagaaatagctgctggtccag
agaagctgagtacggactgtactccatctatcagggggggctgttcgagctcaaagaaaatgacaggatttttgtttccgtgacgaatga
gcatttgatggacctggaccatgaagccagcttctttgga [SEQ ID NO: 95] (corresponding to a nucleotide
sequence from NCBI Accession: NM_145681, encoding amino acids 128-286 of a Rattus
novegicus TRAIL, as set forth in NCBI Accession: NP_663714);
gctcacattaccgggatcactcggagaagcaacttagccttaattccaatctccaaggatggaaagaccttgggccagaaga
tagaaacctgggagtcctctcggagagggcattcatttctcaaccatgtgcacttgagaaacggagagctggtgatccaggaggaggg
cctgtattacatctactcccaaacgtactaccggttcaaggaggctaaagaagcttccaagacagtctcgaaggacggagggaggatc
aaacagatggtgcagtacatctacaaatacaccagctaccccgatcccatactgctgatgaagagtgccagaaatagctgctggtccag
agaagctgagtacggactgtactccatctatcagggggggctgttcgagctcaaagaaaatgacaggatttttgtttccgtgacgaatga
gcatttgatggacctggatcaagaagccagcttctttgga [SEQ ID NO: 97] (corresponding to a nucleotide
sequence from NCBI Accession: EF030546, encoding amino acids 128-286 of a Rattus
novegicus TRAIL, as set forth in NCBI Accession: ABK32522);
gctcacattactgggatcactcggagaagcaactcagctttaattccaatctccaaggatggaaagaccttaggccagaaga
ttgagtcctgggagtcctctcggaaagggcattcatttctcaaccacgtgctctttaggaatggagagctggtcattgagcaggagggcc
tgtattacatctattcccaaacatacttccgatttcaggaagctaaagacgcttccaagatggtctcaaaggacaaggtgagaaccaaac
agctggtgcagtacatctacaagtacaccagctatccggatcccatagtgctcatgaagagcgccagaaacagctgttggtccagagat
gccgagtacggactgtactccatctatcagggaggactgttcgagctaaaaaaaaatgacaggatttttgtttctgtgacaaatgaacattt
gatggacctggatcaagaagccagcttctttgga [SEQ ID NO: 99] (corresponding to a nucleotide
sequence from NCBI Accession: AK157633, encoding amino acids 128-286 of a Mus
musculus TRAIL, as set forth in NCBI Accession: BAE34141); and
gctcacattactgggatcactcggagaagcaactcagctttaattccaatctccaaggatggaaagaccttaggccagaaga
ttgaatcctgggagtcctctcggaaagggcattcatttctcaaccacgtgctctttaggaatggagagctggtcatcgagcaggagggc
ctgtattacatctattcccaaacatacttccgatttcaggaagctgaagacgcttccaagatggtctcaaaggacaaggtgagaaccaaa
cagctggtgcagtacatctacaagtacaccagctatccggatcccatagtgctcatgaagagcgccagaaacagctgttggtccagag
atgccgagtacggactgtactccatctatcagggaggattgttcgagctaaaaaaaaatgacaggatttttgtttctgtgacaaatgaacat
ttgatggacctggatcaagaagccagcttctttgga [SEQ ID NO: 101] (corresponding to a nucleotide
sequence from NCBI Accession: NM_009425, encoding amino acids 128-286 of a Mus
musculus TRAIL-NCBI Accession: NP_033451);
gtgagagaaagaggtcctcagagagtagcagctcacataactgggaccagaggaagaagcaacacattgtcttctccaaa
ctccaagaatgaaaaggctctgggccgcaaaataaactcctgggaatcatcaaggagtgggcattcattcctgagcaacttgcacttga
ggaatggtgaactggtcatccatgaaaaagggttttactacatctattcccaaacatactttcgatttcaggaggaaataaaagaaaacac
aaagaacgacaaacaaatggtccaatatatttacaaatacacaagttatcctgaccctatattgttgatgaaaagtgctagaaatagttgtt
ggtctaaagatgcagaatatggactctattccatctatcaagggggaatatttgagcttaaggaaaatgacagaatttttgtttctgtaacaa
atgagcacttgatagacatggaccatgaagccagtttttttggggcctttttagttggctaa [SEQ ID NO: 1]
(corresponding to a nucleotide sequence from NCBI Accession: NM_003810, encoding
amino acids 114-281 of a human TRAIL isoform 1, as set forth in NCBI Accession:
NP_003801);
gtnmgngarmgnggnccncarmgngtngcngcncayathacnggnacnmgnggnmgnwsnaayacnytnw
snwsnccnaaywsnaaraaygaraargcnytnggnmgnaarathaaywsntgggarwsnwsnmgnwsnggncaywsntt
yytnwsnaayytncayytnmgnaayggngarytngtnathcaygaraarggnttytaytayathtaywsncaracntayttymg
nttycargargarathaargaraayacnaaraaygayaarcaratggtncartayathtayaartayacnwsntayccngayccnat
hytnytnatgaarwsngcnmgnaaywsntgytggwsnaargaygcngartayggnytntaywsnathtaycarggnggnath
ttygarytnaargaraaygaymgnathttygtnwsngtnacnaaygarcayytnathgayatggaycaygargcnwsnttyttyg
gngcnttyytngtnggntrr [SEQ ID NO: 13] (corresponding to a degenerate nucleotide sequence
encoding a human TRAIL fragment, as set forth in NCBI Accession: 1D0G_A); and
atgathytnmgnacnwsngargaracnathwsnacngtncargaraarcarcaraayathwsnccnytngtnmgng
armgnggnccncarmgngtngcngcncayathacnggnacnmgnggnmgnwsnaayacnytnwsnwsnccnaayws
naaraaygaraargcnytnggnmgnaarathaaywsntgggarwsnwsnmgnwsnggncaywsnttyytnwsnaayytnc
ayytnmgnaayggngarytngtnathcaygaraarggnttytaytayathtaywsncaracntayttymgnttycargargarath
aargaraayacnaaraaygayaarcaratggtncartayathtayaartayacnwsntayccngcnccnathytnytnatgaarws
ngcnmgnaaywsntgytggwsnaargaygcngartayggnytntaywsnathtaycarggnggnathttygarytnaargara
aygaymgnathttygtnwsngtnacnaaygarcayytnathgayatggaycaygargcnwsnttyttyggngcnttyytngtng
gntrr [SEQ ID NO: 15] (corresponding to a degenerate nucleotide sequence encoding a human
TRAIL fragment, as set forth in NCBI Accession: 1DG6);

or

a complement of any one of SEQ ID NO: 1, 13, 15, 53, 55, 57, 59, 61, 63, 65, 67, 69, 71, 73, 75, 77, 79, 81, 83, 85, 87, 89, 91, 93, 95, 97, 99 or 101;

The invention also contemplates variants of TRAIL nucleotide sequences. Nucleic acid variants can be naturally-occurring, such as allelic variants (same locus), homologs (different locus), and orthologs (different organism) or can be non naturally-occurring. Naturally-occurring nucleic acid variants (also referred to herein as polynucleotide variants) such as these can be identified with the use of well-known molecular biology techniques, as, for example, with polymerase chain reaction (PCR) and hybridization techniques as known in the art. Non-naturally occurring polynucleotide variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). For nucleotide sequences, conservative variants include those sequences that, because of the degeneracy of the genetic code, encode the amino acid sequence of a reference TRAIL polypeptide. Variant nucleotide sequences also include synthetically derived nucleotide sequences, such as those generated, for example, by using site-directed mutagenesis but which still encode a TRAIL polypeptide. Generally, variants of a particular TRAIL nucleotide sequence will have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to that particular nucleotide sequence as determined by sequence alignment programs described elsewhere herein using default parameters. In some embodiments, the TRAIL nucleotide sequence displays at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a nucleotide sequence selected from any one of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99 or 101, or their complements.

TRAIL nucleotide sequences can be used to isolate corresponding sequences and alleles from other organisms, particularly other vertebrate animals including mammals. Methods are readily available in the art for the hybridization of nucleic acid sequences. Coding sequences from other organisms may be isolated according to well known techniques based on their sequence identity with the coding sequences set forth herein. In these techniques all or part of the known coding sequence is used as a probe which selectively hybridizes to other TRAIL-coding sequences present in a population of cloned genomic DNA fragments or cDNA fragments (i.e., genomic or cDNA libraries) from a chosen organism (e.g., a mammal). Accordingly, the present invention also contemplates polynucleotides that hybridize to reference TRAIL nucleotide sequences, or to their complements, (e.g., SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99 or 101, or their complements) under stringency conditions described below. As used herein, the term “hybridizes under low stringency, medium stringency, high stringency, or very high stringency conditions” describes conditions for hybridization and washing. Guidance for performing hybridization reactions can be found in Ausubel et al., (1998, supra), Sections 6.3.1-6.3.6. Aqueous and non-aqueous methods are described in that reference and either can be used. Reference herein to low stringency conditions include and encompass from at least about 1% v/v to at least about 15% v/v formamide and from at least about 1 M to at least about 2 M salt for hybridization at 42 C, and at least about 1 M to at least about 2 M salt for washing at 42 C. Low stringency conditions also may include 1% Bovine Serum Albumin (BSA), 1 mM EDTA, 0.5 M NaHPO₄ (pH 7.2), 7% SDS for hybridization at 65 C, and (i) 2 SSC, 0.1% SDS; or (ii) 0.5% BSA, 1 mM EDTA, 40 mM NaHPO₄ (pH 7.2), 5% SDS for washing at room temperature. One embodiment of low stringency conditions includes hybridization in 6 sodium chloride/sodium citrate (SSC) at about 45° C., followed by two washes in 0.2 SSC, 0.1% SDS at least at 50 C (the temperature of the washes can be increased to 55 C for low stringency conditions). Medium stringency conditions include and encompass from at least about 16% v/v to at least about 30% v/v formamide and from at least about 0.5 M to at least about 0.9 M salt for hybridization at 42 C, and at least about 0.1 M to at least about 0.2 M salt for washing at 55° C. Medium stringency conditions also may include 1% Bovine Serum Albumin (BSA), 1 mM EDTA, 0.5 M NaHPO₄ (pH 7.2), 7% SDS for hybridization at 65 C, and (i) 2 SSC, 0.1% SDS; or (ii) 0.5% BSA, 1 mM EDTA, 40 mM NaHPO₄ (pH 7.2), 5% SDS for washing at 60-65 C. One embodiment of medium stringency conditions includes hybridizing in 6 SSC at about 45° C., followed by one or more washes in 0.2 SSC, 0.1% SDS at 60 C. High stringency conditions include and encompass from at least about 31% v/v to at least about 50% v/v formamide and from about 0.01 M to about 0.15 M salt for hybridization at 42 C, and about 0.01 M to about 0.02 M salt for washing at 55 C. High stringency conditions also may include 1% BSA, 1 mM EDTA, 0.5 M NaHPO₄ (pH 7.2), 7% SDS for hybridization at 65 C, and (i) 0.2 SSC, 0.1% SDS; or (ii) 0.5% BSA, 1 mM EDTA, 40 mM NaHPO₄ (pH 7.2), 1% SDS for washing at a temperature in excess of 65° C. One embodiment of high stringency conditions includes hybridizing in 6 SSC at about 45° C., followed by one or more washes in 0.2 SSC, 0.1% SDS at 65° C.

In certain embodiments, a TRAIL polypeptide is encoded by a nucleic acid sequence that hybridizes to a disclosed nucleotide sequence under very high stringency conditions. One embodiment of very high stringency conditions includes hybridizing 0.5 M sodium phosphate, 7% SDS at 65 C, followed by one or more washes at 0.2 SSC, 1% SDS at 65° C.

Other stringency conditions are well known in the art and a skilled addressee will recognize that various factors can be manipulated to optimize the specificity of the hybridization. Optimization of the stringency of the final washes can serve to ensure a high degree of hybridization. For detailed examples, see Ausubel et al., supra at pages 2.10.1 to 2.10.16 and Sambrook et al. (1989, supra) at sections 1.101 to 1.104.

While stringent washes are typically carried out at temperatures from about 42° C. to 68 C, one skilled in the art will appreciate that other temperatures may be suitable for stringent conditions. Maximum hybridization rate typically occurs at about 20° C. to 25° C. below the Tₘ for formation of a DNA-DNA hybrid. It is well known in the art that the Tₘ is the melting temperature, or temperature at which two complementary polynucleotide sequences dissociate. Methods for estimating Tₘ are well known in the art (see Ausubel et al., supra at page 2.10.8). In general, the Tₘ of a perfectly matched duplex of DNA may be predicted as an approximation by the formula:

Tₘ=81.5+16.6(log₁₀M)+0.41(% G+C)−0.63(% formamide)−(600/length)

wherein: M is the concentration of Na⁺, preferably in the range of 0.01 molar to 0.4 molar; % G+C is the sum of guanosine and cytosine bases as a percentage of the total number of bases, within the range between 30% and 75% G+C; % formamide is the percent form amide concentration by volume; length is the number of base pairs in the DNA duplex. The Tₘ of a duplex DNA decreases by approximately 1 C with every increase of 1% in the number of randomly mismatched base pairs. Washing is generally carried out at Tₘ−15° C. for high stringency, or Tₘ−30 C for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5 SSC, 5 Denhardt's solution (0.1% ficoll, 0.1% polyvinylpyrrolidone and 0.1% bovine serum albumin), 0.1% SDS and 200 mg/mL denatured salmon sperm DNA) containing labeled probe. The membrane is then subjected to two sequential medium stringency washes (i.e., 2 SSC, 0.1% SDS for 15 min at 45° C., followed by 2 SSC, 0.1% SDS for 15 min at 50° C.), followed by two sequential higher stringency washes (i.e., 0.2 SSC, 0.1% SDS for 12 min at 55° C. followed by 0.2 SSC and 0.1% SDS solution for 12 min at 65-68° C.

The present invention also contemplates the use of TRAIL chimeric or fusion proteins for eliciting at least one of the following activities: (a) stimulating apoptosis of adipose cells or tissues; (b) reducing fasting hyperinsulinemia, (c) reducing glucose levels after a hyperglycemic stimulus; (d) reducing hyperinsulinemia after a hyperglycemic stimulus, (e) enhancing peripheral response to insulin; (f) reducing increased adiposity in response to high fat diet, (g) improving mitochondrial fatty acid oxidative capacity of muscle tissue, (h) reducing circulating levels of the proinflammatory cytokines IL-6, IL-1alpha and MCP, (i) counteracting lipopolysaccaride- and muramildipeptide-induced inflammation and fever for controlling adiposity including the treatment or prevention of adiposity-related conditions. As used herein, a TRAIL “chimeric protein” or “fusion protein” includes a TRAIL polypeptide linked to a non-TRAIL peptide or polypeptide. A “non-TRAIL peptide or polypeptide” refers to a peptide or polypeptide having an amino acid sequence corresponding to a protein which is different from a TRAIL polypeptide and which is derived from the same or a different organism. The TRAIL polypeptide of the fusion protein can correspond to all or a portion e.g., a fragment described herein of a TRAIL polypeptide amino acid sequence. In a specific embodiment, a TRAIL fusion protein includes at least one biologically active portion of a TRAIL polypeptide. The non-TRAIL peptide or polypeptide can be fused to the N-terminus or C-terminus of the TRAIL polypeptide. Non-limiting examples of chimeric TRAIL polypeptides comprise a biologically active TRAIL polypeptide that interacts with a TRAIL DR (e.g., TRAIL-R1 or TRAIL-R2) and an heterologous trimerizing domain, as described infra.

The fusion protein can include a moiety which has a high affinity for a ligand. For example, the fusion protein can be a GST-TRAIL fusion protein in which the TRAIL sequence is fused to the C-terminus of the GST sequence. Such fusion proteins can facilitate the purification of recombinant TRAIL polypeptide.

Alternatively, the fusion protein can be a TRAIL protein containing a heterologous signal sequence at its N-terminus. TRAIL is a type II protein lacking a native signal sequence and thus a heterologous signal sequence or leader functional in mammalian host cells can be added to increase expression and/or secretion of the TRAIL protein. Examples include the signal sequence for interleukin-7 (IL-7) described in U.S. Pat. No. 4,965,195, the signal sequence for interleukin-2 receptor described in Cosman et al., Nature 312:768, 1984; the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. Another option is a leader derived from Ig-κ, such as a leader comprising the amino acid sequence MGTDTLLWVLLLWVPGSTG [SEQ ID NO: 133]. Further alternatives are cytomegalovirus-derived leaders (e.g., MARRLWILSLLAVTLTVALAAPSQKSKRRTSS [SEQ ID NO: 134]) and signal peptides derived from a growth hormone (e.g., MATGSRTSLLLAFGLLCLPWLQEGSA [SEQ ID NO: 135]).

In some embodiments, fusion proteins may include all or a part of a serum protein, e.g., an IgG constant region, or human serum albumin.

The TRAIL fusion proteins of the invention can be incorporated into pharmaceutical compositions and administered to a subject in vivo. They can also be used to modulate the bioavailability of an TRAIL polypeptide.

3.2 TRAIL DR Peptide Agonists

The present invention also contemplates peptide compounds that agonize TRAIL DRs. In some embodiments, the compounds agonize TRAIL-R2 and comprise, consist or consist essentially of the amino acid sequence:

Ac-WDCLDNX₁IGRRQCVX₂L-NH₂ [SEQ ID NO: 136], wherein X₁ and X₂ are each independently selected R and K. Representative compounds of this type are selected from:

| | |
|---|---|
| AcWDCLDNRIGRRQCVKL-NH_{2;} | [SEQ ID NO: 137] |
| AcGGSWDCLDNRIGRRQCVKL-NH_{2;} | [SEQ ID NO: 138] |
| AcWDCLDN(X₃)IGRRQCVKL-NH_{2;} | [SEQ ID NO: 139] |
| AcWDCLDRPGRRQCVK-NH_{2;} | [SEQ ID NO: 140] |
| AcWDCLDNKIGRRQCVRL-NH_{2;} | [SEQ ID NO: 141] |
| AcCLDNRIGRRQCV; | [SEQ ID NO: 142] |
| AcDCLDNRIGRRQCVKL-NH_{2;} | [SEQ ID NO: 143] |
| AcWDCLDNRIGKRQCVRL-NH_{2;} | [SEQ ID NO: 144] |
| AcWDCLDNRIG(X₄)RQCV(X₅)L-NH_{2;} | [SEQ ID NO: 145] |
| AcWDCLDNRIGRRQCVK-NH_{2;} | [SEQ ID NO: 146] |
| AcWDCLVDRPGRRQCVRLEK-NH_{2;} | [SEQ ID NO: 147] |
| AcWDCLVDRPGRRQCVRLERK-NH_{2;} | [SEQ ID NO: 148] |
| AcWDCLVDRPGRRQCVKLER-NH_{2;} | [SEQ ID NO: 149] |
| GGGSWDCLDNRIGRRQCVKL; | [SEQ ID NO: 150] |
| AcCWDLDNRIGRRQVCKL-NH_{2;} | [SEQ ID NO: 151] |
| and | |
| GGGSWDCLD-NRIGRRQCVKL-NH_{2,} | [SEQ ID NO: 152] |

wherein X₃, X₄, and X₅ are independently selected from R and K.

In illustrative examples of this type, the compounds are selected from:

[SEQ ID NO: 153]

Ac-WDC*LDNX1IGRRQC*VX₂LNH₂,

[SEQ ID NO: 154]

Ac-WDC*LDNRIGRRQC*VKLNH₂,

[SEQ ID NO: 155]

Ac-GGSWDC*LDNRIGRRQC*VKLNH₂,

[SEQ ID NO: 156]

Ac-WDC*LDNX₃IGRRQC*VKLNH₂,

[SEQ ID NO: 157]

Ac-WDC*LDRPGRRQC*VKNH₂,

[SEQ ID NO: 158]

Ac-WDC*LDNKIGRRQC*VRLNH₂,

[SEQ ID NO: 159]

Ac-C*LDNRIGRRQC*V,

[SEQ ID NO: 160]

Ac-DC*LDNRIGRRQC*VKL-NH₂,

[SEQ ID NO: 161]

Ac-WDC*LDNRIGKRQC*VRL-NH2,

[SEQ ID NO: 162]

Ac-WDC*LDNRIGX₄RQC*VX₅L-NH₂,

[SEQ ID NO: 163]

Ac-WDC*LDNRIGRRQC*VK-NH₂,

[SEQ ID NO: 164]

Ac-WDC*LVDRPGRRQC*VRLEK-NH₂,

[SEQ ID NO: 165]

Ac-WDC*LVDRPGRRQC*VRLERK-NH₂,

[SEQ ID NO: 166]

Ac-WDC*LVDRPGRRQC*VKLER-NH₂,

[SEQ ID NO: 167]

GGGSWDC*LDNRIGRRQC*VKL-NH₂,
and

[SEQ ID NO: 168]

ACC*WDLDNRIGRRQVC*KL-NH₂,

wherein X₂, X₃, X₄ and X₅ are as defined above and * represents a cysteine residue of a disulfide bond.

In some embodiments, the peptide agonist compounds defined above are suitably in the form of monomers, dimers (e.g., homodimers or heterodimers) or trimers (e.g., homotrimers or heterotrimers). In illustrative examples, the above peptides compounds are multimerized (e.g., dimerized, trimerized, etc.) via a linker (e.g., a peptide bond). Choosing a suitable linker for a specific case where two polypeptide chains are to be connected depends on various parameters, e.g., the nature of the two polypeptide chains (e.g., whether they naturally oligomerize (e.g., form a dimer or trimer, or not), the distance between the N- and the C-termini to be connected if known from three-dimensional structure determination, and/or the stability of the linker towards proteolysis and oxidation. In illustrative examples of this type, a lysine residue is used. In other illustrative examples, other bi-functional linkers are used. Alternatively, or in addition, the compounds or peptides may contain cysteine residues for the purpose of introducing an intramolecular disulfide bridge or constraint at various locations in the amino acid sequence. A skilled artisan will be able to select appropriate linkers from both these and other linker moieties known in the art, as well as from other linkers that may be subsequently developed. In particular, the skilled artisan will recognize that the substitution of a particular linker moiety may be useful for optimizing binding and/or other functional properties.

Representative peptide agonists according to SEQ ID NO: 136 are disclosed, for example, in US 2009/0131317, which is incorporated by reference herein in its entirety.

In some embodiments, the peptide agonist compounds defined above are suitably in the form of monomer, dimers (e.g., homodimers or heterodimers) or trimers (e.g., homotrimers or heterotrimers).

Representative peptide agonists according to SEQ ID NO: 136 and methods for their preparation are disclosed, for example, in US 2009/0131317, which is incorporated by reference herein in its entirety.

3.3 Chimeric Polypeptides that Modulate TRAIL DRs

The present invention also contemplates as TRAIL DR agonists chimeric or non-natural polypeptides, which comprise a trimerizing domain and at least one polypeptide that binds to at least one TRAIL death receptor (e.g., TRAIL-R1 or TRAIL-R2). The trimerizing domain may comprise, consist or consist essentially of a polypeptide of: X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅L₁X₂₆X₂₇X₂₈X₂₉X₃₀L₂X₃₁X₃₂E₁V₁X₃₃X₃₄L₃K₁E₂X₃₅Q₁A₁L₄Q₂T₁V₂C₁L₅X₃₆ (SEQ ID NO: 337), having up to five amino acid substitutions at X₁₉, X₁₇, X₂₀, X₂₁, X₂₄, X₂₅, L₁, X₂₇, X₂₈, X₂₉, X₃₀, L₂, X₃₁, X₃₂, or E₁, wherein each X is independently selected from any amino acid residue and wherein three trimerizing domains form a trimeric complex. In representative examples of this type, the trimerizing domain comprises a trimerizing peptide or polypeptide selected from the group consisting of:

[SEQ ID NO: 169]

NTGLLESQLSRHDQMLSVHDIRLADMDLRFQVLETASYNGVLIW
KIRDYKRRKQEAVM (hTRAF3);

[SEQ ID NO: 170]

AASERKALQTEMARIKKWLTF (hMBP),

[SEQ ID NO: 171]

FDMSCRSRLATLNEKLTALERRIEYIEARVTKGETLT (hSPC300),

[SEQ ID NO: 172]

ADIYKADFQAERQAREKLAEKKELLQEQLEQLQREYSKLKASC
QES(ARIhNEMO),

[SEQ ID NO: 173]

LTGSAQNIEFRTGSLGKIKLNDEDLSECLHQIQKNKEDIIELKGSA
IGLPIYQLNSKLVDLERKFQGLQQT (hcubilin),

[SEQ ID NO: 174]

LRGLRTIVTTLQDSIRKVTEENKELANE hThrombospondins,

[SEQ ID NO: 175]

VASLRQQVEALQGQVQHLQAAFSQYKK (neck region of
human SP-D),

[SEQ ID NO: 338]

VNALRQRVGILEGQLQRLQNAFSQYKK (neck region of
bovine SP-D),

[SEQ ID NO: 176]

SAALRQQMEALNGKLQRLEAAFSRYKK (neck region of
rat SP-D),

[SEQ ID NO: 177]

VNALKQRVTILDGHLRRFQNAFSQYKK (neck region
of bovine conglutinin);

[SEQ ID NO: 178]

VDTLRQRMRNLEGEVQRLQNIVTQYRK (neck region
of bovine collectin);
and

[SEQ ID NO: 179]

GSPGLKGDKGIPGDKGAKGESGLPDVASLRQQVEALQGQ
VQHLQAAFSQYKKVELFPGGIPHRD (neck region of
human SP-D).

In some embodiments, the polypeptide that binds to a TRAIL death receptor comprises a biologically active fragment of TRAIL, which comprises, consists or consists essentially of about 5 to about 50 amino acid residues, or about 5 to about 25, or about 10 to about 20 residues, or about 12 to about 20 amino acid residues of a TRAIL polypeptide as defined herein. Optionally, the TRAIL peptide comprises, consists or consists essentially of no more than 25 amino acid residues (e.g., 25, 23, 21, 19, 17, 15 or less amino acid residues).

In some embodiments, the polypeptide that binds to a TRAIL death receptor comprises C-Type Lectin Like Domain (CLTD) wherein one of loops 1, 2, 3 or 4 of loop segment A or loop segment B comprises a polypeptide sequence that binds at least one of TRAIL-R1 and TRAIL-R2.

Non-limiting examples of polypeptides that bind to TRAIL-R1 comprise a CLTD comprising one of the following combinations of sequences in loops 1 and 4:

| | Loop 1 | | |
|---|---|---|---|
| | SEQ ID | | Loop 4 SEQ |
| Loop 1 Sequence | NO | Loop 4 Sequence | ID NO |
| GWLEGAGW | 180 | DGGWHWRWEN | 181 |
| GWLEGVGW | 182 | DGGEHWGWEN | 183 |
| GYLAGVGW | 184 | DGGRGFRWEN | 185 |
| GWLEGYGW | 186 | DGGTWWEWEN | 187 |
| GYLEGYGW | 188 | DGGATIAWEN | 189 |
| GWLqGVGW | 190 | DGGRGWPWEN | 191 |
| GYLAGYGW | 192 | DGGPSIWREN | 193 |
| GYIEGTGW | 194 | DGGSNWAWEN | 195 |
| GYMSGYGW | 196 | DGGMMARWEN | 197 |
| GFMVGRGW | 198 | DGGSMWPWEN | 199 |
| MVTRPPYW | 200 | DGGWVMSFEN | 201 |
| PFRVPqWW | 202 | DGGYGPVqEN | 203 |
| GWLEGAGW | 204 | DGGWQWRWEN | 205 |
| GYLDGVGW | 206 | DGGQGCRWEN | 207 |
| VLRLAWSW | 208 | DGGKRNGCEN | 209 |
| WLSLFSPW | 210 | DGGRGVRGEN | 211 |
| GWMAGVGW | 212 | DGGRRLPWEN | 213 |
| SYRLHYGW | 214 | DGGRRWLGEN | 215 |
| IWPLRFRW | 216 | DGGFVTRKEN | 217 |
| WqLYYRYW | 218 | DGGVGCMVEN | 219 |
| RCLqGVGW | 220 | DGGRGWPWEN | 221 |
| GCTqGQGW | 222 | DGGKKWKWEN | 223 |
| GFLqGNGW | 224 | DGGMWDRWEN | 225 |
| GVLqRGGW | 226 | DGGPGGEREN | 227 |
| PFRVLqQWW | 228 | DGGCGPVqQEN | 229 |
| PFRGPqQWW | 230 | DGGYGPVGEN | 231 |
| ARFAMWqQW | 232 | DGGRAGVGEN | 233 |
| GWLQGYGW | 234 | DGGqQIGWGEN | 235 |
| AWRSWLNW | 236 | DGGREqQRREN | 237 |
| GWLEGVGW | 238 | DGGWPFSNEN | 239 |
| GWLMGTGW | 240 | DGGWWNRWEN | 241 |
| VRRMGFHW | 242 | DGGRVAVGEN | 243 |
| RYHVQALW | 244 | DGGRVRPREN | 245 |
| IqCSPPLW | 246 | DGGAVqqQEN | 247 |
| GLARQqGW | 248 | DGGKGRPREN | 249 |
| GWLSGVGW | 250 | DGGWAHAWEN | 251 |
| GWLEGVGW | 252 | DGGGGVRWEN | 253 |
| GWLSGYGW | 254 | DGGRVWSWEN | 255 |
| GLLSDWWW | 256 | DGGGNqSREN | 257 |
| QWVAFWSW | 258 | DGGSAVSGEN | 259 |
| PYTSWGLW | 260 | DGGVGGRGEN | 261 |
| VARWLLKW | 262 | DGGMCKPCEN | 263 |
| GFLAGVGW | 264 | DGGWWTRWEN | 265 |
| GYLQGSGW | 266 | DGGWKTRWEN | 267 |
| VRHWLqLW | 268 | DGGGWWKGEN | 269 |

Non-limiting examples of polypeptides that bind to TRAIL-R2 comprise a C-Type Lectin Like Domain (CLTD) comprising one of the following combinations of sequences in loops 1 and 4:

| | Loop 1 | | Loop 4 |
|---|---|---|---|
| | SEQ ID | | SEQ ID |
| Loop 1 Sequence | NO | Loop 4 Sequence | NO |
| RATLRPRW | 270 | DGG----KN | 271 |
| RAMLRSRW | 272 | DGGRWFQGKN | 273 |
| RALFRPRW | 274 | DGGPWYLKEN | 275 |
| RAVLRPRW | 276 | DGGWVLGGKN | 277 |
| RAWLRPRW | 278 | DGGTLVSGEN | 279 |
| RVIRRSMW | 280 | DGGQKWMAEN | 281 |
| RVLQRPVW | 282 | DGGMVWSMEN | 283 |
| RVqLRPRW | 284 | EGGFRRHAKN | 285 |
| RVVRLSEW | 286 | DGGMLWAMEN | 287 |
| RVISAPVW | 288 | DGGQQWAMEN | 289 |
| RVLRRPQW | 290 | NGGDWRIPEN | 291 |
| RVMMRPRW | 292 | DGGMWGAMEN | 293 |
| RVMRRVLW | 294 | DGGRRETMKN | 295 |
| RVMRRPLW | 296 | DGGRGQQWEN | 297 |
| RVMRRREW | 298 | DGAQLMALEN | 299 |
| RVWRRSLW | 300 | DGGHLVKQKN | 301 |
| KRRWYGGW | 302 | DGGVNTVREN | 303 |
| KRVWYRGW | 304 | DGGMRRRREN | 305 |
| AVIRRPLW | 306 | DGGMKYTMEN | 307 |
| ELVTSRLW | 308 | DGGVMqLGEN | 309 |
| ELGTSRLW | 310 | DGGVMqLGEN | 311 |
| FRGWLRWW | 312 | DDGARVLAEN | 313 |
| GRLKGIGW | 314 | DGGRPQWGEN | 315 |
| GVWqSFPW | 316 | DGGLGYLREN | 317 |
| HLVSLAPW | 318 | DGGGMHQGKN | 319 |
| HIFIDWGW | 320 | DGGVMTMGEN | 321 |
| PVMRGVTW | 322 | DGGRSWVWEN | 323 |
| QLVTVGPW | 324 | DGGVMHRTEN | 325 |
| QLVVqMGW | 326 | DGGWMTVGEN | 327 |
| VAIRRSVW | 328 | DGGERAHSEN | 329 |
| WVMRRPLW | 330 | DGGSMGWREN | 331 |
| WRSMVVWW | 332 | DGGKHTLGEN | 333 |
| ELRTDGLW | 334 | DGGVMRRSEN | 335 |

In specific embodiments, the polypeptide that binds to a TRAIL death receptor is positioned at one of the N-terminus and the C-terminus of the trimerizing domain.

In some embodiments, the polypeptide that binds to a TRAIL death receptor does not bind to a TRAIL decoy receptor.

The above TRAIL DR agonist chimeric molecules and methods for their preparation are disclosed in US 2010/0105620, which is incorporated by reference herein in its entirety.

3.4 TRAIL DR Agonist Antigen-Binding Molecules

The present invention also contemplates the use of TRAIL DR agonist antigen-binding molecules for eliciting at least one of activity selected from: (a) stimulating apoptosis of adipose cells or tissues; (b) reducing fasting hyperinsulinemia, (c) reducing glucose levels after a hyperglycemic stimulus; (d) reducing hyperinsulinemia after a hyperglycemic stimulus, (e) enhancing peripheral response to insulin; (f) reducing increased adiposity in response to high fat diet, (g) improving mitochondrial fatty acid oxidative capacity of muscle tissue, (h) reducing circulating levels of the proinflammatory cytokines IL-6, IL-1alpha and MCP-1, (i) counteracting lipopolysaccaride- and muramildipeptide-induced inflammation and elevation of body temperature for in controlling adiposity including in the treatment or prevention of adiposity-related conditions.

Representative antigen-binding molecules include whole antibodies (e.g., polyclonal or monoclonal) that bind to TRAIL receptor (DR4 or DR5). The invention also contemplates as antigen-binding molecules Fv, Fab, Fab and F(ab)₂ immunoglobulin fragments. Alternatively, the antigen-binding molecule may be in the form of a synthetic stabilized Fv fragment, a single variable region domain (also known as a dAbs), a “minibody” and the like as known in the art. The antigen-binding molecules also encompass dimeric antibodies, as well as multivalent forms of antibodies.

In some embodiments, the TRAIL agonist antigen-binding molecules are chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, for example, U.S. Pat. No. 4,816,567; and Morrison et al., Proc Natl Acad Sci USA 81:6851-6855, 1984).

Also contemplated as antigen binding molecules are humanized antibodies. Humanized antibodies are produced by transferring complementary determining regions from heavy and light variable chains of a non human (e.g., rodent, preferably mouse) immunoglobulin into a human variable domain. Typical residues of human antibodies are then substituted in the framework regions of the non human counterparts. The use of antibody components derived from humanized antibodies obviates potential problems associated with the immunogenicity of non human constant regions. General techniques for cloning non human, particularly murine, immunoglobulin variable domains are described, for example, by Orlandi et al., Proc Natl Acad. Sci. USA 86:3833, 1989). Techniques for producing humanized monoclonal antibodies are described, for example, by Jones et al., Nature 321:522, 1986; Carter et al., Proc Natl Acad Sci USA 89:4285, 1992; Sandhu, Crit. Rev Biotech 12:437, 1992; Singer et al., J Immunol 150:2844, 1993; Sudhir, ed., Antibody Engineering Protocols, Humana Press, Inc. 1995; Kelley, “Engineering Therapeutic Antibodies,” in Protein Engineering: Principles and Practice Cleland et al. (eds.), pages 399-434, John Wiley & Sons, Inc., 1996; and by Queen et al., U.S. Pat. No. 5,693,762, 1997. Humanized antibodies include “primatized” antibodies in which the antigen-binding region of the antibody is derived from an antibody produced by immunizing macaque monkeys with the antigen of interest. Also contemplated as antigen binding molecules are humanized antibodies. Humanized antibodies are produced by transferring complementary determining regions from heavy and light variable chains of a non human (e.g., rodent, preferably mouse) immunoglobulin into a human variable domain. Typical residues of human antibodies are then substituted in the framework regions of the non human counterparts. In addition, camelidae single-chain antibodies and their recombinant VHH domains humanized against TRAIL DRs will be considered, according to De Marco, Microbial Cell Factories 10:44, 2011.

Non-limiting examples of antigen-binding molecules that are immuno-interactive with TRAIL DRs and methods for their preparation are described by Sung et al., Mol Cancer Ther 8:2276-2285, 2009; Feng et al., MAbs MAbs. 2:565-570, 2010; Chen et al., Cell Res 19:984-995, 2009.

In specific embodiments, the TRAIL DR agonist antigen-binding molecules are selected from:

(a) an anti-TRAIL-R1 single chain Fv antibody, which comprises the amino acid sequence: EVQLVQSGAEVKMPGASVKLSCRVSGDTFTAYFIHWVRQAPGQGLEWMGWFNPISG TAGSAEKFRGRVAMTRDTSISTAYMELNRLTFDDTAVYYCARQHRGNTFDPWGQGT LVTVSSGGGGSGGGGSGGGGSAQSALTQPASVSGSPGQSITISCTGTSSDIGAYKYVS WYQQHPGKAPKLVIYEVSNRPSGVSSRFSGSKSGQTASLTISGLQADDEADYYCNSY QGYNTWVFGGGTKVTVLG [SEQ ID NO: 336], as disclosed for example in US 2010/0210545, which is incorporated by reference herein in its entirety;

(b) a humanized TRAIL-R1 agonist monoclonal antibody designated HGS-ETR1 or Mapatumumab (Human Genome Sciences, Rockville, Md., USA);

(c) a humanized TRAIL-R2 agonist monoclonal antibody designated HGS-ETR1 (Human Genome Sciences, Rockville, Md., USA);

(d) a humanized TRAIL-R2 agonist monoclonal antibody designated CS-1008 or Tigatuzumab (Daiichi Sankyo Inc. NJ, USA);

(e) a human TRAIL-R2 agonist monoclonal antibody designated AMG655 or Conatumumab (Amgen, CA, USA);

(f) TRAIL-R2 agonist antigen-binding molecules disclosed in US 2007/0179086, which is hereby incorporated by reference in its entirety; and

(g) TRAIL DR agonist antigen-binding molecules disclosed in US 2008/0199423, which is incorporated by reference herein in its entirety.

3.5 Small Molecule TRAIL DR Agonists

The present invention also contemplates small molecule agonists of TRAIL death receptors. In some embodiments, the small molecule TRAIL DR agonists are selected from compounds having either the formula:

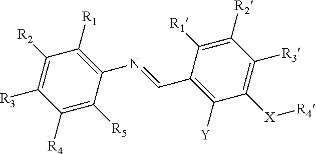

wherein: R₁, R₂, R₃, R₄, R₅, R_{1′}, R_{2′}, and R_{3′} are each independently H, hydroxy, amino, cyano, halo, nitro, mercapto, OPO(OH)₂, PO(OH)₂, OSO₂OH, SO.sub.2OH, or a heteroatom-substituted or heteroatom-unsubstituted C₁-C.sub₃-alkyl, C₂-C₃-alkenyl, C₂-C₃-alkynyl, C₁-C₃-acyl, C₁-C₃-alkoxy, C₁-C₃-acyloxy, C₁-C₃-alkylamino, or C₁-C₃-amido; R_{4′} is H or a heteroatom-substituted or heteroatom-unsubstituted C₁-C₁₀-alkyl, C₁-C₁₀-aryl, C₂-C₁₀-aralkyl, C₂-C₁₀-alkenyl, C₂-C₁₀-alkynyl, or C₁-C₁₀-acyl; X is selected from the group consisting of O, S, and NH, and Y is selected from the groups consisting of hydroxy, amino, and mercapto;

or the formula:

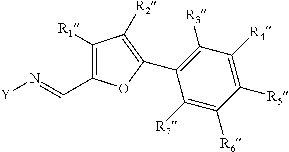

wherein: R_{1″}, R_{2″}, R_{3″}, R_{4″}, R_{5″}, R_{6′} and R_{7″}, are each independently H, hydroxy, amino, cyano, halo, nitro, mercapto, OPO(OH)₂, PO(OH)₂, OSO.₂OH, SO₂OH, or a heteroatom-substituted or heteroatom-unsubstituted C₁-C₈-alkyl, C₂-C₈-alkyl, alkenyl, C₂-C₈-alkynyl, C₁-C₈-aryl, C₁-C₈-aralkyl, C₁-C₈-acyl, C₁-C₈-alkoxy, C₁-C₈-aryloxy, C₂-C₈-aralkoxy, C₁-C₈-acyloxy, C₁-C₈-alkylamino, C₁-C₈-arylamino, C₂-C₈-aralkylamino, or C₁-C₈-amido; Y is selected from the groups consisting of heteroatom-substituted or heteroatom-unsubstituted C₁-C₁₅-alkylamino, C₁-C₁₅-alkenylamino, C₁-C₁₅-alkynylamino, C₁-C₁₅-arylamino, C₂-C₁₅-aralkylamino, and C₁-C₁₅-amido; or a pharmaceutically acceptable salt, hydrate, amine-N-oxide, imine-N-oxide, tautomer, or optical isomer of either of the above formulas.

In specific embodiments, the compounds are represented by the structure:

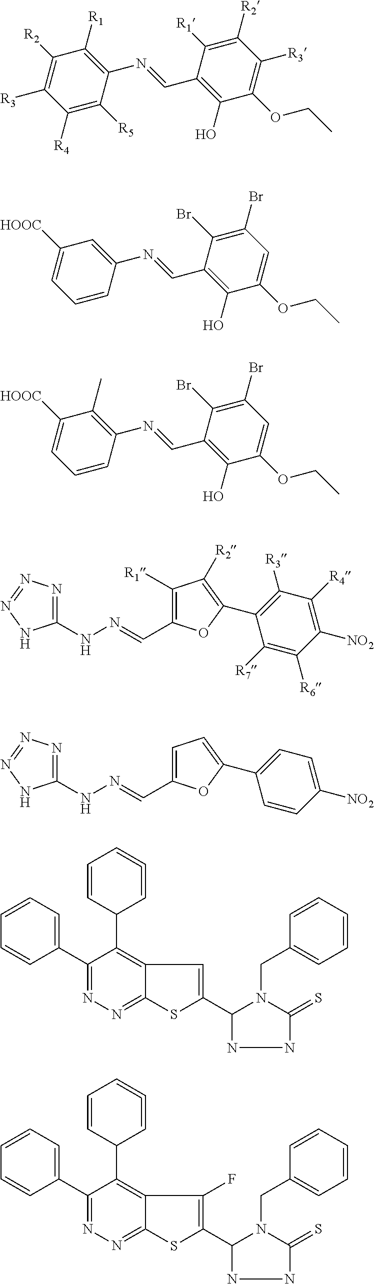

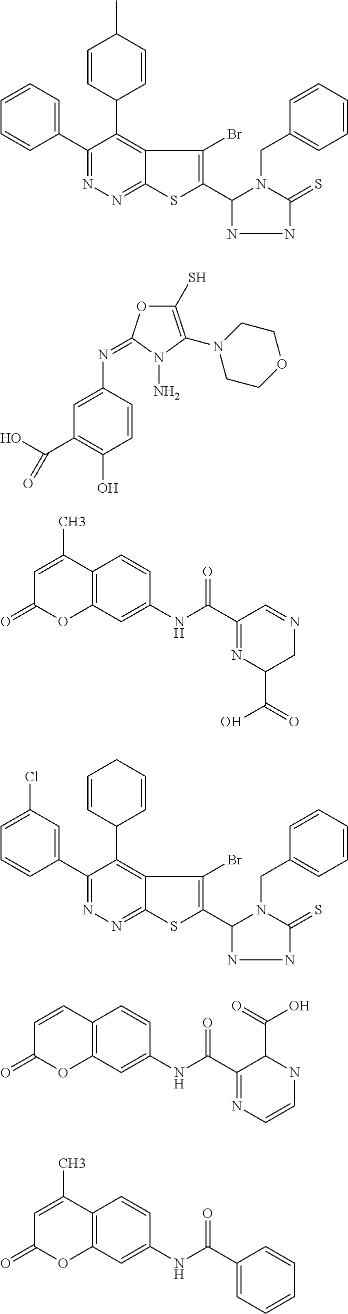

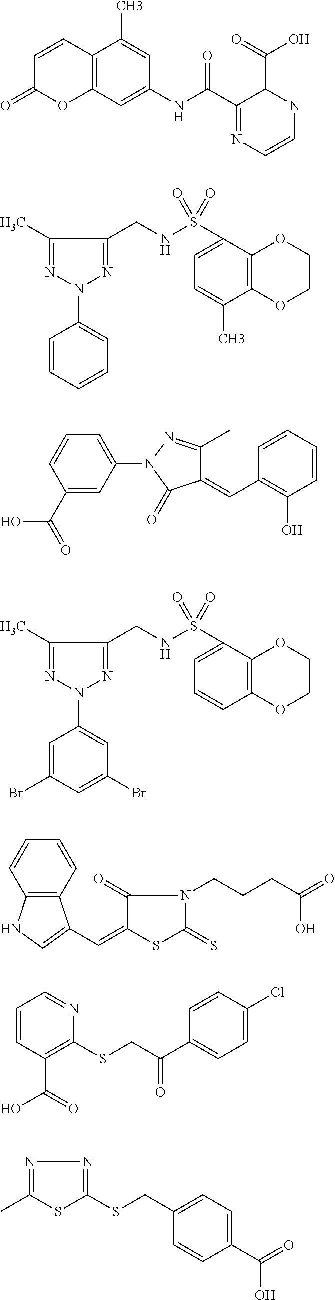

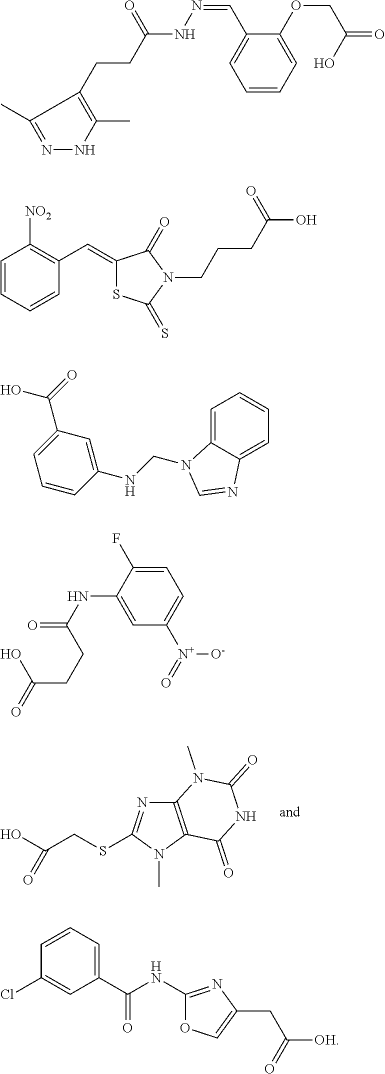 [SMILES: Cc1n[nH]c(C)c1CCC(=O)N/N=C\c1ccccc1OCC(=O)O ; O=C(O)CCCN1C(=O)/C(=C/c2ccccc2[N+](=O)[O-])SC1=S ; O=C(O)c1cccc(NCn2cnc3ccccc32)c1 ; O=C(O)CCC(=O)Nc1cc([N+](=O)[O-])ccc1F ; Cn1c(SCC(=O)O)nc2c1c(=O)[nH]c(=O)n2C ; O=C(O)Cc1coc(NC(=O)c2cccc(Cl)c2)n1]

The above compounds and methods for their preparation are disclosed in US 2008/0214547, which is incorporated herein by reference in its entirety.

3.6 Screening Methods

The present invention also features methods of screening for agents that agonize a TRAIL DR. Candidate agents encompass numerous chemical classes, though typically they are organic molecules, preferably small organic compounds having a molecular weight of more than 50 and less than about 2,500 Dalton. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agent often comprises cyclical carbon or heterocyclic structures or aromatic or polyaromatic structures substituted with one or more of the above functional groups. Candidate agents are also found among biomolecules including, but not limited to: peptides, saccharides, fatty acids, steroids, purines, pyrimidines, derivatives, structural analogues or combinations thereof.

Small (non-peptide) molecule modulators of a TRAIL DR polypeptide are particularly advantageous. In this regard, small molecules are desirable because such molecules are more readily absorbed after oral administration, have fewer potential antigenic determinants, or are more likely to cross the cell membrane than larger, protein-based pharmaceuticals.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening for TRAIL DR agonist agents can be achieved by any suitable assay. The ability of candidate agents to activate or agonize a TRAIL DR can be measured using cultured cells (e.g., cultured adipose cells), including cell lines (e.g., 3T3-L1 cells) or primary cells (e.g., isolated from mouse, rat or human) or in vivo by administering molecules to an appropriate animal model.

In some embodiments, a screening assay for TRAIL DR agonists is employed, which comprises (1) providing a purified preparation of a TRAIL DR polypeptide (e.g., TRAIL-R1, TRAIL-R2 etc.) or a cell or cell membrane in which the TRAIL DR is present on the surface of the cell or cell membrane, (2) incubating the TRAIL DR polypeptide, cell or cell membrane in the presence of a TRAIL polypeptide and a candidate agent, and (3) measuring the binding of the TRAIL to the TRAIL DR polypeptide, cell or cell membrane. The agent tests positive as one that binds or otherwise interacts with the TRAIL DR polypeptide if it reduces binding of the TRAIL polypeptide to the TRAIL DR polypeptide, cell or cell membrane and thus competes with the TRAIL polypeptide for binding to the TRAIL DR polypeptide, cell or cell membrane. In some embodiments in which a cell is used for the screening, the cell may normally expresses the TRAIL DR (e.g., an adipose cell such as but not limited to adipocytes and preadipocytes, including cell lines thereof (e.g., 3T3-L1 cells) and primary adipose cells (e.g., isolated from mouse, rat or human). Alternatively, the cell can be one that has been transformed with a construct from which a TRAIL DR is expressed.

Suitably, the screening assay for TRAIL DR agonists comprises contacting a cell that expresses a TRAIL DR on its surface with a candidate agent and detecting an activity of the TRAIL DR (e.g., caspase activation, apoptosis induction, intracellular signal transduction etc.), whereby the candidate agent tests positive as a TRAIL DR agonist if it activates the TRAIL DR. In illustrative examples of this type, a candidate agent is identified as TRAIL DR agonist, by: culturing a first sample of cells selected from adipocytes or their precursors in the presence of the candidate agent and determining the viability or number of those cells; culturing a second sample of cells selected from adipocytes or their precursors in the absence of the candidate agent and determining the viability, proliferation and differentiation of those cells; comparing the viability or number of the first sample of cells with the viability or number of the second sample of cells, whereby the agent tests positive as a TRAIL DR agonist when the viability or number of the first sample of cells is reduced or lower, as compared to the viability or number of the second sample of cells.

In other embodiments, the screening assay for TRAIL DR agonists comprises administering to an animal model, or a human, the candidate agent and measuring the animal's responsiveness to that agent, whereby the agent tests positive when it reduces or inhibits adiposity in the animal model or human. In vivo evaluation tools, which are well known to practitioners in the art, are available for evaluating adiposity. For example, the amount of adipose tissue can be measured using skin fold measurement (e.g., using an adipometer). This involves the integration of skin fold thicknesses from suitable areas (e.g., triceps, biceps, subscapular and suprailiac regions) to obtain a body fat percentage value. Other in vivo evaluation tools include underwater weighing, bioelectrical impedance, dual energy x-ray absorptiometry and radiological imaging (e.g., computerized tomography or magnetic resonance imaging).

Exemplary screening assays are disclosed for example in US 2010/0210545 and US 20080214547 referred to above.

The present invention further contemplates derivatizing an agent that tests positive for TRAIL DR agonist activity, and optionally formulating the derivatized agent with a pharmaceutically acceptable carrier and/or diluent, to improve the efficacy of the agent for treating or preventing the adiposity-related condition(s).

### 4. TRAIL DR Agonist Derivatives and Conjugates

The present invention also extends to conjugates and derivatives of the adiposity-modulating TRAIL DR agonists. For example, the TRAIL DR agonists may be conjugated with biological targeting agents that enable their activity to be restricted to particular cell types. Such biological-targeting agents include substances that are immuno-interactive with cell-specific surface antigens. In representative examples of this type, a TRAIL DR agonist is conjugated with an agent that is immuno-interactive with a non-TRAIL DR adipose cell surface protein, such as, for example, adipose differentiation related protein (ADRP). The presence of this immuno-interactive conjugate confers adipose cell specificity or preference to the effects of the TRAIL DR agonist. Illustrative molecules of this type include bi-specific antigen-binding molecules that comprise a first antigen-binding portion that is immuno-interactive with the non-TRAIL DR adipose cell surface protein, and a second antigen-binding portion that is immuno-interactive with a TRAIL DR.

The TRAIL DR agonists may include a property-modifying moiety for enhancing biological activity, prolonging blood circulation time, reducing immunogenicity, increasing aqueous solubility, and enhancing resistance to protease digestion. In some embodiments, the property-modifying moiety modifies the property of the TRAIL DR agonist so that it achieves a sufficient hydrodynamic size to prevent clearance by renal filtration in vivo. For example, a property-modifying moiety can be selected that is a polymeric macromolecule, which is substantially straight chain, branched-chain, or dendritic in form. Alternatively, a property-modifying moiety can be selected such that, in vivo, the TRAIL DR agonist will bind to a serum protein to form a complex, such that the complex thus formed avoids substantial renal clearance. The property-modifying moiety can be, for example, a lipid; a cholesterol group (such as a steroid); a carbohydrate or oligosaccharide; or any natural or synthetic protein, polypeptide or peptide that binds to a salvage receptor.

Exemplary property-modifying moieties that can be used, in accordance with the present invention, include an immunoglobulin Fc domain, or a portion thereof, or a biologically suitable polymer or copolymer, for example, a polyalkylene glycol compound, such as a polyethylene glycol or a polypropylene glycol. Other appropriate polyalkylene glycol compounds include, but are not limited to, charged or neutral polymers of the following types: dextran, polylysine, colominic acids or other carbohydrate based polymers, polymers of amino acids, and biotin derivatives.

Other examples of the property-modifying moiety, in accordance with the invention, include a copolymer of ethylene glycol, a copolymer of propylene glycol, a carboxymethylcellulose, a polyvinyl pyrrolidone, a poly-1,3-dioxolane, a poly-1,3,6-trioxane, an ethylene/maleic anhydride copolymer, a polyaminoacid (e.g., polylysine), a dextran n-vinyl pyrrolidone, a poly n-vinyl pyrrolidone, a propylene glycol homopolymer, a propylene oxide polymer, an ethylene oxide polymer, a polyoxyethylated polyol, a polyvinyl alcohol, a linear or branched glycosylated chain, a polyacetal, a long chain fatty acid, a long chain hydrophobic aliphatic group, an immunoglobulin Fc domain or a portion thereof (see, for example, Feige et al., Modified peptides as therapeutic agents, U.S. Pat. No. 6,660,843), a CH2 domain of Fc, an albumin (e.g., human serum albumin (HSA)); see, for example, Rosen et al., Albumin fusion proteins, U.S. Pat. No. 6,926,898 and US 2005/0054051; Bridon et al., Protection of endogenous therapeutic peptides from peptidase activity through conjugation to blood components, U.S. Pat. No. 6,887,470), a transthyretin (TTR; see, for example, Walker et al., Use of transthyretin peptide/protein fusions to increase the serum half-life of pharmacologically active peptides/proteins, US 2003/0195154; 2003/0191056), or a thyroxine-binding globulin (TBG). Thus, exemplary embodiments of the TRAIL DR agonists also include HSA fusion constructs such as but not limited to: HSA fusions with ShK, OSK1, or modified analogs of those toxin peptides. Examples include HSA-L10-ShK(2-35); HSA-L10-OsK1(1-38); HSA-L10-ShK(2-35); and HSA-L10-OsK1(1-38).

Other embodiments of the property-modifying moiety, in accordance with the present invention, include peptide ligands or small (organic) molecule ligands that have binding affinity for a long half-life serum protein under physiological conditions of temperature, pH, and ionic strength. Examples include an albumin-binding peptide or small molecule ligand, a transthyretin-binding peptide or small molecule ligand, a thyroxine-binding globulin-binding peptide or small molecule ligand, an antibody-binding peptide or small molecule ligand, or another peptide or small molecule that has an affinity for a long half-life serum protein. (See, e.g., Blaney et al., Method and compositions for increasing the serum half-life of pharmacologically active agents by binding to transthyretin-selective ligands, U.S. Pat. No. 5,714,142; Sato et al., Serum albumin binding moieties, US 2003/0069395; Jones et al., Pharmaceutical active conjugates, U.S. Pat. No. 6,342,225). A “long half-life serum protein” is one of the hundreds of different proteins dissolved in mammalian blood plasma, including so-called “carrier proteins” (such as albumin, transferrin and haptoglobin), fibrinogen and other blood coagulation factors, complement components, immunoglobulins, enzyme inhibitors, precursors of substances such as angiotensin and bradykinin and many other types of proteins. The invention encompasses the use of any single species of pharmaceutically acceptable property-modifying moiety, such as, but not limited to, those described herein, or the use of a combination of two or more different half-life extending moieties, such as PEG and immunoglobulin Fc domain or a CH2 domain of Fc, albumin (e.g., HSA), an albumin-binding protein, transthyretin or TBG.

In some embodiments, the property-modifying moiety is polyethylene glycol (PEG). For example, the TRAIL DR agonist can be made mono-PEGylated, di-PEGylated, or otherwise multi-PEGylated, by the process of reductive alkylation.

Covalent conjugation of proteins and peptides with PEG has been widely recognized as an approach to significantly extend the in vivo circulating half-lives of therapeutic proteins. PEGylation achieves this effect predominately by retarding renal clearance, since the PEG moiety adds considerable hydrodynamic radius to the protein. (Zalipsky, S., et al., Use of functionalized poly(ethylene glycol)s for modification of polypeptides, in poly(ethylene glycol) chemistry: Biotechnical and biomedical applications., J. M. Harris, Ed., Plenum Press: New York., 347-370, 1992. Additional benefits often conferred by PEGylation of proteins and peptides include increased solubility, resistance to proteolytic degradation, and reduced immunogenicity of the therapeutic polypeptide. The merits of protein PEGylation are evidenced by the commercialization of several PEGylated proteins including PEG-Adenosine deaminase (Adagen™/Enzon Corp.), PEG-L-asparaginase (Oncaspar™/Enzon Corp.), PEG-Interferon α-2b (PEG-Intron™/Schering/Enzon), PEG-Interferon α-2a (PEGASYS™/Roche) and PEG-G-CSF (Neulasta™/Amgen) as well as many others in clinical trials.

Briefly, the PEG groups are generally attached to the peptide portion of a TRAIL DR agonist via acylation or reductive alkylation through a reactive group on the PEG moiety (e.g., an aldehyde, amino, thiol, or ester group) to a reactive group on the inventive compound (e.g., an aldehyde, amino, or ester group).

Any molecular mass for a PEG can be used as practically desired, e.g., from about 1,000 or 2,000 Daltons (Da) to about 100,000 Da (n is 20 to 2300) (the term “about” indicating that in preparations of PEG, some molecules will weigh more, some less, than the stated molecular weight). In illustrative examples of this type, the combined or total molecular mass of PEG used in a PEG-conjugated peptide or polypeptide of the present invention is from about 3,000 Da or 5,000 Da, to about 50,000 Da or 60,000 Da (total n is from 70 to 1,400), suitably from about 10,000 Da to about 40,000 Da (total n is about 230 to about 910).

### 5. Therapeutic and Prophylactic Uses

In accordance with the present invention, it is proposed that TRAIL DR agonists are useful as actives for the treatment or prophylaxis of excess adiposity, including adiposity-related conditions as described above, including conditions such as obesity, diabetes mellitus and metabolic syndrome. Such agonists can be administered to a patient either by themselves, or in pharmaceutical compositions where they are mixed with a suitable pharmaceutically acceptable carrier.

Depending on the specific conditions being treated, the TRAIL DR agonist drugs may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the drugs of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is appropriate, for example, for administration of immunogenic compositions, vaccines and DNA vaccines.

The drugs can be formulated readily using pharmaceutically acceptable carriers well known in the art into dosages suitable for oral administration. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of drug administered to a patient should be sufficient to affect a beneficial response in the patient over time such as an enhancement or reduction in adipogenesis. The quantity of the drug(s) to be administered may depend on the subject to be treated inclusive of the age, sex, weight and general health condition thereof. In this regard, precise amounts of the drug(s) for administration will depend on the judgment of the practitioner. In determining the effective amount of the drug to be administered in the modulation of adiposity, diabetes mellitus and metabolic syndrome, the physician may evaluate tissue or cell levels of a TRAIL DR, degree of adiposity (e.g., using skin folds), glucose levels, insulin levels, blood pressure, High Density Lipoprotein (HDL) levels, triglycerides levels, uric acid levels etc. In any event, those of skill in the art may readily determine suitable dosages of the drugs of the invention.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more drugs as described above with the carrier, which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain arabic gum, talc, polyvinyl pyrrolidone, carbopol gel, polyethylene glycol, or titanium dioxide, lacquer solutions, and suitable organic solvents or solvent mixtures. Dyestuffs or pigments may be added to the tablets or dragee coatings for identification or to characterize different combinations of active compound doses.

Pharmaceutical which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the drugs of the invention may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes or microspheres.

The drugs of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC50 as determined in cell culture (e.g., the concentration of a test agent, which achieves a half-maximal activation of a TRAIL DR polypeptide). Such information can be used to more accurately determine useful doses in humans.

Toxicity and therapeutic efficacy of such drugs can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p 1).

Dosage amount and interval may be adjusted individually to provide plasma levels of the active agent, which are sufficient to maintain TRAIL DR activation. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m²/day, commonly from 0.5-150 mg/m²/day, typically from 5-100 mg/m²/day.

Alternately, one may administer the compound in a local rather than systemic manner, for example, via injection of the compound directly into a tissue, which is suitably subcutaneous or omental tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

### TRAIL Significantly Reduces Fasting Hyperinsulinemia without Affecting Glucose Levels or Circulating Lipids

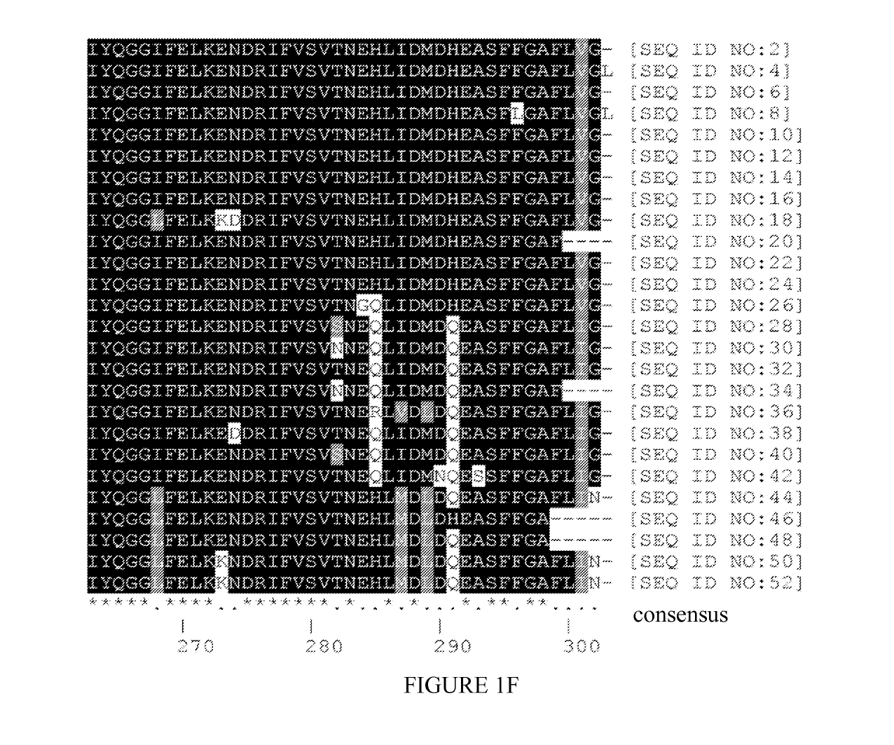

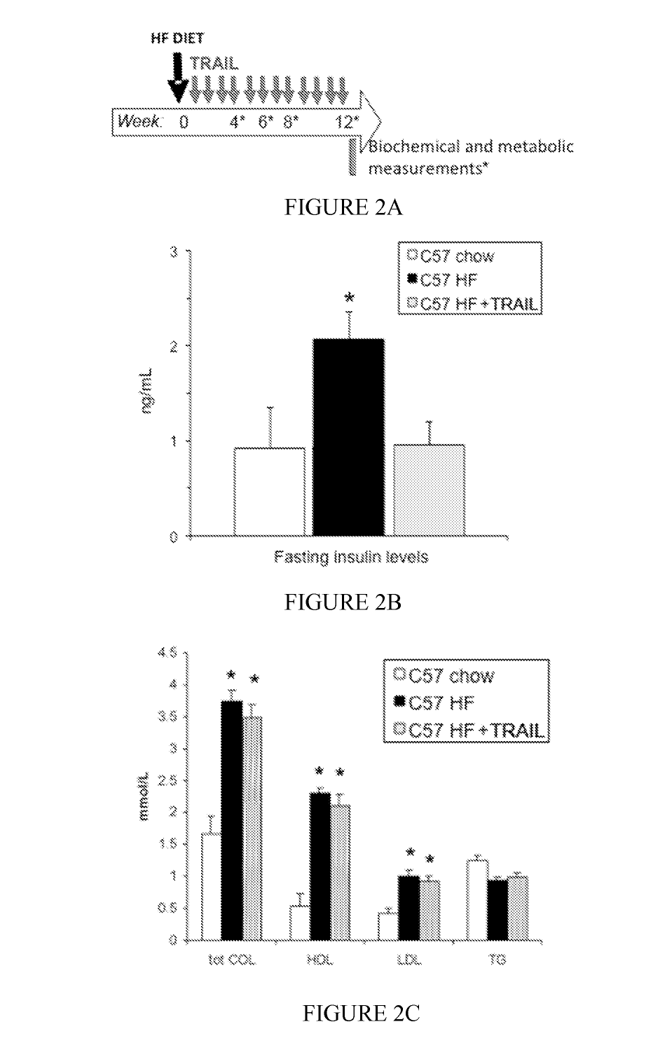

C57 HF mice displayed the fasting hyperinsulinemia featuring a state of insulin-resistance. C57 HF mice treated weekly with TRAIL for 12 weeks (FIG. 2A) had significantly lower fasting insulin levels (p<0.05, vs C57 HF) overlapping those of C57 chow (FIG. 2B). However, fasting glucose levels at the end of the study didn't change between treated and untreated high fat fed mice, being respectively 12.9±0.92 and 13.5±1.07 mmol/L, both significantly (p<0.05) higher with respect to the normal fed mice, having 10.1±0.73 mmol/L.

Serum total cholesterol, HDL and LDL significantly (p<0.05) increased after HFD in both C57 HF and C57 HF+TRAIL animals with respect to C57 chow (FIG. 2C). No serum triglycerides changes were observed among the three groups (FIG. 2C). No free fatty acids changes were observed among the three groups, being 0.59 mmol/L±0.09 in C57 chow; 0.61 mmol/L±0.19 in C57 HF and 0.58 mmol/L±0.225 in C57 HF+TRAIL.

### Example 2

### TRAIL Significantly Reduces Glucose Levels after a Hyperglycemic Stimulus at 12 Weeks

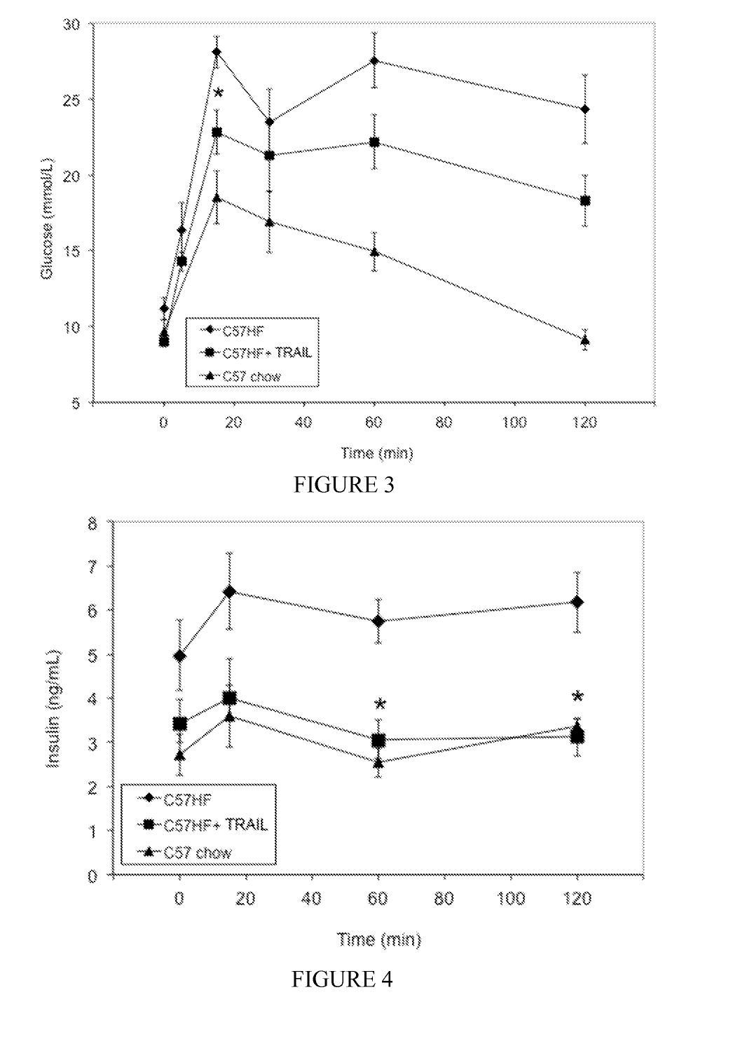

To further examine glucose metabolism, whole-body glucose clearance was studied during an intraperitoneal glucose tolerance test (IPGTT) at 6 (not shown) and 12 weeks time points (FIG. 3). At both time points, high fat feeding resulted in a significant impairment in glucose clearance, leading to hyperglycemia at 15′, 30′, 60′ and 120′ (C57 HF vs C57 chow). TRAIL treatment reduced significantly the glucose levels at 15′ after the hyperglycemic stimulus at 6 weeks of study (p<0.05 vs C57 HF) and at and 15′ at 12 weeks of study (p<0.05 vs C57 HF) (FIG. 3) whereas at 60′ and 120′ after the hyperglycemic stimulus at 12 weeks the p value was equal to 0.052 and 0.055.

### Example 3

### TRAIL Significantly Lowers Hyperinsulinemia after a Hyperglycemic Stimulus at 12 Weeks

At 6 weeks of study the single insulin levels measured during an IPGTT did not differ significantly among the groups as well as the first phase of insulin secretion 2.20 ng/mL/min in C57 chow, 2.67 in C57 HF and 2.56 in C57 HF+TRAIL. At 12 weeks of study (FIG. 4) C57 HF displayed significant hyperinsulinemia with respect to C57 chow, that was significantly reduced by TRAIL during the second phase of the curve (p<0.05 at 60 and 120 min).

### Example 4

### TRAIL Improved the Peripheral Response to Insulin, Lowering Glucose Levels at 60 Minutes after a Hyperinsulinemic Stimulus

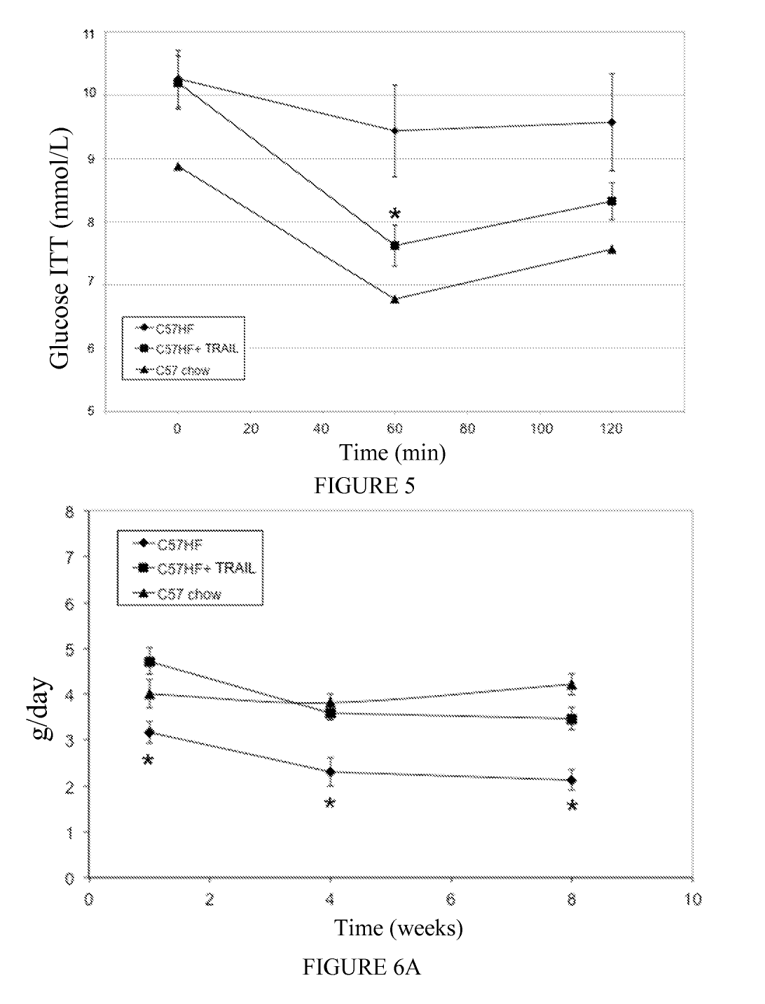

To better characterize the peripheral response to insulin the inventors performed an intraperitoneal insulin tolerance test (IPITT) (FIG. 5), that showed a significant reduction in glucose levels 60 minutes after the insulin load in TRAIL-treated animals (p<0.05 vs C57 HF).

### Example 5

### TRAIL Treatment Significantly Reverses the Changes in Substrate Utilization Induced by High Fat Diet

The energy expenditure was evaluated by indirect calorimetry, measuring whole-body oxygen consumption over 24 hours. When expressed per gram of lean mass the VO₂ (mL/g/hr) was significantly increased only in C57 HF+TRAIL (p<0.05 vs C57 chow and C57 HF).

Heat production and locomotor activity were also analyzed: while heat production increased comparatively in C57 HF and C57 HF+TRAIL at 8 weeks of study, locomotor activity was significantly (p<0.05) reduced in C57 HF at 4 weeks as compared to both C57 chow and C57 HF+TRAIL.

The measurement of the respiratory exchange ratio (RER), ratio between VCO₂ produced and VO₂ burnt, confirmed what has been previously described (Turner et al., Diabetes 56:2085-92, 2007), since HFD significantly shifted the metabolism towards a nearly complete fat consumption as fuel; in this setting TRAIL significantly reversed this feature (p<0.05 vs C57 HF) (Table 4).

TABLE 4
Parameters studied by indirect calorimetry.
All the data are expressed as means ± SEM; *p < 0.05 vs C57 HF.

| | | | | C57 |
|---|---|---|---|---|
| | Parameter | C57 chow | C57 HF | HF + TRAIL |
| 4 weeks | VO₂ adj lean | 5.00 ± 0.16 | 5.01 ± 0.18 | 5.38 ± 0.108 |
| | mass (mL/h) | | | |
| | Heat (Kcal/HR) | 0.69 ± 0.028 | 0.64 ± 0.03 | 0.64 ± 0.02 |
| | Locomotor activity | 24737 ± 899 | 19045 ± 1826 | 2900 ± 1912* |
| | RER | 0.88 ± 0.008 | 0.77 ± 0.003 | 0.81 ± 0.007* |
| | (VCO₂/VO₂) | | | |
| 8 weeks | VO₂ adj lean | 4.26 ± 0.22 | 4.94 ± 0.62 | 6.18 ± 0.22* |
| | mass (mL/h) | | | |
| | Heat (Kcal/HR) | 0.56 ± 0.014 | 0.72 ± 0.035 | 0.81 ± 0.037 |
| | Locomotor activity | 21074 ± 1365 | 17067 ± 2130 | 24468 ± 2055* |
| | RER | 0.89 ± 0.01 | 0.77 ± 0.007 | 0.82 ± 0.0009* |
| | (VCO₂/VO₂) | | | |

### Example 6

### Ex Vivo Palmitate Oxidation Significantly Increases after TRAIL Treatment

Ex vivo palmitate oxidation (nmol/min/g) in skeletal muscle, measured after 12 weeks of HFD, significantly increased in C57 HF vs C57 chow (16.56±1.1 vs 14.3±1.1, respectively, p<0.05). TRAIL treatment further increased it vs C57 HF (19.53±1.8 vs 16.56±1.1, respectively, p<0.05).

### Example 7

### TRAIL Significantly Reduces the Increased Adiposity Due to a HFD

The body composition of male mice C57 chow, C57 HF and C57 HF+TRAIL was examined every four weeks over the 12 weeks of the HFD. Male C57 HF became obese and displayed an increased adiposity after 4, 8 and 12 weeks with respect to age and sex-matched mice fed a standard diet which served as controls (p<0.05 vs C57 chow). The increased adiposity was completely prevented by TRAIL treatment at 4 weeks (p<0.05, vs C57 HF) whereas at 8 and 12 weeks of study TRAIL significantly reduced the increased adiposity without preventing it (p<0.05 vs C57 HF and C57 chow) (Table 5).

TABLE 5
Body mass composition

| | | 4 weeks | C57 | | | C57 |
|---|---|---|---|---|---|---|
| | 0 week | C57 | HF + | | C57 | HF + |
| | C57 | HF | TRAIL | C57 | HF | TRAIL |
| Body | | | | 16.8 ± 2.5 | 34.0 ± 4.7 | 24.0 ± 3.5 |
| mass | | | | | | |
| increase % | | | | | | |
| Fat | 10.0 ± 0.6 | 11.1 ± 1.2 | 8.2 ± 0.5 | 14.9 ± 1.5 | 28.7 ± 1.2 | 15.4 ± 1.4* |
| mass % | | | | | | |
| Lean | 90.5 ± 1.2 | 87.9 ± 1.2 | 90.8 ± 0.9 | 80.2 ± 1.7 | 68.0 ± 1.3 | 81.6 ± 1.5* |
| mass % | | | | | | |

| | |
|---|---|
| 8 weeks | 12 weeks |

| | | | C57 | | | C57 |
|---|---|---|---|---|---|---|
| | | C57 | HF + | | C57 | HF + |
| | C57 | HF | TRAIL | C57 | HF | TRAIL |
| Body | 17.8 ± 1.9 | 40.8 ± 7.5 | 42.8 ± 4.1 | 23.2 ± 2.4 | 49.3 ± 5.6 | 51.1 ± 4.9 |
| mass | | | | | | |
| increase % | | | | | | |
| Fat | 15.7 ± 1.3 | 32.4 ± 1.6 | 22.1 ± 2.0* | 18.0 ± 1.4 | 32.8 ± 1.0 | 26.8 ± 2.4* |
| mass % | | | | | | |
| Lean | 78.8 ± 1.8 | 62.6 ± 1.5 | 74.4 ± 1.8* | 75.9 ± 1.2 | 61.2 ± 0.7 | 71.1 ± 2.4* |
| mass % | | | | | | |

All the data are expressed as means ± SEM;
*p < 0.05 vs C57 HF.

### Example 8

### TRAIL Treatment is not Associated with a Reduction in Appetite and C57 HF+TRAIL Mice Display the Highest Energy Intake

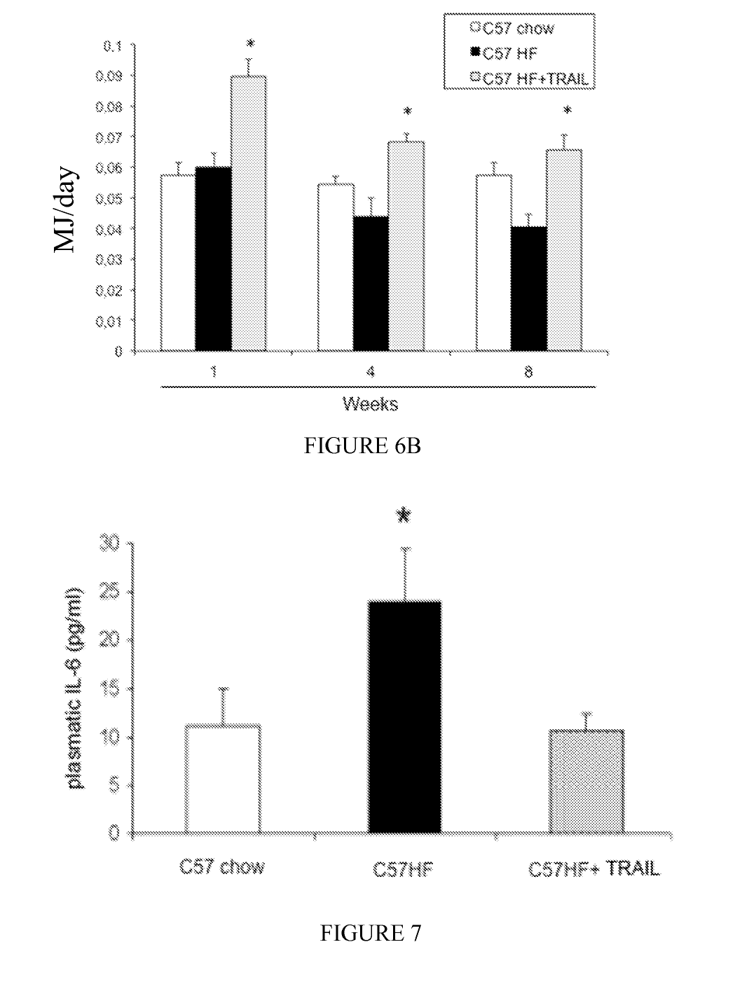

Food intake did not differ between C57 chow and C57 HF+TRAIL, whereas C57 HF ate significantly less during the whole length of the study (p<0.05 vs C57 chow and vs C57 HF+TRAIL) (FIG. 6A). Given the composition of the HFD, C57 HF+TRAIL displayed the highest energy intake among the groups at 4 and 8 weeks (p<0.05 vs C57 chow and vs C57 HF) (FIG. 6B), although C57 HF displayed the lowest food intake at 12 weeks (FIG. 6A).

### Example 9

### TRAIL Treatment Significantly Modifies Adipose Tissue Gene Expression

The present inventors analyzed the adipose tissue gene expression of proinflammatory genes such as Angiotensinogen (Angio), CD36, HO-1, IL-6, MCP1, MIF, NFκb, OPG, PAI-1, PPARalpha, TNFalpha, genes related to adipocyte differentiation, such as BMP7, PPARgamma, to lipid metabolism, such as PPARalpha and CD36 and apoptotic genes, such as BAX, BCL2, caspase3. Pro-inflammatory genes significantly increased in both high fat fed mice groups. Among these genes TRAIL induced only a significant up-regulation of OPG. The major effects of TRAIL were a significant down-regulation of differentiation genes and an up-regulation of the pro-apoptotic ones (Table 6).

TABLE 6
Gene Expression in AdiposeTissue

| gene | group | average | St. Dev | SEM |
|---|---|---|---|---|
| PPARg | chow | 1 063 | 415 | 157 |
| | HF | 4.860 | 4.338 | 1.771 |
| | HF + | 1.918 | 463 | 154 |
| | TRAIL | | | |
| BMP7 | chow | 1.094 | 503 | 252 |
| | HF | 10.214 | 10.715 | 4.374 |
| | HF + | 3.376 | 2.170 | 767 |
| | TRAIL | | | |
| PPARa | chow | 1.295 | 1.009 | 451 |
| | HF | 2.891 | 2.995 | 1.223 |
| | HF + | 3.237 | 2.435 | 812 |
| | TRAIL | | | |
| CD36 | chow | 1.020 | 209 | 93 |
| | HF | 1.616 | 946 | 357 |
| | HF + | 1.560 | 367 | 122 |
| | TRAIL | | | |
| OPG | chow | 1.073 | 472 | 178 |
| | HF | 7.734 | 9.137 | 3.453 |
| | HF + | 14.754 | 4.620 | 1.633 |
| | TRAIL | | | |
| ANGIO | chow | 1.174 | 703 | 314 |
| | HF | 1.653 | 1.231 | 550 |
| | HF + | 2.899 | 952 | 360 |
| | TRAIL | | | |
| PAI1 | chow | 903 | 700 | 313 |
| | HF | 10.254 | 4.311 | 1.760 |
| | HF + | 10.286 | 2.874 | 1.016 |
| | TRAIL | | | |
| TNFalpha | chow | 1.181 | 806 | 329 |
| | HF | 4.510 | 2.837 | 1.072 |
| | HF + | 6.310 | 5.198 | 1.965 |
| | TRAIL | | | |
| IL-6 | chow | 1.200 | 576 | 258 |
| | HF | 5.455 | 5.202 | 1.966 |
| | HF + | 7.623 | 6.298 | 2.099 |
| | TRAIL | | | |
| MCP1 | chow | 1.475 | 1.349 | 603 |
| | HF | 20.441 | 13.519 | 5.110 |
| | HF + | 19.719 | 21.615 | 7.205 |
| | TRAIL | | | |
| MIF | chow | 1.011 | 166 | 74 |
| | HF | 1.161 | 266 | 94 |
| | HF + | 1.093 | 222 | 74 |
| | TRAIL | | | |
| HO1 | chow | 1.189 | 723 | 273 |
| | HF | 3.136 | 1.546 | 584 |
| | HF + | 3.418 | 1.752 | 620 |
| | TRAIL | | | |
| NFkb | chow | 1.349 | 838 | 317 |
| | HF | 2.865 | 1.529 | 684 |
| | HF + | 3.513 | | |
| | TRAIL | | | |
| Caspase 3 | chow | 1.098 | 542 | 271 |
| | HF | 2.157 | 697 | 349 |
| | HF + | 4.296 | 1.323 | 662 |
| | TRAIL | | | |
| BAX | chow | 1.024 | 0.000 | 0.000 |
| | HF | 310061855068.929 | 455534321122.365 | 172175789620.631 |
| | HF + | 1440049553321.970 | 2541832496505.280 | 847277498835.094 |
| | TRAIL | | | |
| BCL2 | chow | 1.351 | 969 | 433 |
| | HF | 2.108 | 1.076 | 538 |
| | HF + | 1.130 | 365 | 183 |
| | TRAIL | | | |

### Example 10

### Adipose Tissue Apoptosis Increases after TRAIL Treatment

The number of apoptotic cells per frame, on adipose tissue frozen sections stained by TUNEL, increased significantly after TRAIL treatment. C57 HF displayed and average of 0.8 apoptotic cells per frame whereas in C57 HF+TRAIL there was an average of 2.25 apoptotic cells (p<0.05 vs C57 HF).

### Example 11

### Circulating IL-6 is Significantly Reduced after TRAIL Treatment at the End of the Study

Circulating IL-6 was significantly (p<0.05) increased after 12 weeks of HFD with respect to C57 chow. On the other hand, TRAIL treatment significantly counteracted the increase of IL-6 (p<0.05 vs C57 HF, FIG. 7).

### Example 12

### Lack of Toxicity by Intraperitoneal TRAIL Treatment

Of significance, TRAIL was detectable in sera up to 4 days after intraperitoneal injections (data not shown) and that repeated intraperitoneal injections were safe, since mice treated with TRAIL did not show gross abnormalities at necroscopic examination, as compared to untreated mice.

### Example 13

### TRAIL Treatment Impairs the Inflammatory Response to Lipopolysaccharide (LPS) or Muramildipeptide (MDP)

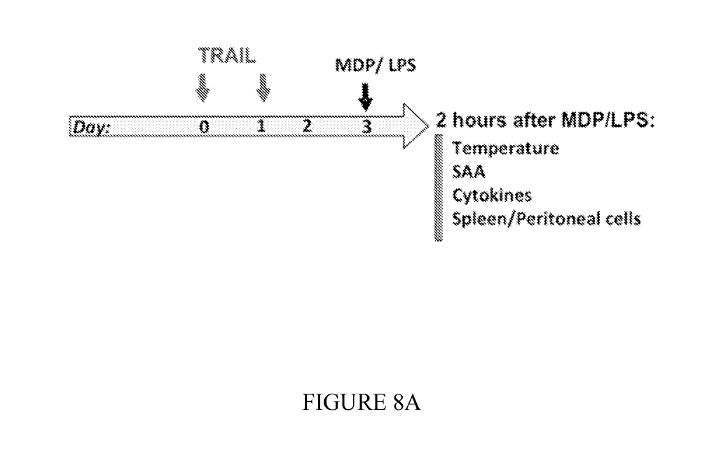

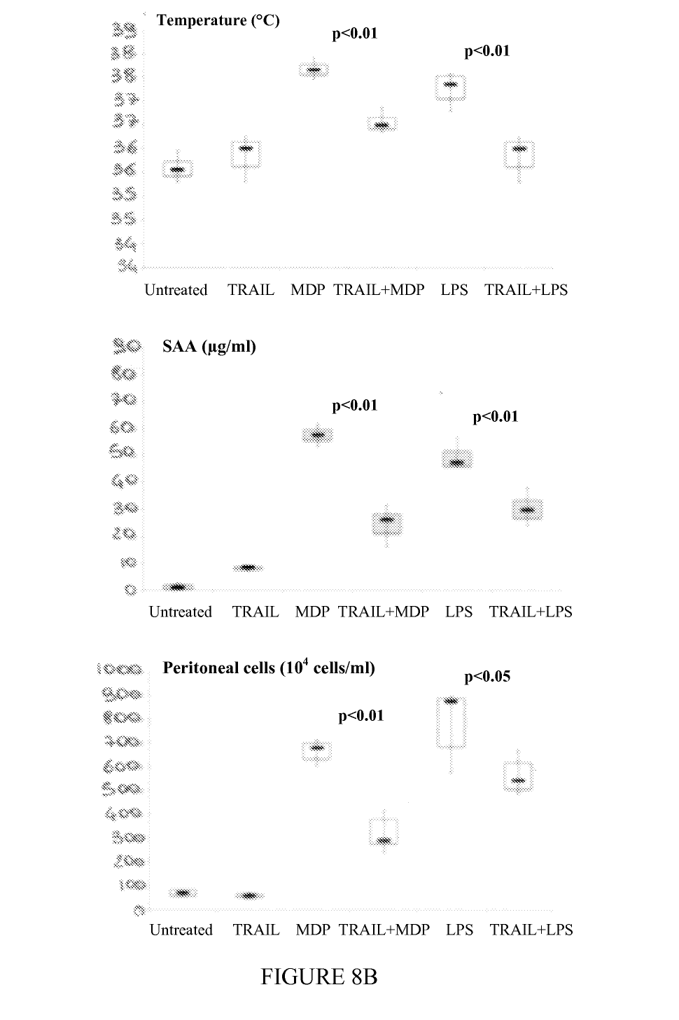

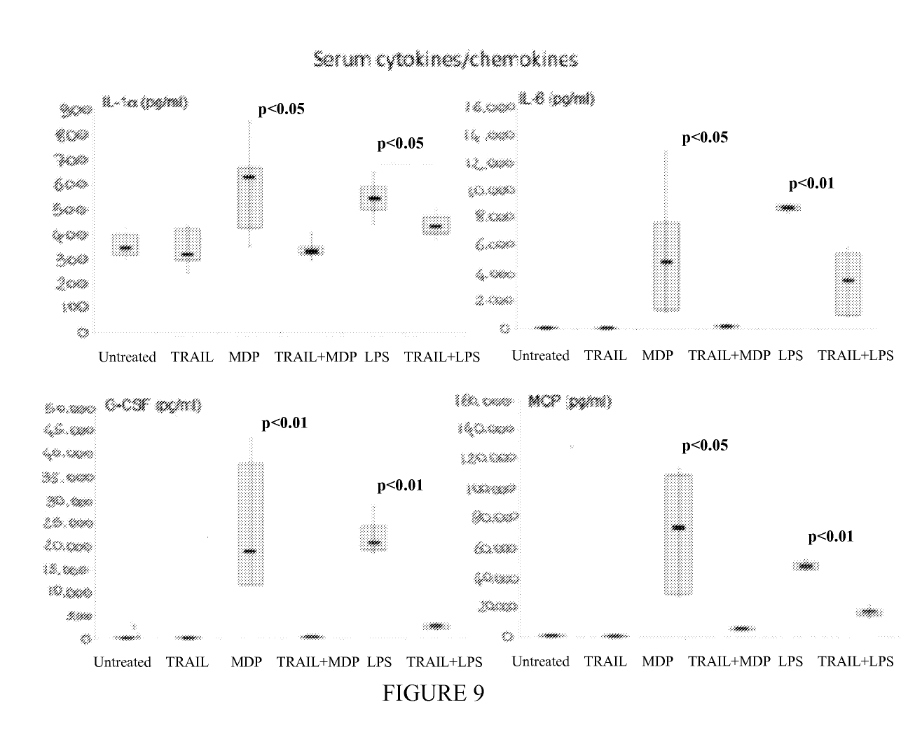

Treatment with TRAIL for 2 consecutive days was followed after 2 additional days by MDP or LPS administration for two hours (FIG. 8A). After, BALB/c mice were sacrificed and analyzed for body temperature, SSA levels, number of intra-peritoneal mononuclear cells, and serum cytokine levels (FIG. 8B). Both MDP and LPS induced a marker increase of body temperature, which was significantly (p<0.05) inhibited by pre-treatment with TRAIL. Similarly, also the raise of the acute phase protein SSA as well as the number of intra-peritoneal mononuclear cells induced by both MDP and LPS was significantly (p<0.05) lower in animals pretreated with rTRAIL (FIG. 8B). The effect of TRAIL pre-treatment was even more impressive on serum cytokines, which are know to be elevated both after acute and chronic inflammation, which occurs in obesity, diabetes mellitus and metabolic syndrome. As shown in FIG. 9, pre-treatment with human recombinant TRAIL almost completely abolished (p<0.05) the increase of serum levels of IL-1alpha, IL-6. G-CSF, MCP-1 induced by either MDP or LPS.

### Discussion of the Examples

The present invention discloses for the first time the ability of TRAIL to significantly reduce the metabolic abnormalities due to an oversupply of lipids: hyperinsulinemia at fasting, increased glucose levels and hyperinsulinemia after a hyperglycemic stimulus, reduced glucose and increased lipid metabolism for fuel, ameliorating the peripheral response to insulin and improving the mitochondrial fatty acid oxidative capacity in muscle, as well as reducing circulating levels of the pro-inflammatory cytokine IL-6 in both prolonged or short-term treatments performed in different strains of mice, C57black and BALB/c, respectively.

The chosen animal model for studying the effects of TRAIL on metabolism relies on the notion that an oversupply of lipids, leading to an abnormal accumulation of fat in adipose and non-adipose tissues such as muscle and liver, plays an important role in the etiology of insulin resistance and later on in the demise of the beta-cell in type II diabetes (McGarry et al., Diabetes 51:7-18, 2002). For this reason, high-fat-fed rodents or animals lacking leptin signaling have extensively been studied to understand the mechanisms underlying the development of insulin resistance. Evidence from these studies has demonstrated that C57Black6J mice fed a high-fat diet (HFD) increased their percentage of fat becoming obese and display a significant impairment in glucose tolerance and a 40% reduction in insulin stimulated glucose uptake in skeletal muscle, where there is a complete utilization of free fatty acid for fuel (Turner et al., Diabetes 56:2085-92, 2007). In this setting, TRAIL was found to have the unexpected ability to significantly reverse this abnormality.

After 12 weeks of high-fat diet, TRAIL treated mice presented a significant reduction of the fasting hyperinsulinemia, which was observed in the untreated mice.

The evaluation of glucose tolerance, which is dependent on insulin action, showed that TRAIL had the ability to significantly reduce the hyperglycemia in the fat-fed mice 15 minutes after a hyperglycemic stimulus, both in the 6- and 12-week study. It also lowered fasting glucose levels at 12 weeks, although at the end of the study that difference was lost. It is believed that this discrepancy is due to the experimental protocol that was employed. In this regard, TRAIL was detectable in sera only up to 4 days after injection and animal sacrifices took place one week after the IPGTT took place and the IPGTT was performed two days after the last injection of TRAIL.

TRAIL was also found to lower the hyperinsulinemia observed during the glucose tolerance test in HFD mice. Particularly, whereas at 6 weeks of study only a tendency could be noted, at 12 weeks of study the reduction was significant at 60 and 120 minutes after a hyperglycemic stimulus. The reduced glucose levels resulted from an improvement of peripheral response to insulin, leading to a better glucose uptake. Since insulin secretion is biphasic (Gerich, Diabetes 51 Suppl 1: S 117-21, 2002) in which subsequent to an hyperglycemic stimulus, circulating insulin concentrations increase rapidly, decrease and then gradually increase progressively, proportionally to the degree of insulin-resistance, it is striking that insulin levels were significantly reduced 2 hours after an hyperglycemic stimulus in the TRAIL treated mice, which is suggestive of a significant improvement of peripheral insulin resistance, which is one of the hallmarks of type II diabetes. On the contrary, the analysis of the morphology of the curves of insulin levels during an IPGTT did not show any difference after TRAIL treatment in the Δ between the levels of the peptide and those measured 15 minutes after the stimulus.

Consistently with that, the IPITT showed that the treatment with TRAIL was associated with significantly reduced glucose levels 60 minutes after insulin injection, suggesting that there was a better peripheral response to the pancreatic peptide. Since the hyperinsulinemia observed initially in type II diabetes relies on the peripheral abnormal response to the peptide, it is not surprising that in both fasting and fed states insulin levels were significantly lower in the mice treated with TRAIL, which therefore slow down significantly the development of type II diabetes.

Another key feature described in these models of insulin-resistance is the increased capacity for peripheral fatty acid oxidation, which has been explained as a compensatory response to elevated fatty acid substrate availability but which could also be explained by the abnormal peripheral insulin response which does not permit the body to burn glucose properly. According to the results presented herein, C57 HF displayed the expected shift towards lipids as preferential substrate for fuel. The respiratory exchange ratio (RER) with the values of 1 or 0.7 indicating respectively 100% CHO or 100% fat oxidation, was indeed significantly decreased in high fat fed mice untreated. In this setting TRAIL treatment had the ability to significantly reverse this abnormality at 4 and 8 weeks of study.

The present inventors also observed that the ex vivo measurement of palmitate oxidation rate in skeletal muscle was significantly increased after TRAIL treatment. To interpret this data and to combine it with the results on the RER, two aspects should be taken into account; first, lipid overload in muscle may be linked to the reduction in lean muscle mass, which is indeed observed in insulin resistance, and which in turn would lead to low rates of palmitate oxidation; second, the ex vivo assessment of the fatty acid oxidation pathways is performed under favorable conditions of substrate availability in an environment free of regulatory factors that may affect this process (Turner et al., 2007, supra). Having said that, mitochondrial dysfunction has been pointed out as one of the earliest defects that predispose to lipid accumulation and insulin resistance, so the increased palmitate oxidation observed in HFD mice treated with TRAIL may be due to a protective effect of this drug against decreased mitochondrial function and therefore lipid accumulation and insulin resistance.

TRAIL treatment was also found to prevent the increased adiposity due to the high fat diet after 4 weeks of study and significantly reduced it during the following weeks. Of interest, the significant reduction in the percentage adiposity gained, observed after TRAIL injection, was not due to a reduction in food intake. The data presented herein clearly shows that C57 HF+TRAIL ate as much as the C57 chow, whereas the C57 HF displayed a reduction in their food intake, possibly related to the increased adiposity which would have led to higher circulating levels of leptin. Thus, since the HF diet is slightly hypercaloric compared to the chow diet, C57 HF+TRAIL displayed the highest caloric intake during the length of the study.

Gene expression analysis also revealed possible mechanisms by which TRAIL treatment could reduce the adipose mass. In evaluating the expression of several genes in the adipose tissue of these mice, TRAIL down-regulated PPAR-gamma and BMP-7, which are markers of white adipose tissue differentiation and brown adipose tissue differentiation, and it modified the expression of the genes related to apoptosis promoting a pro-apoptotic effect on the fat. Indeed, caspase 3 and BAX gene expressions were significantly upregulated. Consistent with these results, adipose tissue staining to detect apoptosis revealed a significant increase in the number of apoptotic nuclei per frame (considering frames with a similar amount of total nuclei).

In another set of experiments performed on BALB/c male mice, we were able to demonstrate that 2 consecutive (day 0 and day 1) intraperitoneal injections of TRAIL followed after 48 hours (day 3) by MDP or LPS administration for 2 hours were able to significantly counteract the whole set of pro-inflammatory reactions elicited by either proinflammatory stimuli (MDP and LPS). In particular, TRAIL treatment significantly reduced the elevation of body temperature, the number of intraperitoneal mononuclear cells, the rise in the serum levels of the acute reactive protein serum amiloid-A (SAA) as well as of several pro-inflammatory cytokines, such as IL-6, IL-1alpha, G-CSF and MCP-1.

### Materials and Methods

### Animals and Experimental Protocol

27 wild-type male C57b16 mice aged 8 weeks were randomly allocated to standard chow diet (C57 chow), high fat diet (C57 HF) or high fat diet+TRAIL (C57 HF+TRAIL), delivered at a dose of 10 μg every week by intraperitoneal injection for 12 weeks. The standard diet had 19.6% of protein, 4.6% of total fat, 4.5% of crude fiber providing a digestible energy of 14.3 MJ/Kg, the high fat diet (HFD) had 22.6% of protein, 23.5% of total fat, 5.4% of crude fiber providing a digestible energy of 19 MJ/Kg fed with a high-fat diet (43% of calories from fat, 21% calories from protein, and 36% calories from carbohydrate). Recombinant (r) histidine 6-tagged hTRAIL (114-281) was produced in bacteria as previously described (Secchiero et al, Circulation 114:1522-30, 2003) and resuspended in buffered saline before the injection. The animals were kept in a temperature-controlled room (22±1° C.) on a 12-h light/dark cycle with free access to food and water and they were fed ad libitum for the length of the study. After 12 weeks of study, after body weight and blood glucose were measured, the animals were anesthetized by an intraperitoneal injection of pentobarbitone at a dose of 100 mg/Kg body weight. Blood was collected from the left ventricle, centrifuged and plasma was stored at −20° C. for analysis.

In a different group of experiments, BALB/c male mice (Harlan, Udine, Italy) aged 6-8 weeks and weighting between 25-30 g were used. The mice (n=24) had free access to tap water and pelleted food and were housed in standard cages with a 12 h light/dark cycle. Environmental temperature was constantly maintained at 21° C. and the mice were kept under pathogen-free conditions. All experiments were carried out in accordance with Italians laws (Ministry of Health registration n 62/2000-B, Oct. 6, 2000) and complied with the Guidelines for Care and Use of International Centre for Genetic Engineering and Biotechnology (ICGEB). Mice were randomly divided in groups of 6 animals each: group 1, controls (saline); group 2, TRAIL 10 μg/mouse on day 0 and 1; group 3, MDP or LPS 500 μg/kg on day 3; group 4, TRAIL 10 μg/mouse on day 0 and day 1 plus MDP or LPS 500 μg/kg on day 3. All the solutions were administered by the intraperitoneal route. Animals were sacrificed on day 3, two hours after MDP administration.

### Metabolic Assays

Glucose tolerance tests (2 g/kg glucose i.p.) were performed in overnight-fasted mice at 6 and 12 weeks. Blood samples were obtained from the tail tip at the indicated times, and glucose levels were measured using a glucometer (AccuCheck II; Roche, NSW, Australia). The bloods were then centrifuged at 6000 g for 6 minutes to obtain the sera where insulin levels were measured at the indicated times by an ELISA kit (Millipore, Cat# EZRMI-13K).

Basal insulin levels and the concentration of non-esterified fatty acids were measured on plasma obtained from the blood collected at the end of the study.

Lipids levels (total cholesterol, LDL, HDL and triglycerides) were measured from 200 μL of serum collected at fasting at the end of the study by COBAS INTEGRA 200. FFAs were determined using a colorimetric kit (Wako Pure Chemical Industries, Osaka, Japan).

IL-6 circulating levels were measured by ELISA in the plasmas collected at the end of the study (R&D, Cat#M6000B).

### Indirect calorimetry Studies

O₂ consumption rate (VO₂) and CO₂ production rate (VCO₂) were monitored by indirect calorimetry (Oxymax, Columbus Instruments) at baseline, 4 and 8 weeks of study with 1 mouse per chamber. Each chamber was equipped with a 3-dimensional (xyz) infrared beam system (OPTO-M3) to record locomotor activity. Conditioned fresh air at 21±0.5° C. and 55±5% relative humidity was pumped into the chambers at 0.61/min. The animals were acclimated to the chambers for 1 day, and VO₂ and VCO₂ measurements taken every 30 minutes were collected and recorded on a computer over the next 24 hours. During the 12-hours dark/12-hour light phases, mice had free access to food and water. The energy expenditure was expressed as VO₂ adjusted per lean body mass as following (VO₂*total body mass)/lean body mass and expressed as mL/kg/h. RER was calculated as VCO₂ production/VO₂ consumption, with the values of 1 or 0.7 indicating 100% CHO or 100% fat oxidation, respectively.

### Homogenate Oxidations

Palmitate oxidation was measured in muscle homogenates using a modified method described by Turner and associates (Turner N, Diabetes, 56(8):2085-92, 2007). Muscles were homogenized in 19 volumes of ice-cold 250 mmol/L sucrose, 10 mmol/L Tris-HCl and 1 mmol/L EDTA, pH 7.4. For assessment of substrate oxidation, 50 μl of muscle homogenate was incubated with 450 μl reaction mixture (pH 7.4). Final concentrations of the reaction mixture were (in mmol/L): 100 sucrose, 80 KCl, 10 Tris-HCl, 5 KH₂PO₄, 1 MgCl₂, 2 malate, 2 ATP, 1 DTT, 0.2 EDTA and 0.3% fatty-acid free BSA. Substrates were 0.2 mmol/L [1-14C]palmitate (0.5 μCi) plus 2 mmol/L carnitine and 0.05 mmol/L coenzyme A. After 90 min of incubation at 30° C., the reaction was stopped by the addition of 100 μL of ice-cold 1 mol/L perchloric acid. CO₂ produced during the incubation was collected in 100 μL of 1 mol/L sodium hydroxide. For palmitate 14° C. counts present in the acid-soluble fraction were also measured and combined with the CO₂ values to give the total palmitate oxidation rate.

### Determination of Body Composition

Fat and lean body mass were measured at the beginning of the study and every four weeks by EchoMRI (Echo Medical Systems, Houston Tex.). The % of body mass increase was calculated as (total body weight-initial body weight)/initial body weight*100, the % adiposity was calculated as (fat mass/total body mass)*100, the % lean mass was calculated as (lean mass/total body mass)*100.

### Food Intake and Energy Intake

The food intake was measured every four weeks placing pellets previously weighed in total in the cages. The food that was left over was then collected and weighed to find the amount eaten. Energy intake was measured according to the digestible energy provided by both diets.

### Gene Expression Quantification by Real-Time PCR

3 micrograms of total RNA extracted from the adipose tissue were used to synthesize cDNA with Superscript First Strand synthesis system for RT-PCR (Gibco BRL). Angiotensinogen (Angio), BAX, BCL2, BMP7, caspase3, CD36, HO-1, IL-6, MCP1, MIF, NFκb, OPG, PAI-1, PPARalpha, PPARgamma, TNFalpha gene expression were analyzed by real-time quantitative RT-PCR using the TaqMan™ system based on real-time detection of accumulated fluorescence. Fluorescence for each cycle was quantitatively analyzed by an ABI Prism 7700 Sequence Detection System (Perkin-Elmer Inc.). Gene expression of the target sequence was normalized in relation to the expression of an endogenous control, 18s ribosomal RNA. Primers and TaqMan™probes were constructed with the help of Primer Express (ABI Prism 7700, Perkin-Elmer Inc) (Table 7).

TABLE 7
Sequences of Probes and Primers for the
Genes of Interest

| | | | SEQ ID NO: |
|---|---|---|---|
| Angiotensinogen | PROBE | 6-FAM CCTCCTCGAACTCAAA | 339 |
| NM_007428 | F primer | AGTGGGAGAGGTTCTCAATAGCA | 340 |
| | R primer | GACGTGGTCGGCTGTTCCT | 341 |
| BAX | Syber | | |
| NM_007527 | F primer | GGCCTTTTTGCTACAGGGTTT | 342 |
| | R primer | GTGTCTCCCCAGCCATCCT | 343 |
| BCL2 | Syber | | |
| NM_009741 | F primer | AAGGGCTTCACACCCAAATCT | 344 |
| | R primer | TTCTACGTCTGCTTGGCTTTGA | 345 |
| BMP7 | PROBE | 6-FAM CACCAGCAACCACTG | 346 |
| NM_007557 | F primer | GGGCTGGTTGGTGTTTGATATC | 347 |
| | R primer | GGTTGTGCCGAGGGTTGAC | 348 |
| CASPASE3 | Syber | | |
| NM_009810 | F primer | GACGGTCCTCCTGGTCTTTG | 349 |
| | R primer | GTGGCTGGCTGCATTGC | 350 |
| IL6 | PROBE | 6-FAM ATTGCCATTGCACAACT | 351 |
| NM_031168 | F primer | GGGAAATCGTGGAAATGAGAAA | 352 |
| | R primer | AAGTGCATCATCGTTGTTCATACA | 353 |
| MCP1 | PROBE | 6-FAM AATGGGTCCAGACATAC | 354 |
| NM_011333 | F primer | GTCTGTGCTGACCCCAAGAAG | 355 |
| | R primer | TGGTTCCGATCCAGGTTTTTA | 356 |
| OPG | PROBE | 6-FAM CGAACCTCACCACAGAG | 357 |
| NM_008764 | F primer | GCGTGCAGCGGCATCT | 358 |
| | R primer | TCAATCTCTTCTGGGCTGATCTT | 359 |
| PPARalpha | PROBE | 6-FAM TGCCAGTACTGCCGTTT | 360 |
| NM_011144 | F primer | GATTCAGAAGAAGAACCGGAACA | 361 |
| | R primer | CCGACAGACAGGCACTTGTG | 362 |
| PPARgamma | PROBE | 6-FAM CTCTGTGGACCTCTC | 363 |
| XM_124785 | F primer | GCCCACCAACTTCGGAATC | 364 |
| | R primer | TGCGAGTGGTCTTCCATCAC | 365 |
| TNFaplha | PROBE | 6-FAM TCACCCACACCGTCAG | 366 |
| NM_013693 | F primer | GGCTGCCCCGACTACGT | 367 |
| | R primer | TTTCTCCTGGTATGAGATAG- | 368 |
| | | CAAATC | |

### In Vivo Apoptosis

Adipose tissue apoptosis was detected by Transferase-mediated dUTP Nick End Labeling (TUNEL) staining. Apoptosis was identified by 3′ in situ end labeling of fragmented DNA with Terminal deoxynucleotidyltransferase (TdT). After fixation and permeabilization with 0.1% Triton X-100 and 0.1% sodium citrate fresh solution, 20 μm frozen sections of adipose tissue were incubated with TUNEL reaction mixture, according to the manufacturer's instructions (Roche diagnostic, Indianapolis, USA) and mounted with DAPI to be seen under fluorescence microscopy. The number of (TUNEL)-positive cells was calculated as TUNEL-positive cells every frame.

### Body Temperature Determination

A handheld, thermocouple thermometer with a digital display (Type J 600-1000, Barnant Company, Barrington, Ill.) was used to measure body temperature just before sacrifice, using of a rectal probe as described elsewhere (Newsom et al., Contemporary topics in laboratory animal science/American Association for Laboratory Animal Science 43:13-18, 2004). Mice were sacrificed 2 hours after MDP or LPS administration, blood was collected and serum was obtained.

### Determination of Serum Amyloid-A (SAA)

Blood was collected directly into test tubes following decapitation. Serum was recovered by centrifugation at 2000×g at 4° C., and then stored at −80° C. until being used. The SAA was assayed using ELISA kits (Cusabio Biotech Co., China), the experimental procedures were performed according to the instruction of the manufacturer, and the amount of SAA expressed as g per ml serum.

### Determination of Cells Number in the Peritoneal Exudate

Peritoneal exudate cells (PEC) were obtained as follows: immediately after decapitation, 2 ml of PBS with BSA (0.1%) were injected into the peritoneal cavity, and the cavity was massaged for 4 minutes. The fluid (about 1.5 ml) was recovered using a syringe and the number of cells was counted after appropriate dilution using a Bürker chamber.

### Cytokines and Chemokines Analysis and Data Analysis

Cytokines (IL1α, ILβ, IL3, IL6, IL10, IL12p40, IL12p70, IL13, TNF-α) and chemokines (Exotaxin, G-CSF, KC, MIP-1α, MIP-1β, RANTES) levels were measured in duplicate, using an Bio-Plex 200 reader (Bio-Rad, Hercules, Calif., USA). Values are reported as mean values±standard deviation (SD) Statistical significance was calculated using one-way analysis of variance (ANOVA), and Tukey post-test for multiple comparison. Statistical analysis have been performed using the GraphPad Prism version 5 software (www.graphpad.com/prism/p5.htm)

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A TRAIL death receptor (DR) agonist for stimulating the death of an adipose cell.

2. The TRAIL DR agonist according to claim 1, which is a TRAIL polypeptide.

3. The TRAIL DR agonist according to claim 2, wherein the TRAIL polypeptide comprises an amino acid sequence selected from the group consisting of:
(a) an amino acid sequence represented by formula I:
(I)
(SEQ ID NO: 369)
AHΦ₁TGX₁X₂X₃X₄X₅X₆X₇X₈X₉X₁₀X₁₁X₁₂X₁₃X₁₄X₁₅X₁₆X₁₇
KX₁₈Φ₂GX₁₉KIX₂₀Σ₁WÅ₁Σ₂Σ₃RX₂₁GHSFX₂₂X₂₃X₂₄Φ₃X₂₅Φ₄
RNGELVIX₂₆X₂₇X₂₈GΦ₅YYIYX₂₉QX₃₀YΦ₆RFX₃₁EX₃₂X₃₃Å₂
X₃₄X₃₅X₃₆X₃₇X₃₈X₃₉X₄₀X₄₁X₄₂X₄₃X₄₄X₄₅X₄₆X₄₇X₄₈X₄₉
X₅₀X₅₁ [symbol] QΦ₇Φ₈QYIYKX₅₂TX₅₃YPX₅₄PIΦ₉LMKSARNX₅₅
CWS [symbol] Å₂Σ₄EYGLYSIYQGGΦ₁₀FELKΔ₁X₅₆DRIFVSVX₅₇NX₅₈X₅₉
LΦ₁₁DΦ₁₂X₆₀X₆₁EΣ₅SFΦ₁₃G
wherein:
Φ₁ is selected from hydrophobic amino acid residues;
X₁ is selected from any amino acid residue;
X₂ is selected from basic amino acid residues or small amino acid residues;
X₃ is selected from any amino acid residue;
X₄ is selected from basic amino acid residues; or small amino acid residues;
X₅ is optionally present and is selected from small amino acid residues or basic amino acid residues;
X₆ is selected from any amino acid residue;
X₇ is optionally present and is selected from hydrophobic amino acid residues;
X₈ is selected from small amino acid residues or hydrophobic amino acid residues;
X₉ is selected from hydrophobic amino acid residues or small amino acid residues;
X₁₀ is selected from small amino acid residues or hydrophobic amino acid residues;
X₁₁ is selected from small amino acid residues or hydrophobic amino acid residues;
X₁₂ is selected from small amino acid residues or hydrophobic amino acid residues;
X₁₃ is selected from any amino acid residue;
X₁₄ is selected from small amino acid residues or basic amino acid residues;
X₁₅ is selected from basic amino acid residues or neutral/polar amino acid residues;
X₁₆ is selected from any amino acid residue;
X₁₇ is selected from any amino acid residue;
X₁₈ is selected from small amino acid residues and neutral/polar amino acid residues;
Φ₂ is selected from hydrophobic residues;
X₁₉ is selected from basic amino acid residues or neutral/polar amino acid residues;
X₂₀ is selected from any amino acid residue;
Σ₁ is selected from small amino acid residues;
Å₁ is selected from acidic amino acid residues;
Σ₂ is selected from small amino acid residues;
Σ₃ is selected from small amino acid residues;
X₂₁ is selected from small amino acid residues or basic amino acid residues;
X22 is selected from hydrophobic amino acid residues or neutral/polar amino acid residues;
X23 is selected from small amino acid residues or neutral/polar amino acid residues;
X24 is selected from neutral/polar amino acid residues or basic amino acid residues;
Φ3 is selected from hydrophobic residues;
X25 is selected from basic amino acid residues or hydrophobic amino acid residues;
Φ4 is selected from hydrophobic amino acid residues;
X26 is selected from any amino acid residue;
X27 is selected from acidic amino acid residues or neutral/polar amino acid residues;
X28 is selected from any amino acid residue;
Φ5 is selected from hydrophobic amino acid residues;
X29 is selected from small amino acid residues or neutral/polar amino acid residues;
X30 is selected from small amino acid residues or hydrophobic amino acid residues;
Φ6 is selected from hydrophobic amino acid residues;
X31 is selected from neutral/polar amino acid residues or basic amino acid residues;
X32 is optionally present and is selected from small amino acid residues or hydrophobic amino acid residues;
X33 is optionally present and is selected from charged amino acid residues;
Å2 is selected from acidic amino acid residues;
X34 is optionally present and is selected from hydrophobic amino acid residues, or small amino acid residues;
X35 is optionally present and is selected from small amino acid residues or hydrophobic amino acid residues;
X36 is optionally present and is selected from small amino acid residues or basic amino acid residues;
X37 is optionally present and is selected from small amino acid residues or hydrophobic amino acid residues;
X38 is optionally present and is selected from hydrophobic amino acid residues;
X39 is optionally present and is selected from small amino acid residues;
X40 is optionally present and is selected from basic amino acid residues or small amino acid residues;
X41 is selected from any amino acid residue;
X42 is selected from any amino acid residue;
X43 is selected from any amino acid residue;
X44 is selected from small amino acid residues or basic amino acid residues;
X45 is optionally present and is selected from any amino acid residue;
X46 is optionally present and is selected from acidic amino acid residues;
X47 is optionally present and is selected from hydrophobic amino acid residues;
X48 is optionally present and is selected from basic amino acid residues;
X49 is optionally present and is selected from basic amino acid residues;
X50 is optionally present and is selected from neutral/polar amino acid residues or basic amino acid residues;
X51 is optionally present and is selected from any amino acid residue, or neutral/polar amino acid residues or hydrophobic amino acid residues; [symbol]
1 is selected from basic amino acid residues;
Φ7 is selected from hydrophobic amino acid residues;
Φ8 is selected from hydrophobic amino acid residues;
X52 is selected from any amino acid residue;
X53 is selected from any amino acid residue;
X54 is selected from acidic amino acid residues or small amino acid residues;
Φ9 is selected from hydrophobic amino acid residues;
X55 is selected from small amino acid residues or neutral/polar amino acid residues; [symbol]
2 is selected from basic amino acid residues;
Å2 is selected from acidic amino acid residues;
Σ4 is selected from small amino acid residues;
Φ10 is selected from hydrophobic amino acid residues;
Δ1 is selected from charged amino acid residues;
X56 is selected from neutral/polar amino acid residues or acidic amino acid residues;
X57 is selected from small amino acid residues or neutral/polar amino acid residues;
X58 is selected from acidic amino acid residues or small amino acid residues;
X59 is selected from basic amino acid residues or neutral/polar amino acid residues;
Φ11 is selected from hydrophobic amino acid residues;
Φ12 is selected from hydrophobic amino acid residues;
X60 is selected from acidic amino acid residues or neutral/polar amino acid residues;
X61 is selected from basic amino acid residues or neutral/polar amino acid residues;
Σ5 is selected from small amino acid residues; and
Φ13 is selected from hydrophobic amino acid residues
and
(b) an amino acid sequence that shares at least 70% sequence similarity or sequence identity with the amino acid sequence represented by formula I.

4. The TRAIL DR agonist according to claim 3, wherein the TRAIL polypeptide comprises an amino acid sequence selected from the group consisting of:
(a) an amino acid sequence selected from:
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFFGAHITGTRGRSNTLSSPNSKNEK
ALGRKINSWESSRSGHSFLSNLHLRNGELVIHEKGFYYIYSQTYFRFQEEIKENTKNDQ
MVQYIYKYTSYPDPILLMKSARNSCWSKDAEYGLYSIYQGGIFELKENDRIFVSVTNE
HLIDMDHEASFFG [SEQ ID NO: 54] (corresponding to amino acids 124-276 of a human
TRAIL isoform 1, as set forth in NCBI Accession: NP_003801);
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFFG [SEQ ID NO: 56] (corresponding
to amino acids 124-276 of a synthetic TRAIL, as set forth in NCBI Accession: AAV38370);
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFLG [SEQ ID NO: 58] (corresponding
to amino acids 124-276 of a synthetic TRAIL, as set forth in NCBI Accession: AAX29952);
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFFG [SEQ ID NO: 60] (corresponding
to amino acids 21-173 of a human TRAIL isoform CRA_b, as set forth in NCBI Accession:
EAW78466);
AHITGTRGRSNTLSSPNSKNEKALGHKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFFG [SEQ ID NO: 62] (corresponding
to amino acids 124-276 of a Pan troglodytes TRAIL, as set forth in NCBI Accession:
XP_516879);
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPDPILLMKSARNSCWSKDAEYGL
YSIYQGGIFELKENDRIFVSVTNEHLIDMDHEASFFG [SEQ ID NO: 64] (corresponding
to amino acids 11-163 of a human TRAIL fragment, as set forth in NCBI Accession:
1D0G_A);
AHITGTRGRSNTLSSPNSKNEKALGRKINSWESSRSGHSFLSNLHLRNGELVIHEKGFY
YIYSQTYFRFQEEIKENTKNDKQMVQYIYKYTSYPAPILLMKSARNSCWSKDAEYGL
YSIYQGGIFELKENDRIFVSVTNEHLIDMDHEASFFG [SEQ ID NO: 66] (corresponding
to amino acids 34-186 of a human TRAIL fragment, as set forth in NCBI Accession: 1DG6);
AHITGTRGRSNTLSSPNSKNEKALGRKINSWESSRSGHSFLSNLHLRNGELVIQEKGFY
YIYSQTYFRFQEEIKENTKNDKQMVQYIYKYTSYPDPILLMKSARNSCWSKDAEYGL
YSIYQGGLFELKKDDRIFVSVTNEHLIDMDHEASFFG [SEQ ID NO: 68] (corresponding
to amino acids 124-276 of a Macaca mulatta TRAIL, as set forth in NCBI Accession:
XP_001084768);
AHITGTRGRSNTLSSPNSKNEKALGRKINSWESSRSGHSFQSNLHLRNGELVIHEKGFY
YIYSQTYFRFQEEIKENAKNDKQMVQYIYKYTSYPDPILLMKSARNSCWSKDAEYGL
YSIYQGGIFELKENDRIFVSVTNEHLIDMDHEASFFG [SEQ ID NO: 70] (corresponding
to amino acids 7-164 of a Crassostrea ariakensis TRAIL, as set forth in NCBI Accession:
ABU39827);
PQRVAAHITGTRGRSNTLSSPNSKNEKALGRKINSWESSRSGHSFLSNLHLRNGELVIH
EKGFYYIYSQTYFRFQEEIKENTKNDKQMVQYIYKYTSYPDPILLMKSARNSCWSKDA
EYGLYSIYQGGIFELKENDRIFVSVTNEHLIDMDHEASFFG [SEQ ID NO: 72]
(corresponding to amino acids 1-158 of a human TRAIL fragment, as set forth in NCBI
Accession: 1D4V_B);
PQRVAAHITGTRGRSNTLSSPSKRNNKXXXRKINSWESSRSGHSFLSNLHLRNGELVI
HEKGFYYIYSQTYFRFQEEIKENTKNDKQMVQYIYKYTSYPDPILLMKSARNSCWSK
DAEYGLYSIYQGGIFELKENDRIFVSVTNEHLIDMDHEASFFG [SEQ ID NO: 74]
(corresponding to amino acids 119-276 of a Pongo abelii TRAIL, as set forth in NCBI
Accession: XP_002814335);
PQRVAAHITGTRGSSNTLPIPNSKNEKALGRKINSWESSRSGHSFLSNLHLRNGELVIH
EKGLYYIYCQVYFRFQEEIQENRKNDKQMVQYIYKYTSYPDPILLMKSARNNCWSKD
AEYGLYSIYQGGIFELKENDRIFVSVTNGQLIDMDHEASFFG [SEQ ID NO: 76]
(corresponding to amino acids 119-276 of a Callithrix jacchus TRAIL, as set forth in NCBI
Accession: XP_002759427);
PQRVAAHITGTSRRRSTFPVPSSKNEKALGQKINSWESSRKGHSFLNNLHLRNGELVIH
QRGFYYIYSQTYFRFQEPEEIPTGQNRKRNKQMVQYIYKHTSYPDPILLMKSARNSCW
SKDSEYGLYSIYQGGIFELKENDRIFVSVSNEQLIDMDQEASFFG [SEQ ID NO: 78]
(corresponding to amino acids 115-276 of a Felis catus TRAIL, as set forth in NCBI
Accession: NP_001124316);
LQRVAAHITGTSRRRSTFPVPSSKNEKALGQKINSWESSRKGHSFLSNLHLRNGELVIH
QSGFYYIYSQTYFRFQEPEETSGPISKEQNRKKNKQMVQYIYKYTSYPDPILLMKSAR
NSCWSKDSEYGLYSIYQGGIFELKENDRIFVSVNNEQLIDMDQEASFFG [SEQ ID NO:
80] (corresponding to amino acids 116-281 of an Ailuropoda melanoleuca TRAIL, as set forth
in NCBI Accession: XP_002921635);
LQRVAAHITGTSRRRSTVSIPRSKNEKALGQKINAWETSRKGHSFLNNLHLRNGELVI
HQTGFYYIYSQTYFRFQEPEEILGTVATEENRRKNKQMVQYIYKSTDYPDPILLMKSA
RNSCWSKDSEYGLYSIYQGGIFELKENDRIFVSVTNEQLIDMDQEASFFG [SEQ ID NO:
82] (corresponding to amino acids 124-284 of an Equus caballus TRAIL, as set forth in NCBI
Accession: XP_001494138);
AHITGTSRRRSTFPVPSSKNEKALGQKINSWESSRKGHSFLSNLHLRNGELVIHQSGFY
YIYSQTYFRFQEPEETSGPISKEQNRKKNKQMVQYIYKYTSYPDPILLMKSARNSCWS
KDSEYGLYSIYQGGIFELKENDRIFVSVNNEQLIDMDQEASFFG [SEQ ID NO: 84]
(corresponding to amino acids 121-276 of an Ailuropoda melanoleuca TRAIL, as set forth in
NCBI Accession: EFB16787);
AHITGSNRKKSTLPVPGSKNEKAVGHKINSWESSRKGHSFLNNLYLRNGELVILQTGF
YYIYSQTYFRFQEPEEVLGTVSTEENRKKIKQMVQYIYKYTNYPDPILLMKSARNSCW
SKDSEYGLYSIYQGGIFELKENDRIFVSVTNERLVDLDQEASFFG [SEQ ID NO: 86]
(corresponding to amino acids 122-282 of a Bos taurus TRAIL, as set forth in NCBI
Accession: XP_583785);
AHITGTSRKRSTFPSLSSKYEKALGQKINSWESSRKGHSFLNNFHLRNGELVIHQTGFY
YIYSQTYFRFQEPEEILGTVSTEGNRKKNRQMIQYIYKWTSYPDPILLMKSARNSCWS
KDSEYGLYSIYQGGIFELKEDDRIFVSVTNEQLIDMDQEASFFG [SEQ ID NO: 88]
(corresponding to amino acids 124-284 of a Sus scrofa TRAIL, as set forth in NCBI
Accession: NP_001019867);
AHITGTSRRSMFPIPSSKNDKALGHKINSWDSTRKGHSFLNNLHLRNGELVIHQRGFY
YIYSQTYFRFQEPEEIPTGQNRKRNKQMVQYIYKHTSYPDPILLMKSARNSCWSKDSE
YGLYSIYQGGIFELKENDRIFVSVSNEQLIDMDQEASFFG [SEQ ID NO: 90]
(corresponding to amino acids 124-276 of a Canis lupis familiaris TRAIL, as set forth in NCBI
Accession: NP_001124308);
AHLTGNSWRSFISVPAPGSQSGKNLGQKISSWESSRKGHSFLNNLHLRNGELVIHQTG
LYYIYSQTYFRFQELEEISGTISREEIKKRNKQMVQYIYKWTSYPDPILLMKSARNSCW
SKDSEYGLYSIYQGGIFELKENDRIFVSVTNEQLIDMNQESSFFG [SEQ ID NO: 92]
(corresponding to amino acids 128-289 of an Oryctolagus cuniculus TRAIL, as set forth in
NCBI Accession: XP_002716472);
AHITGITRRSNLALIPISKDGKTLGQKIETWESSRRGHSFLNHVHLRNGELVIQEEGLY
YIYSQTYYRFKEAKEASKTVSKDGGRIKQMVQYIYKYTSYPDPILLMKSARNSCWSRE
AEYGLYSIYQGGLFELKENDRIFVSVTNEHLMDLDQEASFFG [SEQ ID NO: 94]
(corresponding to amino acids 128-286 of a Rattus novegicus TRAIL, as set forth in NCBI
Accession: EDM01114);
AHITGITRRSNLALIPISKDGKTLGQKIETWESSRRGHSFLNHVHLRNGELVIQEEGLY
YIYSQTYYRFKEAKEASKTVSKDGGRIKQMVQYIYKYTSYPDPILLMKSARNSCWSRE
AEYGLYSIYQGGLFELKENDRIFVSVTNEHLMDLDHEASFFG [SEQ ID NO: 96]
(corresponding to amino acids 128-286 of a Rattus novegicus TRAIL, as set forth in NCBI
Accession: NP_663714);
AHITGITRRSNLALIPISKDGKTLGQKIETWESSRRGHSFLNHVHLRNGELVIQEEGLY
YIYSQTYYRFKEAKEASKTVSKDGGRIKQMVQYIYKYTSYPDPILLMKSARNSCWSRE
AEYGLYSIYQGGLFELKENDRIFVSVTNEHLMDLDQEASFFG [SEQ ID NO: 98]
(corresponding to amino acids 128-286 of a Rattus novegicus TRAIL, as set forth in NCBI
Accession: ABK32522);
AHITGITRRSNSALIPISKDGKTLGQKIESWESSRKGHSFLNHVLFRNGELVIEQEGLYY
IYSQTYFRFQEAKDASKMVSKDKVRTKQLVQYIYKYTSYPDPIVLMKSARNSCWSRD
AEYGLYSIYQGGLFELKKNDRIFVSVTNEHLMDLDQEASFFG [SEQ ID NO: 100]
(corresponding to amino acids 128-286 of a Mus musculus TRAIL, as set forth in NCBI
Accession: BAE34141); and
AHITGITRRSNSALIPISKDGKTLGQKIESWESSRKGHSFLNHVLFRNGELVIEQEGLYY
IYSQTYFRFQEAEDASKMVSKDKVRTKQLVQYIYKYTSYPDPIVLMKSARNSCWSRD
AEYGLYSIYQGGLFELKKNDRIFVSVTNEHLMDLDQEASFFG [SEQ ID NO: 102]
(corresponding to amino acids 128-286 of a Mus musculus TRAIL, as set forth in NCBI
Accession: NP_33451); or
(b) an amino acid sequence that shares at least 70% (and at least 71% to at least 99% and all integer percentages in between) sequence similarity or sequence identity with the sequence set forth in any one of SEQ ID NO: 54, 56, 58, 60, 62, 64, 66, 68, 70, 72, 74, 76, 78, 80, 82, 84, 86, 88, 90, 92, 94, 96, 98, 100 or 102;
(c) an amino acid sequence which is encoded by the nucleotide sequence set forth in any one of:
gctcacataactgggaccagaggaagaagcaacacattgtcttctccaaactccaagaatgaaaaggctctgggccgcaaaataaact
cctgggaatcatcaaggagtgggcattcattcctgagcaacttgcacttgaggaatggtgaactggtcatccatgaaaaagggttttact
acatctattcccaaacatactttcgatttcaggaggaaataaaagaaaacacaaagaacgacaaacaaatggtccaatatatttacaaat
acacaagttatcctgaccctatattgttgatgaaaagtgctagaaatagttgttggtctaaagatgcagaatatggactctattccatctatc
aagggggaatatttgagcttaaggaaaatgacagaatttttgtttctgtaacaaatgagcacttgatagacatggaccatgaagccagttt
ttttggg [SEQ ID NO: 53] (corresponding to a nucleotide sequence from NCBI Accession:
NM_003810, encoding amino acids 124-276 of a human TRAIL isoform 1, as set forth in
NCBI Accession: NP_003801);
gctcacataactgggaccagaggaagaagcaacacattgtcttctccaaactccaagaatgaaaaggctctgggccgcaaaataaact
cctgggaatcatcaaggagtgggcattcattcctgagcaacttgcacttgaggaatggtgaactggtcatccatgaaaaagggttttact
acatctattcccaaacatactttcgatttcaggaggaaataaaagaaaacacaaagaacgacaaacaaatggtccaatatatttacaaat
acacaagttatcctgaccctatattgttgatgaaaagtgctagaaatagttgttggtctaaagatgcagaatatggactctattccatctatc
aagggggaatatttgagcttaaggaaaatgacagaatttttgtttctgtaacaaatgagcacttgatagacatggaccatgaagccagttt
tttcggg [SEQ ID NO: 55] (corresponding to a nucleotide sequence from NCBI Accession:
BT019563, encoding amino acids 124-276 of a synthetic TRAIL, as set forth in NCBI
Accession: AAV38370);
gctcacataactgggaccagaggaagaagcaacacattgtcttctccaaactccaagaatgaaaaggctctgggccgcaaaataaact
cctgggaatcatcaaggagtgggcattcattcctgagcaacttgcacttgaggaatggtgaactggtcatccatgaaaaagggttttact
acatctattcccaaacatactttcgatttcaggaggaaataaaagaaaacacaaagaacgacaaacaaatggtccaatatatttacaaat
acacaagttatcctgaccctatattgttgatgaaaagtgctagaaatagttgttggtctaaagatgcagaatatggactctattccatctatc
aagggggaatatttgagcttaaggaaaatgacagaatttttgtttctgtaacaaatgagcacttgatagacatggaccatgaagccagttt
tttaggg [SEQ ID NO: 57] (corresponding to a nucleotide sequence from NCBI Accession:
AY893035, encoding amino acids 124-276 of a synthetic TRAIL, as set forth in NCBI
Accession: AAX29952);
gctcacataactgggaccagaggaagaagcaacacattgtcttctccaaactccaagaatgaaaaggctctgggccgcaaaataaact
cctgggaatcatcaaggagtgggcattcattcctgagcaacttgcacttgaggaatggtgaactggtcatccatgaaaaagggttttact
acatctattcccaaacatactttcgatttcaggaggaaataaaagaaaacacaaagaacgacaaacaaatggtccaatatatttacaaat
acacaagttatcctgaccctatattgttgatgaaaagtgctagaaatagttgttggtctaaagatgcagaatatggactctattccatctatc
aagggggaatatttgagcttaaggaaaatgacagaatttttgtttctgtaacaaatgagcacttgatagacatggaccatgaagccagttt
ttttggg [SEQ ID NO: 59] (corresponding to a nucleotide sequence from NCBI Accession:
CH471052, encoding amino acids 21-173 of a human TRAIL isoform CRA_b, as set forth in
NCBI Accession: EAW78466);
gctcacataactggaaccagaggaagaagcaacacattgtcttctccaaactccaagaatgaaaaggctctgggccacaaaataaact
cctgggaatcatcaaggagtgggcattcattcctgagcaacttgcacttgaggaatggcgaactggtcatccatgaaaaagggttttac
tacatctattcccaaacatactttcgatttcaggaggaaataaaagaaaacacaaagaacgacaaacaaatggtccaatatatttacaaat
acacaagttatcctgaccctatattgttgatgaaaagcgctagaaatagttgttggtctaaagatgcagaatatggactctattccatctat
caagggggaatatttgagcttaaggaaaatgacagaatttttgtttctgtaacaaatgagcacttgatagacatggaccatgaagccagt
tttttcggg [SEQ ID NO: 61] (corresponding to a nucleotide sequence from NCBI Accession:
XM_516879, encoding amino acids 124-276 of a Pan troglodytes TRAIL, as set forth in NCBI
Accession: XP_516879);
gcncayathacnggnacnmgnggnmgnwsnaayacnytnwsnwsnccnaaywsnaaraaygaraargcnytnggnmgna
arathaaywsntgggamsnwsnmgnwsnggncaywsnttyytnwsnaayytncayytnmgnaayggngarytngtnathc
aygaraarggnttytaytayathtaywsncaracntayttymgnttycargargarathaargaraayacnaaraaygayaarcaratg
gtncartayathtayaartayacnwsntayccngayccnathytnytnatgaarwsngcnmgnaaywsntgytggwsnaargay
gcngartayggnytntaywsnathtaycarggnggnathttygarytnaargaraaygaymgnathttygtnwsngtnacnaayg
arcayytnathgayatggaycaygargcnwsnttyttyggn [SEQ ID NO: 63] (degenerate nucleotide
sequence encoding amino acids 11-163 of a human TRAIL fragment, as set forth in NCBI
Accession: 1D0G_A);
gcncayathacnggnacnmgnggnmgnwsnaayacnytnwsnwsnccnaaywsnaaraaygaraargcnytnggnmgna
arathaaywsntgggamsnwsnmgnwsnggncaywsnttyytnwsnaayytncayytnmgnaayggngarytngtnathc
aygaraarggnttytaytayathtaywsncaracntayttymgnttycargargarathaargaraayacnaaraaygayaarcaratg
gtncartayathtayaartayacnwsntayccngcnccnathytnytnatgaamsngcnmgnaaywsntgytggwsnaargay
gcngartayggnytntaywsnathtaycarggnggnathttygaiytnaargaraaygaymgnathttygtnwsngtnacnaayg
arcayytnathgayatggaycaygargcnwsnttyttyggn [SEQ ID NO: 65] (degenerate nucleotide
sequence encoding amino acids 34-186 of a human TRAIL fragment, as set forth in NCBI
Accession: 1DG6);
gctcacataactgggaccagaggaagaagcaacacattgtcttctccaaactccaagaatgaaaaggctctgggccgcaaaataaact
cctgggaatcatcaaggagtgggcattcattcctgagcaacttgcacttgaggaatggcgaactggtcatccaagaaaaggggttttac
tacatctattcccaaacatactttcgatttcaggaggaaataaaagaaaacacaaagaacgacaaacaaatggtccaatatatttacaaat
acacaagttatcctgaccctatactgctgatgaaaagcgctagaaatagttgttggtctaaagatgcagaatacggactctattccatcta
tcaagggggattatttgagcttaagaaagatgacagaatttttgtttctgtaacaaatgagcacttgatagacatggaccatgaagccag
ctttttcggg [SEQ ID NO: 67] (corresponding to a nucleotide sequence from NCBI Accession:
XM_001084768, encoding amino acids 124-276 of a Macaca mulatta TRAIL, as set forth in
NCBI Accession: XP_001084768);
gctcacataactgggaccagaggaagaagcaacacattgtcttctccaaactccaagaatgaaaaggctctgggccgcaaaataaact
cctgggaatcatcaaggagtgggcattcattccagagcaacttgcacttgaggaatggtgaactggtcatccatgaaaaagggttttac
tacatctattcccaaacatactttcgatttcaggaggaaataaaagaaaacgcaaagaacgacaaacaaatggtccaatatatttacaaat
acacaagttatcctgaccctatattgttgatgaaaagtgctagaaatagttgttggtctaaagatgcagaatatggactctattccatctatc
aagggggaatatttgagcttaaggaaaatgacagaatttttgtttctgtaacaaatgagcacttgatagacatggaccatgaagccagttt
tttcggg [SEQ ID NO: 69] (corresponding to a nucleotide sequence from NCBI Accession:
EF541151, encoding amino acids 11-164 of a Crassostrea ariakensis TRAIL, as set forth in
NCBI Accession: ABU39827);
gcncayathacnggnacnmgnggnmgnwsnaayacnytnwsnwsnccnaaywsnaaraaygaraargcnytnggnmgna
arathaaywsntgggamsnwsnmgnwsnggncaywsnttyytnwsnaayytncayytnmgnaayggngarytngtnathc
aygaraarggnttytaytayathtaywsncaracntayttymgnttycargargarathaargaraayacnaaraaygayaarcaratg
gtncartayathtayaartayacnwsntayccngayccnathytnytnatgaarwsngcnmgnaaywsntgytggwsnaargay
gcngartayggnytntaywsnathtaycarggnggnathttygarytnaargaraaygaymgnathttygtnwsngtnacnaayg
arcayytnathgayatggaycaygargcnwsnttyttyggn [SEQ ID NO: 71] (degenerate nucleotide
sequence encoding amino acids 6-158 of a human TRAIL fragment, as set forth in NCBI
Accession: 1D4V_B);
gctcacataactgggaccagaggaagaagcaacacattgtcttctccaagtaagagaaacaacaaannnnnnnnncgcaaaataaa
ctcctgggaatcatcaaggagtgggcattcattcctgagcaacttgcacttgaggaatggcgaactggtcatccatgaaaaagggtttt
actacatctattcccaaacatactttcgatttcaggaggaaataaaagaaaacacaaagaacgacaaacaaatggtccaatatatttaca
aatacacaagttatcctgatcctatattgctgatgaaaagcgctagaaatagttgttggtctaaagatgcagaatatggactctattccatc
tatcaagggggaatatttgagcttaaggaaaatgacagaatttttgtttctgtaacaaatgagcacttgatagacatggaccatgaagcca
gttttttcggg [SEQ ID NO: 73] (corresponding to a nucleotide sequence from NCBI Accession:
XM_002814289, encoding amino acids 124-276 of a Pongo abelii TRAIL, as set forth in
NCBI Accession: XP_002814335);
gctcacataactgggaccagaggaagtagcaacacgttgcctattccaaactccaagaatgaaaaggctctgggccgcaaaataaact
cctgggaatcatcaaggagtggacattctttcctgagcaacttgcacttgaggaatggcgagctggtcatccatgaaaaagggctgtat
tacatctattgccaagtatactttcgatttcaggaggaaatccaagaaaacagaaagaacgacaaacaaatggtccagtatatttacaaat
acacaagttatcctgaccccatactgctgatgaagagtgctagaaataattgttggtctaaagatgcagaatatggactctattccatctat
caagggggaatatttgagcttaaggaaaacgacagaatttttgtttctgtaacaaatgggcagttgatagacatggaccatgaagccag
ttttttcggg [SEQ ID NO: 75] (corresponding to a nucleotide sequence from NCBI Accession:
XM_002759381, encoding amino acids 124-276 of a Callithrix jacchus TRAIL, as set forth in
NCBI Accession: XP_002759427);
gctcacataactggaaccagtcggagaagaagcacattcccagttccaagctccaagaatgaaaaagctttgggtcagaaaataaact
cctgggagtcatcaagaaaaggacattcattcttgaataatttgcacttgaggaatggtgagctggttattcatcagagggggttttatta
catctattcccaaacatactttcgatttcaggaacctgaggaaattccaacaggacagaacagaaagagaaacaaacaaatggtccaat
atatttacaaacacacgagttatccggaccctatactgctgatgaaaagtgctagaaatagttgttggtctaaagattctgaatatggactc
tattccatctatcaaggtgggatatttgagcttaaggaaaacgatagaatttttgtctctgtatctaacgagcaattgattgacatggacca
agaagccagttttttcggg [SEQ ID NO: 77] (corresponding to a nucleotide sequence from NCBI
Accession: NM_001130844, encoding amino acids 120-276 of a Felis catus TRAIL, as set
forth in NCBI Accession: NP_001124316);
gctcacataactggaaccagtcggagaagaagcacgtttccagttccaagctccaagaatgaaaaagctttgggccagaaaataaact
cctgggagtcatcaagaaaaggacattcattcttgagtaatttgcacttgaggaatggagagctggttatccatcaaagtgggttttatta
catctattcccaaacatactttcgatttcaggaacctgaggaaacttcgggaccaatttcaaaggaacaaaacagaaagaaaaacaaac
aaatggtacaatatatttacaaatacacaagttatcctgaccctatactgctgatgaaaagtgctagaaatagttgctggtctaaagattct
gagtatggactctattccatctatcaaggtgggatatttgagcttaaggaaaatgatagaatttttgtctctgtaaataatgagcaattgatt
gacatggaccaagaagccagttttttcggg [SEQ ID NO: 79 (corresponding to a nucleotide sequence
from NCBI Accession: XM_002921589, encoding amino acids 121-281 of an Ailuropoda
melanoleuca TRAIL, as set forth in NCBI Accession: XP_002921635);
gctcacataactgggaccagtcggagaagaagcacagtctcaattccacgctccaagaatgaaaaagcactgggccagaaaataaac
gcctgggagacatcaagaaaaggacattcgttcttgaataatttacacttgaggaatggagagctggttatccatcaaacagggttttatt
acatctattcccaaacatactttcgatttcaggaacctgaggaaattttgggaacagttgcaacagaagagaacagaaggaaaaataaa
caaatggtacaatatatttacaaaagcacagactatcctgaccctatactgctgatgaaaagtgctagaaatagttgttggtctaaagattc
agaatacggactctattccatctatcaaggtggaatatttgagcttaaggaaaatgacagaatttttgtctctgtaactaatgagcaattgat
tgacatggaccaagaagccagtttcttcggg [SEQ ID NO: 81] (corresponding to a nucleotide sequence
from NCBI Accession: XM_001494088, encoding amino acids 124-284 of an Equus caballus
TRAIL, as set forth in NCBI Accession: XP_001494138);
gctcacataactggaaccagtcggagaagaagcacgtttccagttccaagctccaagaatgaaaaagctttgggccagaaaataaact
cctgggagtcatcaagaaaaggacattcattcttgagtaatttgcacttgaggaatggagagctggttatccatcaaagtgggttttatta
catctattcccaaacatactttcgatttcaggaacctgaggaaacttcgggaccaatttcaaaggaacaaaacagaaagaaaaacaaac
aaatggtacaatatatttacaaatacacaagttatcctgaccctatactgctgatgaaaagtgctagaaatagttgctggtctaaagattct
gagtatggactctattccatctatcaaggtgggatatttgagcttaaggaaaatgatagaatttttgtctctgtaaataatgagcaattgatt
gacatggaccaagaagccagttttttcggg [SEQ ID NO: 83] (corresponding to a nucleotide sequence
from NCBI Accession: GL192841, encoding amino acids 121-276 of an Ailuropoda
melanoleuca TRAIL, as set forth in NCBI Accession: EFB16787);
gctgctcatataactggaagcaatcggaaaaaaagtacgttgccagttccaggctccaagaatgaaaaagctgtgggccataaaataa
attcctgggagtcatcaagaaaaggacattcgttcttgaataatttgtacttaaggaatggagagctggttatccttcaaacaggattttatt
acatctattcccaaacatactttcgatttcaggaacctgaggaagttttgggaactgtttcaacagaagagaacagaaaaaaaatcaaac
aaatggtacaatatatttacaaatacacaaactatcctgaccctatactgctgatgaaaagtgctagaaatagttgttggtctaaagattca
gaatatggactctattccatctatcaaggaggaatatttgagcttaaggaaaatgatcgaatttttgtctctgtaactaatgaacgattggtt
gacctggaccaagaagccagttttttcgga [SEQ ID NO: 85] (corresponding to a nucleotide sequence
from NCBI Accession: XM_583785, encoding amino acids 122-282 of a Bos taurus TRAIL,
as set forth in NCBI Accession: XP_583785);
gctcacataactggaaccagtaggaaaagaagcacatttccatctctaagctccaaatatgaaaaagctttgggccagaaaataaactc
ctgggaatcatcaagaaaaggacattcattcttgaataattttcacttgaggaatggagagctggttatccatcaaacagggttttactac
atctattcccaaacatactttcgatttcaggaacctgaggaaattttgggaacggtttctacagaagggaacagaaagaaaaacaggca
aatgatacagtatatttacaaatggacaagctatcctgaccctatactgctgatgaaaagtgctagaaatagttgttggtctaaagattcag
aatatggactctattccatctatcaaggtggaatatttgagcttaaggaagatgaccgaatttttgtctctgttactaatgagcaactgattg
acatggaccaagaagccagttttttcggg [SEQ ID NO: 87] (corresponding to a nucleotide sequence
from NCBI Accession: NM_001024696, encoding amino acids 124-284 of a Sus scrofa
TRAIL, as set forth in NCBI Accession: NP_001019867);
gctcacataactggaaccagtcggagaagcatgtttccaattccaagctccaagaatgataaagctttgggccacaaaataaactcctg
ggattccacaagaaaaggacattcattcttgaataatttgcacttgaggaacggagagctggttatccatcaaagggggttttattacatc
tattcccaaacatactttcgatttcaggaacctgaggaaattccaacaggacagaacagaaagagaaacaaacaaatggtccaatatatt
tacaaacacacgagttatccggaccctatactgctgatgaaaagtgctagaaatagttgttggtctaaagattctgaatatggactctattc
catctatcaaggtgggatatttgagcttaaggaaaacgatagaatttttgtctctgtatctaacgagcaattgattgacatggaccaagaa
gccagttttttcggg [SEQ ID NO: 89] (corresponding to a nucleotide sequence from NCBI
Accession: NM_001130836, encoding amino acids 121-276 of a Canis lupis familiaris TRAIL,
as set forth in NCBI Accession: NP_001124308);
gctcacctaactgggaacagctggagaagctttatctcagtccctgctccaggctcccagagtggaaagaatttgggccagaaaataa
gctcctgggaatcatcaaggaaaggacattcattcctgaacaatttgcacctgaggaatggagagctggttatccatcaaacaggacttt
attacatctactcccaaacatactttcgatttcaggaacttgaagaaatttcaggaacaatttcaagagaagagatcaaaaagaggaaca
aacaaatggtacaatatatttacaaatggacaagctaccctgaccctatacttctgatgaaaagtgctagaaatagttgttggtctaagga
ttcggaatatggactctattccatctatcaaggaggaatatttgagcttaaggaaaatgaccgaattttcgtctctgtaacgaatgagcagt
tgattgacatgaaccaagaatccagtttttttggg [SEQ ID NO: 91] (corresponding to a nucleotide
sequence from NCBI Accession: XM_002716426, encoding amino acids 128-289 of an
Oryctolagus cuniculus TRAIL, as set forth in NCBI Accession: XP_002716472);
gctcacattaccgggatcactcggagaagcaacttagccttaattccaatctccaaggatggaaagaccttgggccagaagatagaaa
cctgggagtcctctcggagagggcattcatttctcaaccatgtgcacttgagaaacggagagctggtgatccaggaggagggcctgta
ttacatctactcccaaacgtactaccggttcaaggaggctaaagaagcttccaagacagtctcgaaggacggagggaggatcaaaca
gatggtgcagtacatctacaaatacaccagctaccccgatcccatactgctgatgaagagtgccagaaatagctgctggtccagagaa
gctgagtacggactgtactccatctatcagggggggctgttcgagctcaaagaaaatgacaggatttttgtttccgtgacgaatgagcat
ttgatggacctggatcaagaagccagtttctttgga [SEQ ID NO: 93] (corresponding to a nucleotide
sequence from NCBI Accession: CH473961, encoding amino acids 128-286 of a Rattus
novegicus TRAIL, as set forth in NCBI Accession: EDM01114);
gctcacattaccgggatcactcggagaagcaacttagccttaattccaatctccaaggatggaaagaccttgggccagaagatagaaa
cctgggagtcctctcggagagggcattcatttctcaaccatgtgcacttgagaaacggagagctggtgatccaggaggagggcctgta
ttacatctactcccaaacgtactaccggttcaaggaggctaaagaagcttccaagacagtctcgaaggacggagggaggatcaaaca
gatggtgcagtacatctacaaatacaccagctaccccgatcccatactgctgatgaagagtgccagaaatagctgctggtccagagaa
gctgagtacggactgtactccatctatcagggggggctgttcgagctcaaagaaaatgacaggatttttgtttccgtgacgaatgagcat
ttgatggacctggaccatgaagccagcttctttgga [SEQ ID NO: 95] (corresponding to a nucleotide
sequence from NCBI Accession: NM_145681, encoding amino acids 128-286 of a Rattus
novegicus TRAIL, as set forth in NCBI Accession: NP_663714);
gctcacattaccgggatcactcggagaagcaacttagccttaattccaatctccaaggatggaaagaccttgggccagaagatagaaa
cctgggagtcctctcggagagggcattcatttctcaaccatgtgcacttgagaaacggagagctggtgatccaggaggagggcctgta
ttacatctactcccaaacgtactaccggttcaaggaggctaaagaagcttccaagacagtctcgaaggacggagggaggatcaaaca
gatggtgcagtacatctacaaatacaccagctaccccgatcccatactgctgatgaagagtgccagaaatagctgctggtccagagaa
gctgagtacggactgtactccatctatcagggggggctgttcgagctcaaagaaaatgacaggatttttgtttccgtgacgaatgagcat
ttgatggacctggatcaagaagccagcttctttgga [SEQ ID NO: 97] (corresponding to a nucleotide
sequence from NCBI Accession: EF030546, encoding amino acids 128-286 of a Rattus
novegicus TRAIL, as set forth in NCBI Accession: ABK32522);
gctcacattactgggatcactcggagaagcaactcagctttaattccaatctccaaggatggaaagaccttaggccagaagattgagtc
ctgggagtcctctcggaaagggcattcatttctcaaccacgtgctctttaggaatggagagctggtcattgagcaggagggcctgtatt
acatctattcccaaacatacttccgatttcaggaagctaaagacgcttccaagatggtctcaaaggacaaggtgagaaccaaacagctg
gtgcagtacatctacaagtacaccagctatccggatcccatagtgctcatgaagagcgccagaaacagctgttggtccagagatgccg
agtacggactgtactccatctatcagggaggactgttcgagctaaaaaaaaatgacaggatttttgtttctgtgacaaatgaacatttgat
ggacctggatcaagaagccagcttctttgga [SEQ ID NO: 99] (corresponding to a nucleotide sequence
from NCBI Accession: AK157633, encoding amino acids 128-286 of a Mus musculus TRAIL,
as set forth in NCBI Accession: BAE34141); and
gctcacattactgggatcactcggagaagcaactcagctttaattccaatctccaaggatggaaagaccttaggccagaagattgaatc
ctgggagtcctctcggaaagggcattcatttctcaaccacgtgctctttaggaatggagagctggtcatcgagcaggagggcctgtatt
acatctattcccaaacatacttccgatttcaggaagctgaagacgcttccaagatggtctcaaaggacaaggtgagaaccaaacagctg
gtgcagtacatctacaagtacaccagctatccggatcccatagtgctcatgaagagcgccagaaacagctgttggtccagagatgccg
agtacggactgtactccatctatcagggaggattgttcgagctaaaaaaaaatgacaggatttttgtttctgtgacaaatgaacatttgat
ggacctggatcaagaagccagcttctttgga [SEQ ID NO: 101] (corresponding to a nucleotide sequence
from NCBI Accession: NM_009425, encoding amino acids 128-286 of a Mus musculus
TRAIL-NCBI Accession: NP_033451);
or
a complement of any one of SEQ ID NO: 53, 55, 57, 59, 61, 63, 65, 67, 69, 71, 73, 75, 77, 79, 81, 83, 85, 87, 89, 91, 93, 95, 97, 99 or 101;
(d) an amino acid sequence which is encoded by a nucleotide sequence that shares at least 70% (and at least 71% to at least 99% and all integer percentages in between) sequence identity with the sequence set forth in any one of SEQ ID NO: 53, 55, 57, 59, 61, 63, 65, 67, 69, 71, 73, 75, 77, 79, 81, 83, 85, 87, 89, 91, 93, 95, 97, 99 or 101, or a complement thereof; and
(e) an amino acid sequence which is encoded by a nucleotide sequence that hybridizes under at least medium or high stringency conditions to the sequence set forth in any one of SEQ ID NO: 53, 55, 57, 59, 61, 63, 65, 67, 69, 71, 73, 75, 77, 79, 81, 83, 85, 87, 89, 91, 93, 95, 97, 99 or 101, or a complement thereof,
wherein the amino acid sequence of (a), (b), (c), (d) or (e) has any one or more activities selected from the group consisting of: inducing apoptosis of adipose tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, reducing circulating levels of the proinflammatory cytokines IL-6, IL-1alpha and MCP, counteracting lipopolysaccaride- and muramildipeptide-induced inflammation.

5. The TRAIL DR agonist according to claim 3, wherein the TRAIL polypeptide further comprises upstream of the sequence represented by formula I an amino acid sequence represented by formula II:
(II)
(SEQ ID NO: 370)
X₆₁X₆₂X₆₃X₆₄X₆₅X₆₆X₆₇X₆₈X₆₉X₇₀X₇₁ [symbol] VA
wherein:
X61 is optionally present and is selected from any amino acid residues, with the proviso that optionally X61 is present when X62 is present;
X62 is optionally present and is selected from any amino acid residues with the proviso that optionally X62 is present when X63 is present;
X63 is absent or is selected from acidic amino acid residues or small amino acid residues with the proviso that optionally X63 is present when X64 or X68 is present;
X64 is optionally present and is selected from basic amino acid residues;
X65 is optionally present and is selected from acidic amino acid residues;
X66 is optionally present and is selected from basic amino acid residues;
X67 is optionally present and is selected from acidic amino acid residues;
X68 is optionally present and is selected from basic amino acid residues or small amino acid residues with the proviso that optionally X68 is present when X69 is present;
X69 is optionally present and is selected from small amino acid residues or basic amino acid residues with the proviso that optionally X69 is present when X70 is present;
X70 is optionally present and is selected from small amino acid residues or hydrophobic amino acid residues with the proviso that optionally X70 is present when X71 is present;
X71 optionally present and is selected from neutral/polar amino acid residues or basic amino acid residues with the proviso that optionally X71 is present when [symbol] 3 is present; and [symbol]
3 is selected from basic amino acid residues.

6. The TRAIL DR agonist according to claim 5, wherein the sequence represented by formula II comprises an amino acid sequence selected from the group consisting of:
(a) an amino acid sequence selected from the group consisting of VRERGPQRVA [SEQ ID NO:104], PQRVA [SEQ ID NO: 106], VRERGLQRVA [SEQ ID NO: 108], VRERGLQRVA [SEQ ID NO: 110], EREKGPKRVA [SEQ D NO: 112], EREKGPQRVA [SEQ ID NO: 114], VSDRGSQRVA [SEQ ID NO: 116], VREKERERGPQRVA [SEQ ID NO: 118], PRGRRPQRVA [SEQ ID NO: 120] and PRGGRPQRVA [SEQ ID NO: 122];
(b) an amino acid sequence that shares at least 70% sequence similarity or sequence identity with the sequence set forth in any one of SEQ ID NO: 104, 106, 108, 110, 112, 114, 116, 118, 120 or 122;
(c) an amino acid sequence which is encoded by the nucleotide sequence set forth in any one of: gtgagagaaagaggtcctcagagagtagca [SEQ ID NO: 103], ccncarmgngtngcn [SEQ ID NO: 105], gtaaatgaaagaggtcttcagagagtagca [SEQ ID NO: 107], gtaagagaaagaggtcttcagagagtagca [SEQ ID NO: 109], gagagagaaaagggtcctaagagggtagct [SEQ ID NO: 111], gaaagagaaaaaggtccacagagagtggct [SEQ ID NO:113], gtaagcgaccaggttctcagagagtagct [SEQ ID NO: 115], gtaagagaaaaagaaagagaaagagggcctcagagagtagca [SEQ ID NO: 117], cccagaggtagaagaccccagagagtggca [SEQ ID NO: 119] or cccagaggtggaagaccccagagagtggca [SEQ ID NO: 121], or a complement of any one of SEQ ID NO: 103, 105, 107, 109, 111, 113, 115, 117, 119 or 121;
(d) an amino acid sequence which is encoded by a nucleotide sequence that shares at least 70% sequence identity with the sequence set forth in any one of SEQ ID NO: 103, 105, 107, 109, 111, 113, 115, 117, 119 or 121, or a complement thereof; and
(e) an amino acid sequence which is encoded by a nucleotide sequence that hybridizes under at least medium or high stringency conditions to the sequence set forth in any one of SEQ ID NO: 103, 105, 107, 109, 111, 113, 115, 117, 119 or 121, or a complement thereof,
The TRAIL DR agonist according to claim 3, wherein the TRAIL polypeptide further comprises downstream of the sequence represented by formula I an amino acid sequence represented by formula III:
(III)
(SEQ ID NO: 371)
AFX72X73X74X75
wherein:
X72 is optionally present and is selected from hydrophobic amino acid residues, wherein optionally X73 is present with the proviso that X72 is present;
X73 is optionally present and is selected from hydrophobic amino acid residues, wherein optionally X74 is present with the proviso that X73 is present;
X74 is optionally present and is selected from small amino acid residues or neutral/polar amino acid residues, wherein optionally X75 is present with the proviso that X74 is present; and
X75 is optionally present and is selected from hydrophobic amino acid residues, wherein optionally X76 is present with the proviso that X75 is present.

7. The TRAIL DR agonist according to claim 7, wherein the sequence represented by formula III comprises an amino acid sequence selected from the group consisting of:
(a) an amino acid sequence selected from the group consisting of AFLVG [SEQ ID NO: 124], AFLVGL [SEQ ID NO: 126], AF [SEQ ID NO: 128], AFLIG [SEQ ID NO: 130] and AFLIN [SEQ ID NO: 132];
(b) an amino acid sequence that shares at least 70% sequence similarity or sequence identity with the sequence set forth in any one of SEQ ID NO: 124, 126, 128, 130 or 132;
(c) an amino acid sequence which is encoded by the nucleotide sequence set forth in any one of: gcctttttagttggc [SEQ ID NO: 123], gcctttttagttggcttg [SEQ ID NO: 125], gccttt [SEQ ID NO: 127], gcctttttaatcggc [SEQ ID NO: 129] or gcctttttaattaac [SEQ ID NO: 131], or a complement of any one of SEQ ID NO: 123, 125, 127, 129 or 131;
(d) an amino acid sequence which is encoded by a nucleotide sequence that shares at least 70% sequence identity with the sequence set forth in any one of SEQ ID NO: 123, 125, 127, 129 or 131, or a complement thereof; and
(e) an amino acid sequence which is encoded by a nucleotide sequence that hybridizes under at least medium or high stringency conditions to the sequence set forth in any one of SEQ ID NO: 123, 125, 127, 129 or 131, or a complement thereof.

8. The TRAIL DR agonist according to claim 1, wherein the TRAIL polypeptide comprises an amino acid sequence selected from the group consisting of:
(a) an amino acid sequence selected from:
VRERGPQRVAAHITGTRGRSNTLSSPNSKNEKALGRKINSWESSRSGHSFLSNLHL
RNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVG
[SEQ ID NO: 2] (corresponding to amino acids 114-281 of a human TRAIL isoform 1, as
set forth in NCBI Accession: NP_003801);
MAMMEVQGGPSLGQTCVLIVIFTVLLQSLCVAVTYVYFTNELKQMQDKYSKSGI
ACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLS
NLHLRNGELVIHEKGFYYTYSQTYFRFQEEIKENTKNDKQMVQYIYKYTSYPDPIL
LMKSARNSCWSKDAEYGLYSIYQGGIFELKENDRIFVSVTNEHLIDMDHEASFFGA
FLVG [SEQ ID NO: 4]; (corresponding to a putative full-length synthetic TRAIL, as set
forth in NCBI Accession: AAV38370);
MAMMEVQGGPSLGQTCVLIVIFTVLLQSLCVAVTYVYFTNELKQMQDKYSKSGI
ACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLS
NLHLRNGELVIHEKGFYYTYSQTYFRFQEEIKENTKNDKQMVQYIYKYTSYPDPIL
LMKSARNSCWSKDAEYGLYSIYQGGIFELKENDRIFVSVTNEHLIDMDHEASFFGA
FLVGL [SEQ ID NO: 6] (corresponding to a putative full-length human TRAIL isoform
1, as set forth in NCBI Accession: NP_003801);
MAMMEVQGGPSLGQTCVLIVIFTVLLQSLCVAVTYVYFTNELKQMQDKYSKSGI
ACFLKEDDSYWDPNDEESMNSPCWQVKWQLRQLVRKMILRTSEETISTVQEKQQ
NISPLVRERGPQRVAAHITGTRGRSNTLSSPNSKNEKALGRKINSWESSRSGHSFLS
NLHLRNGELVIHEKGFYYTYSQTYFRFQEEIKENTKNDKQMVQYIYKYTSYPDPIL
LMKSARNSCWSKDAEYGLYSIYQGGIFELKENDRIFVSVTNEHLIDMDHEASFLGA
FLVGL [SEQ ID NO: 8] (corresponding to a putative full-length synthetic TRAIL, as set
forth in NCBI Accession: AAX29952);
KEKQQNISPLVRERGPQRVAAHITGTRGRSNTLSSPNSKNEKALGRKINSWESSRS
GHSFLSNLHLRNGELVIHEKGFYYIYSQTYFRFQEEIKENTKNDKQMVQYIYKYTS
YPDPILLMKSARNSCWSKDAEYGLYSIYQGGIFELKENDRIFVSVTNEHLIDMDHE
ASFFGAFLVG [SEQ ID NO: 10] (corresponding to a putative full-length human TRAIL
isoform CRA_b, as set forth in NCBI Accession: EAW78466);
MAMMEVQGGPSLGQTCVLIVVFTVLLQSLCVAVTYVYFTNELKQMQDKYSKSGI
ACFLKEDDSYWDPNDEDSMNSPCWQVKWQLRQLVRKMILRTSEETISTVQEKQQ
NISPLVRERGPQRVAAHITGTRGRSNTLSSPNSKNEKALGHKINSWESSRSGHSFLS
NLHLRNGELVIHEKGFYYIYSQTYFRFQEEIKENTKNDKQMVQYIYKYTSYPDPIL
LMKSARNSCWSKDAEYGLYSIYQGGIFELKENDRIFVSVTNEHLIDMDHEASFFGA
FLVG [SEQ ID NO: 12] (corresponding to a putative full-length Pan troglodytes TRAIL,
as set forth in NCBI Accession: XP_516879);
VRERGPQRVAAHITGTRGRSNTLSSPNSKNEKALGRKINSWESSRSGHSFLSNLHL
RNGELVIHEKGFYYIYSQTYFRFQEEIKENTKNDKQMVQYIYKYTSYPDPILLMKS
ARNSCWSKDAEYGLYSIYQGGIFELKENDRIFVSVTNEHLIDMDHEASFFGAFLVG
[SEQ ID NO: 14] (corresponding to a human TRAIL fragment, as set forth in NCBI
Accession: 1D0G_A);
MILRTSEETISTVQEKQQNISPLVRERGPQRVAAHITGTRGRSNTLSSPNSKNEKAL
GRKINSWESSRSGHSFLSNLHLRNGELVIHEKGFYYIYSQTYFRFQEEIKENTKNDK
QMVQYIYKYTSYPAPILLMKSARNSCWSKDAEYGLYSIYQGGIFELKENDRIFVSV
TNEHLIDMDHEASFFGAFLVG [SEQ ID NO: 16] (corresponding to a human TRAIL
fragment, as set forth in NCBI Accession: 1DG6);
MAMMEAQGGPSPGQTCVLILIFTVLLQSLCAAVTYVYFTNELKQMQDKYSKSGIA
CFLKEDDSSWDPNDEESMKSPCWQVKWQLRQLVRKMILRTSEETISTVQEKQQN
TSPLVRERGPQRVAAHITGTRGRSNTLSSPNSKNEKALGRKINSWESSRSGHSFLS
NLHLRNGELVIQEKGFYYIYSQTYFRFQEEIKENTKNDKQMVQYIYKYTSYPDPIL
LMKSARNSCWSKDAEYGLYSIYQGGLFELKKDDRIFVSVTNEHLIDMDHEASFFG
AFLVG [SEQ ID NO: 18] (corresponding to a putative full-length Macaca mulatta
TRAIL, as set forth in NCBI Accession: XP_001084768);
MVRERGPQRVAAHITGTRGRSNTLSSPNSKNEKALGRKINSWESSRSGHSFQSNL
HLRNGELVIHEKGFYYIYSQTYFRFQEEIKENAKNDKQMVQYIYKYTSYPDPILLM
KSARNSCWSKDAEYGLYSIYQGGIFELKENDRIFVSVTNEHLIDMDHEASFFGAF
[SEQ ID NO: 20] (corresponding a putative full-length Crassostrea ariakensis TRAIL, as
set forth in NCBI Accession: ABU39827);
PQRVAAHITGTRGRSNTLSSPNSKNEKALGRKINSWESSRSGHSFLSNLHLRNGEL
VIHEKGFYYIYSQTYFRFQEEIKENTKNDKQMVQYIYKYTSYPDPILLMKSARNSC
WSKDAEYGLYSIYQGGIFELKENDRIFVSVTNEHLIDMDHEASFFGAFLVG[SEQ
ID NO: 22] (corresponding to a human TRAIL fragment, as set forth in NCBI Accession:
1D4V_B);
MAMMEVQGGPSLGQTCVLIVIFTVLLQSLCVAVTYVYFTNELKQMQDKYSKSGI
ACFLKEDDSSWDPNDEDSMNSPCWQVKWQLRQLVRKMILRTSEETISTVQEKQQ
NVSPLVRERGPQRVAAHITGTRGRSNTLSSPSKRNNKXXXRKINSWESSRSGHSFL
SNLHLRNGELVIHEKGFYYIYSQTYFRFQEEIKENTKNDKQMVQYIYKYTSYPDPI
LLMKSARNSCWSKDAEYGLYSIYQGGIFELKENDRIFVSVTNEHLIDMDHEASFFG
AFLVG SEQ ID NO: 24] (corresponding to a putative full-length Pongo abelii TRAIL, as
set forth in NCBI Accession: XP_002814335);
MAMMEGQGGPSPGQTCVLILIFTVLLQSLCVAVTYLYFTNELKQMQDKYSKSGIA
CFLKEDGSSWDPSDEESMNSPCWEVKWQLRQLVRKMILRTSEETISTVQEKQRGI
SPQVRERGPQRVAAHITGTRGSSNTLPIPNSKNEKALGRKINSWESSRSGHSFLSNL
HLRNGELVIHEKGLYYIYCQVYFRFQEEIQENRKNDKQMVQYIYKYTSYPDPILL
MKSARNNCWSKDAEYGLYSIYQGGIFELKENDRIFVSVTNGQLIDMDHEASFFGA
FLVG [SEQ ID NO: 26] (corresponding to a putative full-length Callithrix jacchus
TRAIL, as set forth in NCBI Accession: XP_002759427);
MQAPAGPSPGQTCVLILIFTVLLQSLCVAVTYMYFTSELRQMQDKYSQSGIACFLK
EDDIPWDPNDEESMNTPCWQVKWQLRQFVRKILRTYEETIPTVPEKQLNIPYLVR
ERGPQRVAAHITGTSRRRSTFPVPSSKNEKALGQKINSWESSRKGHSFLNNLHLRN
GELVIHQRGFYYIYSQTYFRFQEPEEIPTGQNRKRNKQMVQYIYKHTSYPDPILLM
KSARNSCWSKDSEYGLYSIYQGGIFELKENDRIFVSVSNEQLIDMDQEASFFGAFLI
G [SEQ ID NO: 28] (corresponding to a putative full-length Felis catus TRAIL, as set
forth in NCBI Accession: NP_001124316);
MQAPGGPSPGQTCVLTLIFTVLLQSLCVAVTYMYFTRELKQMQDKYSQSGIACFL
KEDDIPWDPNDEESMNNPCWQVKWQLRQFVRKMILKTYEETIPSIPEKQLNIPYV
VNERGLQRVAAHITGTSRRRSTFPVPSSKNEKALGQKINSWESSRKGHSFLSNLHL
RNGELVIHQSGFYYIYSQTYFRFQEPEETSGPISKEQNRKKNKQMVQYIYKYTSYP
DPILLMKSARNSCWSKDSEYGLYSIYQGGIFELKENDRIFVSVNNEQLIDMDQEAS
FFGAFLIG [SEQ ID NO: 30] (corresponding to a putative full-length Ailuropoda
melanoleuca TRAIL, as set forth in NCBI Accession: XP_002921635);
MAMMQASGGPSPGQTCVLILIFTVLLQALCVAVTYLYFTNELKQMQIKYSKSGIA
CFLKEDDSDWDPNDEESMNSPCWQVKWQLRQFVRKMILRTYEESIPTTSEKRQNI
PPLVRERGLQRVAAHITGTSRRRSTVSIPRSKNEKALGQKINAWETSRKGHSFLNN
LHLRNGELVIHQTGFYYIYSQTYFRFQEPEEILGTVATEENRRKNKQMVQYIYKST
DYPDPILLMKSARNSCWSKDSEYGLYSIYQGGIFELKENDRIFVSVTNEQLIDMDQ
EASFFGAFLIG [SEQ ID NO: 32] (corresponding to a putative full-length Equus caballus
TRAIL, as set forth in NCBI Accession: XP_001494138);
MQAPGGPSPGQTCVLTLIFTVLLQSLCVAVTYMYFTRELKQMQDKYSQSGIACFL
KEDDIPWDPNDEESMNNPCWQVKWQLRQFVRKMILKTYEETIPSIPEKQLNIPYV
VNERGLQRVAAHITGTSRRRSTFPVPSSKNEKALGQKINSWESSRKGHSFLSNLHL
RNGELVIHQSGFYYIYSQTYFRFQEPEETSGPISKEQNRKKNKQMVQYIYKYTSYP
DPILLMKSARNSCWSKDSEYGLYSIYQGGIFELKENDRIFVSVNNEQLIDMDQEAS
FFGAF [SEQ ID NO: 34] (corresponding to a putative full-length Ailuropoda melanoleuca
TRAIL, as set forth in NCBI Accession: EFB16787);
MALKQAPGSRLGQICMPILIFTVLLQAFGMAVFYMYFNKELKQMQNKYFKSGLAC
FLEEDDRSWDSRDDESIINPCWELKSQLYLFVKKMTLRTFEEMIPTNPEKQYNPYL
EREKGPKRVAAHITGSNRKKSTLPVPGSKNEKAVGHKINSWESSRKGHSFLNNLY
LRNGELVILQTGFYYIYSQTYFRFQEPEEVLGTVSTEENRKKIKQMVQYIYKYTNY
PDPILLMKSARNSCWSKDSEYGLYSIYQGGIFELKENDRIFVSVTNERLVDLDQEA
SFFGAFLIG [SEQ ID NO: 36] (corresponding to a putative full-length Bos taurus
TRAIL, as set forth in NCBI Accession: XP_583785);
MAVMQTPGGPSPGQTCVLILIFTVLLQALCVALTYVYFTNELKQMQDKYSKSGIA
CFLKEDDSFWDPTDDERMLSPCWQVKWQLRQFVRKMILRTYEETISTVSEKQQGI
PHLEREKGPQRVAAHITGTSRKRSTFPSLSSKYEKALGQKINSWESSRKGHSFLNN
FHLRNGELVIHQTGFYYIYSQTYFRFQEPEEILGTVSTEGNRKKNRQMIQYIYKWT
SYPDPILLMKSARNSCWSKDSEYGLYSIYQGGIFELKEDDRIFVSVTNEQLIDMDQ
EASFFGAFLIG [SEQ ID NO: 38] (corresponding to a putative full-length Sus scrofa
TRAIL, as set forth in NCBI Accession: NP_001019867);
MQAPGGPSLGLTCVLILIFTVLLQSLCVAVTYMYFTRELKQMQDKYSQSGIACFLK
EDDIPWDPSDEESMNNPCWQVKWQLRQFVRKMILKTYEETIPTAPEKQLNIPYVV
SDRGSQRVAAHITGTSRRSMFPIPSSKNDKALGHKINSWDSTRKGHSFLNNLHLRN
GELVIHQRGFYYIYSQTFRFQEPEEIPTGQNRKRNKQMVQYIYKHTSYPDPILLM
KSARNSCWSKDSEYGLYSIYQGGIFELKENDRIFVSVSNEQLIDMDQEASFFGAFLI
G [SEQ ID NO: 40] (corresponding to a putative full-length Canis lupis familiaris TRAIL,
as set forth in NCBI Accession: NP_001124308);
MSSVQALGGPSAGQTCVLILIFTVLLQSLCVAVTYLYFTNELKQMQDKYSKSGIAC
LLKEDDSSWDSIDEENMNSPCWQAKWQLRQFIRKMLLRTYEETIPTVEEKPQTIPS
LVREKERERGPQRVAAHLTGNSWRSFISVPAPGSQSGKNLGQKISSWESSRKGHSF
LNNLHLRNGELVIHQTGLYYIYSQTYFRFQELEEISGTISREEIKKRNKQMVQYIYK
WTSYPDPILLMKSARNSCWSKDSEYGLYSIYQGGIFELKENDRIFVSVTNEQLIDM
NQESSFFGAFLIG [SEQ ID NO: 42] (corresponding to a putative full-length Oryctolagus
cuniculus TRAIL, as set forth in NCBI Accession: XP_002716472);
MPSTGNLKGPSFSQHFTMTVICIVLLQVLLQALTVAVTYMYFNNEVKQLQDNYSK
IGLACFSKEDGDFWDSTDEGILNRPCLQVKRQLYQLIEEVTLRTFEKTISTVPEKQL
STPPLPRGRRPQRVAAHITGITRRSNLALIPISKDGKTLGQKIETWESSRRGHSFLNH
VHLRNGELVIQEEGLYYIYSQTYYRFKEAKEASKTVSKDGGRIKQMVQYIYKYTS
YPDPILLMKSARNSCWSREAEYGLYSIYQGGLFELKENDRIFVSVTNEHLMDLDQ
EASFFGAFLIN [SEQ ID NO: 44] (corresponding to a putative full-length Rattus
novegicus TRAIL, as set forth in NCBI Accession: EDM01114);
MASTGNLKGPSFSQHFTMTVICIVLLQVLLQALTVAVTYMYFNNEVKQLQDNYS
KIGLACFSKEDGDFWDSTDEGILNRPCLQVKRQLYQLIEEVTLRTFEKTISTVPEKQ
LSTPPLPRGRRPQRVAAHITGITRRSNLALIPISKDGKTLGQKIETWESSRRGHSFLN
HVHLRNGELVIQEEGLYYIYSQTYYRFKEAKEASKTVSKDGGRIKQMVQYIYKYT
SYPDPILLMKSARNSCWSREAEYGLYSIYQGGLFELKENDRIFVSVTNEHLMDLDH
EASFFGA [SEQ ID NO: 46] (corresponding to a putative full-length Rattus novegicus
TRAIL, as set forth in NCBI Accession: NP_663714);
MASTGNLKGPSFSQHFTMTVICIVLLQVLLQALTVAVTYMYFNNEVKQLQDNYS
KIGLACFSKEDGDFWDSTDEGILNRPCLQVKRQLYQLIEEVTLRTFEKTISTVPEKQ
LSTPPLPRGRRPQRVAAHITGITRRSNLALIPISKDGKTLGQKIETWESSRRGHSFLN
HVHLRNGELVIQEEGLYYIYSQTYYRFKEAKEASKTVSKDGGRIKQMVQYIYKYT
SYPDPILLMKSARNSCWSREAEYGLYSIYQGGLFELKENDRIFVSVTNEHLMDLDQ
EASFFGA [SEQ ID NO: 48] (corresponding to a putative full-length Rattus novegicus
TRAIL, as set forth in NCBI Accession: ABK32522);
MPSSGALKDLSFSQHFRMMVICIVLLQVLLQAVSVAVTYMYFTSEMKQLQDNYS
KIGLACFSKTDEDFWDSTDGEILNRPCLQVKRQLYQLIEEVTLRTFQDTISTVPEKQ
LSTPPLPRGGRPQKVAAHITGITRRSNSALIPISKDGKTLGQKIESWESSRKGHSFLN
HVLFRNGELVIEQEGLYYIYSQTYFRFQEAKDASKMVSKDKVRTKQLVQYIYKYT
SYPDPIVLMKSARNSCWSRDAEYGLYSIYQGGLFELKKNDRIFVSVTNEHLMDLD
QEASFFGAFLIN [SEQ ID NO: 50] (corresponding to a putative full-length Mus
musculus TRAIL, as set forth in NCBI Accession: BAE34141);
MPSSGALKDLSFSQHFRMMVICIVLLQVLLQAVSVAVTYMYFTNEMKQLQDNYS
KIGLACFSKTDEDFWDSTDGEILNRPCLQVKRQLYQLIEEVTLRTFQDTISTVPEKQ
LSTPPLPRGGRPQKVAAHITGITRRSNSALIPISKDGKTLGQKIESWESSRKGHSFLN
HVLFRNGELVIEQEGLYYIYSQTYFRFQEAEDASKMVSKDKVRTKQLVQYIYKYT
SYPDPIVLMKSARNSCWSRDAEYGLYSIYQGGLFELKKNDRIFVSVTNEHLMDLD
QEASFFGAFLIN [SEQ ID NO: 52] (corresponding to a putative full-length Mus
musculus TRAIL, as set forth in NCBI Accession: NP_033451); or
(b) an amino acid sequence that shares at least 70% (and at least 71% to at least 99% and all integer percentages in between) sequence similarity or sequence identity with the sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50 or 52, or to a soluble fragment thereof; or
(c) an amino acid sequence which is encoded by the nucleotide sequence set forth in any one of:
gtgagagaaagaggtcctcagagagtagcagctcacataactgggaccagaggaagaagcaacacattgtcttctccaaactcc
aagaatgaaaaggctctgggccgcaaaataaactcctgggaatcatcaaggagtgggcattcattcctgagcaacttgcacttga
ggaatggtgaactggtcatccatgaaaaagggttttactacatctattcccaaacatactttcgatttcaggaggaaataaaagaaa
acacaaagaacgacaaacaaatggtccaatatatttacaaatacacaagttatcctgaccctatattgttgatgaaaagtgctagaa
atagttgttggtctaaagatgcagaatatggactctattccatctatcaagggggaatatttgagcttaaggaaaatgacagaattttt
gtttctgtaacaaatgagcacttgatagacatggaccatgaagccagtttttttggggcctttttagttggctaa [SEQ ID NO:
1] (corresponding to a nucleotide sequence from NCBI Accession: NM_003810, encoding
amino acids 114-281 of a human TRAIL isoform 1, as set forth in NCBI Accession:
NP_003801);
atggctatgatggaggtccaggggggacccagcctgggacagacctgcgtgctgatcgtgatcttcacagtgctcctgcagtct
ctctgtgtggctgtaacttacgtgtactttaccaacgagctgaagcagatgcaggacaagtactccaaaagtggcattgcttgtttc
ttaaaagaagatgacagttattgggaccccaatgacgaagagagtatgaacagcccctgctggcaagtcaagtggcaactccgt
cagctcgttagaaagatgattttgagaacctctgaggaaaccatttctacagttcaagaaaagcaacaaaatatttctcccctagtg
agagaaagaggtcctcagagagtagcagctcacataactgggaccagaggaagaagcaacacattgtcttctccaaactccaa
gaatgaaaaggctctgggccgcaaaataaactcctgggaatcatcaaggagtgggcattcattcctgagcaacttgcacttgagg
aatggtgaactggtcatccatgaaaaagggttttactacatctattcccaaacatactttcgatttcaggaggaaataaaagaaaac
acaaagaacgacaaacaaatggtccaatatatttacaaatacacaagttatcctgaccctatattgttgatgaaaagtgctagaaat
agttgttggtctaaagatgcagaatatggactctattccatctatcaagggggaatatttgagcttaaggaaaatgacagaatttttg
tttctgtaacaaatgagcacttgatagacatggaccatgaagccagttttttcggggcctttttagttggcttg [SEQ ID NO:
3]; (corresponding to a nucleotide sequence from NCBI Accession: BT019563, encoding a
putative full-length synthetic TRAIL, as set forth in NCBI Accession: AAV38370);
atggctatgatggaggtccaggggggacccagcctgggacagacctgcgtgctgatcgtgatcttcacagtgctcctgcagtct
ctctgtgtggctgtaacttacgtgtactttaccaacgagctgaagcagatgcaggacaagtactccaaaagtggcattgcttgtttc
ttaaaagaagatgacagttattgggaccccaatgacgaagagagtatgaacagcccctgctggcaagtcaagtggcaactccgt
cagctcgttagaaagatgattttgagaacctctgaggaaaccatttctacagttcaagaaaagcaacaaaatatttctcccctagtg
agagaaagaggtcctcagagagtagcagctcacataactgggaccagaggaagaagcaacacattgtcttctccaaactccaa
gaatgaaaaggctctgggccgcaaaataaactcctgggaatcatcaaggagtgggcattcattcctgagcaacttgcacttgagg
aatggtgaactggtcatccatgaaaaagggttttactacatctattcccaaacatactttcgatttcaggaggaaataaaagaaaac
acaaagaacgacaaacaaatggtccaatatatttacaaatacacaagttatcctgaccctatattgttgatgaaaagtgctagaaat
agttgttggtctaaagatgcagaatatggactctattccatctatcaagggggaatatttgagcttaaggaaaatgacagaatttttg
tttctgtaacaaatgagcacttgatagacatggaccatgaagccagtttttttggggcctttttagttggctaa [SEQ ID NO:
5] (corresponding to a nucleotide sequence from NCBI Accession: NM_003810, encoding
a putative full-length human TRAIL isoform 1, as set forth in NCBI Accession:
NP_003801);
atggctatgatggaggtccaggggggacccagcctgggacagacctgcgtgctgatcgtgatcttcacagtgctcctgcagtct
ctctgtgtggctgtaacttacgtgtactttaccaacgagctgaagcagatgcaggacaagtactccaaaagtggcattgcttgtttc
ttaaaagaagatgacagttattgggaccccaatgacgaagagagtatgaacagcccctgctggcaagtcaagtggcaactccgt
cagctcgttagaaagatgattttgagaacctctgaggaaaccatttctacagttcaagaaaagcaacaaaatatttctcccctagtg
agagaaagaggtcctcagagagtagcagctcacataactgggaccagaggaagaagcaacacattgtcttctccaaactccaa
gaatgaaaaggctctgggccgcaaaataaactcctgggaatcatcaaggagtgggcattcattcctgagcaacttgcacttgagg
aatggtgaactggtcatccatgaaaaagggttttactacatctattcccaaacatactttcgatttcaggaggaaataaaagaaaac
acaaagaacgacaaacaaatggtccaatatatttacaaatacacaagttatcctgaccctatattgttgatgaaaagtgctagaaat
agttgttggtctaaagatgcagaatatggactctattccatctatcaagggggaatatttgagcttaaggaaaatgacagaatttttg
tttctgtaacaaatgagcacttgatagacatggaccatgaagccagttttttaggggcctttttagttggcttg [SEQ ID NO:
7] (corresponding to a nucleotide sequence from NCBI Accession: AY893035, encoding a
putative full-length synthetic TRAIL, as set forth in NCBI Accession: AAX29952);
caggatcatggctatgatggaggtccaggggggacccagcctgggacagacctgcgtgctgatcgtgatcttcacagtgctcct
gcagtctctctgtgtggctgtaacttacgtgtactttaccaacgagctgaagcagaaaagcaacaaaatatttctcccctagtgaga
gaaagaggtcctcagagagtagcagctcacataactgggaccagaggaagaagcaacacattgtcttctccaaactccaagaat
gaaaaggctctgggccgcaaaataaactcctgggaatcatcaaggagtgggcattcattcctgagcaacttgcacttgaggaatg
gtgaactggtcatccatgaaaaagggttttactacatctattcccaaacatactttcgatttcaggaggaaataaaagaaaacacaa
agaacgacaaacaaatggtccaatatatttacaaatacacaagttatcctgaccctatattgttgatgaaaagtgctagaaatagttg
ttggtctaaagatgcagaatatggactctattccatctatcaagggggaatatttgagcttaaggaaaatgacagaatttttgtttctg
taacaaatgagcacttgatagacatggaccatgaagccagtttttttggggcctttttagttggctaa [SEQ ID NO: 9]
(corresponding to a nucleotide sequence from NCBI Accession: CH471052, encoding a
putative full-length human TRAIL isoform CRA_b, as set forth in NCBI Accession:
EAW78466);
atggctatgatggaggtccaggggggacccagcctgggacagacctgcgtgctgatcgtggtcttcacagtgctcctgcagtct
ctctgtgtggctgtaacttacgtgtactttaccaacgagctgaagcagatgcaggacaagtactccaaaagtggcattgcttgtttc
ttaaaagaagatgacagttattgggaccccaatgacgaagacagtatgaacagcccctgctggcaagtcaagtggcaactccgt
cagctcgttagaaagatgattttgagaacctctgaggaaaccatttctacagttcaagaaaagcaacaaaatatttctcccctagtg
agagaaagaggtcctcagagagtagcagctcacataactggaaccagaggaagaagcaacacattgtcttctccaaactccaag
aatgaaaaggctctgggccacaaaataaactcctgggaatcatcaaggagtgggcattcattcctgagcaacttgcacttgagga
atggcgaactggtcatccatgaaaaagggttttactacatctattcccaaacatactttcgatttcaggaggaaataaaagaaaaca
caaagaacgacaaacaaatggtccaatatatttacaaatacacaagttatcctgaccctatattgttgatgaaaagcgctagaaata
gttgttggtctaaagatgcagaatatggactctattccatctatcaagggggaatatttgagcttaaggaaaatgacagaatttttgtt
tctgtaacaaatgagcacttgatagacatggaccatgaagccagttttttcggggcctttttagttggctaa [SEQ ID NO:
11] (corresponding to a nucleotide sequence from NCBI Accession: XM_516879,
encoding a putative full-length Pan troglodytes TRAIL, as set forth in NCBI Accession:
XP_516879);
gtnmgngarmgnggnccncarmgngtngcngcncayathacnggnacnmgnggnmgnwsnaayacnytnwsnwsn
ccnaaywsnaaraaygaraargcnytnggnmgnaarathaaywsntgggarwsnwsnmgnwsnggncaywsnttyytn
wsnaayytncayytnmgnaayggngarytngtnathcaygaraarggnttytaytayathtaywsncaracntayttymgntt
ycargargarathaargaraayacnaaraaygayaarcaratggtncartayathtayaartayacnwsntayccngayccnat
hytnytnatgaarwsngcnmgnaaywsntgytggwsnaargaygcngartayggnytntaywsnathtaycarggnggna
thttygarytnaargaraaygaymgnathttygtnwsngtnacnaaygarcayytnathgayatggaycaygargcnwsntty
ttyggngcnttyytngtnggntrr [SEQ ID NO: 13] (corresponding to a degenerate nucleotide
sequence encoding a human TRAIL fragment, as set forth in NCBI Accession: 1D0G_A);
atgathytnmgnacnwsngargaracnathwsnacngtncargaraarcarcaraayathwsnccnytngtnmgngarmg
nggnccncarmgngtngcngcncayathacnggnacnmgnggnmgnwsnaayacnytnwsnwsnccnaaywsnaar
aaygaraargcnytnggnmgnaarathaaywsntgggarwsnwsnmgnwsnggncaywsnttyytnwsnaayytncay
ytnmgnaayggngarytngtnathcaygaraarggnttytaytayathtaywsncaracntayttymgnttycargargarath
aargaraayacnaaraaygayaarcaratggtncartayathtayaartayacnwsntayccngcnccnathytnytnatgaar
wsngcnmgnaaywsntgytggwsnaargaygcngartayggnytntaywsnathtaycarggnggnathttygarytnaa
rgaraaygaymgnathttygtnwsngtnacnaaygarcayytnathgayatggaycaygargcnwsnttyttyggngcntty
ytngtnggntrr [SEQ ID NO: 15] (corresponding to a degenerate nucleotide sequence
encoding a human TRAIL fragment, as set forth in NCBI Accession: 1DG6);
atggctatgatggaggcccaggggggacccagcccggggcagacctgcgtgctgatcctgatcttcacggtgctcctgcagtc
cctctgtgcagctgtaacttacgtgtacttcaccaacgagctgaagcagatgcaggacaagtactccaaaagtggcattgcttgttt
cttgaaagaagatgacagttcttgggatcccaatgacgaagagagtatgaagagcccctgctggcaagtcaagtggcaactccg
tcaactcgttagaaagatgattttgagaacctctgaggaaaccatttctacagttcaagaaaagcaacaaaatacttctcccctagt
gagagaaagaggtcctcagagagtagcagctcacataactgggaccagaggaagaagcaacacattgtcttctccaaactcca
agaatgaaaaggctctgggccgcaaaataaactcctgggaatcatcaaggagtgggcattcattcctgagcaacttgcacttgag
gaatggcgaactggtcatccaagaaaaggggttttactacatctattcccaaacatactttcgatttcaggaggaaataaaagaaa
acacaaagaacgacaaacaaatggtccaatatatttacaaatacacaagttatcctgaccctatactgctgatgaaaagcgctaga
aatagttgttggtctaaagatgcagaatacggactctattccatctatcaagggggattatttgagcttaagaaagatgacagaattt
ttgtttctgtaacaaatgagcacttgatagacatggaccatgaagccagctttttcggggcctttttggttggctaa [SEQ ID
NO: 17] (corresponding to a nucleotide sequence from NCBI Accession: XM_001084768,
encoding a putative full-length Macaca mulatta TRAIL, as set forth in NCBI Accession:
XP_001084768);
atggtgagagaaagaggtcctcagagagtagcagctcacataactgggaccagaggaagaagcaacacattgtcttctccaaa
ctccaagaatgaaaaggctctgggccgcaaaataaactcctgggaatcatcaaggagtgggcattcattccagagcaacttgca
cttgaggaatggtgaactggtcatccatgaaaaagggttttactacatctattcccaaacatactttcgatttcaggaggaaataaaa
gaaaacgcaaagaacgacaaacaaatggtccaatatatttacaaatacacaagttatcctgaccctatattgttgatgaaaagtgct
agaaatagttgttggtctaaagatgcagaatatggactctattccatctatcaagggggaatatttgagcttaaggaaaatgacaga
atttttgtttctgtaacaaatgagcacttgatagacatggaccatgaagccagttttttcggggccttttaa [SEQ ID NO:
19] (corresponding a nucleotide sequence from NCBI Accession: EF541151, encoding a
putative full-length Crassostrea ariakensis TRAIL, as set forth in NCBI Accession:
ABU39827);
ccncarmgngtngcngcncayathacnggnacnmgnggnmgnwsnaayacnytnwsnwsnccnaaywsnaaraayg
araargcnytnggnmgnaarathaaywsntgggamsnwsnmgnwsnggncaywsnttyytnwsnaayytncayytn
mgnaayggngarytngtnathcaygaraarggnttytaytayathtaywsncaracntayttymgnttycargargarathaar
garaayacnaaraaygayaarcaratggtncartayathtayaartayacnwsntayccngayccnathytnytnatgaarwsn
gcnmgnaaywsntgytggwsnaargaygcngartayggnytntaywsnathtaycarggnggnathttygarytnaargar
aaygaymgnathttygtnwsngtnacnaaygarcayytnathgayatggaycaygargcnwsnttyttyggngcnttyytng
tnggntrr [SEQ ID NO: 21] (corresponding to a degenerate nucleotide sequence encoding a
human TRAIL fragment, as set forth in NCBI Accession: 1D4V_B);
atggctatgatggaggtccaggggggacccagcctggggcagacctgcgtgctgatcgtgatcttcacagtgctcctgcagtct
ctctgtgtggctgtaacttacgtgtactttaccaacgagctgaagcagatgcaggacaagtactccaaaagtggcattgcttgtttc
ttaaaagaagatgacagctcttgggaccctaatgacgaagacagtatgaacagcccctgctggcaagtcaagtggcaactccgt
cagctcgttagaaagatgattttgagaacctctgaggaaaccatttctacagttcaagaaaagcaacaaaatgtttctcccctagtg
agagaaagaggtcctcagagagtagcagctcacataactgggaccagaggaagaagcaacacattgtcttctccaagtaagag
aaacaacaaannnnnnnnncgcaaaataaactcctgggaatcatcaaggagtgggcattcattcctgagcaacttgcacttgag
gaatggcgaactggtcatccatgaaaaagggttttactacatctattcccaaacatactttcgatttcaggaggaaataaaagaaaa
cacaaagaacgacaaacaaatggtccaatatatttacaaatacacaagttatcctgatcctatattgctgatgaaaagcgctagaaa
tagttgttggtctaaagatgcagaatatggactctattccatctatcaagggggaatatttgagcttaaggaaaatgacagaattttt
gtttctgtaacaaatgagcacttgatagacatggaccatgaagccagttttttcggggcctttttagttggctaa [SEQ ID
NO: 23] (corresponding to a nucleotide sequence from NCBI Accession: XM_002814289,
encoding a putative full-length Pongo abelii TRAIL, as set forth in NCBI Accession:
XP_002814335);
atggctatgatggagggtcaggggggacccagcccggggcagacctgcgtgctgatcctgatcttcacagtgctcctgcagtc
cctctgtgtggccgtaacttacctgtacttcaccaatgagctgaagcagatgcaggacaagtactccaaaagcggcattgcttgttt
cttaaaagaagatggcagctcctgggaccccagtgacgaagagagtatgaatagcccctgctgggaagtcaagtggcaactcc
gtcagctcgttagaaagatgattttgagaacctctgaagaaaccatttctacagttcaagaaaagcaacgaggtatttctccccaag
tgagagaaagaggtcctcagagagtagcagctcacataactgggaccagaggaagtagcaacacgttgcctattccaaactcca
agaatgaaaaggctctgggccgcaaaataaactcctgggaatcatcaaggagtggacattctttcctgagcaacttgcacttgag
gaatggcgagctggtcatccatgaaaaagggctgtattacatctattgccaagtatactttcgatttcaggaggaaatccaagaaa
acagaaagaacgacaaacaaatggtccagtatatttacaaatacacaagttatcctgaccccatactgctgatgaagagtgctaga
aataattgttggtctaaagatgcagaatatggactctattccatctatcaagggggaatatttgagcttaaggaaaacgacagaattt
ttgtttctgtaacaaatgggcagttgatagacatggaccatgaagccagttttttcggggcctttttagttggctaa [SEQ ID
NO: 25] (corresponding to a nucleotide sequence from NCBI Accession: XM_002814289,
encoding a putative full-length Callithrix jacchus TRAIL, as set forth in NCBI Accession:
XP_002759427);
atgcaggccccggcgggccccagtcccgggcagacctgcgtgctgatcctgatcttcactgtgctcctgcagtccctctgcgtg
gccgtgacttacatgtacttcaccagtgaactgaggcagatgcaggacaaatactcccaaagtggcattgcttgtttcttaaagga
agacgatatcccttgggaccccaatgatgaagagagtatgaacaccccgtgctggcaagtgaaatggcagctccgtcagtttgtt
agaaagattttgagaacctatgaggaaaccattcctacagttccagaaaagcagctaaatattccttacctagtaagagaaagagg
tcctcagagagtagcagctcacataactggaaccagtcggagaagaagcacattcccagttccaagctccaagaatgaaaaagc
tttgggtcagaaaataaactcctgggagtcatcaagaaaaggacattcattcttgaataatttgcacttgaggaatggtgagctggt
tattcatcagagggggttttattacatctattcccaaacatactttcgatttcaggaacctgaggaaattccaacaggacagaacag
aaagagaaacaaacaaatggtccaatatatttacaaacacacgagttatccggaccctatactgctgatgaaaagtgctagaaata
gttgttggtctaaagattctgaatatggactctattccatctatcaaggtgggatatttgagcttaaggaaaacgatagaatttttgtct
ctgtatctaacgagcaattgattgacatggaccaagaagccagttttttcggggcctttttaatcggctaa [SEQ ID NO:
27] (corresponding to a nucleotide sequence from NCBI Accession: NM_001130844,
encoding a putative full-length Felis catus TRAIL, as set forth in NCBI Accession:
NP_001124316);
atgcaggccccggggggccccagccctgggcagacgtgcgtgttgaccctcatcttcacagtgctcctgcagtccctctgtgtg
gcggtgacctacatgtacttcaccagggagctgaagcagatgcaggacaagtactcccaaagcggcatcgcttgtttcttaaagg
aagatgatattccttgggacccaaatgatgaagagagtatgaacaatccttgctggcaagtgaagtggcaactccgtcagtttgtt
agaaagatgattttgaaaacctatgaggaaaccattccttcaattccagaaaagcagctaaatattccttacgtagtaaatgaaaga
ggtcttcagagagtagcagctcacataactggaaccagtcggagaagaagcacgtttccagttccaagctccaagaatgaaaaa
gctttgggccagaaaataaactcctgggagtcatcaagaaaaggacattcattcttgagtaatttgcacttgaggaatggagagct
ggttatccatcaaagtgggttttattacatctattcccaaacatactttcgatttcaggaacctgaggaaacttcgggaccaatttcaa
aggaacaaaacagaaagaaaaacaaacaaatggtacaatatatttacaaatacacaagttatcctgaccctatactgctgatgaaa
agtgctagaaatagttgctggtctaaagattctgagtatggactctattccatctatcaaggtgggatatttgagcttaaggaaaatg
atagaatttttgtctctgtaaataatgagcaattgattgacatggaccaagaagccagttttttcggggcctttttaattggctaa
[SEQ ID NO: 29] (corresponding to a nucleotide sequence from NCBI Accession:
XM_002921589, encoding a putative full-length Ailuropoda melanoleuca TRAIL, as set
forth in NCBI Accession: XP_002921635);
atggccatgatgcaggcatcagggggtcccagccccgggcagacctgcgtgctgatcctgatcttcacagtgctcctgcaggcc
ctctgtgtggctgtgacttatttgtacttcaccaacgagctgaagcagatgcagatcaaatactccaaaagtggcattgcctgtttct
taaaggaagatgacagcgattgggacccaaatgacgaagagagtatgaacagcccctgctggcaagtcaagtggcagctgcgt
cagtttgttagaaagatgattttgagaacctatgaggaatccattcctacaacttcagaaaagcgacaaaatattcctcccttagtaa
gagaaagaggtcttcagagagtagcagctcacataactgggaccagtcggagaagaagcacagtctcaattccacgctccaag
aatgaaaaagcactgggccagaaaataaacgcctgggagacatcaagaaaaggacattcgttcttgaataatttacacttgagga
atggagagctggttatccatcaaacagggttttattacatctattcccaaacatactttcgatttcaggaacctgaggaaattttggg
aacagttgcaacagaagagaacagaaggaaaaataaacaaatggtacaatatatttacaaaagcacagactatcctgaccctata
ctgctgatgaaaagtgctagaaatagttgttggtctaaagattcagaatacggactctattccatctatcaaggtggaatatttgagc
ttaaggaaaatgacagaatttttgtctctgtaactaatgagcaattgattgacatggaccaagaagccagtttcttcggggccttttta
atcggctaa [SEQ ID NO: 31] (corresponding to a nucleotide sequence from NCBI
Accession: XM_001494088, encoding a putative full-length Equus caballus TRAIL, as set
forth in NCBI Accession: XP_001494138);
atgcaggccccggggggccccagccctgggcagacgtgcgtgttgaccctcatcttcacagtgctcctgcagtccctctgtgtg
gcggtgacctacatgtacttcaccagggagctgaagcagatgcaggacaagtactcccaaagcggcatcgcttgtttcttaaagg
aagatgatattccttgggacccaaatgatgaagagagtatgaacaatccttgctggcaagtgaagtggcaactccgtcagtttgtt
agaaagatgattttgaaaacctatgaggaaaccattccttcaattccagaaaagcagctaaatattccttacgtagtaaatgaaaga
ggtcttcagagagtagcagctcacataactggaaccagtcggagaagaagcacgtttccagttccaagctccaagaatgaaaaa
gctttgggccagaaaataaactcctgggagtcatcaagaaaaggacattcattcttgagtaatttgcacttgaggaatggagagct
ggttatccatcaaagtgggttttattacatctattcccaaacatactttcgatttcaggaacctgaggaaacttcgggaccaatttcaa
aggaacaaaacagaaagaaaaacaaacaaatggtacaatatatttacaaatacacaagttatcctgaccctatactgctgatgaaa
agtgctagaaatagttgctggtctaaagattctgagtatggactctattccatctatcaaggtgggatatttgagcttaaggaaaatg
atagaatttttgtctctgtaaataatgagcaattgattgacatggaccaagaagccagttttttcggggccttt [SEQ ID NO:
33] (corresponding to a nucleotide sequence from NCBI Accession: GL192841, encoding
a putative full-length Ailuropoda melanoleuca TRAIL, as set forth in NCBI Accession:
EFB16787);
atggccctgaagcaggctccgggctccagacttgggcagatctgcatgccgatcctcatcttcacagtgctgctgcaggcttttg
gtatggccgtgttttacatgtatttcaacaaagagctgaagcagatgcagaacaaatacttcaaaagtggcttggcttgcttcttgg
aggaagatgaccgttcctgggactccagagatgatgagagtataatcaatccctgctgggaactaaagtcccaactctatctgttt
gttaaaaagatgactttgagaacctttgaggaaatgattcctacaaatccagaaaagcaatataatccttacctagagagagaaaa
gggtcctaagagggtagctgctcatataactggaagcaatcggaaaaaaagtacgttgccagttccaggctccaagaatgaaaa
agctgtgggccataaaataaattcctgggagtcatcaagaaaaggacattcgttcttgaataatttgtacttaaggaatggagagct
ggttatccttcaaacaggattttattacatctattcccaaacatactttcgatttcaggaacctgaggaagttttgggaactgtttcaac
agaagagaacagaaaaaaaatcaaacaaatggtacaatatatttacaaatacacaaactatcctgaccctatactgctgatgaaaa
gtgctagaaatagttgttggtctaaagattcagaatatggactctattccatctatcaaggaggaatatttgagcttaaggaaaatga
tcgaatttttgtctctgtaactaatgaacgattggttgacctggaccaagaagccagttttttcggagcctttttaattggctaa
[SEQ ID NO: 35] (corresponding to a nucleotide sequence from NCBI Accession:
GL192841, encoding a putative full-length Bos taurus TRAIL, as set forth in NCBI
Accession: XP_583785);
atggcggtgatgcagactccaggaggccccagccccgggcagacctgtgtgttgatcctgatcttcacagtgctcctgcaagcc
ctctgtgtggccttgacttacgtgtacttcaccaatgaactgaaacagatgcaggacaagtactccaaaagcggtatagcttgcttc
ttaaaggaagatgacagtttctgggatcccaccgatgacgagagaatgctcagcccctgctggcaggtgaagtggcagctacgt
cagtttgtgagaaagatgattttgagaacctatgaggaaaccatttctacagtttcagaaaagcaacaaggcattcctcacctagaa
agagaaaaaggtccacagagagtggctgctcacataactggaaccagtaggaaaagaagcacatttccatctctaagctccaaa
tatgaaaaagctttgggccagaaaataaactcctgggaatcatcaagaaaaggacattcattcttgaataattttcacttgaggaat
ggagagctggttatccatcaaacagggttttactacatctattcccaaacatactttcgatttcaggaacctgaggaaattttgggaa
cggtttctacagaagggaacagaaagaaaaacaggcaaatgatacagtatatttacaaatggacaagctatcctgaccctatact
gctgatgaaaagtgctagaaatagttgttggtctaaagattcagaatatggactctattccatctatcaaggtggaatatttgagctta
aggaagatgaccgaatttttgtctctgttactaatgagcaactgattgacatggaccaagaagccagttttttcggggcctttttaatt
ggctaa [SEQ ID NO: 37] (corresponding to a nucleotide sequence from NCBI Accession:
GL192841, encoding a putative full-length Sus scrofa TRAIL, as set forth in NCBI
Accession: NP_001019867);
atgcaggccccggggggccccagcctcgggctgacgtgcgtgctgatcctcatcttcactgtgctgctccagtccctctgcgtg
gccgtcacctacatgtacttcaccagggagctgaagcagatgcaggacaagtactcccaaagtggcatcgcttgtttcttaaagg
aagatgatatcccctgggaccccagtgatgaagagagtatgaacaacccctgctggcaagtgaagtggcaactccgccagtttg
ttagaaagatgattttgaaaacctatgaggaaaccattcctacagctccagaaaagcagctaaatattccttacgtagtaagcgacc
gaggttctcagagagtagctgctcacataactggaaccagtcggagaagcatgtttccaattccaagctccaagaatgataaagc
tttgggccacaaaataaactcctgggattccacaagaaaaggacattcattcttgaataatttgcacttgaggaacggagagctgg
ttatccatcaaagggggttttattacatctattcccaaacatactttcgatttcaggaacctgaggaaattccaacaggacagaacag
aaagagaaacaaacaaatggtccaatatatttacaaacacacgagttatccggaccctatactgctgatgaaaagtgctagaaata
gttgttggtctaaagattctgaatatggactctattccatctatcaaggtgggatatttgagcttaaggaaaacgatagaatttttgtct
ctgtatctaacgagcaattgattgacatggaccaagaagccagttttttcggggcctttttaatcggctaa [SEQ ID NO:
39] (corresponding to a nucleotide sequence from NCBI Accession: NM_001130836,
encoding a putative full-length Canis lupis familiaris TRAIL, as set forth in NCBI
Accession: NP_001124308);
atgtcctctgtgcaggccctggggggccccagtgccgggcagacctgcgtgctgatcctgatcttcacagtgctcctgcagtccc
tctgtgtggccgtgacttacctgtacttcaccaacgaactgaagcagatgcaggacaagtactccaaaagtggcatcgcttgtctc
ttaaaggaggatgacagttcctgggactccatcgacgaagagaacatgaacagcccctgctggcaggccaagtggcagctgcg
gcagttcattcgaaagatgcttttgagaacctatgaggaaaccattcctacggttgaagaaaagccacaaactattccttccctagt
aagagaaaaagaaagagaaagagggcctcagagagtagcagctcacctaactgggaacagctggagaagctttatctcagtcc
ctgctccaggctcccagagtggaaagaatttgggccagaaaataagctcctgggaatcatcaaggaaaggacattcattcctgaa
caatttgcacctgaggaatggagagctggttatccatcaaacaggactttattacatctactcccaaacatactttcgatttcaggaa
cttgaagaaatttcaggaacaatttcaagagaagagatcaaaaagaggaacaaacaaatggtacaatatatttacaaatggacaa
gctaccctgaccctatacttctgatgaaaagtgctagaaatagttgttggtctaaggattcggaatatggactctattccatctatcaa
ggaggaatatttgagcttaaggaaaatgaccgaattttcgtctctgtaacgaatgagcagttgattgacatgaaccaagaatccag
tttttttggggcctttttgattggctaa [SEQ ID NO: 41] (corresponding to a nucleotide sequence
from NCBI Accession: XM_002716426, encoding a putative full-length Oryctolagus
cuniculus TRAIL, as set forth in NCBI Accession: XP_002716472);
atgccttccaccgggaacctgaagggccccagcttcagtcagcacttcacgatgacggtgatctgcatagtgctcctgcaggtgc
tcctgcaggccttgactgtggctgtgacttacatgtacttcaacaacgaggtgaaacagctacaggacaattactccaaaatcgga
ctagcttgcttctcaaaagaagatggggatttttgggactccactgacgaggggattttgaacagaccttgcttgcaggtcaagag
gcaactgtatcagctcattgaagaggtgactttgagaacctttgagaaaaccatctctacagttccagaaaagcagctaagcactc
ctcccttgcccagaggtagaagaccccagagagtggcagctcacattaccgggatcactcggagaagcaacttagccttaattc
caatctccaaggatggaaagaccttgggccagaagatagaaacctgggagtcctctcggagagggcattcatttctcaaccatgt
gcacttgagaaacggagagctggtgatccaggaggagggcctgtattacatctactcccaaacgtactaccggttcaaggaggc
taaagaagcttccaagacagtctcgaaggacggagggaggatcaaacagatggtgcagtacatctacaaatacaccagctacc
ccgatcccatactgctgatgaagagtgccagaaatagctgctggtccagagaagctgagtacggactgtactccatctatcaggg
ggggctgttcgagctcaaagaaaatgacaggatttttgtttccgtgacgaatgagcatttgatggacctggatcaagaagccagtt
tctttggagcctttttaattaactag [SEQ ID NO: 43] (corresponding to a nucleotide sequence from
NCBI Accession: CH473961, encoding a putative full-length Rattus novegicus TRAIL, as
set forth in NCBI Accession: EDM01114);
atggcttccaccgggaacctgaagggccccagcttcagtcagcacttcacgatgacggtgatctgcatagtgctcctgcaggtg
ctcctgcaggccttgactgtggctgtgacttacatgtacttcaacaacgaggtgaaacagctacaggacaattactccaaaatcgg
actagcttgcttctcaaaagaagatggggatttttgggactccactgacgaggggattttgaacagaccttgcttgcaggtcaaga
ggcaactgtatcagctcattgaagaggtgactttgagaacctttgagaaaaccatctctacagttccagaaaagcagctaagcact
cctcccttgcccagaggtagaagaccccagagagtggcagctcacattaccgggatcactcggagaagcaacttagccttaatt
ccaatctccaaggatggaaagaccttgggccagaagatagaaacctgggagtcctctcggagagggcattcatttctcaaccat
gtgcacttgagaaacggagagctggtgatccaggaggagggcctgtattacatctactcccaaacgtactaccggttcaaggag
gctaaagaagcttccaagacagtctcgaaggacggagggaggatcaaacagatggtgcagtacatctacaaatacaccagcta
ccccgatcccatactgctgatgaagagtgccagaaatagctgctggtccagagaagctgagtacggactgtactccatctatcag
ggggggctgttcgagctcaaagaaaatgacaggatttttgtttccgtgacgaatgagcatttgatggacctggaccatgaagcca
gcttctttggagcctaa [SEQ ID NO: 45] (corresponding to a nucleotide sequence from NCBI
Accession: NM_145681, encoding a putative full-length Rattus novegicus TRAIL, as set
forth in NCBI Accession: NP_663714);
atggcttccaccgggaacctgaagggccccagcttcagtcagcacttcacgatgacggtgatctgcatagtgctcctgcaggtg
ctcctgcaggccttgactgtggctgtgacttacatgtacttcaacaacgaggtgaaacagctacaggacaattactccaaaatcgg
actagcttgcttctcaaaagaagatggggatttttgggactccactgacgaggggattttgaacagaccttgcttgcaggtcaaga
ggcaactgtatcagctcattgaagaggtgactttgagaacctttgagaaaaccatctctacagttccagaaaagcagctaagcact
cctcccttgcccagaggtagaagaccccagagagtggcagctcacattaccgggatcactcggagaagcaacttagccttaatt
ccaatctccaaggatggaaagaccttgggccagaagatagaaacctgggagtcctctcggagagggcattcatttctcaaccat
gtgcacttgagaaacggagagctggtgatccaggaggagggcctgtattacatctactcccaaacgtactaccggttcaaggag
gctaaagaagcttccaagacagtctcgaaggacggagggaggatcaaacagatggtgcagtacatctacaaatacaccagcta
ccccgatcccatactgctgatgaagagtgccagaaatagctgctggtccagagaagctgagtacggactgtactccatctatcag
ggggggctgttcgagctcaaagaaaatgacaggatttttgtttccgtgacgaatgagcatttgatggacctggatcaagaagcca
gcttctttggagcctaa [SEQ ID NO: 47] (corresponding to a nucleotide sequence from NCBI
Accession: EF030546, encoding a putative full-length Rattus novegicus TRAIL, as set
forth in NCBI Accession: ABK32522);
atgccttcctcaggggccctgaaggacctcagcttcagtcagcacttcaggatgatggtgatttgcatagtgctcctgcaggtgct
cctgcaggctgtgtctgtggctgtgacttacatgtacttcaccagcgagatgaagcagctgcaggacaattactccaaaattggac
tagcttgcttctcaaagacggatgaggatttctgggactccactgatggagagatcttgaacagaccctgcttgcaggttaagagg
caactgtatcagctcattgaagaggtgactttgagaacctttcaggacaccatttctacagttccagaaaagcagctaagtactcct
cccttgcccagaggtggaagacctcagaaagtggcagctcacattactgggatcactcggagaagcaactcagctttaattccaa
tctccaaggatggaaagaccttaggccagaagattgagtcctgggagtcctctcggaaagggcattcatttctcaaccacgtgct
ctttaggaatggagagctggtcattgagcaggagggcctgtattacatctattcccaaacatacttccgatttcaggaagctaaag
acgcttccaagatggtctcaaaggacaaggtgagaaccaaacagctggtgcagtacatctacaagtacaccagctatccggatc
ccatagtgctcatgaagagcgccagaaacagctgttggtccagagatgccgagtacggactgtactccatctatcagggaggac
tgttcgagctaaaaaaaaatgacaggatttttgtttctgtgacaaatgaacatttgatggacctggatcaagaagccagcttctttgg
agcctttttaattaactaa [SEQ ID NO: 49] (corresponding to a nucleotide sequence from NCBI
Accession: AK157633, encoding a putative full-length Mus musculus TRAIL, as set forth
in NCBI Accession: BAE34141);
atgccttcctcaggggccctgaaggacctcagcttcagtcagcacttcaggatgatggtgatttgcatagtgctcctgcaggtgct
cctgcaggctgtgtctgtggctgtgacttacatgtacttcaccaacgagatgaagcagctgcaggacaattactccaaaattggac
tagcttgcttctcaaagacggatgaggatttctgggactccactgatggagagatcttgaacagaccctgcttgcaggttaagagg
caactgtatcagctcattgaagaggtgactttgagaacctttcaggacaccatttctacagttccagaaaagcagctaagtactcct
cccttgcccagaggtggaagacctcagaaagtggcagctcacattactgggatcactcggagaagcaactcagctttaattccaa
tctccaaggatggaaagaccttaggccagaagattgaatcctgggagtcctctcggaaagggcattcatttctcaaccacgtgctc
tttaggaatggagagctggtcatcgagcaggagggcctgtattacatctattcccaaacatacttccgatttcaggaagctgaaga
cgcttccaagatggtctcaaaggacaaggtgagaaccaaacagctggtgcagtacatctacaagtacaccagctatccggatcc
catagtgctcatgaagagcgccagaaacagctgttggtccagagatgccgagtacggactgtactccatctatcagggaggatt
gttcgagctaaaaaaaaatgacaggatttttgtttctgtgacaaatgaacatttgatggacctggatcaagaagccagcttctttgga
gcctttttaattaactaa [SEQ ID NO: 51] (corresponding to a nucleotide sequence from NCBI
Accession: NM_009425, encoding a putative full-length Mus musculus TRAIL, as set
forth in NCBI Accession: NP_033451),
or
a complement of any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51;
(d) an amino acid sequence which is encoded by a nucleotide sequence that shares at least 70% sequence identity with the nucleic acid sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51, or with a complement thereof; and
(e) an amino acid sequence which is encoded by a nucleotide sequence that hybridizes under at least medium or high stringency conditions to the nucleic acid sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51, or to a complement thereof,
wherein the amino acid sequence of (a), (b), (c), (d) or (e) has any one or more activities selected from the group consisting of: inducing apoptosis of adipose tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, reducing circulating levels of the proinflammatory cytokines IL-6, IL-1alpha, G-CSF and MCP-1, counteracting lipopolysaccaride- and muramildipeptide-induced inflammation.

9. The TRAIL DR agonist according to claim 3, wherein the TRAIL polypeptide comprises a soluble fragment of an amino acid sequence selected from the group consisting of:
(1) an amino acid sequence as set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50 or 52;
(2) an amino acid sequence that shares at least 70% sequence similarity or sequence identity with the sequence set forth in any one of SEQ ID NO: 2, 4, 6, 8, 10, 12, 14, 16, 18, 20, 22, 24, 26, 28, 30, 32, 34, 36, 38, 40, 42, 44, 46, 48, 50 or 52;
(3) an amino acid sequence which is encoded by the nucleotide sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51, or a complement thereof;
(4) an amino acid sequence which is encoded by a nucleotide sequence that shares at least 70% sequence identity with the nucleic acid sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51, or with a complement thereof; and
(5) an amino acid sequence which is encoded by a nucleotide sequence that hybridizes under at least medium or high stringency conditions to the nucleic acid sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49 or 51, or to a complement thereof,
wherein the soluble fragment of the amino acid sequence of (1), (2), (3), (4) or (5) corresponds to all or part of a TRAIL extracellular domain and has any one or more activities selected from the group consisting of: inducing apoptosis of adipose tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, reducing circulating levels of the proinflammatory cytokines IL-6, IL-1alpha, G-CSF and MCP-1, counteracting lipopolysaccaride- and muramildipeptide-induced inflammation.

10. The TRAIL DR agonist according to claim 10, wherein the TRAIL extracellular domain comprises from about amino acid 43 to about amino acid 301 (relative to the consensus numbering shown in FIG. 1).

11. The TRAIL DR agonist according to claim 10, wherein the soluble fragment comprises amino acids X to 301 (relative to the consensus numbering shown in FIG. 1), wherein X represents any of the amino acids at positions 43 to 132 relative to the same consensus numbering.

12. The TRAIL DR agonist according to claim 2, wherein the TRAIL DR agonist is selected from TRAIL polynucleotides, which are suitably in isolated, synthetic, recombinant or purified form, wherein the polynucleotides comprise a nucleotide sequence encoding the TRAIL polypeptide of claim 2.

13. The TRAIL DR agonist according to claim 12, wherein the TRAIL polynucleotides comprise a nucleotide sequence selected from the group consisting of:
(i) a nucleotide sequence selected from any one of SEQ ID NO: 1, 3, 5, 7, 9, 11, 13, 15, 17, 19, 21, 23, 25, 27, 29, 31, 33, 35, 37, 39, 41, 43, 45, 47, 49, 53, 55, 57, 59, 61, 63, 65, 67, 69, 71, 73, 75, 77, 79, 81, 83, 85, 87, 89, 91, 93, 95, 97, 99 or 101, or a complement thereof;
(ii) a nucleotide sequence that shares at least 70% sequence identity with the sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49, 53, 55, 57, 59, 61, 63, 65, 67, 69, 71, 73, 75, 77, 79, 81, 83, 85, 87, 89, 91, 93, 95, 97, 99 or 101, or a complement thereof; and
(iii) a nucleotide sequence that hybridizes under at least medium or high stringency conditions to the sequence set forth in any one of SEQ ID NO: 1, 3, 5, 7, 9, 11, 13, 15, 17, 19, 21, 23, 25, 27, 29, 31, 33, 35, 37, 39, 41, 43, 45, 47, 49, 53, 55, 57, 59, 61, 63, 65, 67, 69, 71, 73, 75, 77, 79, 81, 83, 85, 87, 89, 91, 93, 95, 97, 99 or 101, or a complement thereof,
wherein the amino acid sequence encoded by the nucleotide sequence of (i), (ii) or (iii) has any one or more activities selected from the group consisting of: inducing apoptosis of adipose tissue; reducing fasting hyperinsulinemia, reducing glucose levels after a hyperglycemic stimulus; reducing hyperinsulinemia after a hyperglycemic stimulus, enhancing peripheral response to insulin; reducing increased adiposity in response to high fat diet, improving mitochondrial fatty acid oxidative capacity of muscle tissue, reducing circulating levels of the proinflammatory cytokines IL-6, IL-1alpha, G-CSF and MCP, counteracting lipopolysaccaride- and muramildipeptide-induced inflammation.

14. The TRAIL DR agonist according to claim 13, wherein the TRAIL polynucleotides are in the form of nucleic acid constructs in which the polynucleotides are operably connected to a regulatory sequence.

15. The TRAIL DR agonist according to claim 1, wherein the TRAIL DR agonist is a peptide compound that agonizes a TRAIL-R2 and comprise the amino acid sequence:
[SEQ ID NO: 136]
Ac-WDCLDNX₁IGRRQCVX₂L-NH₂,
wherein X₁ and X₂ are each independently selected R and K.

16. The TRAIL DR agonist according to claim 15, wherein the peptide compound is selected from the group consisting of:
[SEQ ID NO: 137]
AcWDCLDNRIGRRQCVKL-NH₂;
[SEQ ID NO: 138]
AcGGSWDCLDNRIGRRQCVKL-NH₂;
[SEQ ID NO: 139]
AcWDCLDN(X₃)IGRRQCVKL-NH₂;
[SEQ ID NO: 140]
AcWDCLDRPGRRQCVK-NH₂;
[SEQ ID NO: 141]
AcWDCLDNKIGRRQCVRL-NH₂;
[SEQ ID NO: 142]
AcCLDNRIGRRQCV;
[SEQ ID NO: 143]
AcDCLDNRIGRRQCVKL-NH₂;
[SEQ ID NO: 144]
AcWDCLDNRIGKRQCVRL-NH₂;
[SEQ ID NO: 145]
AcWDCLDNRIG(X₄)RQCV(X₅)L-NH₂;
[SEQ ID NO: 146]
AcWDCLDNRIGRRQCVK-NH2;
[SEQ ID NO: 147]
AcWDCLVDRPGRRQCVRLEK-NH2;
[SEQ ID NO: 148]
AcWDCLVDRPGRRQCVRLERK-NH2;
[SEQ ID NO: 149]
AcWDCLVDRPGRRQCVKLER-NH2;
[SEQ ID NO: 150]
GGGSWDCLDNRIGRRQCVKL;
[SEQ ID NO: 151]
AcCWDLDNRIGRRQVCKL-NH2;
and
[SEQ ID NO: 152]
GGGSWDCLDNRIGRRQCVKL-NH2,
wherein X₃, X₄, and X₅ are independently selected from R and K.

17. The TRAIL DR agonist according to claim 16, wherein the peptide compound is selected from the group consisting of:
[SEQ ID NO: 153]
Ac-WDC*LDNX1IGRRQC*VX₂LNH₂,
[SEQ ID NO: 154]
Ac-WDC*LDNRIGRRQC*VKLNH₂,
[SEQ ID NO: 155]
Ac-GGSWDC*LDNRIGRRQC*VKLNH₂,
[SEQ ID NO: 156]
Ac-WDC*LDNX₃IGRRQC*VKLNH₂,
[SEQ ID NO: 157]
Ac-WDC*LDRPGRRQC*VKNH₂,
[SEQ ID NO: 158]
Ac-WDC*LDNKIGRRQC*VRLNH₂,
[SEQ ID NO: 159]
Ac-C*LDNRIGRRQC*V,
[SEQ ID NO: 160]
Ac-DC*LDNRIGRRQC*VKL-NH₂,
[SEQ ID NO: 161]
Ac-WDC*LDNRIGKRQC*VRL-NH2,
[SEQ ID NO: 162]
Ac-WDC*LDNRIGX₄RQC*VX₅L-NH₂,
[SEQ ID NO: 163]
Ac-WDC*LDNRIGRRQC*VK-NH₂,
[SEQ ID NO: 164]
Ac-WDC*LVDRPGRRQC*VRLEK-NH₂,
[SEQ ID NO: 165]
Ac-WDC*LVDRPGRRQC*VRLERK-NH₂,
[SEQ ID NO: 166]
Ac-WDC*LVDRPGRRQC*VKLER-NH₂,
[SEQ ID NO: 167]
GGGSWDC*LDNRIGRRQC*VKL-NH₂,
and
[SEQ ID NO: 168]
ACC*WDLDNRIGRRQVC*KL-NH₂,
wherein * represents a cysteine residue of a disulfide bond.

18. The TRAIL DR agonist according to claim 1, wherein the TRAIL DR agonist is a chimeric or non-natural polypeptide, which comprise a trimerizing domain and at least one polypeptide that binds to at least one TRAIL death receptor.

19. The TRAIL DR agonist according to claim 18, wherein the trimerizing domain comprises a polypeptide of: X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅L₁X₂₆X₂₇X₂₈X₂₉X₃₀L₂X₃₁X₃₂E₁V₁X₃₃X₃₄L₃K₁E₂X₃₅Q₁A₁L₄Q₂T₁V₂C₁L₅X36 (SEQ ID NO: 337), having up to five amino acid substitutions at X₁₀, X₁₇, X₂₀, X₂₁, X₂₄, X₂₅, L₁, X₂₇, X₂₈, X₂₉, X₃₀, L₂, X₃₁, X₃₂, or E₁, wherein each X is independently selected from any amino acid residue and wherein three trimerizing domains form a trimeric complex.

20. The TRAIL DR agonist according to claim 19, wherein the trimerizing domain comprises a trimerizing peptide selected from the group consisting of:
[SEQ ID NO: 169]
(a) NTGLLESQLSRHDQMLSVHDIRLADMDLRFQVLETASYNGVL
IWKIRDYKRRKQEAVM (hTRAF3);
[SEQ ID NO: 170]
(b) AASERKALQTEMARIKKWLTF (hMBP),
[SEQ ID NO: 171]
(c) FDMSCRSRLATLNEKLTALERRIEYIEARVTKGETLT
(hSPC300),
[SEQ ID NO: 172]
(d) ADIYKADFQAERQAREKLAEKKELLQEQLEQLQREYSK
LKASCQES(ARIhNEMO),
[SEQ ID NO: 173]
(e) LTGSAQNIEFRTGSLGKIKLNDEDLSECLHQIQKNKEDIIELKG
SAIGLPIYQLNSKLVDLERKFQGLQQT (hcubilin),
[SEQ ID NO: 174]
(f) LRGLRTIVTTLQDSIRKVTEENKELANE hThrombospondins,
[SEQ ID NO: 175]
(g) VASLRQQVEALQGQVQHLQAAFSQYKK (neck region
of human SP-D),
[SEQ ID NO: 338170]
(h) VNALRQRVGILEGQLQRLQNAFSQYKK (neck region
of bovine SP-D),
[SEQ ID NO: 176]
(i) SAALRQQMEALNGKLQRLEAAFSRYKK (neck region
of rat SP-D),
[SEQ ID NO: 177]
(j) VNALKQRVTILDGHLRRFQNAFSQYKK (neck region of
bovine conglutinin);
[SEQ ID NO: 178]
(k) VDTLRQRMRNLEGEVQRLQNIVTQYRK (neck region of
bovine collectin);
and
[SEQ ID NO: 179]
(l) GSPGLKGDKGIPGDKGAKGESGLPDVASLRQQVEALQGQ
VQHLQAAFSQYKKVELFPGGIPHRD (neck region of
human SP-D).

21. The TRAIL DR agonist according to claim 19, wherein the polypeptide that binds to a TRAIL death receptor comprises a biologically active fragment of TRAIL, which comprises about 5 to about 50 amino acid residues.

22. The TRAIL DR agonist according to claim 19, wherein the polypeptide that binds to a TRAIL death receptor comprises C-Type Lectin Like Domain (CLTD) wherein one of loops 1, 2, 3 or 4 of loop segment A or loop segment B comprises a polypeptide sequence that binds at least one of TRAIL-R1 and TRAIL-R2.

23. The TRAIL DR agonist according to claim 19, wherein the polypeptide binds to TRAIL-R1 and comprises a CLTD comprising one of the following combinations of sequences in loops 1 and 4:

| | Loop 1 | | Loop 4 |
|---|---|---|---|
| Loop 1 | SEQ ID | | SEQ |
| Sequence | NO | Loop 4 Sequence | ID NO |
| GWLEGAGW | 180 | DGGWHWRWEN | 181 |
| GWLEGVGW | 182 | DGGEHWGWEN | 183 |
| GYLAGVGW | 184 | DGGRGFRWEN | 185 |
| GWLEGYGW | 186 | DGGTWWEWEN | 187 |
| GYLEGYGW | 188 | DGGATIAWEN | 189 |
| GWLqGVGW | 190 | DGGRGWPWEN | 191 |
| GYLAGYGW | 192 | DGGPSIWREN | 193 |
| GYIEGTGW | 194 | DGGSNWAWEN | 195 |
| GYMSGYGW | 196 | DGGMMARWEN | 197 |
| GFMVGRGW | 198 | DGGSMWPWEN | 199 |
| MVTRPPYW | 200 | DGGWVMSFEN | 201 |
| PFRVPqWW | 202 | DGGYGPVqEN | 203 |
| GWLEGAGW | 204 | DGGWQWRWEN | 205 |
| GYLDGVGW | 206 | DGGQGCRWEN | 207 |
| VLRLAWSW | 208 | DGGKRNGCEN | 209 |
| WLSLFSPW | 210 | DGGRGVRGEN | 211 |
| GWMAGVGW | 212 | DGGRRLPWEN | 213 |
| SYRLHYGW | 214 | DGGRRWLGEN | 215 |
| IWPLRFRW | 216 | DGGFVTRKEN | 217 |
| WqLYYRYW | 218 | DGGVGCMVEN | 219 |
| RCLqGVGW | 220 | DGGRGWPWEN | 221 |
| GCTqGQGW | 222 | DGGKKWKWEN | 223 |
| GFLqGNGW | 224 | DGGMWDRWEN | 225 |
| GVLqRGGW | 226 | DGGPGGEREN | 227 |
| PFRVLqQWW | 228 | DGGCGPVqQEN | 229 |
| PFRGPqQWW | 230 | DGGYGPVGEN | 231 |
| ARFAMWqQW | 232 | DGGRAGVGEN | 233 |
| GWLQGYGW | 234 | DGGqQIGWGEN | 235 |
| AWRSWLNW | 236 | DGGREqQRREN | 237 |
| GWLEGVGW | 238 | DGGWPFSNEN | 239 |
| GWLMGTGW | 240 | DGGWWNRWEN | 241 |
| VRRMGFHW | 242 | DGGRVAVGEN | 243 |
| RYHVQALW | 244 | DGGRVRPREN | 245 |
| IqCSPPLW | 246 | DGGAVqqQEN | 247 |
| GLARQqGW | 248 | DGGKGRPREN | 249 |
| GWLSGVGW | 250 | DGGWAHAWEN | 251 |
| GWLEGVGW | 252 | DGGGGVRWEN | 253 |
| GWLSGYGW | 254 | DGGRVWSWEN | 255 |
| GLLSDWWW | 256 | DGGGNqSREN | 257 |
| QWVAFWSW | 258 | DGGSAVSGEN | 259 |
| PYTSWGLW | 260 | DGGVGGRGEN | 261 |
| VARWLLKW | 262 | DGGMCKPCEN | 263 |
| GFLAGVGW | 264 | DGGWWTRWEN | 265 |
| GYLQGSGW | 266 | DGGWKTRWEN | 267 |
| VRHWLqLW | 268 | DGGGWWKGEN | 269 |

24. The TRAIL DR agonist according to claim 19, wherein the polypeptide binds to TRAIL-R2 comprise a C-Type Lectin Like Domain (CLTD) comprising one of the following combinations of sequences in loops 1 and 4:

| | Loop 1 | | Loop 4 |
|---|---|---|---|
| | SEQ ID | | SEQ ID |
| Loop 1 Sequence | NO | Loop 4 Sequence | NO |
| RATLRPRW | 270 | DGG----KN | 271 |
| RAMLRSRW | 272 | DGGRWFQGKN | 273 |
| RALFRPRW | 274 | DGGPWYLKEN | 275 |
| RAVLRPRW | 276 | DGGWVLGGKN | 277 |
| RAWLRPRW | 278 | DGGTLVSGEN | 279 |
| RVIRRSMW | 280 | DGGQKWMAEN | 281 |
| RVLQRPVW | 282 | DGGMVWSMEN | 283 |
| RVqLRPRW | 284 | EGGFRRHAKN | 285 |
| RVVRLSEW | 286 | DGGMLWAMEN | 287 |
| RVISAPVW | 288 | DGGQQWAMEN | 289 |
| RVLRRPQW | 290 | NGGDWRIPEN | 291 |
| RVMMRPRW | 292 | DGGMWGAMEN | 293 |
| RVMRRVLW | 294 | DGGRRETMKN | 295 |
| RVMRRPLW | 296 | DGGRGQQWEN | 297 |
| RVMRRREW | 298 | DGAQLMALEN | 299 |
| RVWRRSLW | 300 | DGGHLVKQKN | 301 |
| KRRWYGGW | 302 | DGGVNTVREN | 303 |
| KRVWYRGW | 304 | DGGMRRRREN | 305 |
| AVIRRPLW | 306 | DGGMKYTMEN | 307 |
| ELVTSRLW | 308 | DGGVMqLGEN | 309 |
| ELGTSRLW | 310 | DGGVMqLGEN | 311 |
| FRGWLRWW | 312 | DDGARVLAEN | 313 |
| GRLKGIGW | 314 | DGGRPQWGEN | 315 |
| GVWqSFPW | 316 | DGGLGYLREN | 317 |
| HLVSLAPW | 318 | DGGGMHQGKN | 319 |
| HIFIDWGW | 320 | DGGVMTMGEN | 321 |
| PVMRGVTW | 322 | DGGRSWVWEN | 323 |
| QLVTVGPW | 324 | DGGVMHRTEN | 325 |
| QLVVqMGW | 326 | DGGWMTVGEN | 327 |
| VAIRRSVW | 328 | DGGERAHSEN | 329 |
| WVMRRPLW | 330 | DGGSMGWREN | 331 |
| WRSMVVWW | 332 | DGGKHTLGEN | 333 |
| ELRTDGLW | 334 | DGGVMRRSEN | 335 |

25. The TRAIL DR agonist according to claim 1, wherein the TRAIL DR agonist is a TRAIL DR agonist antigen-binding molecule.

26. The TRAIL DR agonist according to claim 25, wherein the TRAIL DR agonist antigen-binding molecule is selected from the group consisting of:
(a) an anti-TRAIL-R1 single chain Fv antibody, which comprises the amino acid sequence: EVQLVQSGAEVKMPGASVKLSCRVSGDTFTAYFIHWVRQAPGQGLEWMGWFNPI SGTAGSAEKFRGRVAMTRDTSISTAYMELNRLTFDDTAVYYCARQHRGNTFDPW GQGTLVTVSSGGGGSGGGGSGGGGSAQSALTQPASVSGSPGQSITISCTGTSSDIG AYKYVSWYQQHPGKAPKLVIYEVSNRPSGVSSRFSGSKSGQTASLTISGLQADDE ADYYCNSYQGYNTWVFGGGTKVTVLG [SEQ ID NO: 336], as disclosed for example in US 2010/0210545, which is incorporated by reference herein in its entirety;
(b) a humanized TRAIL-R1 agonist monoclonal antibody designated HGS-ETR1 or Mapatumumab (Human Genome Sciences, Rockville, Md., USA);
(c) a humanized TRAIL-R2 agonist monoclonal antibody designated HGS-ETR1 (Human Genome Sciences, Rockville, Md., USA);
(d) a humanized TRAIL-R2 agonist monoclonal antibody designated CS-1008 or Tigatuzumab (Daiichi Sankyo Inc. NJ, USA);
(e) a human TRAIL-R2 agonist monoclonal antibody designated AMG655 or Conatumumab (Amgen, CA, USA);
(f) TRAIL-R2 agonist antigen-binding molecules disclosed in US 2007/0179086; and
(g) TRAIL DR agonist antigen-binding molecules disclosed in US 2008/0199423.

27. The TRAIL DR agonist according to claim 1, wherein the TRAIL DR agonist is a small molecule TRAIL DR agonist selected from compounds having either the formula: 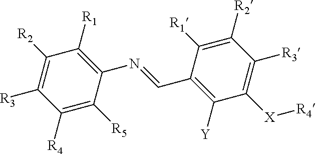
wherein: R₁, R₂, R₃, R₄, R₅, R_{1′}, R_{2′}, and R_{3′} are each independently H, hydroxy, amino, cyano, halo, nitro, mercapto, OPO(OH)₂, PO(OH)₂, OSO₂OH, SO.sub.2OH, or a heteroatom-substituted or heteroatom-unsubstituted C₁-C.sub₃-alkyl, C₂-C₃-alkenyl, C₂-C₃-alkynyl, C₁-C₃-acyl, C₁-C₃-alkoxy, C₁-C₃-acyloxy, C₁-C₃-alkylamino, or C₁-C₃-amido; R_{4′} is H or a heteroatom-substituted or heteroatom-unsubstituted C₁-C₁₀-alkyl, C₁-C₁₀-aryl, C₂-C₁₀-aralkyl, C₂-C₁₀-alkenyl, C₂-C₁₀-alkynyl, or C₁-C₁₀-acyl; X is selected from the group consisting of O, S, and NH, and Y is selected from the groups consisting of hydroxy, amino, and mercapto;
or the formula: 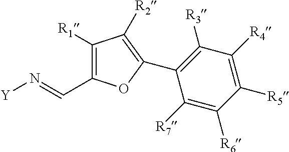
wherein: R_{1″}, R_{2″}, R_{3″}, R_{5″}, R_{6′} and R_{7″}, are each independently H, hydroxy, amino, cyano, halo, nitro, mercapto, OPO(OH)₂, PO(OH)₂, OSO.₂OH, SO₂OH, or a heteroatom-substituted or heteroatom-unsubstituted C₁-C₈-alkyl, C₂-C₈-alkyl, alkenyl, C₂-C₈-alkynyl, C₁-C₈-aryl, C₁-C₈-aralkyl, C₁-C₈-acyl, C₁-C₈-alkoxy, C₁-C₈-aryloxy, C₂-C₈-aralkoxy, C₁-C₈-acyloxy, C₁-C₈-alkylamino, C₁-C₈-arylamino, C₂-C₈-aralkylamino, or C₁-C₈-amido; Y is selected from the groups consisting of heteroatom-substituted or heteroatom-unsubstituted C₁-C₁₅-alkylamino, C₁-C₁₅-alkenylamino, C₁-C₁₅-alkynylamino, C₁-C₁₅-arylamino, C₂-C₁₅-aralkylamino, and C₁-C₁₅-amido; or a pharmaceutically acceptable salt, hydrate, amine-N-oxide, imine-N-oxide, tautomer, or optical isomer of either of the above formulas.

28. The TRAIL DR agonist according to claim 27, wherein the compounds are represented by the structure:

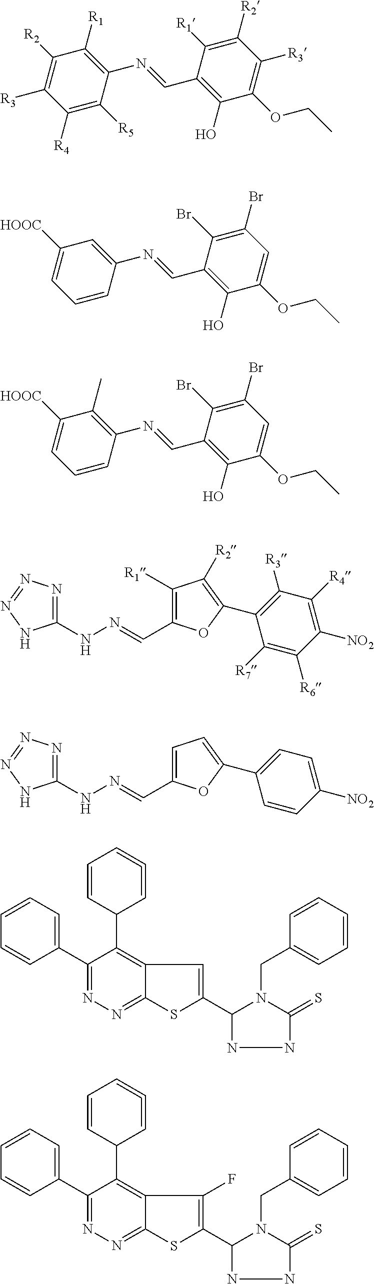

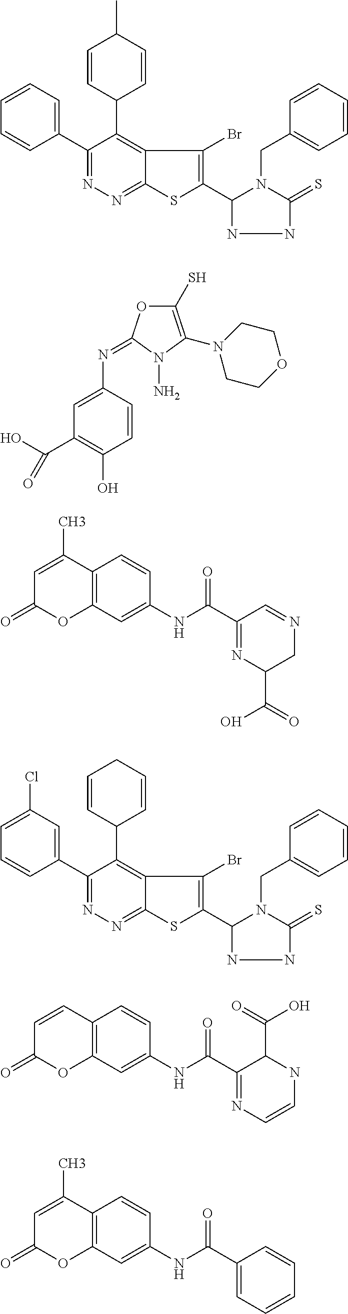

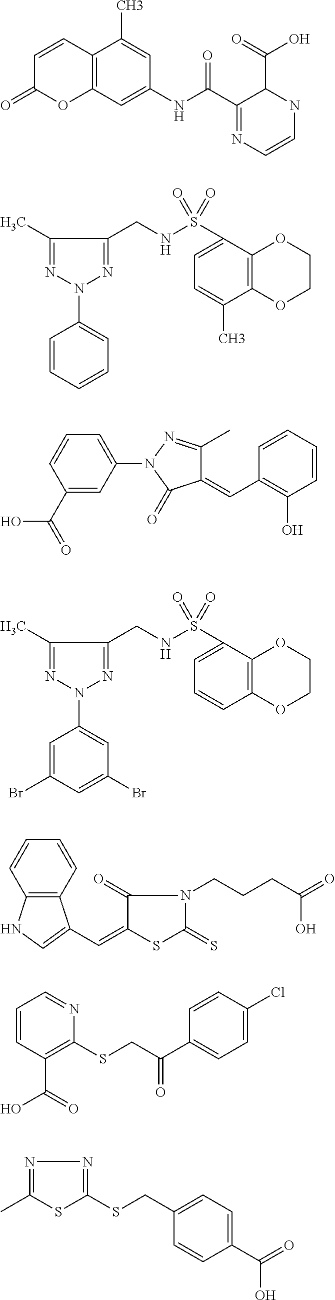

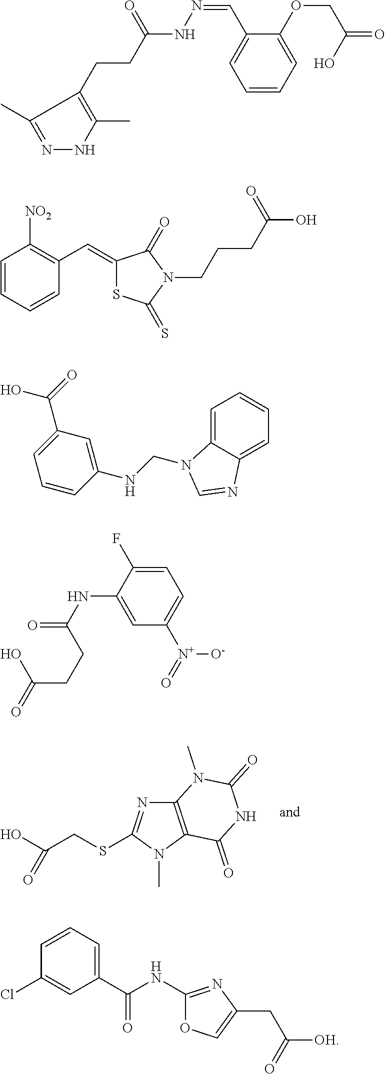 [SMILES: Cc1n[nH]c(C)c1CCC(=O)N/N=C\c1ccccc1OCC(=O)O ; O=C(O)CCCN1C(=O)/C(=C/c2ccccc2[N+](=O)[O-])SC1=S ; O=C(O)c1cccc(NCn2cnc3ccccc32)c1 ; O=C(O)CCC(=O)Nc1cc([N+](=O)[O-])ccc1F ; Cn1c(SCC(=O)O)nc2c1c(=O)[nH]c(=O)n2C ; O=C(O)Cc1coc(NC(=O)c2cccc(Cl)c2)n1]

29. (canceled)

30. (canceled)

31. (canceled)

32. (canceled)

33. A method for controlling adiposity in a subject, comprising administering to the subject an effective amount of a TRAIL DR agonist, and optionally a pharmaceutically acceptable carrier or diluent.

34. A method for treating or reducing an occurrence of an adiposity-related condition in a subject, comprising administering to the subject an effective amount of a TRAIL DR agonist, and optionally a pharmaceutically acceptable carrier or diluent.

35. A method according to claim 34, wherein the adiposity-related condition is selected from the group consisting of obesity, diabetes mellitus and metabolic syndrome.

36. (canceled)

37. (canceled)

38. A method for stimulating the death of an adipose cell, comprising contacting the adipose cell with a cell death-stimulating effective amount of a TRAIL death receptor (DR) agonist.

39. The TRAIL death receptor (DR) agonist according to claim 1 wherein the adipose cell is selected from the group consisting of an adipocyte and precursor thereof.

40. The TRAIL death receptor (DR) agonist according to claim 1 wherein the adipose cell comprises a preadipocyte.

41. A method of preparing a medicament for controlling adiposity comprising:
formulating the TRAIL DR agonist according to claim 1 with a pharmaceutically acceptable carrier or diluent.

42. The method according to claim 42, wherein the adiposity-related condition is selected from the group consisting of obesity, diabetes mellitus and metabolic syndrome.


SEQUENCE LISTING (371 sequences)

SEQ ID NO: 1 - DNA, 507 bp, Homo sapiens
gtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 2 - protein, 168 aa, Homo sapiens
VRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 3 - DNA, 846 bp, Artificial Sequence
  source: /note=“Description of Artificial Sequence Synthetic polynucleotide”
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggcttg

SEQ ID NO: 4 - protein, 281 aa, Artificial Sequence [sequence omitted]

SEQ ID NO: 5 - DNA, 846 bp, Homo sapiens
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 6 - protein, 282 aa, Homo sapiens
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGL

SEQ ID NO: 7 - DNA, 846 bp, Artificial Sequence
  source: /note=“Description of Artificial Sequence Synthetic polynucleotide”
atggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttaggggcctttttagttggcttg

SEQ ID NO: 8 - protein, 282 aa, Artificial Sequence
  source: /note=“Description of Artificial Sequence Synthetic polypeptide”
MAMMEVQGGPSLGQTCVLIVIFTVLLQSLCVAVTYVYFTNELKQMQDKYSKSGIACFLKEDDSYWDPNDEESMNSPCWQVKWQLRQLVRKMILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFLGAFLVGL

SEQ ID NO: 9 - DNA, 672 bp, Homo sapiens
caggatcatggctatgatggaggtccaggggggacccagcctgggacagacctgcgtgctgatcgtgatcttcacagtgctcctgcagtctctctgtgtggctgtaacttacgtgtactttaccaacgagctgaagcagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaa

SEQ ID NO: 10 - protein, 178 aa, Homo sapiens
KEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 11 - DNA, 846 bp, Pan troglodytes
atggctatgatggaggtccaggggggacccagcctgggacagacctgcgtgctgatcgtggtcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagacagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactggaaccagaggaagaagcaacacattgtcttctccaaactccaagaatgaaaaggctctgggccacaaaataaactcctgggaatcatcaaggagtgggcattcattcctgagcaacttgcacttgaggaatggcgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagcgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 12 - protein, 281 aa, Pan troglodytes
MAMMEVQGGPSLGQTCVLIVVFTVLLQSLCVAVTYVYFTNELKQMQDKYSKSGIACFLKEDDSYWDPNDEDSMNSPCWQVKWQLRQLVRKMILRTSEETISTVQEKQQNISPLVRERGPQRVAAHITGTRGRSNTLSSPNSKNEKALGHKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 13 - DNA, 507 bp, Homo sapiens
  modified_base (3)..(3): a, c, t, g, unknown or other
gtnmgngarmgnggnccncarmgngtngcngcncayathacnggnacnmgnggnmgnwsnaayacnytnwsnwsnccnaaywsnaaraaygaraargcnytnggnmgnaarathaaywsntgggarwsnwsnmgnwsnggncaywsnttyytnwsnaayytncayytnmgnaayggngarytngtnathcaygaraarggnttytaytayathtaywsncaracntayttymgnttycargargarathaargaraayacnaaraaygayaarcaratggtncartayathtayaartayacnwsntayccngayccnathytnytnatgaarwsngcnmgnaaywsntgytggwsnaargaygcngartayggnytntaywsnathtaycarggnggnathttygarytnaargaraaygaymgnathttygtnwsngtnacnaaygarcayytnathgayatggaycaygargcnwsnttyttyggngcnttyytngtnggntrr

SEQ ID NO: 14 - protein, 168 aa, Homo sapiens; identical to SEQ ID NO: 2

SEQ ID NO: 15 - DNA, 576 bp, Homo sapiens
  modified_base (9)..(9): a, c, t, g, unknown or other
atgathytnmgnacnwsngargaracnathwsnacngtncargaraarcarcaraayathwsnccnytngtnmgngarmgnggnccncarmgngtngcngcncayathacnggnacnmgnggnmgnwsnaayacnytnwsnwsnccnaaywsnaaraaygaraargcnytnggnmgnaarathaaywsntgggarwsnwsnmgnwsnggncaywsnttyytnwsnaayytncayytnmgnaayggngarytngtnathcaygaraarggnttytaytayathtaywsncaracntayttymgnttycargargarathaargaraayacnaaraaygayaarcaratggtncartayathtayaartayacnwsntayccngcnccnathytnytnatgaarwsngcnmgnaaywsntgytggwsnaargaygcngartayggnytntaywsnathtaycarggnggnathttygarytnaargaraaygaymgnathttygtnwsngtnacnaaygarcayytnathgayatggaycaygargcnwsnttyttyggngcnttyytngtnggntrr

SEQ ID NO: 16 - protein, 191 aa, Homo sapiens
MILRTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPAPILLMKSARNSCWSKDAEYGLYSIYQGGIFELKENDRIFVSVTNEHLIDMDHEASFFGAFLVG

SEQ ID NO: 17 - DNA, 846 bp, Macaca mulatta
atggctatgatggaggcccaggggggacccagcccggggcagacctgcgtgctgatcctgatcttcacggtgctcctgcagtccctctgtgcagctgtaacttacgtgtacttcaccaacgagctgaagcagatgcaggacaagtactccaaaagtggcattgcttgtttcttgaaagaagatgacagttcttgggatcccaatgacgaagagagtatgaagagcccctgctggcaagtcaagtggcaactccgtcaactcgttagaaagatgattttgagaacctctgaggaaaccatttctacagttcaagaaaagcaacaaaatacttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggcgaactggtcatccaagaaaaggggttttactacatctattcccaaacatactttcgatttcaggaggaaataaaagaaaacacaaagaacgacaaacaaatggtccaatatatttacaaatacacaagttatcctgaccctatactgctgatgaaaagcgctagaaatagttgttggtctaaagatgcagaatacggactctattccatctatcaagggggattatttgagcttaagaaagatgacagaatttttgtttctgtaacaaatgagcacttgatagacatggaccatgaagccagctttttcggggcctttttggttggctaa

SEQ ID NO: 18 - protein, 281 aa, Macaca mulatta
MAMMEAQGGPSPGQTCVLILIFTVLLQSLCAAVTYVYFTNELKQMQDKYSKSGIACFLKEDDSSWDPNDEESMKSPCWQVKWQLRQLVRKMILRTSEETISTVQEKQQNTSPLVRERGPQRVAAHITGTRGRSNTLSSPNSKNEKALGRKINSWESSRSGHSFLSNLHLRNGELVIQEKGFYYIYSQTYFRFQEEIKENTKNDKQMVQYIYKYTSYPDPILLMKSARNSCWSKDAEYGLYSIYQGGLFELKKDDRIFVSVTNEHLIDMDHEASFFGAFLVG

SEQ ID NO: 19 - DNA, 501 bp, Crassostrea ariakensis
atggtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattccagagcaacttgcacttgaggaatggtgaactggtcatccatgaaaaagggttttactacatctattcccaaacatactttcgatttcaggaggaaataaaagaaaacgcaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggccttttaa

SEQ ID NO: 20 - protein, 166 aa, Crassostrea ariakensis
MVRERGPQRVAAHITGTRGRSNTLSSPNSKNEKALGRKINSWESSRSGHSFQSNLHLRNGELVIHEKGFYYIYSQTYFRFQEEIKENAKNDKQMVQYIYKYTSYPDPILLMKSARNSCWSKDAEYGLYSIYQGGIFELKENDRIFVSVTNEHLIDMDHEASFFGAF

SEQ ID NO: 21 - DNA, 492 bp, Homo sapiens
  modified_base (3)..(3): a, c, t, g, unknown or other
ccncarmgngtngcngcncayathacnggnacnmgnggnmgnwsnaayacnytnwsnwsnccnaaywsnaaraaygaraargcnytnggnmgnaarathaaywsntgggarwsnwsnmgnwsnggncaywsnttyytnwsnaayytncayytnmgnaayggngarytngtnathcaygaraarggnttytaytayathtaywsncaracntayttymgnttycargargarathaargaraayacnaaraaygayaarcaratggtncartayathtayaartayacnwsntayccngayccnathytnytnatgaarwsngcnmgnaaywsntgytggwsnaargaygcngartayggnytntaywsnathtaycarggnggnathttygarytnaargaraaygaymgnathttygtnwsngtnacnaaygarcayytnathgayatggaycaygargcnwsnttyttyggngcnttyytngtnggntrr

SEQ ID NO: 22 - protein, 163 aa, Homo sapiens
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 23 - DNA, 846 bp, Pongo abelii
  modified_base (436)..(444): a, c, t, g, unknown or other
atggctatgatggaggtccaggggggacccagcctggggcagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagctcttgggaccctaatgacgaagacagtatgaacagcccctgctggcaagtcaagtggcaactccgtcagctcgttagaaagatgattttgagaacctctgaggaaaccatttctacagttcaagaaaagcaacaaaatgtttctcccctagtgagagaaagaggtcctcagagagtagcagctcacataactgggaccagaggaagaagcaacacattgtcttctccaagtaagagaaacaacaaannnnnnnnncgcaaaataaactcctgggaatcatcaaggagtgggcattcattcctgagcaacttgcacttgaggaatggcgaactggtcatccatgaaaaagggttttactacatctattcccaaacatactttcgatttcaggaggaaataaaagaaaacacaaagaacgacaaacaaatggtccaatatatttacaaatacacaagttatcctgatcctatattgctgatgaaaagcgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggggcctttttagttggctaa

SEQ ID NO: 24 - protein, 281 aa, Pongo abelii
  MOD_RES (146)..(148): Any amino acid
MAMMEVQGGPSLGQTCVLIVIFTVLLQSLCVAVTYVYFTNELKQMQDKYSKSGIACFLKEDDSSWDPNDEDSMNSPCWQVKWQLRQLVRKMILRTSEETISTVQEKQQNVSPLVRERGPQRVAAHITGTRGRSNTLSSPSKRNNKXXXRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVG

SEQ ID NO: 25 - DNA, 846 bp, Callithrix jacchus
atggctatgatggagggtcaggggggacccagcccggggcagacctgcgtgctgatcctgatcttcacagtgctcctgcagtccctctgtgtggccgtaacttacctgtacttcaccaatgagctgaagcagatgcaggacaagtactccaaaagcggcattgcttgtttcttaaaagaagatggcagctcctgggaccccagtgacgaagagagtatgaatagcccctgctgggaagtcaagtggcaactccgtcagctcgttagaaagatgattttgagaacctctgaagaaaccatttctacagttcaagaaaagcaacgaggtatttctccccaagtgagagaaagaggtcctcagagagtagcagctcacataactgggaccagaggaagtagcaacacgttgcctattccaaactccaagaatgaaaaggctctgggccgcaaaataaactcctgggaatcatcaaggagtggacattctttcctgagcaacttgcacttgaggaatggcgagctggtcatccatgaaaaagggctgtattacatctattgccaagtatactttcgatttcaggaggaaatccaagaaaacagaaagaacgacaaacaaatggtccagtatatttacaaatacacaagttatcctgaccccatactgctgatgaagagtgctagaaataattgttggtctaaagatgcagaatatggactctattccatctatcaagggggaatatttgagcttaaggaaaacgacagaatttttgtttctgtaacaaatgggcagttgatagacatggaccatgaagccagttttttcggggcctttttagttggctaa

SEQ ID NO: 26 - protein, 281 aa, Callithrix jacchus
MAMMEGQGGPSPGQTCVLILIFTVLLQSLCVAVTYLYFTNELKQMQDKYSKSGIACFLKEDGSSWDPSDEESMNSPCWEVKWQLRQLVRKMILRTSEETISTVQEKQRGISPQVRERGPQRVAAHITGTRGSSNTLPIPNSKNEKALGRKINSWESSRSGHSFLSNLHLRNGELVIHEKGLYYIYCQVYFRFQEEIQENRKNDKQMVQYIYKYTSYPDPILLMKSARNNCWSKDAEYGLYSIYQGGIFELKENDRIFVSVTNGQLIDMDHEASFFGAFLVG

SEQ ID NO: 27 - DNA, 846 bp, Felis catus
atgcaggccccggcgggccccagtcccgggcagacctgcgtgctgatcctgatcttcactgtgctcctgcagtccctctgcgtggccgtgacttacatgtacttcaccagtgaactgaggcagatgcaggacaaatactcccaaagtggcattgcttgtttcttaaaggaagacgatatcccttgggaccccaatgatgaagagagtatgaacaccccgtgctggcaagtgaaatggcagctccgtcagtttgttagaaagattttgagaacctatgaggaaaccattcctacagttccagaaaagcagctaaatattccttacctagtaagagaaagaggtcctcagagagtagcagctcacataactggaaccagtcggagaagaagcacattcccagttccaagctccaagaatgaaaaagctttgggtcagaaaataaactcctgggagtcatcaagaaaaggacattcattcttgaataatttgcacttgaggaatggtgagctggttattcatcagagggggttttattacatctattcccaaacatactttcgatttcaggaacctgaggaaattccaacaggacagaacagaaagagaaacaaacaaatggtccaatatatttacaaacacacgagttatccggaccctatactgctgatgaaaagtgctagaaatagttgttggtctaaagattctgaatatggactctattccatctatcaaggtgggatatttgagcttaaggaaaacgatagaatttttgtctctgtatctaacgagcaattgattgacatggaccaagaagccagttttttcggggcctttttaatcggctaa

SEQ ID NO: 28 - protein, 281 aa, Felis catus
MQAPAGPSPGQTCVLILIFTVLLQSLCVAVTYMYFTSELRQMQDKYSQSGIACFLKEDDIPWDPNDEESMNTPCWQVKWQLRQFVRKILRTYEETIPTVPEKQLNIPYLVRERGPQRVAAHITGTSRRRSTFPVPSSKNEKALGQKINSWESSRKGHSFLNNLHLRNGELVIHQRGFYYIYSQTYFRFQEPEEIPTGQNRKRNKQMVQYIYKHTSYPDPILLMKSARNSCWSKDSEYGLYSIYQGGIFELKENDRIFVSVSNEQLIDMDQEASFFGAFLIG

SEQ ID NO: 29 - DNA, 861 bp, Ailuropoda melanoleuca
atgcaggccccggggggccccagccctgggcagacgtgcgtgttgaccctcatcttcacagtgctcctgcagtccctctgtgtggcggtgacctacatgtacttcaccagggagctgaagcagatgcaggacaagtactcccaaagcggcatcgcttgtttcttaaaggaagatgatattccttgggacccaaatgatgaagagagtatgaacaatccttgctggcaagtgaagtggcaactccgtcagtttgttagaaagatgattttgaaaacctatgaggaaaccattccttcaattccagaaaagcagctaaatattccttacgtagtaaatgaaagaggtcttcagagagtagcagctcacataactggaaccagtcggagaagaagcacgtttccagttccaagctccaagaatgaaaaagctttgggccagaaaataaactcctgggagtcatcaagaaaaggacattcattcttgagtaatttgcacttgaggaatggagagctggttatccatcaaagtgggttttattacatctattcccaaacatactttcgatttcaggaacctgaggaaacttcgggaccaatttcaaaggaacaaaacagaaagaaaaacaaacaaatggtacaatatatttacaaatacacaagttatcctgaccctatactgctgatgaaaagtgctagaaatagttgctggtctaaagattctgagtatggactctattccatctatcaaggtgggatatttgagcttaaggaaaatgatagaatttttgtctctgtaaataatgagcaattgattgacatggaccaagaagccagttttttcggggcctttttaattggctaa

SEQ ID NO: 30 - protein, 286 aa, Ailuropoda melanoleuca
MQAPGGPSPGQTCVLTLIFTVLLQSLCVAVTYMYFTRELKQMQDKYSQSGIACFLKEDDIPWDPNDEESMNNPCWQVKWQLRQFVRKMILKTYEETIPSIPEKQLNIPYVVNERGLQRVAAHITGTSRRRSTFPVPSSKNEKALGQKINSWESSRKGHSFLSNLHLRNGELVIHQSGFYYIYSQTYFRFQEPEETSGPISKEQNRKKNKQMVQYIYKYTSYPDPILLMKSARNSCWSKDSEYGLYSIYQGGIFELKENDRIFVSVNNEQLIDMDQEASFFGAFLIG

SEQ ID NO: 31 - DNA, 870 bp, Equus caballus
atggccatgatgcaggcatcagggggtcccagccccgggcagacctgcgtgctgatcctgatcttcacagtgctcctgcaggccctctgtgtggctgtgacttatttgtacttcaccaacgagctgaagcagatgcagatcaaatactccaaaagtggcattgcctgtttcttaaaggaagatgacagcgattgggacccaaatgacgaagagagtatgaacagcccctgctggcaagtcaagtggcagctgcgtcagtttgttagaaagatgattttgagaacctatgaggaatccattcctacaacttcagaaaagcgacaaaatattcctcccttagtaagagaaagaggtcttcagagagtagcagctcacataactgggaccagtcggagaagaagcacagtctcaattccacgctccaagaatgaaaaagcactgggccagaaaataaacgcctgggagacatcaagaaaaggacattcgttcttgaataatttacacttgaggaatggagagctggttatccatcaaacagggttttattacatctattcccaaacatactttcgatttcaggaacctgaggaaattttgggaacagttgcaacagaagagaacagaaggaaaaataaacaaatggtacaatatatttacaaaagcacagactatcctgaccctatactgctgatgaaaagtgctagaaatagttgttggtctaaagattcagaatacggactctattccatctatcaaggtggaatatttgagcttaaggaaaatgacagaatttttgtctctgtaactaatgagcaattgattgacatggaccaagaagccagtttcttcggggcctttttaatcggctaa

SEQ ID NO: 32 - protein, 289 aa, Equus caballus
MAMMQASGGPSPGQTCVLILIFTVLLQALCVAVTYLYFTNELKQMQIKYSKSGIACFLKEDDSDWDPNDEESMNSPCWQVKWQLRQFVRKMILRTYEESIPTTSEKRQNIPPLVRERGLQRVAAHITGTSRRRSTVSIPRSKNEKALGQKINAWETSRKGHSFLNNLHLRNGELVIHQTGFYYIYSQTYFRFQEPEEILGTVATEENRRKNKQMVQYIYKSTDYPDPILLMKSARNSCWSKDSEYGLYSIYQGGIFELKENDRIFVSVTNEQLIDMDQEASFFGAFLIG

SEQ ID NO: 33 - DNA, 849 bp, Ailuropoda melanoleuca
atgcaggccccggggggccccagccctgggcagacgtgcgtgttgaccctcatcttcacagtgctcctgcagtccctctgtgtggcggtgacctacatgtacttcaccagggagctgaagcagatgcaggacaagtactcccaaagcggcatcgcttgtttcttaaaggaagatgatattccttgggacccaaatgatgaagagagtatgaacaatccttgctggcaagtgaagtggcaactccgtcagtttgttagaaagatgattttgaaaacctatgaggaaaccattccttcaattccagaaaagcagctaaatattccttacgtagtaaatgaaagaggtcttcagagagtagcagctcacataactggaaccagtcggagaagaagcacgtttccagttccaagctccaagaatgaaaaagctttgggccagaaaataaactcctgggagtcatcaagaaaaggacattcattcttgagtaatttgcacttgaggaatggagagctggttatccatcaaagtgggttttattacatctattcccaaacatactttcgatttcaggaacctgaggaaacttcgggaccaatttcaaaggaacaaaacagaaagaaaaacaaacaaatggtacaatatatttacaaatacacaagttatcctgaccctatactgctgatgaaaagtgctagaaatagttgctggtctaaagattctgagtatggactctattccatctatcaaggtgggatatttgagcttaaggaaaatgatagaatttttgtctctgtaaataatgagcaattgattgacatggaccaagaagccagttttttcggggccttt

SEQ ID NO: 34 - protein, 283 aa, Ailuropoda melanoleuca
MQAPGGPSPGQTCVLTLIFTVLLQSLCVAVTYMYFTRELKQMQDKYSQSGIACFLKEDDIPWDPNDEESMNNPCWQVKWQLRQFVRKMILKTYEETIPSIPEKQLNIPYVVNERGLQRVAAHITGTSRRRSTFPVPSSKNEKALGQKINSWESSRKGHSFLSNLHLRNGELVIHQSGFYYIYSQTYFRFQEPEETSGPISKEQNRKKNKQMVQYIYKYTSYPDPILLMKSARNSCWSKDSEYGLYSIYQGGIFELKENDRIFVSVNNEQLIDMDQEASFFGAF

SEQ ID NO: 35 - DNA, 864 bp, Bos taurus
atggccctgaagcaggctccgggctccagacttgggcagatctgcatgccgatcctcatcttcacagtgctgctgcaggcttttggtatggccgtgttttacatgtatttcaacaaagagctgaagcagatgcagaacaaatacttcaaaagtggcttggcttgcttcttggaggaagatgaccgttcctgggactccagagatgatgagagtataatcaatccctgctgggaactaaagtcccaactctatctgtttgttaaaaagatgactttgagaacctttgaggaaatgattcctacaaatccagaaaagcaatataatccttacctagagagagaaaagggtcctaagagggtagctgctcatataactggaagcaatcggaaaaaaagtacgttgccagttccaggctccaagaatgaaaaagctgtgggccataaaataaattcctgggagtcatcaagaaaaggacattcgttcttgaataatttgtacttaaggaatggagagctggttatccttcaaacaggattttattacatctattcccaaacatactttcgatttcaggaacctgaggaagttttgggaactgtttcaacagaagagaacagaaaaaaaatcaaacaaatggtacaatatatttacaaatacacaaactatcctgaccctatactgctgatgaaaagtgctagaaatagttgttggtctaaagattcagaatatggactctattccatctatcaaggaggaatatttgagcttaaggaaaatgatcgaatttttgtctctgtaactaatgaacgattggttgacctggaccaagaagccagttttttcggagcctttttaattggctaa

SEQ ID NO: 36 - protein, 287 aa, Bos taurus
MALKQAPGSRLGQICMPILIFTVLLQAFGMAVFYMYFNKELKQMQNKYFKSGLACFLEEDDRSWDSRDDESIINPCWELKSQLYLFVKKMTLRTFEEMIPTNPEKQYNPYLEREKGPKRVAAHITGSNRKKSTLPVPGSKNEKAVGHKINSWESSRKGHSFLNNLYLRNGELVILQTGFYYIYSQTYFRFQEPEEVLGTVSTEENRKKIKQMVQYIYKYTNYPDPILLMKSARNSCWSKDSEYGLYSIYQGGIFELKENDRIFVSVTNERLVDLDQEASFFGAFLIG

SEQ ID NO: 37 - DNA, 870 bp, Sus scrofa
atggcggtgatgcagactccaggaggccccagccccgggcagacctgtgtgttgatcctgatcttcacagtgctcctgcaagccctctgtgtggccttgacttacgtgtacttcaccaatgaactgaaacagatgcaggacaagtactccaaaagcggtatagcttgcttcttaaaggaagatgacagtttctgggatcccaccgatgacgagagaatgctcagcccctgctggcaggtgaagtggcagctacgtcagtttgtgagaaagatgattttgagaacctatgaggaaaccatttctacagtttcagaaaagcaacaaggcattcctcacctagaaagagaaaaaggtccacagagagtggctgctcacataactggaaccagtaggaaaagaagcacatttccatctctaagctccaaatatgaaaaagctttgggccagaaaataaactcctgggaatcatcaagaaaaggacattcattcttgaataattttcacttgaggaatggagagctggttatccatcaaacagggttttactacatctattcccaaacatactttcgatttcaggaacctgaggaaattttgggaacggtttctacagaagggaacagaaagaaaaacaggcaaatgatacagtatatttacaaatggacaagctatcctgaccctatactgctgatgaaaagtgctagaaatagttgttggtctaaagattcagaatatggactctattccatctatcaaggtggaatatttgagcttaaggaagatgaccgaatttttgtctctgttactaatgagcaactgattgacatggaccaagaagccagttttttcggggcctttttaattggctaa

SEQ ID NO: 38 - protein, 289 aa, Sus scrofa
MAVMQTPGGPSPGQTCVLILIFTVLLQALCVALTYVYFTNELKQMQDKYSKSGIACFLKEDDSFWDPTDDERMLSPCWQVKWQLRQFVRKMILRTYEETISTVSEKQQGIPHLEREKGPQRVAAHITGTSRKRSTFPSLSSKYEKALGQKINSWESSRKGHSFLNNFHLRNGELVIHQTGFYYIYSQTYFRFQEPEEILGTVSTEGNRKKNRQMIQYIYKWTSYPDPILLMKSARNSCWSKDSEYGLYSIYQGGIFELKEDDRIFVSVTNEQLIDMDQEASFFGAFLIG

SEQ ID NO: 39 - DNA, 846 bp, Canis lupis
atgcaggccccggggggccccagcctcgggctgacgtgcgtgctgatcctcatcttcactgtgctgctccagtccctctgcgtggccgtcacctacatgtacttcaccagggagctgaagcagatgcaggacaagtactcccaaagtggcatcgcttgtttcttaaaggaagatgatatcccctgggaccccagtgatgaagagagtatgaacaacccctgctggcaagtgaagtggcaactccgccagtttgttagaaagatgattttgaaaacctatgaggaaaccattcctacagctccagaaaagcagctaaatattccttacgtagtaagcgaccgaggttctcagagagtagctgctcacataactggaaccagtcggagaagcatgtttccaattccaagctccaagaatgataaagctttgggccacaaaataaactcctgggattccacaagaaaaggacattcattcttgaataatttgcacttgaggaacggagagctggttatccatcaaagggggttttattacatctattcccaaacatactttcgatttcaggaacctgaggaaattccaacaggacagaacagaaagagaaacaaacaaatggtccaatatatttacaaacacacgagttatccggaccctatactgctgatgaaaagtgctagaaatagttgttggtctaaagattctgaatatggactctattccatctatcaaggtgggatatttgagcttaaggaaaacgatagaatttttgtctctgtatctaacgagcaattgattgacatggaccaagaagccagttttttcggggcctttttaatcggctaa

SEQ ID NO: 40 - protein, 281 aa, Canis lupis
MQAPGGPSLGLTCVLILIFTVLLQSLCVAVTYMYFTRELKQMQDKYSQSGIACFLKEDDIPWDPSDEESMNNPCWQVKWQLRQFVRKMILKTYEETIPTAPEKQLNIPYVVSDRGSQRVAAHITGTSRRSMFPIPSSKNDKALGHKINSWDSTRKGHSFLNNLHLRNGELVIHQRGFYYIYSQTYFRFQEPEEIPTGQNRKRNKQMVQYIYKHTSYPDPILLMKSARNSCWSKDSEYGLYSIYQGGIFELKENDRIFVSVSNEQLIDMDQEASFFGAFLIG

SEQ ID NO: 41 - DNA, 885 bp, Oryctolagus cuniculus
atgtcctctgtgcaggccctggggggccccagtgccgggcagacctgcgtgctgatcctgatcttcacagtgctcctgcagtccctctgtgtggccgtgacttacctgtacttcaccaacgaactgaagcagatgcaggacaagtactccaaaagtggcatcgcttgtctcttaaaggaggatgacagttcctgggactccatcgacgaagagaacatgaacagcccctgctggcaggccaagtggcagctgcggcagttcattcgaaagatgcttttgagaacctatgaggaaaccattcctacggttgaagaaaagccacaaactattccttccctagtaagagaaaaagaaagagaaagagggcctcagagagtagcagctcacctaactgggaacagctggagaagctttatctcagtccctgctccaggctcccagagtggaaagaatttgggccagaaaataagctcctgggaatcatcaaggaaaggacattcattcctgaacaatttgcacctgaggaatggagagctggttatccatcaaacaggactttattacatctactcccaaacatactttcgatttcaggaacttgaagaaatttcaggaacaatttcaagagaagagatcaaaaagaggaacaaacaaatggtacaatatatttacaaatggacaagctaccctgaccctatacttctgatgaaaagtgctagaaatagttgttggtctaaggattcggaatatggactctattccatctatcaaggaggaatatttgagcttaaggaaaatgaccgaattttcgtctctgtaacgaatgagcagttgattgacatgaaccaagaatccagtttttttggggcctttttgattggctaa

SEQ ID NO: 42 - protein, 294 aa, Oryctolagus cuniculus
MSSVQALGGPSAGQTCVLILIFTVLLQSLCVAVTYLYFTNELKQMQDKYSKSGIACLLKEDDSSWDSIDEENMNSPCWQAKWQLRQFIRKMLLRTYEETIPTVEEKPQTIPSLVREKERERGPQRVAAHLTGNSWRSFISVPAPGSQSGKNLGQKISSWESSRKGHSFLNNLHLRNGELVIHQTGLYYIYSQTYFRFQELEEISGTISREEIKKRNKQMVQYIYKWTSYPDPILLMKSARNSCWSKDSEYGLYSIYQGGIFELKENDRIFVSVTNEQLIDMNQESSFFGAFLIG

SEQ ID NO: 43 - DNA, 876 bp, Rattus norvegicus
atgccttccaccgggaacctgaagggccccagcttcagtcagcacttcacgatgacggtgatctgcatagtgctcctgcaggtgctcctgcaggccttgactgtggctgtgacttacatgtacttcaacaacgaggtgaaacagctacaggacaattactccaaaatcggactagcttgcttctcaaaagaagatggggatttttgggactccactgacgaggggattttgaacagaccttgcttgcaggtcaagaggcaactgtatcagctcattgaagaggtgactttgagaacctttgagaaaaccatctctacagttccagaaaagcagctaagcactcctcccttgcccagaggtagaagaccccagagagtggcagctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggatcaagaagccagtttctttggagcctttttaattaactag

SEQ ID NO: 44 - protein, 291 aa, Rattus norvegicus
MPSTGNLKGPSFSQHFTMTVICIVLLQVLLQALTVAVTYMYFNNEVKQLQDNYSKIGLACFSKEDGDFWDSTDEGILNRPCLQVKRQLYQLIEEVTLRTFEKTISTVPEKQLSTPPLPRGRRPQRVAAHITGITRRSNLALIPISKDGKTLGQKIETWESSRRGHSFLNHVHLRNGELVIQEEGLYYIYSQTYYRFKEAKEASKTVSKDGGRIKQMVQYIYKYTSYPDPILLMKSARNSCWSREAEYGLYSIYQGGLFELKENDRIFVSVTNEHLMDLDQEASFFGAFLIN

SEQ ID NO: 45 - DNA, 864 bp, Rattus norvegicus
atggcttccaccgggaacctgaagggccccagcttcagtcagcacttcacgatgacggtgatctgcatagtgctcctgcaggtgctcctgcaggccttgactgtggctgtgacttacatgtacttcaacaacgaggtgaaacagctacaggacaattactccaaaatcggactagcttgcttctcaaaagaagatggggatttttgggactccactgacgaggggattttgaacagaccttgcttgcaggtcaagaggcaactgtatcagctcattgaagaggtgactttgagaacctttgagaaaaccatctctacagttccagaaaagcagctaagcactcctcccttgcccagaggtagaagaccccagagagtggcagctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggaccatgaagccagcttctttggagcctaa

SEQ ID NO: 46 - protein, 287 aa, Rattus norvegicus
MASTGNLKGPSFSQHFTMTVICIVLLQVLLQALTVAVTYMYFNNEVKQLQDNYSKIGLACFSKEDGDFWDSTDEGILNRPCLQVKRQLYQLIEEVTLRTFEKTISTVPEKQLSTPPLPRGRRPQRVAAHITGITRRSNLALIPISKDGKTLGQKIETWESSRRGHSFLNHVHLRNGELVIQEEGLYYIYSQTYYRFKEAKEASKTVSKDGGRIKQMVQYIYKYTSYPDPILLMKSARNSCWSREAEYGLYSIYQGGLFELKENDRIFVSVTNEHLMDLDHEASFFGA

SEQ ID NO: 47 - DNA, 864 bp, Rattus norvegicus
atggcttccaccgggaacctgaagggccccagcttcagtcagcacttcacgatgacggtgatctgcatagtgctcctgcaggtgctcctgcaggccttgactgtggctgtgacttacatgtacttcaacaacgaggtgaaacagctacaggacaattactccaaaatcggactagcttgcttctcaaaagaagatggggatttttgggactccactgacgaggggattttgaacagaccttgcttgcaggtcaagaggcaactgtatcagctcattgaagaggtgactttgagaacctttgagaaaaccatctctacagttccagaaaagcagctaagcactcctcccttgcccagaggtagaagaccccagagagtggcagctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggatcaagaagccagcttctttggagcctaa

SEQ ID NO: 48 - protein, 287 aa, Rattus norvegicus
MASTGNLKGPSFSQHFTMTVICIVLLQVLLQALTVAVTYMYFNNEVKQLQDNYSKIGLACFSKEDGDFWDSTDEGILNRPCLQVKRQLYQLIEEVTLRTFEKTISTVPEKQLSTPPLPRGRRPQRVAAHITGITRRSNLALIPISKDGKTLGQKIETWESSRRGHSFLNHVHLRNGELVIQEEGLYYIYSQTYYRFKEAKEASKTVSKDGGRIKQMVQYIYKYTSYPDPILLMKSARNSCWSREAEYGLYSIYQGGLFELKENDRIFVSVTNEHLMDLDQEASFFGA

SEQ ID NO: 49 - DNA, 876 bp, Mus musculus
atgccttcctcaggggccctgaaggacctcagcttcagtcagcacttcaggatgatggtgatttgcatagtgctcctgcaggtgctcctgcaggctgtgtctgtggctgtgacttacatgtacttcaccagcgagatgaagcagctgcaggacaattactccaaaattggactagcttgcttctcaaagacggatgaggatttctgggactccactgatggagagatcttgaacagaccctgcttgcaggttaagaggcaactgtatcagctcattgaagaggtgactttgagaacctttcaggacaccatttctacagttccagaaaagcagctaagtactcctcccttgcccagaggtggaagacctcagaaagtggcagctcacattactgggatcactcggagaagcaactcagctttaattccaatctccaaggatggaaagaccttaggccagaagattgagtcctgggagtcctctcggaaagggcattcatttctcaaccacgtgctctttaggaatggagagctggtcattgagcaggagggcctgtattacatctattcccaaacatacttccgatttcaggaagctaaagacgcttccaagatggtctcaaaggacaaggtgagaaccaaacagctggtgcagtacatctacaagtacaccagctatccggatcccatagtgctcatgaagagcgccagaaacagctgttggtccagagatgccgagtacggactgtactccatctatcagggaggactgttcgagctaaaaaaaaatgacaggatttttgtttctgtgacaaatgaacatttgatggacctggatcaagaagccagcttctttggagcctttttaattaactaa

SEQ ID NO: 50 - protein, 291 aa, Mus musculus
MPSSGALKDLSFSQHFRMMVICIVLLQVLLQAVSVAVTYMYFTSEMKQLQDNYSKIGLACFSKTDEDFWDSTDGEILNRPCLQVKRQLYQLIEEVTLRTFQDTISTVPEKQLSTPPLPRGGRPQKVAAHITGITRRSNSALIPISKDGKTLGQKIESWESSRKGHSFLNHVLFRNGELVIEQEGLYYIYSQTYFRFQEAKDASKMVSKDKVRTKQLVQYIYKYTSYPDPIVLMKSARNSCWSRDAEYGLYSIYQGGLFELKKNDRIFVSVTNEHLMDLDQEASFFGAFLIN

SEQ ID NO: 51 - DNA, 876 bp, Mus musculus
atgccttcctcaggggccctgaaggacctcagcttcagtcagcacttcaggatgatggtgatttgcatagtgctcctgcaggtgctcctgcaggctgtgtctgtggctgtgacttacatgtacttcaccaacgagatgaagcagctgcaggacaattactccaaaattggactagcttgcttctcaaagacggatgaggatttctgggactccactgatggagagatcttgaacagaccctgcttgcaggttaagaggcaactgtatcagctcattgaagaggtgactttgagaacctttcaggacaccatttctacagttccagaaaagcagctaagtactcctcccttgcccagaggtggaagacctcagaaagtggcagctcacattactgggatcactcggagaagcaactcagctttaattccaatctccaaggatggaaagaccttaggccagaagattgaatcctgggagtcctctcggaaagggcattcatttctcaaccacgtgctctttaggaatggagagctggtcatcgagcaggagggcctgtattacatctattcccaaacatacttccgatttcaggaagctgaagacgcttccaagatggtctcaaaggacaaggtgagaaccaaacagctggtgcagtacatctacaagtacaccagctatccggatcccatagtgctcatgaagagcgccagaaacagctgttggtccagagatgccgagtacggactgtactccatctatcagggaggattgttcgagctaaaaaaaaatgacaggatttttgtttctgtgacaaatgaacatttgatggacctggatcaagaagccagcttctttggagcctttttaattaactaa

SEQ ID NO: 52 - protein, 291 aa, Mus musculus
MPSSGALKDLSFSQHFRMMVICIVLLQVLLQAVSVAVTYMYFTNEMKQLQDNYSKIGLACFSKTDEDFWDSTDGEILNRPCLQVKRQLYQLIEEVTLRTFQDTISTVPEKQLSTPPLPRGGRPQKVAAHITGITRRSNSALIPISKDGKTLGQKIESWESSRKGHSFLNHVLFRNGELVIEQEGLYYIYSQTYFRFQEAEDASKMVSKDKVRTKQLVQYIYKYTSYPDPIVLMKSARNSCWSRDAEYGLYSIYQGGLFELKKNDRIFVSVTNEHLMDLDQEASFFGAFLIN

SEQ ID NO: 53 - DNA, 459 bp, Homo sapiens
gctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggg

SEQ ID NO: 54 - protein, 153 aa, Homo sapiens
AHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFG

SEQ ID NO: 55 - DNA, 459 bp, Artificial Sequence
  source: /note=“Description of Artificial Sequence Synthetic polynucleotide”
gctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggg

SEQ ID NO: 56 - protein, 153 aa, Artificial Sequence; identical to SEQ ID NO: 54
  source: /note=“Description of Artificial Sequence Synthetic polypeptide”

SEQ ID NO: 57 - DNA, 459 bp, Artificial Sequence
  source: /note=“Description of Artificial Sequence Synthetic polynucleotide”
gctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttaggg

SEQ ID NO: 58 - protein, 153 aa, Artificial Sequence
  source: /note=“Description of Artificial Sequence Synthetic polypeptide”
AHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFLG

SEQ ID NO: 59 - DNA, 459 bp, Homo sapiens; identical to SEQ ID NO: 53

SEQ ID NO: 60 - protein, 153 aa, Homo sapiens; identical to SEQ ID NO: 54

SEQ ID NO: 61 - DNA, 459 bp, Pan troglodytes
gctcacataactggaaccagaggaagaagcaacacattgtcttctccaaactccaagaatgaaaaggctctgggccacaaaataaactcctgggaatcatcaaggagtgggcattcattcctgagcaacttgcacttgaggaatggcgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagcgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggg

SEQ ID NO: 62 - protein, 153 aa, Pan troglodytes
AHITGTRGRSNTLSSPNSKNEKALGHKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFG

SEQ ID NO: 63 - DNA, 459 bp, Homo sapiens
  modified_base (3)..(3): a, c, t, g, unknown or other
gcncayathacnggnacnmgnggnmgnwsnaayacnytnwsnwsnccnaaywsnaaraaygaraargcnytnggnmgnaarathaaywsntgggarwsnwsnmgnwsnggncaywsnttyytnwsnaayytncayytnmgnaayggngarytngtnathcaygaraarggnttytaytayathtaywsncaracntayttymgnttycargargarathaargaraayacnaaraaygayaarcaratggtncartayathtayaartayacnwsntayccngayccnathytnytnatgaarwsngcnmgnaaywsntgytggwsnaargaygcngartayggnytntaywsnathtaycarggnggnathttygarytnaargaraaygaymgnathttygtnwsngtnacnaaygarcayytnathgayatggaycaygargcnwsnttyttyggn

SEQ ID NO: 64 - protein, 153 aa, Homo sapiens; identical to SEQ ID NO: 54

SEQ ID NO: 65 - DNA, 459 bp, Homo sapiens
  modified_base (3)..(3): a, c, t, g, unknown or other
gcncayathacnggnacnmgnggnmgnwsnaayacnytnwsnwsnccnaaywsnaaraaygaraargcnytnggnmgnaarathaaywsntgggarwsnwsnmgnwsnggncaywsnttyytnwsnaayytncayytnmgnaayggngarytngtnathcaygaraarggnttytaytayathtaywsncaracntayttymgnttycargargarathaargaraayacnaaraaygayaarcaratggtncartayathtayaartayacnwsntayccngcnccnathytnytnatgaarwsngcnmgnaaywsntgytggwsnaargaygcngartayggnytntaywsnathtaycarggnggnathttygarytnaargaraaygaymgnathttygtnwsngtnacnaaygarcayytnathgayatggaycaygargcnwsnttyttyggn

SEQ ID NO: 66 - protein, 153 aa, Homo sapiens
AHITGTRGRSNTLSSPNSKNEKALGRKINSWESSRSGHSFLSNLHLRNGELVIHEKGFYYIYSQTYFRFQEEIKENTKNDKQMVQYIYKYTSYPAPILLMKSARNSCWSKDAEYGLYSIYQGGIFELKENDRIFVSVTNEHLIDMDHEASFFG

SEQ ID NO: 67 - DNA, 459 bp, Macaca mulatta
gctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggcgaactggtcatccaagaaaaggggttttactacatctattcccaaacatactttcgatttcaggaggaaataaaagaaaacacaaagaacgacaaacaaatggtccaatatatttacaaatacacaagttatcctgaccctatactgctgatgaaaagcgctagaaatagttgttggtctaaagatgcagaatacggactctattccatctatcaagggggattatttgagcttaagaaagatgacagaatttttgtttctgtaacaaatgagcacttgatagacatggaccatgaagccagctttttcggg

SEQ ID NO: 68 - protein, 153 aa, Macaca mulatta
AHITGTRGRSNTLSSPNSKNEKALGRKINSWESSRSGHSFLSNLHLRNGELVIQEKGFYYIYSQTYFRFQEEIKENTKNDKQMVQYIYKYTSYPDPILLMKSARNSCWSKDAEYGLYSIYQGGLFELKKDDRIFVSVTNEHLIDMDHEASFFG

SEQ ID NO: 69 - DNA, 459 bp, Crassostrea ariakensis
gctcacataactgggaccagaggaagaagcaacacattgtcttctccaaactccaagaatgaaaaggctctgggccgcaaaataaactcctgggaatcatcaaggagtgggcattcattccagagcaacttgcacttgaggaatggtgaactggtcatccatgaaaaagggttttactacatctattcccaaacatactttcgatttcaggaggaaataaaagaaaacgcaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggg

SEQ ID NO: 70 - protein, 153 aa, Crassostrea ariakensis
AHITGTRGRSNTLSSPNSKNEKALGRKINSWESSRSGHSFQSNLHLRNGELVIHEKGFYYIYSQTYFRFQEEIKENAKNDKQMVQYIYKYTSYPDPILLMKSARNSCWSKDAEYGLYSIYQGGIFELKENDRIFVSVTNEHLIDMDHEASFFG

SEQ ID NO: 71 - DNA, 459 bp, Homo sapiens; identical to SEQ ID NO: 63
  modified_base (3)..(3): a, c, t, g, unknown or other

SEQ ID NO: 72 - protein, 158 aa, Homo sapiens
PQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFG

SEQ ID NO: 73 - DNA, 459 bp, Pongo abelii
  modified_base (67)..(75): a, c, t, g, unknown or other
gctcacataactgggaccagaggaagaagcaacacattgtcttctccaagtaagagaaacaacaaannnnnnnnncgcaaaataaactcctgggaatcatcaaggagtgggcattcattcctgagcaacttgcacttgaggaatggcgaactggtcatccatgaaaaagggttttactacatctattcccaaacatactttcgatttcaggaggaaataaaagaaaacacaaagaacgacaaacaaatggtccaatatatttacaaatacacaagttatcctgatcctatattgctgatgaaaagcgctagaaatagttgttggtctaaagatgcagaatatggactctattccatctatcaagggggaatatttgagcttaaggaaaatgacagaatttttgtttctgtaacaaatgagcacttgatagacatggaccatgaagccagttttttcggg

SEQ ID NO: 74 - protein, 158 aa, Pongo abelii
  MOD_RES (28)..(30): Any amino acid
PQRVAAHITGTRGRSNTLSSPSKRNNKXXXRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFG

SEQ ID NO: 75 - DNA, 459 bp, Callithrix jacchus
gctcacataactgggaccagaggaagtagcaacacgttgcctattccaaactccaagaatgaaaaggctctgggccgcaaaataaactcctgggaatcatcaaggagtggacattctttcctgagcaacttgcacttgaggaatggcgagctggtcatccatgaaaaagggctgtattacatctattgccaagtatactttcgatttcaggaggaaatccaagaaaacagaaagaacgacaaacaaatggtccagtatatttacaaatacacaagttatcctgaccccatactgctgatgaagagtgctagaaataattgttggtctaaagatgcagaatatggactctattccatctatcaagggggaatatttgagcttaaggaaaacgacagaatttttgtttctgtaacaaatgggcagttgatagacatggaccatgaagccagttttttcggg

SEQ ID NO: 76 - protein, 158 aa, Callithrix jacchus
PQRVAAHITGTRGSSNTLPIPNSKNEKALGRKINSWESSRSGHSFLSNLHLRNGELVIHEKGLYYIYCQVYFRFQEEIQENRKNDKQMVQYIYKYTSYPDPILLMKSARNNCWSKDAEYGLYSIYQGGIFELKENDRIFVSVTNGQLIDMDHEASFFG

SEQ ID NO: 77 - DNA, 471 bp, Felis catus
gctcacataactggaaccagtcggagaagaagcacattcccagttccaagctccaagaatgaaaaagctttgggtcagaaaataaactcctgggagtcatcaagaaaaggacattcattcttgaataatttgcacttgaggaatggtgagctggttattcatcagagggggttttattacatctattcccaaacatactttcgatttcaggaacctgaggaaattccaacaggacagaacagaaagagaaacaaacaaatggtccaatatatttacaaacacacgagttatccggaccctatactgctgatgaaaagtgctagaaatagttgttggtctaaagattctgaatatggactctattccatctatcaaggtgggatatttgagcttaaggaaaacgatagaatttttgtctctgtatctaacgagcaattgattgacatggaccaagaagccagttttttcggg

SEQ ID NO: 78 - protein, 162 aa, Felis catus
PQRVAAHITGTSRRRSTFPVPSSKNEKALGQKINSWESSRKGHSFLNNLHLRNGELVIHQRGFYYIYSQTYFRFQEPEEIPTGQNRKRNKQMVQYIYKHTSYPDPILLMKSARNSCWSKDSEYGLYSIYQGGIFELKENDRIFVSVSNEQLIDMDQEASFFG

SEQ ID NO: 79 - DNA, 483 bp, Ailuropoda melanoleuca
gctcacataactggaaccagtcggagaagaagcacgtttccagttccaagctccaagaatgaaaaagctttgggccagaaaataaactcctgggagtcatcaagaaaaggacattcattcttgagtaatttgcacttgaggaatggagagctggttatccatcaaagtgggttttattacatctattcccaaacatactttcgatttcaggaacctgaggaaacttcgggaccaatttcaaaggaacaaaacagaaagaaaaacaaacaaatggtacaatatatttacaaatacacaagttatcctgaccctatactgctgatgaaaagtgctagaaatagttgctggtctaaagattctgagtatggactctattccatctatcaaggtgggatatttgagcttaaggaaaatgatagaatttttgtctctgtaaataatgagcaattgattgacatggaccaagaagccagttttttcggg

SEQ ID NO: 80 - protein, 166 aa, Ailuropoda melanoleuca
LQRVAAHITGTSRRRSTFPVPSSKNEKALGQKINSWESSRKGHSFLSNLHLRNGELVIHQSGFYYIYSQTYFRFQEPEETSGPISKEQNRKKNKQMVQYIYKYTSYPDPILLMKSARNSCWSKDSEYGLYSIYQGGIFELKENDRIFVSVNNEQLIDMDQEASFFG

SEQ ID NO: 81 - DNA, 483 bp, Equus caballus
gctcacataactgggaccagtcggagaagaagcacagtctcaattccacgctccaagaatgaaaaagcactgggccagaaaataaacgcctgggagacatcaagaaaaggacattcgttcttgaataatttacacttgaggaatggagagctggttatccatcaaacagggttttattacatctattcccaaacatactttcgatttcaggaacctgaggaaattttgggaacagttgcaacagaagagaacagaaggaaaaataaacaaatggtacaatatatttacaaaagcacagactatcctgaccctatactgctgatgaaaagtgctagaaatagttgttggtctaaagattcagaatacggactctattccatctatcaaggtggaatatttgagcttaaggaaaatgacagaatttttgtctctgtaactaatgagcaattgattgacatggaccaagaagccagtttcttcggg

SEQ ID NO: 82 - protein, 166 aa, Equus caballus
LQRVAAHITGTSRRRSTVSIPRSKNEKALGQKINAWETSRKGHSFLNNLHLRNGELVIHQTGFYYIYSQTYFRFQEPEEILGTVATEENRRKNKQMVQYIYKSTDYPDPILLMKSARNSCWSKDSEYGLYSIYQGGIFELKENDRIFVSVTNEQLIDMDQEASFFG

SEQ ID NO: 83 - DNA, 483 bp, Ailuropoda melanoleuca; identical to SEQ ID NO: 79

SEQ ID NO: 84 - protein, 161 aa, Ailuropoda melanoleuca
AHITGTSRRRSTFPVPSSKNEKALGQKINSWESSRKGHSFLSNLHLRNGELVIHQSGFYYIYSQTYFRFQEPEETSGPISKEQNRKKNKQMVQYIYKYTSYPDPILLMKSARNSCWSKDSEYGLYSIYQGGIFELKENDRIFVSVNNEQLIDMDQEASFFG

SEQ ID NO: 85 - DNA, 486 bp, Bos taurus
gctgctcatataactggaagcaatcggaaaaaaagtacgttgccagttccaggctccaagaatgaaaaagctgtgggccataaaataaattcctgggagtcatcaagaaaaggacattcgttcttgaataatttgtacttaaggaatggagagctggttatccttcaaacaggattttattacatctattcccaaacatactttcgatttcaggaacctgaggaagttttgggaactgtttcaacagaagagaacagaaaaaaaatcaaacaaatggtacaatatatttacaaatacacaaactatcctgaccctatactgctgatgaaaagtgctagaaatagttgttggtctaaagattcagaatatggactctattccatctatcaaggaggaatatttgagcttaaggaaaatgatcgaatttttgtctctgtaactaatgaacgattggttgacctggaccaagaagccagttttttcgga

SEQ ID NO: 86 - protein, 161 aa, Bos taurus
AHITGSNRKKSTLPVPGSKNEKAVGHKINSWESSRKGHSFLNNLYLRNGELVILQTGFYYIYSQTYFRFQEPEEVLGTVSTEENRKKIKQMVQYIYKYTNYPDPILLMKSARNSCWSKDSEYGLYSIYQGGIFELKENDRIFVSVTNERLVDLDQEASFFG

SEQ ID NO: 87 - DNA, 483 bp, Sus scrofa
gctcacataactggaaccagtaggaaaagaagcacatttccatctctaagctccaaatatgaaaaagctttgggccagaaaataaactcctgggaatcatcaagaaaaggacattcattcttgaataattttcacttgaggaatggagagctggttatccatcaaacagggttttactacatctattcccaaacatactttcgatttcaggaacctgaggaaattttgggaacggtttctacagaagggaacagaaagaaaaacaggcaaatgatacagtatatttacaaatggacaagctatcctgaccctatactgctgatgaaaagtgctagaaatagttgttggtctaaagattcagaatatggactctattccatctatcaaggtggaatatttgagcttaaggaagatgaccgaatttttgtctctgttactaatgagcaactgattgacatggaccaagaagccagttttttcggg

SEQ ID NO: 88 - protein, 161 aa, Sus scrofa
AHITGTSRKRSTFPSLSSKYEKALGQKINSWESSRKGHSFLNNFHLRNGELVIHQTGFYYIYSQTYFRFQEPEEILGTVSTEGNRKKNRQMIQYIYKWTSYPDPILLMKSARNSCWSKDSEYGLYSIYQGGIFELKEDDRIFVSVTNEQLIDMDQEASFFG

SEQ ID NO: 89 - DNA, 468 bp, Canis lupis
gctcacataactggaaccagtcggagaagcatgtttccaattccaagctccaagaatgataaagctttgggccacaaaataaactcctgggattccacaagaaaaggacattcattcttgaataatttgcacttgaggaacggagagctggttatccatcaaagggggttttattacatctattcccaaacatactttcgatttcaggaacctgaggaaattccaacaggacagaacagaaagagaaacaaacaaatggtccaatatatttacaaacacacgagttatccggaccctatactgctgatgaaaagtgctagaaatagttgttggtctaaagattctgaatatggactctattccatctatcaaggtgggatatttgagcttaaggaaaacgatagaatttttgtctctgtatctaacgagcaattgattgacatggaccaagaagccagttttttcggg

SEQ ID NO: 90 - protein, 156 aa, Canis lupis
AHITGTSRRSMFPIPSSKNDKALGHKINSWDSTRKGHSFLNNLHLRNGELVIHQRGFYYIYSQTYFRFQEPEEIPTGQNRKRNKQMVQYIYKHTSYPDPILLMKSARNSCWSKDSEYGLYSIYQGGIFELKENDRIFVSVSNEQLIDMDQEASFFG

SEQ ID NO: 91 - DNA, 486 bp, Oryctolagus cuniculus
gctcacctaactgggaacagctggagaagctttatctcagtccctgctccaggctcccagagtggaaagaatttgggccagaaaataagctcctgggaatcatcaaggaaaggacattcattcctgaacaatttgcacctgaggaatggagagctggttatccatcaaacaggactttattacatctactcccaaacatactttcgatttcaggaacttgaagaaatttcaggaacaatttcaagagaagagatcaaaaagaggaacaaacaaatggtacaatatatttacaaatggacaagctaccctgaccctatacttctgatgaaaagtgctagaaatagttgttggtctaaggattcggaatatggactctattccatctatcaaggaggaatatttgagcttaaggaaaatgaccgaattttcgtctctgtaacgaatgagcagttgattgacatgaaccaagaatccagtttttttggg

SEQ ID NO: 92 - protein, 162 aa, Oryctolagus cuniculus
AHLTGNSWRSFISVPAPGSQSGKNLGQKISSWESSRKGHSFLNNLHLRNGELVIHQTGLYYIYSQTYFRFQELEEISGTISREEIKKRNKQMVQYIYKWTSYPDPILLMKSARNSCWSKDSEYGLYSIYQGGIFELKENDRIFVSVTNEQLIDMNQESSFFG

SEQ ID NO: 93 - DNA, 477 bp, Rattus norvegicus
gctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggatcaagaagccagtttctttgga

SEQ ID NO: 94 - protein, 159 aa, Rattus norvegicus
AHITGITRRSNLALIPISKDGKTLGQKIETWESSRRGHSFLNHVHLRNGELVIQEEGLYYIYSQTYYRFKEAKEASKTVSKDGGRIKQMVQYIYKYTSYPDPILLMKSARNSCWSREAEYGLYSIYQGGLFELKENDRIFVSVTNEHLMDLDQEASFFG

SEQ ID NO: 95 - DNA, 477 bp, Rattus norvegicus
gctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggaccatgaagccagcttctttgga

SEQ ID NO: 96 - protein, 159 aa, Rattus norvegicus
AHITGITRRSNLALIPISKDGKTLGQKIETWESSRRGHSFLNHVHLRNGELVIQEEGLYYIYSQTYYRFKEAKEASKTVSKDGGRIKQMVQYIYKYTSYPDPILLMKSARNSCWSREAEYGLYSIYQGGLFELKENDRIFVSVTNEHLMDLDHEASFFG

SEQ ID NO: 97 - DNA, 477 bp, Rattus norvegicus
gctcacattaccgggatcactcggagaagcaacttagccttaattccaatctccaaggatggaaagaccttgggccagaagatagaaacctgggagtcctctcggagagggcattcatttctcaaccatgtgcacttgagaaacggagagctggtgatccaggaggagggcctgtattacatctactcccaaacgtactaccggttcaaggaggctaaagaagcttccaagacagtctcgaaggacggagggaggatcaaacagatggtgcagtacatctacaaatacaccagctaccccgatcccatactgctgatgaagagtgccagaaatagctgctggtccagagaagctgagtacggactgtactccatctatcagggggggctgttcgagctcaaagaaaatgacaggatttttgtttccgtgacgaatgagcatttgatggacctggatcaagaagccagcttctttgga

SEQ ID NO: 98 - protein, 159 aa, Rattus norvegicus; identical to SEQ ID NO: 94

SEQ ID NO: 99 - DNA, 477 bp, Mus musculus
gctcacattactgggatcactcggagaagcaactcagctttaattccaatctccaaggatggaaagaccttaggccagaagattgagtcctgggagtcctctcggaaagggcattcatttctcaaccacgtgctctttaggaatggagagctggtcattgagcaggagggcctgtattacatctattcccaaacatacttccgatttcaggaagctaaagacgcttccaagatggtctcaaaggacaaggtgagaaccaaacagctggtgcagtacatctacaagtacaccagctatccggatcccatagtgctcatgaagagcgccagaaacagctgttggtccagagatgccgagtacggactgtactccatctatcagggaggactgttcgagctaaaaaaaaatgacaggatttttgtttctgtgacaaatgaacatttgatggacctggatcaagaagccagcttctttgga

SEQ ID NO: 100 - protein, 159 aa, Mus musculus
AHITGITRRSNSALIPISKDGKTLGQKIESWESSRKGHSFLNHVLFRNGELVIEQEGLYYIYSQTYFRFQEAKDASKMVSKDKVRTKQLVQYIYKYTSYPDPIVLMKSARNSCWSRDAEYGLYSIYQGGLFELKKNDRIFVSVTNEHLMDLDQEASFFG

SEQ ID NO: 101 - DNA, 477 bp, Mus musculus
gctcacattactgggatcactcggagaagcaactcagctttaattccaatctccaaggatggaaagaccttaggccagaagattgaatcctgggagtcctctcggaaagggcattcatttctcaaccacgtgctctttaggaatggagagctggtcatcgagcaggagggcctgtattacatctattcccaaacatacttccgatttcaggaagctgaagacgcttccaagatggtctcaaaggacaaggtgagaaccaaacagctggtgcagtacatctacaagtacaccagctatccggatcccatagtgctcatgaagagcgccagaaacagctgttggtccagagatgccgagtacggactgtactccatctatcagggaggattgttcgagctaaaaaaaaatgacaggatttttgtttctgtgacaaatgaacatttgatggacctggatcaagaagccagcttctttgga

SEQ ID NO: 102 - protein, 159 aa, Mus musculus
AHITGITRRSNSALIPISKDGKTLGQKIESWESSRKGHSFLNHVLFRNGELVIEQEGLYYIYSQTYFRFQEAEDASKMVSKDKVRTKQLVQYIYKYTSYPDPIVLMKSARNSCWSRDAEYGLYSIYQGGLFELKKNDRIFVSVTNEHLMDLDQEASFFG

SEQ ID NO: 103 - DNA, 30 bp, Unknown
  source: /note=“Description of Unknown Illustrative oligonucleotide”
gtgagagaaagaggtcctcagagagtagca

SEQ ID NO: 104 - protein, 10 aa, Unknown
  source: /note=“Description of Unknown Illustrative peptide”
VRERGPQRVA

SEQ ID NO: 105 - DNA, 15 bp, Unknown
  source: /note=“Description of Unknown Illustrative oligonucleotide”
ccncarmgngtngcn

SEQ ID NO: 106 - protein, 5 aa, Unknown
  source: /note=“Description of Unknown Illustrative peptide”
PQRVA

SEQ ID NO: 107 - DNA, 30 bp, Unknown
  source: /note=“Description of Unknown Illustrative oligonucleotide”
gtaaatgaaagaggtcttcagagagtagca

SEQ ID NO: 108 - protein, 10 aa, Unknown
  source: /note=“Description of Unknown Illustrative peptide”
VNERGLQRVA

SEQ ID NO: 109 - DNA, 30 bp, Unknown
  source: /note=“Description of Unknown Illustrative oligonucleotide”
gtaagagaaagaggtcttcagagagtagca

SEQ ID NO: 110 - protein, 10 aa, Unknown
  source: /note=“Description of Unknown Illustrative peptide”
VRERGLQRVA

SEQ ID NO: 111 - DNA, 30 bp, Unknown
  source: /note=“Description of Unknown Illustrative oligonucleotide”
gagagagaaaagggtcctaagagggtagct

SEQ ID NO: 112 - protein, 10 aa, Unknown
  source: /note=“Description of Unknown Illustrative peptide”
EREKGPKRVA

SEQ ID NO: 113 - DNA, 30 bp, Unknown
  source: /note=“Description of Unknown Illustrative oligonucleotide”
gaaagagaaaaaggtccacagagagtggct

SEQ ID NO: 114 - protein, 10 aa, Unknown
  source: /note=“Description of Unknown Illustrative peptide”
EREKGPQRVA

SEQ ID NO: 115 - DNA, 29 bp, Unknown
  source: /note=“Description of Unknown Illustrative oligonucleotide”
gtaagcgaccaggttctcagagagtagct

SEQ ID NO: 116 - protein, 10 aa, Unknown
  source: /note=“Description of Unknown Illustrative peptide”
VSDRGSQRVA

SEQ ID NO: 117 - DNA, 42 bp, Unknown
  source: /note=“Description of Unknown Illustrative oligonucleotide”
gtaagagaaaaagaaagagaaagagggcctcagagagtagca

SEQ ID NO: 118 - protein, 14 aa, Unknown
  source: /note=“Description of Unknown Illustrative peptide”
VREKERERGPQRVA

SEQ ID NO: 119 - DNA, 30 bp, Unknown
  source: /note=“Description of Unknown Illustrative oligonucleotide”
cccagaggtagaagaccccagagagtggca

SEQ ID NO: 120 - protein, 10 aa, Unknown
  source: /note=“Description of Unknown Illustrative peptide”
PRGRRPQRVA

SEQ ID NO: 121 - DNA, 30 bp, Unknown
  source: /note=“Description of Unknown Illustrative oligonucleotide”
cccagaggtggaagaccccagagagtggca

SEQ ID NO: 122 - protein, 10 aa, Unknown
  source: /note=“Description of Unknown Illustrative peptide”
PRGGRPQRVA

SEQ ID NO: 123 - DNA, 15 bp, Unknown
  source: /note=“Description of Unknown Illustrative oligonucleotide”
gcctttttagttggc

SEQ ID NO: 124 - protein, 5 aa, Unknown
  source: /note=“Description of Unknown Illustrative peptide”
AFLVG

SEQ ID NO: 125 - DNA, 18 bp, Unknown
  source: /note=“Description of Unknown Illustrative oligonucleotide”
gcctttttagttggcttg

SEQ ID NO: 126 - protein, 6 aa, Unknown
  source: /note=“Description of Unknown Illustrative peptide”
AFLVGL

SEQ ID NO: 127 - DNA, 6 bp, Unknown
  source: /note=“Description of Unknown Illustrative oligonucleotide”
gccttt

SEQ ID NO: 128 - protein, 2 aa, Unknown
  source: /note=“Description of Unknown Illustrative peptide”
AF

SEQ ID NO: 129 - DNA, 15 bp, Unknown
  source: /note=“Description of Unknown Illustrative oligonucleotide”
gcctttttaatcggc

SEQ ID NO: 130 - protein, 5 aa, Unknown
  source: /note=“Description of Unknown Illustrative peptide”
AFLIG

SEQ ID NO: 131 - DNA, 15 bp, Unknown
  source: /note=“Description of Unknown Illustrative oligonucleotide”
gcctttttaattaac

SEQ ID NO: 132 - protein, 5 aa, Unknown
  source: /note=“Description of Unknown Illustrative peptide”
AFLIN

SEQ ID NO: 133 - protein, 19 aa, Artificial Sequence
  source: /note=“Description of Artificial Sequence Synthetic peptide”
MGTDTLLWVLLLWVPGSTG

SEQ ID NO: 134 - protein, 32 aa, Artificial Sequence
  source: /note=“Description of Artificial Sequence Synthetic polypeptide”
MARRLWILSLLAVTLTVALAAPSQKSKRRTSS

SEQ ID NO: 135 - protein, 26 aa, Artificial Sequence
  source: /note=“Description of Artificial Sequence Synthetic peptide”
MATGSRTSLLLAFGLLCLPWLQEGSA

SEQ ID NO: 136 - protein, 16 aa, Artificial Sequence
  source: /note=“Description of Artificial Sequence Synthetic peptide”
WDCLDNRIGRRQCVRL

SEQ ID NO: 137 - protein, 16 aa, Artificial Sequence
  source: /note=“Description of Artificial Sequence Synthetic peptide”
WDCLDNRIGRRQCVKL

SEQ ID NO: 138 - protein, 19 aa, Artificial Sequence
  source: /note=“Description of Artificial Sequence Synthetic peptide”
GGSWDCLDNRIGRRQCVKL

SEQ ID NO: 139 - protein, 16 aa, Artificial Sequence
  source: /note=“Description of Artificial Sequence Synthetic peptide”
WDCLDNRIGRRQCVKL

SEQ ID NO: 140 - protein, 14 aa, Artificial Sequence
  source: /note=“Description of Artificial Sequence Synthetic peptide”
WDCLDRPGRRQCVK

SEQ ID NO: 141 - protein, 16 aa, Artificial Sequence
  source: /note=“Description of Artificial Sequence Synthetic peptide”
WDCLDNKIGRRQCVRL

SEQ ID NO: 142 - protein, 12 aa, Artificial Sequence
  source: /note=“Description of Artificial Sequence Synthetic peptide”
CLDNRIGRRQCV

SEQ ID NO: 143 - protein, 15 aa, Artificial Sequence
  source: /note=“Description of Artificial Sequence Synthetic peptide”
DCLDNRIGRRQCVKL

SEQ ID NO: 144 - protein, 16 aa, Artificial Sequence
  source: /note=“Description of Artificial Sequence Synthetic peptide”
WDCLDNRIGKRQCVRL

SEQ ID NO: 145 - protein, 16 aa, Artificial Sequence
  source: /note=“Description of Artificial Sequence Synthetic peptide”
WDCLDNRIGRRQCVRL

SEQ ID NO: 146 - protein, 15 aa, Artificial Sequence
  source: /note=“Description of Artificial Sequence Synthetic peptide”
WDCLDNRIGRRQCVK

SEQ ID NO: 147 - protein, 18 aa, Artificial Sequence
  source: /note=“Description of Artificial Sequence Synthetic peptide”
WDCLVDRPGRRQCVRLEK

SEQ ID NO: 148 - protein, 19 aa, Artificial Sequence
  source: /note=“Description of Artificial Sequence Synthetic peptide”
WDCLVDRPGRRQCVRLERK

SEQ ID NO: 149 - protein, 18 aa, Artificial Sequence
  source: /note=“Description of Artificial Sequence Synthetic peptide”
WDCLVDRPGRRQCVKLER

SEQ ID NO: 150 - protein, 20 aa, Artificial Sequence
  source: /note=“Description of Artificial Sequence Synthetic peptide”
GGGSWDCLDNRIGRRQCVKL

SEQ ID NO: 151 - protein, 16 aa, Artificial Sequence
  source: /note=“Description of Artificial Sequence Synthetic peptide”
CWDLDNRIGRRQVCKL

SEQ ID NO: 152 - protein, 20 aa, Artificial Sequence
  source: /note=“Description of Artificial Sequence Synthetic peptide”
GGGSWDCLDNRIGRRQCVKL

SEQ ID NO: 153 - protein, 16 aa, Artificial Sequence
  source: /note=“Description of Artificial Sequence Synthetic peptide”
WDCLDNRIGRRQCVRL

SEQ ID NO: 154 - protein, 16 aa, Artificial Sequence
  source: /note=“Description of Artificial Sequence Synthetic peptide”
WDCLDNRIGRRQCVKL

SEQ ID NO: 155 - protein, 19 aa, Artificial Sequence
  source: /note=“Description of Artificial Sequence Synthetic peptide”
GGSWDCLDNRIGRRQCVKL

SEQ ID NO: 156 - protein, 16 aa, Artificial Sequence
  source: /note=“Description of Artificial Sequence Synthetic peptide”
WDCLDNRIGRRQCVKL

SEQ ID NO: 157 - protein, 14 aa, Artificial Sequence
  source: /note=“Description of Artificial Sequence Synthetic peptide”
WDCLDRPGRRQCVK

SEQ ID NO: 158 - protein, 16 aa, Artificial Sequence
  source: /note=“Description of Artificial Sequence Synthetic peptide”
WDCLDNKIGRRQCVRL

SEQ ID NO: 159 - protein, 12 aa, Artificial Sequence
  source: /note=“Description of Artificial Sequence Synthetic peptide”
CLDNRIGRRQCV

SEQ ID NO: 160 - protein, 15 aa, Artificial Sequence
  source: /note=“Description of Artificial Sequence Synthetic peptide”
DCLDNRIGRRQCVKL

SEQ ID NO: 161 - protein, 16 aa, Artificial Sequence
  source: /note=“Description of Artificial Sequence Synthetic peptide”
WDCLDNRIGKRQCVRL

SEQ ID NO: 162 - protein, 16 aa, Artificial Sequence
  source: /note=“Description of Artificial Sequence Synthetic peptide”
WDCLDNRIGRRQCVRL

SEQ ID NO: 163 - protein, 15 aa, Artificial Sequence
  source: /note=“Description of Artificial Sequence Synthetic peptide”
WDCLDNRIGRRQCVK

SEQ ID NO: 164 - protein, 18 aa, Artificial Sequence
  source: /note=“Description of Artificial Sequence Synthetic peptide”
WDCLVDRPGRRQCVRLEK

SEQ ID NO: 165 - protein, 19 aa, Artificial Sequence
  source: /note=“Description of Artificial Sequence Synthetic peptide”
WDCLVDRPGRRQCVRLERK

SEQ ID NO: 166 - protein, 18 aa, Artificial Sequence
  source: /note=“Description of Artificial Sequence Synthetic peptide”
WDCLVDRPGRRQCVKLER

SEQ ID NO: 167 - protein, 20 aa, Artificial Sequence
  source: /note=“Description of Artificial Sequence Synthetic peptide”
GGGSWDCLDNRIGRRQCVKL

SEQ ID NO: 168 - protein, 16 aa, Artificial Sequence
  source: /note=“Description of Artificial Sequence Synthetic peptide”
CWDLDNRIGRRQVCKL

SEQ ID NO: 169 - protein, 58 aa, Homo sapiens
NTGLLESQLSRHDQMLSVHDIRLADMDLRFQVLETASYNGVLIWKIRDYKRRKQEAVM

SEQ ID NO: 170 - protein, 21 aa, Homo sapiens
AASERKALQTEMARIKKWLTF

SEQ ID NO: 171 - protein, 37 aa, Homo sapiens
FDMSCRSRLATLNEKLTALERRIEYIEARVTKGETLT

SEQ ID NO: 172 - protein, 46 aa, Homo sapiens
ADIYKADFQAERQAREKLAEKKELLQEQLEQLQREYSKLKASCQES

SEQ ID NO: 173 - protein, 71 aa, Homo sapiens
LTGSAQNIEFRTGSLGKIKLNDEDLSECLHQIQKNKEDIIELKGSAIGLPIYQLNSKLVDLERKFQGLQQT

SEQ ID NO: 174 - protein, 28 aa, Homo sapiens
LRGLRTIVTTLQDSIRKVTEENKELANE

SEQ ID NO: 175 - protein, 27 aa, Homo sapiens
VASLRQQVEALQGQVQHLQAAFSQYKK

SEQ ID NO: 176 - protein, 27 aa, Rattus sp.
SAALRQQMEALNGKLQRLEAAFSRYKK

SEQ ID NO: 177 - protein, 27 aa, Bos sp.
VNALKQRVTILDGHLRRFQNAFSQYKK

SEQ ID NO: 178 - protein, 27 aa, Bos sp.
VDTLRQRMRNLEGEVQRLQNIVTQYRK

SEQ ID NO: 179 - protein, 64 aa, Homo sapiens
GSPGLKGDKGIPGDKGAKGESGLPDVASLRQQVEALQGQVQHLQAAFSQYKKVELFPGGIPHRD

SEQ ID NO: 180 - protein, 8 aa, Artificial Sequence
  source: /note=“Description of Artificial Sequence Synthetic peptide”
GWLEGAGW

SEQ ID NO: 181 - protein, 10 aa, Artificial Sequence
  source: /note=“Description of Artificial Sequence Synthetic peptide”
DGGWHWRWEN

SEQ ID NO: 182 - protein, 8 aa, Artificial Sequence
  source: /note=“Description of Artificial Sequence Synthetic peptide”
GWLEGVGW

SEQ ID NO: 183 - protein, 10 aa, Artificial Sequence
  source: /note=“Description of Artificial Sequence Synthetic peptide”
DGGEHWGWEN

SEQ ID NO: 184 - protein, 8 aa, Artificial Sequence
  source: /note=“Description of Artificial Sequence Synthetic peptide”
GYLAGVGW

SEQ ID NO: 185 - protein, 10 aa, Artificial Sequence
  source: /note=“Description of Artificial Sequence Synthetic peptide”
DGGRGFRWEN

SEQ ID NO: 186 - protein, 8 aa, Artificial Sequence
  source: /note=“Description of Artificial Sequence Synthetic peptide”
GWLEGYGW

SEQ ID NO: 187 - protein, 10 aa, Artificial Sequence
  source: /note=“Description of Artificial Sequence Synthetic peptide”
DGGTWWEWEN

SEQ ID NO: 188 - protein, 8 aa, Artificial Sequence
  source: /note=“Description of Artificial Sequence Synthetic peptide”
GYLEGYGW

SEQ ID NO: 189 - protein, 10 aa, Artificial Sequence
  source: /note=“Description of Artificial Sequence Synthetic peptide”
DGGATIAWEN

SEQ ID NO: 190 - protein, 8 aa, Artificial Sequence
  source: /note=“Description of Artificial Sequence Synthetic peptide”
GWLQGVGW

SEQ ID NO: 191 - protein, 10 aa, Artificial Sequence
  source: /note=“Description of Artificial Sequence Synthetic peptide”
DGGRGWPWEN

SEQ ID NO: 192 - protein, 8 aa, Artificial Sequence
  source: /note=“Description of Artificial Sequence Synthetic peptide”
GYLAGYGW

SEQ ID NO: 193 - protein, 10 aa, Artificial Sequence
  source: /note=“Description of Artificial Sequence Synthetic peptide”
DGGPSIWREN

SEQ ID NO: 194 - protein, 8 aa, Artificial Sequence
  source: /note=“Description of Artificial Sequence Synthetic peptide”
GYIEGTGW

SEQ ID NO: 195 - protein, 10 aa, Artificial Sequence
  source: /note=“Description of Artificial Sequence Synthetic peptide”
DGGSNWAWEN

SEQ ID NO: 196 - protein, 8 aa, Artificial Sequence
  source: /note=“Description of Artificial Sequence Synthetic peptide”
GYMSGYGW

SEQ ID NO: 197 - protein, 10 aa, Artificial Sequence
  source: /note=“Description of Artificial Sequence Synthetic peptide”
DGGMMARWEN

SEQ ID NO: 198 - protein, 8 aa, Artificial Sequence
  source: /note=“Description of Artificial Sequence Synthetic peptide”
GFMVGRGW

SEQ ID NO: 199 - protein, 10 aa, Artificial Sequence
  source: /note=“Description of Artificial Sequence Synthetic peptide”
DGGSMWPWEN

SEQ ID NO: 200 - protein, 8 aa, Artificial Sequence
  source: /note=“Description of Artificial Sequence Synthetic peptide”
MVTRPPYW

SEQ ID NO: 201 - protein, 10 aa, Artificial Sequence
  source: /note=“Description of Artificial Sequence Synthetic peptide”
DGGWVMSFEN

SEQ ID NO: 202 - protein, 8 aa, Artificial Sequence
  source: /note=“Description of Artificial Sequence Synthetic peptide”
PFRVPQWW

SEQ ID NO: 203 - protein, 10 aa, Artificial Sequence
  source: /note=“Description of Artificial Sequence Synthetic peptide”
DGGYGPVQEN

SEQ ID NO: 204 - protein, 8 aa, Artificial Sequence
  source: /note=“Description of Artificial Sequence Synthetic peptide”
GWLEGAGW

SEQ ID NO: 205 - protein, 10 aa, Artificial Sequence
  source: /note=“Description of Artificial Sequence Synthetic peptide”
DGGWQWRWEN

SEQ ID NO: 206 - protein, 8 aa, Artificial Sequence
  source: /note=“Description of Artificial Sequence Synthetic peptide”
GYLDGVGW

SEQ ID NO: 207 - protein, 10 aa, Artificial Sequence
  source: /note=“Description of Artificial Sequence Synthetic peptide”
DGGQGCRWEN

SEQ ID NO: 208 - protein, 8 aa, Artificial Sequence
  source: /note=“Description of Artificial Sequence Synthetic peptide”
VLRLAWSW

SEQ ID NO: 209 - protein, 10 aa, Artificial Sequence
  source: /note=“Description of Artificial Sequence Synthetic peptide”
DGGKRNGCEN

SEQ ID NO: 210 - protein, 8 aa, Artificial Sequence
  source: /note=“Description of Artificial Sequence Synthetic peptide”
WLSLFSPW

SEQ ID NO: 211 - protein, 10 aa, Artificial Sequence
  source: /note=“Description of Artificial Sequence Synthetic peptide”
DGGRGVRGEN

SEQ ID NO: 212 - protein, 8 aa, Artificial Sequence
  source: /note=“Description of Artificial Sequence Synthetic peptide”
GWMAGVGW

SEQ ID NO: 213 - protein, 10 aa, Artificial Sequence
  source: /note=“Description of Artificial Sequence Synthetic peptide”
DGGRRLPWEN

SEQ ID NO: 214 - protein, 8 aa, Artificial Sequence
  source: /note=“Description of Artificial Sequence Synthetic peptide”
SYRLHYGW

SEQ ID NO: 215 - protein, 10 aa, Artificial Sequence
  source: /note=“Description of Artificial Sequence Synthetic peptide”
DGGRRWLGEN

SEQ ID NO: 216 - protein, 8 aa, Artificial Sequence
  source: /note=“Description of Artificial Sequence Synthetic peptide”
IWPLRFRW

SEQ ID NO: 217 - protein, 10 aa, Artificial Sequence
  source: /note=“Description of Artificial Sequence Synthetic peptide”
DGGFVTRKEN

SEQ ID NO: 218 - protein, 8 aa, Artificial Sequence
  source: /note=“Description of Artificial Sequence Synthetic peptide”
WQLYYRYW

SEQ ID NO: 219 - protein, 10 aa, Artificial Sequence
  source: /note=“Description of Artificial Sequence Synthetic peptide”
DGGVGCMVEN

SEQ ID NO: 220 - protein, 8 aa, Artificial Sequence
  source: /note=“Description of Artificial Sequence Synthetic peptide”
RCLQGVGW

SEQ ID NO: 221 - protein, 10 aa, Artificial Sequence
  source: /note=“Description of Artificial Sequence Synthetic peptide”
DGGRGWPWEN

SEQ ID NO: 222 - protein, 8 aa, Artificial Sequence
  source: /note=“Description of Artificial Sequence Synthetic peptide”
GCTQGQGW

SEQ ID NO: 223 - protein, 10 aa, Artificial Sequence
  source: /note=“Description of Artificial Sequence Synthetic peptide”
DGGKKWKWEN

SEQ ID NO: 224 - protein, 8 aa, Artificial Sequence
  source: /note=“Description of Artificial Sequence Synthetic peptide”
GFLQGNGW

SEQ ID NO: 225 - protein, 10 aa, Artificial Sequence
  source: /note=“Description of Artificial Sequence Synthetic peptide”
DGGMWDRWEN

SEQ ID NO: 226 - protein, 8 aa, Artificial Sequence
  source: /note=“Description of Artificial Sequence Synthetic peptide”
GVLQRGGW

SEQ ID NO: 227 - protein, 10 aa, Artificial Sequence
  source: /note=“Description of Artificial Sequence Synthetic peptide”
DGGPGGEREN

SEQ ID NO: 228 - protein, 9 aa, Artificial Sequence
  source: /note=“Description of Artificial Sequence Synthetic peptide”
PFRVLQQWW

SEQ ID NO: 229 - protein, 11 aa, Artificial Sequence
  source: /note=“Description of Artificial Sequence Synthetic peptide”
DGGCGPVQQEN

SEQ ID NO: 230 - protein, 9 aa, Artificial Sequence
  source: /note=“Description of Artificial Sequence Synthetic peptide”
PFRGPQQWW

SEQ ID NO: 231 - protein, 10 aa, Artificial Sequence
  source: /note=“Description of Artificial Sequence Synthetic peptide”
DGGYGPVGEN

SEQ ID NO: 232 - protein, 9 aa, Artificial Sequence
  source: /note=“Description of Artificial Sequence Synthetic peptide”
ARFAMWQQW

SEQ ID NO: 233 - protein, 10 aa, Artificial Sequence
  source: /note=“Description of Artificial Sequence Synthetic peptide”
DGGRAGVGEN

SEQ ID NO: 234 - protein, 8 aa, Artificial Sequence
  source: /note=“Description of Artificial Sequence Synthetic peptide”
GWLQGYGW

SEQ ID NO: 235 - protein, 11 aa, Artificial Sequence
  source: /note=“Description of Artificial Sequence Synthetic peptide”
DGGQQIGWGEN

SEQ ID NO: 236 - protein, 8 aa, Artificial Sequence
  source: /note=“Description of Artificial Sequence Synthetic peptide”
AWRSWLNW

SEQ ID NO: 237 - protein, 11 aa, Artificial Sequence
  source: /note=“Description of Artificial Sequence Synthetic peptide”
DGGREQQRREN

SEQ ID NO: 238 - protein, 8 aa, Artificial Sequence
  source: /note=“Description of Artificial Sequence Synthetic peptide”
GWLEGVGW

SEQ ID NO: 239 - protein, 10 aa, Artificial Sequence
  source: /note=“Description of Artificial Sequence Synthetic peptide”
DGGWPFSNEN

SEQ ID NO: 240 - protein, 8 aa, Artificial Sequence
  source: /note=“Description of Artificial Sequence Synthetic peptide”
GWLMGTGW

SEQ ID NO: 241 - protein, 10 aa, Artificial Sequence
  source: /note=“Description of Artificial Sequence Synthetic peptide”
DGGWWNRWEN

SEQ ID NO: 242 - protein, 8 aa, Artificial Sequence
  source: /note=“Description of Artificial Sequence Synthetic peptide”
VRRMGFHW

SEQ ID NO: 243 - protein, 10 aa, Artificial Sequence
  source: /note=“Description of Artificial Sequence Synthetic peptide”
DGGRVAVGEN

SEQ ID NO: 244 - protein, 8 aa, Artificial Sequence
  source: /note=“Description of Artificial Sequence Synthetic peptide”
RYHVQALW

SEQ ID NO: 245 - protein, 10 aa, Artificial Sequence
  source: /note=“Description of Artificial Sequence Synthetic peptide”
DGGRVRPREN

SEQ ID NO: 246 - protein, 8 aa, Artificial Sequence
  source: /note=“Description of Artificial Sequence Synthetic peptide”
IQCSPPLW

SEQ ID NO: 247 - protein, 10 aa, Artificial Sequence
  source: /note=“Description of Artificial Sequence Synthetic peptide”
DGGAVQQQEN

SEQ ID NO: 248 - protein, 8 aa, Artificial Sequence
  source: /note=“Description of Artificial Sequence Synthetic peptide”
GLARQQGW

SEQ ID NO: 249 - protein, 10 aa, Artificial Sequence
  source: /note=“Description of Artificial Sequence Synthetic peptide”
DGGKGRPREN

SEQ ID NO: 250 - protein, 8 aa, Artificial Sequence
  source: /note=“Description of Artificial Sequence Synthetic peptide”
GWLSGVGW

SEQ ID NO: 251 - protein, 10 aa, Artificial Sequence
  source: /note=“Description of Artificial Sequence Synthetic peptide”
DGGWAHAWEN

SEQ ID NO: 252 - protein, 8 aa, Artificial Sequence
  source: /note=“Description of Artificial Sequence Synthetic peptide”
GWLEGVGW

SEQ ID NO: 253 - protein, 10 aa, Artificial Sequence
  source: /note=“Description of Artificial Sequence Synthetic peptide”
DGGGGVRWEN

SEQ ID NO: 254 - protein, 8 aa, Artificial Sequence
  source: /note=“Description of Artificial Sequence Synthetic peptide”
GWLSGYGW

SEQ ID NO: 255 - protein, 10 aa, Artificial Sequence
  source: /note=“Description of Artificial Sequence Synthetic peptide”
DGGRVWSWEN

SEQ ID NO: 256 - protein, 8 aa, Artificial Sequence
  source: /note=“Description of Artificial Sequence Synthetic peptide”
GLLSDWWW

SEQ ID NO: 257 - protein, 10 aa, Artificial Sequence
  source: /note=“Description of Artificial Sequence Synthetic peptide”
DGGGNQSREN

SEQ ID NO: 258 - protein, 8 aa, Artificial Sequence
  source: /note=“Description of Artificial Sequence Synthetic peptide”
QWVAFWSW

SEQ ID NO: 259 - protein, 10 aa, Artificial Sequence
  source: /note=“Description of Artificial Sequence Synthetic peptide”
DGGSAVSGEN

SEQ ID NO: 260 - protein, 8 aa, Artificial Sequence
  source: /note=“Description of Artificial Sequence Synthetic peptide”
PYTSWGLW

SEQ ID NO: 261 - protein, 10 aa, Artificial Sequence
  source: /note=“Description of Artificial Sequence Synthetic peptide”
DGGVGGRGEN

SEQ ID NO: 262 - protein, 8 aa, Artificial Sequence
  source: /note=“Description of Artificial Sequence Synthetic peptide”
VARWLLKW

SEQ ID NO: 263 - protein, 10 aa, Artificial Sequence
  source: /note=“Description of Artificial Sequence Synthetic peptide”
DGGMCKPCEN

SEQ ID NO: 264 - protein, 8 aa, Artificial Sequence
  source: /note=“Description of Artificial Sequence Synthetic peptide”
GFLAGVGW

SEQ ID NO: 265 - protein, 10 aa, Artificial Sequence
  source: /note=“Description of Artificial Sequence Synthetic peptide”
DGGWWTRWEN

SEQ ID NO: 266 - protein, 8 aa, Artificial Sequence
  source: /note=“Description of Artificial Sequence Synthetic peptide”
GYLQGSGW

SEQ ID NO: 267 - protein, 10 aa, Artificial Sequence
  source: /note=“Description of Artificial Sequence Synthetic peptide”
DGGWKTRWEN

SEQ ID NO: 268 - protein, 8 aa, Artificial Sequence
  source: /note=“Description of Artificial Sequence Synthetic peptide”
VRHWLQLW

SEQ ID NO: 269 - protein, 10 aa, Artificial Sequence
  source: /note=“Description of Artificial Sequence Synthetic peptide”
DGGGWWKGEN

SEQ ID NO: 270 - protein, 8 aa, Artificial Sequence
  source: /note=“Description of Artificial Sequence Synthetic peptide”
RATLRPRW

SEQ ID NO: 271 - protein, 9 aa, Artificial Sequence
  source: /note=“Description of Artificial Sequence Synthetic peptide”
DGGXXXXKN

SEQ ID NO: 272 - protein, 8 aa, Artificial Sequence
  source: /note=“Description of Artificial Sequence Synthetic peptide”
RAMLRSRW

SEQ ID NO: 273 - protein, 10 aa, Artificial Sequence
  source: /note=“Description of Artificial Sequence Synthetic peptide”
DGGRWFQGKN

SEQ ID NO: 274 - protein, 8 aa, Artificial Sequence
  source: /note=“Description of Artificial Sequence Synthetic peptide”
RALFRPRW

SEQ ID NO: 275 - protein, 10 aa, Artificial Sequence
  source: /note=“Description of Artificial Sequence Synthetic peptide”
DGGPWYLKEN

SEQ ID NO: 276 - protein, 8 aa, Artificial Sequence
  source: /note=“Description of Artificial Sequence Synthetic peptide”
RAVLRPRW

SEQ ID NO: 277 - protein, 10 aa, Artificial Sequence
  source: /note=“Description of Artificial Sequence Synthetic peptide”
DGGWVLGGKN

SEQ ID NO: 278 - protein, 8 aa, Artificial Sequence
  source: /note=“Description of Artificial Sequence Synthetic peptide”
RAWLRPRW

SEQ ID NO: 279 - protein, 10 aa, Artificial Sequence
  source: /note=“Description of Artificial Sequence Synthetic peptide”
DGGTLVSGEN

SEQ ID NO: 280 - protein, 8 aa, Artificial Sequence
  source: /note=“Description of Artificial Sequence Synthetic peptide”
RVIRRSMW

SEQ ID NO: 281 - protein, 10 aa, Artificial Sequence
  source: /note=“Description of Artificial Sequence Synthetic peptide”
DGGQKWMAEN

SEQ ID NO: 282 - protein, 8 aa, Artificial Sequence
  source: /note=“Description of Artificial Sequence Synthetic peptide”
RVLQRPVW

SEQ ID NO: 283 - protein, 10 aa, Artificial Sequence
  source: /note=“Description of Artificial Sequence Synthetic peptide”
DGGMVWSMEN

SEQ ID NO: 284 - protein, 8 aa, Artificial Sequence
  source: /note=“Description of Artificial Sequence Synthetic peptide”
RVQLRPRW

SEQ ID NO: 285 - protein, 10 aa, Artificial Sequence
  source: /note=“Description of Artificial Sequence Synthetic peptide”
EGGFRRHAKN

SEQ ID NO: 286 - protein, 8 aa, Artificial Sequence
  source: /note=“Description of Artificial Sequence Synthetic peptide”
RVVRLSEW

SEQ ID NO: 287 - protein, 10 aa, Artificial Sequence
  source: /note=“Description of Artificial Sequence Synthetic peptide”
DGGMLWAMEN

SEQ ID NO: 288 - protein, 8 aa, Artificial Sequence
  source: /note=“Description of Artificial Sequence Synthetic peptide”
RVISAPVW

SEQ ID NO: 289 - protein, 10 aa, Artificial Sequence
  source: /note=“Description of Artificial Sequence Synthetic peptide”
DGGQQWAMEN

SEQ ID NO: 290 - protein, 8 aa, Artificial Sequence
  source: /note=“Description of Artificial Sequence Synthetic peptide”
RVLRRPQW

SEQ ID NO: 291 - protein, 10 aa, Artificial Sequence
  source: /note=“Description of Artificial Sequence Synthetic peptide”
NGGDWRIPEN

SEQ ID NO: 292 - protein, 8 aa, Artificial Sequence
  source: /note=“Description of Artificial Sequence Synthetic peptide”
RVMMRPRW

SEQ ID NO: 293 - protein, 10 aa, Artificial Sequence
  source: /note=“Description of Artificial Sequence Synthetic peptide”
DGGMWGAMEN

SEQ ID NO: 294 - protein, 8 aa, Artificial Sequence
  source: /note=“Description of Artificial Sequence Synthetic peptide”
RVMRRVLW

SEQ ID NO: 295 - protein, 10 aa, Artificial Sequence
  source: /note=“Description of Artificial Sequence Synthetic peptide”
DGGRRETMKN

SEQ ID NO: 296 - protein, 8 aa, Artificial Sequence
  source: /note=“Description of Artificial Sequence Synthetic peptide”
RVMRRPLW

SEQ ID NO: 297 - protein, 10 aa, Artificial Sequence
  source: /note=“Description of Artificial Sequence Synthetic peptide”
DGGRGQQWEN

SEQ ID NO: 298 - protein, 8 aa, Artificial Sequence
  source: /note=“Description of Artificial Sequence Synthetic peptide”
RVMRRREW

SEQ ID NO: 299 - protein, 10 aa, Artificial Sequence
  source: /note=“Description of Artificial Sequence Synthetic peptide”
DGAQLMALEN

SEQ ID NO: 300 - protein, 8 aa, Artificial Sequence
  source: /note=“Description of Artificial Sequence Synthetic peptide”
RVWRRSLW

SEQ ID NO: 301 - protein, 10 aa, Artificial Sequence
  source: /note=“Description of Artificial Sequence Synthetic peptide”
DGGHLVKQKN

SEQ ID NO: 302 - protein, 8 aa, Artificial Sequence
  source: /note=“Description of Artificial Sequence Synthetic peptide”
KRRWYGGW

SEQ ID NO: 303 - protein, 10 aa, Artificial Sequence
  source: /note=“Description of Artificial Sequence Synthetic peptide”
DGGVNTVREN

SEQ ID NO: 304 - protein, 8 aa, Artificial Sequence
  source: /note=“Description of Artificial Sequence Synthetic peptide”
KRVWYRGW

SEQ ID NO: 305 - protein, 10 aa, Artificial Sequence
  source: /note=“Description of Artificial Sequence Synthetic peptide”
DGGMRRRREN

SEQ ID NO: 306 - protein, 8 aa, Artificial Sequence
  source: /note=“Description of Artificial Sequence Synthetic peptide”
AVIRRPLW

SEQ ID NO: 307 - protein, 10 aa, Artificial Sequence
  source: /note=“Description of Artificial Sequence Synthetic peptide”
DGGMKYTMEN

SEQ ID NO: 308 - protein, 8 aa, Artificial Sequence
  source: /note=“Description of Artificial Sequence Synthetic peptide”
ELVTSRLW

SEQ ID NO: 309 - protein, 10 aa, Artificial Sequence
  source: /note=“Description of Artificial Sequence Synthetic peptide”
DGGVMQLGEN

SEQ ID NO: 310 - protein, 8 aa, Artificial Sequence
  source: /note=“Description of Artificial Sequence Synthetic peptide”
ELGTSRLW

SEQ ID NO: 311 - protein, 10 aa, Artificial Sequence
  source: /note=“Description of Artificial Sequence Synthetic peptide”
DGGVMQLGEN

SEQ ID NO: 312 - protein, 8 aa, Artificial Sequence
  source: /note=“Description of Artificial Sequence Synthetic peptide”
FRGWLRWW

SEQ ID NO: 313 - protein, 10 aa, Artificial Sequence
  source: /note=“Description of Artificial Sequence Synthetic peptide”
DDGARVLAEN

SEQ ID NO: 314 - protein, 8 aa, Artificial Sequence
  source: /note=“Description of Artificial Sequence Synthetic peptide”
GRLKGIGW

SEQ ID NO: 315 - protein, 10 aa, Artificial Sequence
  source: /note=“Description of Artificial Sequence Synthetic peptide”
DGGRPQWGEN

SEQ ID NO: 316 - protein, 8 aa, Artificial Sequence
  source: /note=“Description of Artificial Sequence Synthetic peptide”
GVWQSFPW

SEQ ID NO: 317 - protein, 10 aa, Artificial Sequence
  source: /note=“Description of Artificial Sequence Synthetic peptide”
DGGLGYLREN

SEQ ID NO: 318 - protein, 8 aa, Artificial Sequence
  source: /note=“Description of Artificial Sequence Synthetic peptide”
HLVSLAPW

SEQ ID NO: 319 - protein, 10 aa, Artificial Sequence
  source: /note=“Description of Artificial Sequence Synthetic peptide”
DGGGMHQGKN

SEQ ID NO: 320 - protein, 8 aa, Artificial Sequence
  source: /note=“Description of Artificial Sequence Synthetic peptide”
HIFIDWGW

SEQ ID NO: 321 - protein, 10 aa, Artificial Sequence
  source: /note=“Description of Artificial Sequence Synthetic peptide”
DGGVMTMGEN

SEQ ID NO: 322 - protein, 8 aa, Artificial Sequence
  source: /note=“Description of Artificial Sequence Synthetic peptide”
PVMRGVTW

SEQ ID NO: 323 - protein, 10 aa, Artificial Sequence
  source: /note=“Description of Artificial Sequence Synthetic peptide”
DGGRSWVWEN

SEQ ID NO: 324 - protein, 8 aa, Artificial Sequence
  source: /note=“Description of Artificial Sequence Synthetic peptide”
QLVTVGPW

SEQ ID NO: 325 - protein, 10 aa, Artificial Sequence
  source: /note=“Description of Artificial Sequence Synthetic peptide”
DGGVMHRTEN

SEQ ID NO: 326 - protein, 8 aa, Artificial Sequence
  source: /note=“Description of Artificial Sequence Synthetic peptide”
QLVVQMGW

SEQ ID NO: 327 - protein, 10 aa, Artificial Sequence
  source: /note=“Description of Artificial Sequence Synthetic peptide”
DGGWMTVGEN

SEQ ID NO: 328 - protein, 8 aa, Artificial Sequence
  source: /note=“Description of Artificial Sequence Synthetic peptide”
VAIRRSVW

SEQ ID NO: 329 - protein, 10 aa, Artificial Sequence
  source: /note=“Description of Artificial Sequence Synthetic peptide”
DGGERAHSEN

SEQ ID NO: 330 - protein, 8 aa, Artificial Sequence
  source: /note=“Description of Artificial Sequence Synthetic peptide”
WVMRRPLW

SEQ ID NO: 331 - protein, 10 aa, Artificial Sequence
  source: /note=“Description of Artificial Sequence Synthetic peptide”
DGGSMGWREN

SEQ ID NO: 332 - protein, 8 aa, Artificial Sequence
  source: /note=“Description of Artificial Sequence Synthetic peptide”
WRSMVVWW

SEQ ID NO: 333 - protein, 10 aa, Artificial Sequence
  source: /note=“Description of Artificial Sequence Synthetic peptide”
DGGKHTLGEN

SEQ ID NO: 334 - protein, 8 aa, Artificial Sequence
  source: /note=“Description of Artificial Sequence Synthetic peptide”
ELRTDGLW

SEQ ID NO: 335 - protein, 10 aa, Artificial Sequence
  source: /note=“Description of Artificial Sequence Synthetic peptide”
DGGVMRRSEN

SEQ ID NO: 336 - protein, 245 aa, Artificial Sequence
  source: /note=“Description of Artificial Sequence Synthetic polypeptide”
EVQLVQSGAEVKMPGASVKLSCRVSGDTFTAYFIHWVRQAPGQGLEWMGWFNPISGTAGSAEKFRGRVAMTRDTSISTAYMELNRLTFDDTAVYYCARQHRGNTFDPWGQGTLVTVSSGGGGSGGGGSGGGGSAQSALTQPASVSGSPGQSITISCTGTSSDIGAYKYVSWYQQHPGKAPKLVIYEVSNRPSGVSSRFSGSKSGQTASLTISGLQADDEADYYCNSYQGYNTWVFGGGTKVTVLG

SEQ ID NO: 337 - protein, 111 aa, Artificial Sequence
  source: /note=“Description of Artificial Sequence Synthetic polypeptide”
XXXXXXXXXXXXXXXXXXXXXXXXXXXXXXXXXXXXXXXXXXXXXXXXXLLLLLXXXXXXXXXXXXXXXXXXXXXLLLLLXXXXXXXXXXEEEEEVXXLKEXQALQTVCLX

SEQ ID NO: 338 - protein, 27 aa, Bos sp.
VNALRQRVGILEGQLQRLQNAFSQYKK

SEQ ID NO: 339 - DNA, 16 bp, Artificial Sequence
  source: /note=“Description of Artificial Sequence Synthetic probe”
cctcctcgaactcaaa

SEQ ID NO: 340 - DNA, 23 bp, Artificial Sequence
  source: /note=“Description of Artificial Sequence Synthetic forward primer”
agtgggagaggttctcaatagca

SEQ ID NO: 341 - DNA, 19 bp, Artificial Sequence
  source: /note=“Description of Artificial Sequence Synthetic reverse primer”
gacgtggtcggctgttcct

SEQ ID NO: 342 - DNA, 21 bp, Artificial Sequence
  source: /note=“Description of Artificial Sequence Synthetic forward primer”
ggcctttttgctacagggttt

SEQ ID NO: 343 - DNA, 19 bp, Artificial Sequence
  source: /note=“Description of Artificial Sequence Synthetic reverse primer”
gtgtctccccagccatcct

SEQ ID NO: 344 - DNA, 21 bp, Artificial Sequence
  source: /note=“Description of Artificial Sequence Synthetic forward primer”
aagggcttcacacccaaatct

SEQ ID NO: 345 - DNA, 22 bp, Artificial Sequence
  source: /note=“Description of Artificial Sequence Synthetic reverse primer”
ttctacgtctgcttggctttga

SEQ ID NO: 346 - DNA, 15 bp, Artificial Sequence
  source: /note=“Description of Artificial Sequence Synthetic probe”
caccagcaaccactg

SEQ ID NO: 347 - DNA, 22 bp, Artificial Sequence
  source: /note=“Description of Artificial Sequence Synthetic forward primer”
gggctggttggtgtttgatatc

SEQ ID NO: 348 - DNA, 19 bp, Artificial Sequence
  source: /note=“Description of Artificial Sequence Synthetic reverse primer”
ggttgtgccgagggttgac

SEQ ID NO: 349 - DNA, 20 bp, Artificial Sequence
  source: /note=“Description of Artificial Sequence Synthetic forward primer”
gacggtcctcctggtctttg

SEQ ID NO: 350 - DNA, 17 bp, Artificial Sequence
  source: /note=“Description of Artificial Sequence Synthetic reverse primer”
gtggctggctgcattgc

SEQ ID NO: 351 - DNA, 17 bp, Artificial Sequence
  source: /note=“Description of Artificial Sequence Synthetic probe”
attgccattgcacaact

SEQ ID NO: 352 - DNA, 22 bp, Artificial Sequence
  source: /note=“Description of Artificial Sequence Synthetic forward primer”
gggaaatcgtggaaatgagaaa

SEQ ID NO: 353 - DNA, 24 bp, Artificial Sequence
  source: /note=“Description of Artificial Sequence Synthetic reverse primer”
aagtgcatcatcgttgttcataca

SEQ ID NO: 354 - DNA, 17 bp, Artificial Sequence
  source: /note=“Description of Artificial Sequence Synthetic probe”
aatgggtccagacatac

SEQ ID NO: 355 - DNA, 21 bp, Artificial Sequence
  source: /note=“Description of Artificial Sequence Synthetic forward primer”
gtctgtgctgaccccaagaag

SEQ ID NO: 356 - DNA, 21 bp, Artificial Sequence
  source: /note=“Description of Artificial Sequence Synthetic reverse primer”
tggttccgatccaggttttta

SEQ ID NO: 357 - DNA, 17 bp, Artificial Sequence
  source: /note=“Description of Artificial Sequence Synthetic probe”
cgaacctcaccacagag

SEQ ID NO: 358 - DNA, 16 bp, Artificial Sequence
  source: /note=“Description of Artificial Sequence Synthetic forward primer”
gcgtgcagcggcatct

SEQ ID NO: 359 - DNA, 23 bp, Artificial Sequence
  source: /note=“Description of Artificial Sequence Synthetic reverse primer”
tcaatctcttctgggctgatctt

SEQ ID NO: 360 - DNA, 17 bp, Artificial Sequence
  source: /note=“Description of Artificial Sequence Synthetic probe”
tgccagtactgccgttt

SEQ ID NO: 361 - DNA, 23 bp, Artificial Sequence
  source: /note=“Description of Artificial Sequence Synthetic forward primer”
gattcagaagaagaaccggaaca

SEQ ID NO: 362 - DNA, 20 bp, Artificial Sequence
  source: /note=“Description of Artificial Sequence Synthetic reverse primer”
ccgacagacaggcacttgtg

SEQ ID NO: 363 - DNA, 15 bp, Artificial Sequence
  source: /note=“Description of Artificial Sequence Synthetic probe”
ctctgtggacctctc

SEQ ID NO: 364 - DNA, 19 bp, Artificial Sequence
  source: /note=“Description of Artificial Sequence Synthetic forward primer”
gcccaccaacttcggaatc

SEQ ID NO: 365 - DNA, 20 bp, Artificial Sequence
  source: /note=“Description of Artificial Sequence Synthetic reverse primer”
tgcgagtggtcttccatcac

SEQ ID NO: 366 - DNA, 16 bp, Artificial Sequence
  source: /note=“Description of Artificial Sequence Synthetic probe”
tcacccacaccgtcag

SEQ ID NO: 367 - DNA, 17 bp, Artificial Sequence
  source: /note=“Description of Artificial Sequence Synthetic forward primer”
ggctgccccgactacgt

SEQ ID NO: 368 - DNA, 26 bp, Artificial Sequence
  source: /note=“Description of Artificial Sequence Synthetic reverse primer”
tttctcctggtatgagatagcaaatc

SEQ ID NO: 369 - protein, 166 aa, Artificial Sequence
  source: /note=“Description of Artificial Sequence Synthetic polypeptide”
AHXTGXXXXXXXXXXXXXXXXXKXXGXKIXXWXXXRXGHSFXXXXXXRNGELVIXXXGXYYIYXQXYXRFXEXXXXXXXXXXXXXXXXXXXXXXQXXQYIYKXTXYPXPIXLMKSARNXCWSXXXEYGLYSIYQGGXFELKXXDRIFVSVXNXXLXDXXXEXSFXG

SEQ ID NO: 370 - protein, 14 aa, Artificial Sequence
  source: /note=“Description of Artificial Sequence Synthetic peptide”
XXXXXXXXXXXXVA

SEQ ID NO: 371 - protein, 6 aa, Artificial Sequence
  source: /note=“Description of Artificial Sequence Synthetic peptide”
AFXXXX